(19) United States
(12) Patent Application Publication
(10) Pub. No.: US 2024/0110206 A1
(43) Pub. Date: Apr. 4, 2024

(54) BIOSYNTHESIS OF CANNABINOIDS AND CANNABINOID PRECURSORS

(71) Applicant: Ginkgo Bioworks, Inc., Boston, MA (US)

(72) Inventors: Dylan Alexander Carlin, Jamaica Plain, MA (US); Brian Carvalho, Somerville, MA (US); Nicholas Flores, Cambridge, MA (US); Katrina Forrest, Seattle, WA (US); Gabriel Rodriguez, Central, SC (US); Nathan W. Schmidt, Milton, MA (US); Michelle Spencer, Boston, MA (US)

(73) Assignee: Ginkgo Bioworks, Inc., Boston, MA (US)

(21) Appl. No.: 18/031,522

(22) PCT Filed: Oct. 12, 2021
(86) PCT No.: PCT/US2021/054641

§ 371 Date: Apr. 12, 2023

Related U.S. Application Data
(60) Provisional application No. 63/188,442, filed on May 13, 2021.
(60) Provisional application No. 63/091,292, filed on Oct. 13, 2020.

(51) Int. Cl. C12P 7/42; C12N 9/10; C12N 15/62; C12N 15/81

(52) U.S. Cl. CPC C12P 7/42; C12N 9/1029; C12N 9/1085; C12N 15/62; C12N 15/81; C12Y 203/01206; C12Y 205/0101

(57) ABSTRACT

Aspects of the disclosure relate to biosynthesis of cannabinoids and cannabinoid precursors in recombinant cells and in vitro. Specifically, the disclosure is directed to a prenyltransferase variant, a chimeric prenyltransferase comprising one or more portions of at least two different prenyltransferase proteins, and a fusion polypeptide comprising a CBG-type producing prenyltransferase and farnesyl pyrophosphate synthase.

### CROSS REFERENCE TO RELATED APPLICATIONS

This application claims the benefit under 35 U.S.C. § 119(e) of U.S. Provisional Application No. 63/091,292, filed Oct. 13, 2020, entitled “BIOSYNTHESIS OF CANNABINOIDS AND CANNABINOID PRECURSORS” and U.S. Provisional Application No. 63/188,442, filed May 13, 2021, entitled “BIOSYNTHESIS OF CANNABINOIDS AND CANNABINOID PRECURSORS,” the entire disclosures of each of which are hereby incorporated by reference in their entireties.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Oct. 8, 2021, is named G091970063WO00-SEQ-OMJ, and is 3,122,581 bytes in size.

### FIELD OF INVENTION

The present disclosure relates to the biosynthesis of cannabinoids and cannabinoid precursors, such as in recombinant cells.

### BACKGROUND

Cannabinoids are chemical compounds that may act as ligands for endocannabinoid receptors and have multiple medical applications. Traditionally, cannabinoids have been isolated from plants of the genus Cannabis. The use of plants for producing cannabinoids is inefficient, however, with isolated products often limited to the two most prevalent endogenous cannabinoids. THC and CBD, as other cannabinoids are typically produced in very low concentrations in Cannabis plants. Further, the cultivation of Cannabis plants is restricted in many jurisdictions. In addition, in order to obtain consistent results, Cannabis plants are often grown in a controlled environment, such as indoor grow rooms without windows, to provide flexibility in modulating growing conditions such as lighting, temperature, humidity, airflow, etc. Growing Cannabis plants in such controlled environments can result in high energy usage per gram of cannabinoid produced, especially for rare cannabinoids that the plants produce only in small amounts. For example, lighting in such grow rooms is provided by artificial sources, such as high-powered sodium lights. As many species of Cannabis have a vegetative cycle that requires 18 or more hours of light per day, powering such lights can result in significant energy expenditures. It has been estimated that between 0.88-1.34 kWh of energy is required to produce one gram of THC in dried Cannabis flower form (e.g., before any extraction or purification). Additionally, concern has been raised over agricultural practices in certain jurisdictions, such as California, where the growing season coincides with the dry season such that the water usage may impact connected surface water in streams (Dillis, Christopher, Connor McIntee, Van Butsic, Lance Le, Kason Grady, and Theodore Grantham. “Water storage and irrigation practices for Cannabis drive seasonal patterns of water extraction and use in Northern California.” Journal of Environmental Management 272 (2020): 110955).

Cannabinoids can also be produced through chemical synthesis (see, e.g., U.S. Pat. No. 7,323,576 to Souza et al). However, such methods suffer from low yields and high cost.

Production of cannabinoids, cannabinoid analogs, and cannabinoid precursors using engineered organisms may provide an advantageous approach to meet the increasing demand for these compounds.

### SUMMARY

Aspects of the present disclosure provide methods for production of cannabinoids and cannabinoid precursors from fatty acid substrates using genetically modified host cells.

Aspects of the disclosure relate to chimeric prenyltransferases (PTs), wherein the chimeric PT comprises one or more portions of at least two different PTs and wherein the chimeric PT is capable of producing a CBG-type cannabinoid from a resorcylic acid. In some embodiments, the CBG-type cannabinoid and the resorcylic acid are: cannabigerolic acid (CBGA) and olivetolic acid; or cannabigerovarinic acid (CBGVA) and divaric acid (DA).

In some embodiments, the chimeric PT comprises one or more portions of CsPT1. In some embodiments, the chimeric PT comprises one or more portions of CsPT4. In some embodiments, the chimeric PT comprises one or more portions of CsPT6. In some embodiments, the chimeric PT comprises one or more portions of CsPT7.

In some embodiments, the chimeric PT comprises multiple transmembrane helices, and at least one transmembrane helix of the multiple transmembrane helices comprises one or more portions of at least two different CsPTs. In some embodiments, at least one transmembrane helix of the multiple transmembrane helices comprises both a portion of CsPT4 and a portion of CsPT1, CsPT6 or CsPT7. In some embodiments, all the transmembrane helices comprise both a portion of CsPT4 and a portion of CsPT1, CsPT6 or CsPT7.

In some embodiments, the chimeric PT comprises one or more of the following motifs: MTVMGMT (SEQ ID NO: 11); [EV][LMW][RS]P[SAP]F[ST]F[IL][IL]AF (SEQ ID NO: 12); QFFEFIW (SEQ ID NO: 13), HNTNL (SEQ ID NO: 14); TCWKL (SEQ ID NO: 15); M[IL]LSHAILAFC (SEQ ID NO: 16); HVG[LV][AN]FT[SCF]Y[YS]A[ST][RT][AS]A[LF] (SEQ ID NO: 17); GLIVT (SEQ ID NO: 18); L[YH]YAEY[LF]V (SEQ ID NO: 19); KAFFAL (SEQ ID NO: 20); KLGARNMT (SEQ ID NO: 21); QAF[NK]SN (SEQ ID NO: 22); LIFQT (SEQ ID NO: 23), SIIVALT (SEQ ID NO: 24); MSIETAW (SEQ ID NO: 25); VVSGV (SEQ ID NO: 26); RPYVV (SEQ ID NO: 27); KPDLP (SEQ ID NO: 28); RWKQY (SEQ ID NO: 29); FLITI (SEQ ID NO: 30); DIEGD (SEQ ID NO: 31); and KYGVST (SEQ ID NO: 32).

In some embodiments, the chimeric PT comprises the structure: X1-X2-X3-X4-X5-X6-X7-X8-X9-X10, wherein at least one of X1, X2, X3, X4, X5, X6, X7, X8, X9 or X10 comprises a portion of CsPT4. In some embodiments, at least one of X1, X3, X5, X7, and X9 comprises a portion of CsPT4. In some embodiments, all of X1, X3, X5, X7, and X9 comprise portions of CsPT4. In some embodiments, at least one of X2, X4, X6, X8, and X10 comprises a portion of CsPT1, CsPT6, or CsPT7. In some embodiments, all of X2, X4, X6, X8, and X10 comprise portions of CsPT1, CsPT6 or CsPT7.

In some embodiments, the chimeric PT comprises the structure: X1-X2-X3-X4-X5-X6-X7-X8-X9-X10, and: the sequence of X1 comprises any of SEQ ID NOs: 33-39 or a sequence that comprises no more than 2 amino acid substitutions, insertions, additions or deletions relative to any one of SEQ ID NOs: 33-39; the sequence of X2 comprises any of SEQ ID NOs: 40-46 or a sequence that comprises no more than 2 amino acid substitutions, insertions, additions or deletions relative to any one of SEQ ID NOs: 40-46; the sequence of X3 comprises any of SEQ ID NOs: 47-53 or a sequence that comprises no more than 2 amino acid substitutions, insertions, additions or deletions relative to any one of SEQ ID NOs: 47-53; the sequence of X4 comprises any of SEQ ID NOs: 54-60 or a sequence that comprises no more than 2 amino acid substitutions, insertions, additions or deletions relative to any one of SEQ ID NOs: 54-60; the sequence of X5 comprises any of SEQ ID NOs: 61-67 or a sequence that comprises no more than 2 amino acid substitutions, insertions, additions or deletions relative to any one of SEQ ID NOs: 61-67; the sequence of X6 comprises any of SEQ ID NOs: 68-74 or a sequence that comprises no more than 2 amino acid substitutions, insertions, additions or deletions relative to any one of SEQ ID NOs: 68-74; the sequence of X7 comprises any of SEQ ID NOs: 75-81 or a sequence that comprises no more than 2 amino acid substitutions, insertions, additions or deletions relative to any one of SEQ ID NOs: 75-81; the sequence of X8 comprises any of SEQ ID NOs: 82-88 or a sequence that comprises no more than 2 amino acid substitutions, insertions, additions or deletions relative to any one of SEQ ID NOs: 82-88; the sequence of X9 comprises any of SEQ ID NOs: 89-95 or a sequence that comprises no more than 2 amino acid substitutions, insertions, additions or deletions relative to any one of SEQ ID NOs: 89-95; and/or the sequence of X10 comprises any of SEQ ID NOs: 96-102 or a sequence that comprises no more than 2 amino acid substitutions, insertions, additions or deletions relative to any one of SEQ ID NOs: 96-102.

In some embodiments, the chimeric PT comprises a sequence that is at least 90% identical to any one of SEQ ID NOs: 113-121, 757-868, and 982-1081. In some embodiments, the chimeric PT comprises any one of SEQ ID NOs: 113-118, 757-868, and 982-1081.

In some embodiments, the chimeric PT comprises an amino acid substitution relative to SEQ ID NO: 5 at one or more of the following positions within SEQ ID NO: 5: C31, M43, M75, I46, F82, F83, I86, M87, D94, E113, F145, I147, F151, Q162, A227, S232, F245, Q267, Q288, and L311. In some embodiments, the chimeric PT comprises one or more of the following amino acid substitutions relative to SEQ ID NO: 5: C31F, M43V, M43L, I46C, M75V, F82G, F83Y, I86S, I86A, I86G, I86V, I86S, M87V, M87I, D94E, E113R, I140L, F145T, F145L, F145S, I147L, F151T, A227K. S232R, F245R, F245W, T254N, Q267F, Q288R, L331N, and L311R. In some embodiments, the chimeric PT is capable of producing more CBGA from olivetolic acid or more CBGVA from divaric acid than a chimeric PT that comprises SEQ ID NO:324.

Further aspects of the disclosure relate to polynucleotides encoding any of the chimeric PTs of the disclosure. In some embodiments, the polynucleotide comprises a sequence that is at least 90% identical to any one of SEQ ID NOs: 136-144, 869-980, and 1083-1182. In some embodiments, the polynucleotide comprises the sequence of any one of SEQ ID NOs: 136-144, 869-980, and 1083-1182.

Further aspects of the disclosure relate to fusion proteins comprising chimeric PTs of the disclosure wherein the fusion protein further comprises a farnesyl pyrophosphate synthase. In some embodiments, the farnesyl pyrophosphate synthase comprises a mutation that increases the production of geranylpyrophosphate relative to farnesylpyrophosphate. In some embodiments, the farnesyl pyrophosphate synthase sequence comprises a tryptophan residue at a residue corresponding to residues 96, 127, or both 96 and 127, in wild-type ERG20 (SEQ ID NO: 424).

In some embodiments, the farnesyl pyrophosphate synthase is amino terminal to the chimeric prenyltransferase within the fusion protein. In some embodiments, the farnesyl pyrophosphate synthase and the chimeric prenyltransferase are separated by a linker sequence. In some embodiments, the linker comprises any one of SEQ ID NOs: 104-109, or a sequence that comprises no more than 2 amino acid substitutions, insertions, additions or deletions relative to any one of SEQ ID NOs: 104-109.

In some embodiments, the sequence of the farnesyl pyrophosphate synthase comprises one or more of the following motifs: NVPGGKLNR (SEQ ID NO: 647); FYLPVALA[LM]H (SEQ ID NO: 648); A[EH]D[IV]LIPLG (SEQ ID NO: 651); LGW[CL][ITV]ELLQA[FY]FL (SEQ ID NO: 655); KKEV[FL][ET][SA]FL[AGN]KIYK (SEQ ID NO: 663), QRK[VI]L[DE]ENYG (SEQ ID NO: 667); VGMIAIWD (SEQ ID NO: 672); TDI[QK]DNKCSW (SEQ ID NO: 673); TAYYSFYLP (SEQ ID NO: 676); GKIGTDI[QK]DNKCSW (SEQ ID NO: 677); ILIP[LM]GEYFQ (SEQ ID NO: 680); IL[VM][EP][ML]G[ET][YF]FQ (SEQ ID NO: 683); AKIYKRSK (SEQ ID NO: 685); DPEVIGKI (SEQ ID NO: 686); RGQPCW[YF]RVP[EQ] (SEQ ID NO: 687); IVKYKTA[YF]Y[ST]FYLP (SEQ ID NO: 689); WC[IV]E[LW]LQA[YF][WF]LV[ALW]D (SEQ ID NO: 692); CSWLV[VN]Q[AC]L[AQ][RI][AC][ST]P[ED]Q (SEQ ID NO: 699).

In some embodiments, the farnesyl pyrophosphate synthase comprises a sequence that is at least 90% identical to any one of SEQ ID NOs: 103, 426-476, or 753. In some embodiments, the farnesyl pyrophosphate synthase comprises any one of SEQ ID NOs: 426-476 or 753.

In some embodiments, the fusion protein comprises a sequence that is at least 90% identical to any one of SEQ ID NOs: 532-582 or 755. In some embodiments, the fusion protein comprises any one of SEQ ID NOs: 532-582 or 755.

Further aspects of the disclosure relate to host cells comprising any of the chimeric PTs or fusion proteins associated with the disclosure. In some embodiments, the host cell comprises one or more copies of a heterologous farnesyl pyrophosphate synthase. In some embodiments, one or more copies of the farnesyl pyrophosphate synthase are integrated into the genome of the host cell. In some embodiments, the host cell is a plant cell, an algal cell, a yeast cell, a bacterial cell, or an animal cell. In some embodiments, the host cell is a yeast cell. In some embodiments, the yeast cell is a Saccharomyces cell, a Yarrowia cell, a Komagataella cell, or a Pichia cell. In some embodiments, the Saccharomyces cell is a Saccharomyces cerevisiae cell. In some embodiments, the host cell is a bacterial cell. In some embodiments, the bacterial cell is an E. coli cell.

In some embodiments, the host cell further comprises one or more heterologous polynucleotides encoding one or more of: an acyl activating enzyme (AAE), a polyketide synthase (PKS), a polyketide cyclase (PKC), and/or a terminal synthase (TS). In some embodiments, the PKS is an olivetol synthase (OLS).

Further aspects of the disclosure relate to methods comprising culturing any of the host cells associated with the disclosure.

Further aspects of the disclosure relate to host cells that comprises a heterologous polynucleotide encoding a farnesyl pyrophosphate synthase wherein the sequence of the farnesyl pyrophosphate synthase comprises one or more of the following motifs: NVPGGKLNR (SEQ ID NO: 647); FYLPVALA[LM]H (SEQ ID NO: 648); A[EH]D[IV]LIPLG (SEQ ID NO: 651); LGW[CL][ITV]ELLQA[FY]FL (SEQ ID NO: 655); KKEV[FL][ET][SA]FL[AGN]KIYK (SEQ ID NO: 663); QRK[VI]L[DE]ENYG (SEQ ID NO: 667); VGMIAIWD (SEQ ID NO: 672); TDI[QK]DNKCSW (SEQ ID NO: 673); TAYYSFYLP (SEQ ID NO: 676); GKIGTDI[QK]DNKCSW (SEQ ID NO: 677); ILIP[LM]GEYFQ (SEQ ID NO: 680); IL[VM][EP][ML]G[ET][YF]FQ (SEQ ID NO: 683); AKIYKRSK (SEQ ID NO: 685); DPEVIGKI (SEQ ID NO: 686); RGQPCW[YF]RVP[EQ](SEQ ID NO: 687); IVKYKTA[YF]Y[ST]FYLP (SEQ ID NO: 689); WC[IV]E[LW]LQA[YF][WF]LV[ALW]D (SEQ ID NO: 692); CSWLV[VN]Q[AC]L[AQ][RI][AC][ST]P[ED]Q (SEQ ID NO: 699); wherein the farnesyl pyrophosphate synthase does not comprise SEQ ID NO: 103 or SEQ ID NO: 424.

In some embodiments, the farnesyl pyrophosphate synthase comprises a sequence that is at least 90% identical to any one of SEQ ID NOs: 426-476 or 753. In some embodiments, the farnesyl pyrophosphate synthase comprises any one of SEQ ID NOs: 426-476 or 753.

Further aspects of the disclosure relate to polynucleotides encoding a chimeric PT, wherein the polynucleotide comprises a sequence that is at least 90% identical to any one of SEQ ID NOs: 136-144, 869-980, and 1083-1182.

Further aspects of the disclosure relate to non-naturally occurring polynucleotides encoding a farnesyl pyrophosphate synthase, wherein the non-naturally occurring polynucleotide comprises a sequence that is at least 90% identical to any one of SEQ ID NOs: 479-529 or 754.

Further aspects of the disclosure relate to polynucleotides encoding a fusion protein, wherein the polynucleotide comprises a sequence that is at least 90% identical to any one of SEQ ID NOs: 585-635, 728-752 or 756.

Further aspects of the disclosure relate to vectors comprising any of the polynucleotides associated with the disclosure. Further aspects of the disclosure relate to expression cassettes comprising any of the polynucleotides associated with the disclosure. Further aspects of the disclosure relate to host cells transformed with any of the polynucleotides associated with the disclosure, any of the vectors associated with the disclosure, or any of the expression cassettes associated with the disclosure.

Further aspects of the disclosure relate to variant PTs or active fragments thereof comprising a non-naturally occurring amino acid sequence relative to a wild-type PT, wherein the variant PT or active fragment thereof acts on a substrate to produce an altered amount of a cannabinoid relative to the amount of the cannabinoid produced by the wild-type PT. In some embodiments, the variant PT or active fragment thereof comprises an amino acid substitution relative to a prenyltransferase of SEQ ID NO: 5. In some embodiments, the variant PT or active fragment thereof comprises an amino acid substitution relative to SEQ ID NO: 5 at one or more of the following positions within SEQ ID NO: 5: C31, M43, I46, F82, F83, I86, M87, D94, E113, S119, V122, F145, I147, F151, Q162, S232, F245, Q267, Q288, and L311. In some embodiments, the PT comprises one or more of the following amino acid substitutions relative to SEQ ID NO: 5: C31F, M43V, M43L, I46C, F82G, F83Y, I86S, I86A, I86G, I86V, I86S, M87V, M87I, D94E, E113R, F145T, F145L, F145S, I147L, F151T, S232R, F245R, F245W, Q267F, Q288R, L331N, and L311R.

In some embodiments, the variant PT or active fragment thereof produces an increased amount of CBGA relative to the amount of CBGA produced by the wild-type PT. In some embodiments, the variant PT or active fragment thereof produces an increased amount of CBGVA relative to the amount of CBGVA produced by the wild-type PT.

Further aspects of the disclosure relate to polynucleotides encoding variant PTs or active fragments thereof. Further aspects of the disclosure relate to vectors comprising variant PTs or active fragments thereof. Further aspects of the disclosure relate to expression cassettes comprising variant PTs or active fragments thereof. Further aspects of the disclosure relate to host cells transformed with polynucleotides, vectors, or expression cassettes comprising variant PTs or active fragments thereof.

Further aspects of the disclosure relate to methods of producing a cannabinoid comprising reacting:
a) a CBG-type compound, and
b) a prenyl pyrophosphate, in the presence of: a chimeric PT associated with the disclosure, a PT encoded by a polynucleotide associated with the disclosure, a fusion protein associated with the disclosure, or a variant PT associated with the disclosure.

In some embodiments, the compound of Formula (6) is CBGA or CBGVA. In some embodiments, the prenyl pyrophosphate is geranyl pyrophosphate.

Further aspects of the disclosure relate to bioreactors for producing a cannabinoid compound. In some embodiments, the bioreactors comprise a chimeric PT associated with the disclosure, a PT encoded by a polynucleotide associated with the disclosure, a fusion protein associated with the disclosure, a variant PT associated with the disclosure, and/or a host cell associated with the disclosure.

Each of the limitations of the invention can encompass various embodiments of the invention. It is, therefore, anticipated that each of the limitations of the invention involving any one element or combinations of elements can be included in each aspect of the invention. This disclosure is not limited in its application to the details of construction and the arrangement of components set forth in the following description or illustrated in the drawings. The invention is capable of other embodiments and of being practiced or of being carried out in various ways. Also, the phraseology and terminology used in this application is for the purpose of description and should not be regarded as limiting. The use of “including,” “comprising,” or “having,” “containing,” “involving,” and variations thereof, is meant to encompass the items listed thereafter and equivalents thereof as well as additional items.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a schematic depicting the native Cannabis biosynthetic pathway for production of cannabinoid compounds, including five enzymatic steps mediated by: (R1a) acyl activating enzymes (AAE); (R2a) olivetol synthase enzymes (OLS); (R3a) olivetolic acid cyclase enzymes (OAC); (R4a) prenyltransferase enzymes (PT); and (R5a) terminal synthase enzymes (TS). Formulae 1a-11a correspond to hexanoic acid (1a), hexanoyl-CoA (2a), malonyl-CoA (3a), 3,5,7-trioxododecanoyl-CoA (4a), olivetol (5a), olivetolic acid (6a), geranyl pyrophosphate (7a), cannabigerolic acid (8a), cannabidiolic acid (9a), tetrahydrocannabinolic acid (10a), and cannabichromenic acid (11a). Hexanoic acid is an exemplary carboxylic acid substrate; other carboxylic acids may also be used (e.g., butyric acid, isovaleric acid, octanoic acid, decanoic acid, etc.; see e.g., FIG. 3 below). The enzymes that catalyze the synthesis of 3,5,7-trioxododecanoyl-CoA and olivetolic acid are shown in R2a and R3a, respectively, and can include multi-functional enzymes that catalyze the synthesis of 3,5,7-trioxododecanoyl-CoA and olivetolic acid. The enzymes cannabidiolic acid synthase (CBDAS), tetrahydrocannabinolic acid synthase (THCAS), and cannabichromenic acid synthase (CBCAS) that catalyze the synthesis of cannabidiolic acid, tetrahydrocannabinolic acid, and cannabichromenic acid, respectively, are shown in step R5a. FIG. 1 is adapted from Carvalho et al. “Designing Microorganisms for Heterologous Biosynthesis of Cannabinoids” (2017) FEMS Yeast Research June 1:17(4), which is incorporated by reference in its entirety.

FIG. 2 is a schematic depicting a heterologous biosynthetic pathway for production of cannabinoid compounds, including five enzymatic steps mediated by: (RI) acyl activating enzymes (AAE); (R2) polyketide synthase enzymes (PKS) or bifunctional polyketide synthase-polyketide cyclase enzymes (PKS-PKC); (R3) polyketide cyclase enzymes (PKC) or bifunctional PKS-PKC enzymes; (R4) prenyltransferase enzymes (PT); and (R5) terminal synthase enzymes (TS). Any carboxylic acid of varying chain lengths, structures (e.g., aliphatic, alicyclic, or aromatic) and functionalization (e.g., hydroxylic-, keto-, amino-, thiol-, aryl-, or alogeno-) may also be used as precursor substrates (e.g., thiopropionic acid, hydroxy phenyl acetic acid, norleucine, bromodecanoic acid, butyric acid, isovaleric acid, octanoic acid, decanoic acid, etc).

FIG. 3 is a non-exclusive representation of select putative precursors for the cannabinoid pathway in FIG. 2.

FIG. 4 is a schematic showing a reaction catalyzed by a PT enzyme wherein Olivetolic Acid (OA, Formula (6a)) and Geranyl Pyrophosphate (GPP, Formula (7a)) are condensed to form either the major cannabinoid Cannabigerolic Acid (CBGA, Formula (8a)) or 2-O-Geranyl Olivetolic Acid (OGOA, Formula (8b)).

FIGS. 5A-5B depict 3-D structural models showing regions that were targeted for mutagenesis in a representative C. sativa PT (CsPT) protein. FIG. 5A depicts an approach whereby point mutations were generated at locations (depicted in black) spread throughout the whole sequence of a CsPT protein based on bioinformatics analysis. FIG. 5B depicts an approach whereby point mutations were focused within regions (depicted in black) near the active site of a representative CsPT protein. The active site is located around the pair of Mg2+ ions (depicted as spheres) and GPP substrate (depicted as sticks).

FIG. 6 depicts the crystal structure of the PT AfUbiA from A. fulgidus (corresponding to PDB ID 4TQ3; UniProt Accession No. 028625).

FIGS. 7A-7B depict approaches used to generate chimeras involving CsPT enzymes. FIG. 7A depicts an example of a “within membrane” approach for generating chimeras in which the cross-over points between different CsPT proteins occur within the membrane. FIG. 7B depicts an example of a “through membrane” approach for generating chimeras in which there is a single cross-over point between two helices. In the example shown in FIG. 7B, the cross-over point is between helices 6&7 of the CsPT protein.

FIG. 8 is a schematic showing a plasmid bearing the transcriptional unit encoding each PT. The coding sequence for the PT enzymes (labeled “Library gene”) was driven by the GAL1 promoter. The plasmid contains markers for both yeast (URA3) and bacteria (ampR), as well as origins of replication for yeast (2 micron), and bacteria (pBR322).

FIGS. 9A-9B depict graphs showing secondary screening activity data of PT enzymes, including point mutations, chimeric PTs, and PT fusion proteins based on an in vivo activity assay in S. cerevisiae described in Example 2. FIG. 9A depicts results for CBGA production and FIG. 9B depicts results for CBGVA production. Strain t444508, expressing a truncated CsPT4 protein (SEQ ID NO: 5), was used as a positive control and for determining hit ranking of the library members. Strain t444525, expressing GFP, was used as a negative control. The data represent the average of four bioreplicates±one standard deviation of the mean. Strain IDs and their corresponding activity from these graphs are shown in Table 5.

FIGS. 10A-10B depict graphs showing secondary screening activity data of PT fusion proteins based on an in vivo activity assay in S. cerevisiae described in Example 2. FIG. 10A depicts results for CBGA production, and FIG. 10B depicts results for CBGVA production. The data represent the average of four bioreplicates±one standard deviation of the mean. Strain IDs and their corresponding activity from these graphs are shown in Table 5.

FIGS. 11A-11B depict graphs showing secondary screening activity data of chimeric PTs based on an in vivo activity assay in S. cerevisiae described in Example 2. FIG. 11A depicts results for CBGA production, and FIG. 11B depicts results for CBGVA production. Strain IDs and their corresponding activity from these graphs are shown in Table 5.

FIGS. 12A-12B depict graphs showing activity data from a second-generation library of chimeric PTs and chimeric fusion proteins (Gen 2 library) based on an in vivo activity assay in S. cerevisiae described in Examples 3-4. Strain t612212, expressing a truncated CsPT4 protein (SEQ ID NO: 5), was used as a positive control and for determining hit ranking of the library members. FIG. 12A depicts results for CBGA production in the presence of 1 mM olivetolic acid (OA), and FIG. 12B depicts results for CBGVA production in the presence of 1 mM divaric acid (DA). Strain IDs and their corresponding activity from these graphs are shown in Table 7.

FIG. 13 depicts a graph showing activity data from a third-generation library of chimeric fusion proteins (Gen3 PT library) for CBGA production based on an in vivo activity assay in S. cerevisiae described in Example 5. Strain t704346, which comprises an ERG20ww-CsPT chimera identified in Example 4, was used as a benchmark for determining hit ranking of the library members. Strain IDs and their corresponding activity from this graph are shown in Table 8.

FIG. 14 depicts a graph showing library screening activity data of chimeric fusions including ERG20 homologs based on an in vivo activity assay for CBGA production in S. cerevisiae described in Example 6. Strains t756346 and t56349 were used as positive controls. Strain IDs and their corresponding activity from this graph are shown in Table 9.

FIGS. 15A-15B depict graphs showing activity data from a fourth-generation library of chimeric PTs (Gen 4 library) based on an in vivo activity assay in S. cerevisiae described in Example 7. The Gen4 library contained chimeric PTs from strains t523834 (SEQ ID NO: 114, corresponding to a CsPT1-CsPT4 chimera) and t524816 (SEQ ID NO: 116, corresponding to a CsPT4-CsPT7 chimera), described in Examples 1 and 2, which were modified to include point mutations characterized in Example 1. FIG. 15A depicts results for CBGA production and FIG. 15B depicts results for CBGVA production. Strain t827885, expressing a chimeric PT corresponding to SEQ ID NO: 324, was used as a positive control and for determining hit ranking of the library members. Strain t819232, expressing RFP, was used as a negative control. The data represent the average of four bioreplicates±one standard deviation of the mean. Strain IDs and their corresponding activity from these graphs are shown in Table 11.

FIG. 16 depicts a graph showing activity data from a fifth-generation library of chimeric PTs (Gen 5 library) based on an in vivo activity assay in S. cerevisiae described in Example 8. The Gen 5 library contained chimeric PTs from the Gen 4 library described in Example 7 that were modified to include additional point mutations. Strain t819140, expressing RFP, was used as a negative control. Strains t818980 and t819132 were used as positive controls. Strain IDs and their corresponding activity from this graph are shown in Table 12.

### DETAILED DESCRIPTION

This disclosure provides methods for production of cannabinoids and cannabinoid precursors from fatty acid substrates using genetically modified host cells. Methods include heterologous expression of a prenyltransferase (PT). The application describes the identification of multiple PTs that can be functionally expressed in host cells such as S. cerevisiae cells. As demonstrated in Examples 1-8, synthetic chimeric PTs were generated that contain portions of different C. sativa PT proteins. Surprisingly, chimeric PTs, and fusion proteins including chimeric PTs, were identified that were capable of producing more cannabigerolic acid (CBGA) and/or cannabigerovarinic acid (CBGVA) than CsPT4.

### Definitions

While the following terms are believed to be well understood by one of ordinary skill in the art, the following definitions are set forth to facilitate explanation of the disclosed subject matter.

The term “a” or “an” refers to one or more of an entity, i.e., can identify a referent as plural. Thus, the terms “a” or “an,” “one or more” and “at least one” are used interchangeably in this application. In addition, reference to “an element” by the indefinite article “a” or “an” does not exclude the possibility that more than one of the elements is present, unless the context clearly requires that there is one and only one of the elements.

The terms “microorganism” or “microbe” should be taken broadly. These terms are used interchangeably and include, but are not limited to, the two prokaryotic domains, Bacteria and Archaea, as well as certain eukaryotic fungi and protists. In some embodiments, the disclosure may refer to the “microorganisms” or “microbes” of lists/tables and figures present in the disclosure. This characterization can refer to not only the identified taxonomic genera of the tables and figures, but also the identified taxonomic species, as well as the various novel and newly identified or designed strains of any organism in the tables or figures. The same characterization holds true for the recitation of these terms in other parts of the specification, such as in the Examples.

The term “prokaryotes” is recognized in the art and refers to cells that contain no nucleus or other cell organelles. The prokaryotes are generally classified in one of two domains, the Bacteria and the Archaea.

“Bacteria” or “eubacteria” refers to a domain of prokaryotic organisms. Bacteria include at least 11 distinct groups as follows: (1) Gram-positive (gram+) bacteria, of which there are two major subdivisions: (a) high G+C group (Actinomycetes, Mycobacteria, Micrococcus, others) and (b) low G+C group (Bacillus, Clostridia, Lactobacillus, Staphylococci, Streptococci, Mycoplasmas); (2) Proteobacteria, e.g., Purple photosynthetic+non-photosynthetic Gram-negative bacteria (includes most “common” Gram-negative bacteria); (3) Cyanobacteria, e.g., oxygenic phototrophs; (4) Spirochetes and related species; (5) Planctomyces; (6) Bacteroides, Flavobacteria; (7) Chlamydia; (8) Green sulfur bacteria; (9) Green non-sulfur bacteria (also anaerobic phototrophs); (10) Radioresistant micrococci and relatives; and (11) Thermotoga and Thermosipho thermophiles.

The term “Archaea” refers to a taxonomic classification of prokaryotic organisms with certain properties that make them distinct from Bacteria in physiology and phylogeny.

The term “Cannabis” refers to a genus in the family Cannabaceae. Cannabis is a dioecious plant. Glandular structures located on female flowers of Cannabis, called trichomes, accumulate relatively high amounts of a class of terpeno-phenolic compounds known as phytocannabinoids (described in further detail below). Cannabis has conventionally been cultivated for production of fibre and seed (commonly referred to as “hemp-type”), or for production of intoxicants (commonly referred to as “drug-type”). In drug-type Cannabis, the trichomes contain relatively high amounts of tetrahydrocannabinolic acid (THCA), which can convert to tetrahydrocannabinol (THC) via a decarboxylation reaction, for example upon combustion of dried Cannabis flowers, to provide an intoxicating effect. Drug-type Cannabis often contains other cannabinoids in lesser amounts. In contrast, hemp-type Cannabis contains relatively low concentrations of THCA, often less than 0.3% THC by dry weight. Hemp-type Cannabis may contain non-THC and non-THCA cannabinoids, such as cannabidiolic acid (CBDA), cannabidiol (CBD), and other cannabinoids. Presently, there is a lack of consensus regarding the taxonomic organization of the species within the genus. Unless context dictates otherwise, the term “Cannabis” is intended to include all putative species within the genus, such as, without limitation, Cannabis sativa, Cannabis indica, and Cannabis ruderalis and without regard to whether the Cannabis is hemp-type or drug-type.

The term “cyclase activity” in reference to a polyketide synthase (PKS) enzyme (e.g., an olivetol synthase (OLS) enzyme) or a polyketide cyclase (PKC) enzyme (e.g., an olivetolic acid cyclase (OAC) enzyme), refers to the activity of catalyzing the cyclization of an oxo fatty acyl-CoA (e.g., 3,5,7-trioxododecanoyl-COA, 3,5,7-trioxodecanoyl-COA) to the corresponding intramolecular cyclization product (e.g., olivetolic acid, divarinic acid). In some embodiments, the PKS or PKC catalyzes the C2-C7 aldol condensation of an acyl-COA with three additional ketide moieties added thereto.

A “cytosolic” or “soluble” enzyme refers to an enzyme that is predominantly localized (or predicted to be localized) in the cytosol of a host cell.

A “eukaryote” is any organism whose cells contain a nucleus and other organelles enclosed within membranes. Eukaryotes belong to the taxon Eukarya or Eukaryota. The defining feature that sets eukaryotic cells apart from prokaryotic cells (i.e., bacteria and archaea) is that they have membrane-bound organelles, especially the nucleus, which contains the genetic material, and is enclosed by the nuclear envelope.

The term “host cell” refers to a cell that can be used to express a polynucleotide, such as a polynucleotide that encodes an enzyme used in biosynthesis of cannabinoids or cannabinoid precursors. The terms “genetically modified host cell,” “recombinant host cell,” and “recombinant strain” are used interchangeably and refer to host cells that have been genetically modified by, e.g., cloning and transformation methods, or by other methods known in the art (e.g., selective editing methods, such as CRISPR). Thus, the terms include a host cell (e.g., bacterial cell, yeast cell, fungal cell, insect cell, plant cell, mammalian cell, human cell, etc.) that has been genetically altered, modified, or engineered, so that it exhibits an altered, modified, or different genotype and/or phenotype, as compared to the naturally-occurring cell from which it was derived. It is understood that in some embodiments, the terms refer not only to the particular recombinant host cell in question, but also to the progeny or potential progeny of such a host cell.

The term “control host cell,” or the term “control” when used in relation to a host cell, refers to an appropriate comparator host cell for determining the effect of a genetic modification or experimental treatment. In some embodiments, the control host cell is a wild type cell. In other embodiments, a control host cell is genetically identical to the genetically modified host cell, except for the genetic modification(s) differentiating the genetically modified or experimental treatment host cell. In some embodiments, the control host cell has been genetically modified to express a wild type or otherwise known variant of an enzyme being tested for activity in other test host cells.

The term “heterologous” with respect to a polynucleotide, such as a polynucleotide comprising a gene, is used interchangeably with the term “exogenous” and the term “recombinant” and refers to: a polynucleotide that has been artificially supplied to a biological system; a polynucleotide that has been modified within a biological system, or a polynucleotide whose expression or regulation has been manipulated within a biological system. A heterologous polynucleotide that is introduced into or expressed in a host cell may be a polynucleotide that comes from a different organism or species from the host cell, or may be a synthetic polynucleotide, or may be a polynucleotide that is also endogenously expressed in the same organism or species as the host cell. For example, a polynucleotide that is endogenously expressed in a host cell may be considered heterologous when it is situated non-naturally in the host cell; expressed recombinantly in the host cell, either stably or transiently; modified within the host cell; selectively edited within the host cell; expressed in a copy number that differs from the naturally occurring copy number within the host cell; or expressed in a non-natural way within the host cell, such as by manipulating regulatory regions that control expression of the polynucleotide. In some embodiments, a heterologous polynucleotide is a polynucleotide that is endogenously expressed in a host cell but whose expression is driven by a promoter that does not naturally regulate expression of the polynucleotide. In other embodiments, a heterologous polynucleotide is a polynucleotide that is endogenously expressed in a host cell and whose expression is driven by a promoter that does naturally regulate expression of the polynucleotide, but the promoter or another regulatory region is modified. In some embodiments, the promoter is recombinantly activated or repressed. For example, gene-editing based techniques may be used to regulate expression of a polynucleotide, including an endogenous polynucleotide, from a promoter, including an endogenous promoter. See, e.g., Chavez el al., Nat Methods. 2016 July; 13(7): 563-567. A heterologous polynucleotide may comprise a wild-type sequence or a mutant sequence as compared with a reference polynucleotide sequence.

The term “at least a portion” or “at least a fragment” of a nucleic acid or polypeptide means a portion having the minimal size characteristics of such sequences, or any larger fragment of the full length molecule, up to and including the full length molecule. A fragment of a polynucleotide of the disclosure may encode a biologically active portion of an enzyme, such as a catalytic domain. A biologically active portion of a genetic regulatory element may comprise a portion or fragment of a full length genetic regulatory element and have the same type of activity as the full length genetic regulatory element, although the level of activity of the biologically active portion of the genetic regulatory element may vary compared to the level of activity of the full length genetic regulatory element.

A coding sequence and a regulatory sequence are said to be “operably joined” or “operably linked” when the coding sequence and the regulatory sequence are covalently linked and the expression or transcription of the coding sequence is under the influence or control of the regulatory sequence. If the coding sequence is to be translated into a functional protein, the coding sequence and the regulatory sequence are said to be operably joined if induction of a promoter in the 5′ regulatory sequence promotes transcription of the coding sequence and if the nature of the linkage between the coding sequence and the regulatory sequence does not (1) result in the introduction of a frame-shift mutation, (2) interfere with the ability of the promoter region to direct the transcription of the coding sequence, or (3) interfere with the ability of the corresponding RNA transcript to be translated into a protein.

The terms “link,” “linked,” or ‘linkage’ means two entities (e.g., two polynucleotides or two proteins) are bound to one another by any physicochemical means. Any linkage known to those of ordinary skill in the art, covalent or non-covalent, is embraced. In some embodiments, a nucleic acid sequence encoding an enzyme of the disclosure is linked to a nucleic acid encoding a signal peptide. In some embodiments, an enzyme of the disclosure is linked to a signal peptide. Linkage can be direct or indirect.

The terms “transformed” or “transform” with respect to a host cell refer to a host cell in which one or more nucleic acids have been introduced, for example on a plasmid or vector or by integration into the genome. In some instances where one or more nucleic acids are introduced into a host cell on a plasmid or vector, one or more of the nucleic acids, or fragments thereof, may be retained in the cell, such as by integration into the genome of the cell, while the plasmid or vector itself may be removed from the cell. In such instances, the host cell is considered to be transformed with the nucleic acids that were introduced into the cell regardless of whether the plasmid or vector is retained in the cell or not.

The term “volumetric productivity” or “production rate” refers to the amount of product formed per volume of medium per unit of time. Volumetric productivity can be reported in gram per liter per hour (g/L/h).

The term “specific productivity” of a product refers to the rate of formation of the product normalized by unit volume or mass or biomass and has the physical dimension of a quantity of substance per unit time per unit mass or volume [M·T⁻¹·M⁻¹ or M·T⁻¹·L⁻³, where M is mass or moles, T is time, L is length].

The term “biomass specific productivity” refers to the specific productivity in gram product per gram of cell dry weight (CDW) per hour (g/g CDW/h) or in mmol of product per gram of cell dry weight (CDW) per hour (mmol/g CDW/h). Using the relation of CDW to OD600 for the given microorganism, specific productivity can also be expressed as gram product per liter culture medium per optical density of the culture broth at 600 nm (OD) per hour (g/L/h/OD). Also, if the elemental composition of the biomass is known, biomass specific productivity can be expressed in mmol of product per C-mole (carbon mole) of biomass per hour (mmol/C-mol/h).

The term “yield” refers to the amount of product obtained per unit weight of a certain substrate and may be expressed as g product per g substrate (g/g) or moles of product per mole of substrate (mol/mol). Yield may also be expressed as a percentage of the theoretical yield. “Theoretical yield” is defined as the maximum amount of product that can be generated per a given amount of substrate as dictated by the stoichiometry of the metabolic pathway used to make the product and may be expressed as g product per g substrate (g/g) or moles of product per mole of substrate (mol/mol).

The term “titer” refers to the strength of a solution or the concentration of a substance in solution. For example, the titer of a product of interest (e.g., small molecule, peptide, synthetic compound, fuel, alcohol, etc.) in a fermentation broth is described as g of product of interest in solution per liter of fermentation broth or cell-free broth (g/L) or as g of product of interest in solution per kg of fermentation broth or cell-free broth (g/Kg).

The term “total titer” refers to the sum of all products of interest produced in a process, including but not limited to the products of interest in solution, the products of interest in gas phase if applicable, and any products of interest removed from the process and recovered relative to the initial volume in the process or the operating volume in the process. For example, the total titer of products of interest (e.g., small molecule, peptide, synthetic compound, fuel, alcohol, etc.) in a fermentation broth is described as g of products of interest in solution per liter of fermentation broth or cell-free broth (g/L) or as g of products of interest in solution per kg of fermentation broth or cell-free broth (g/Kg).

The term “amino acid” refers to organic compounds that comprise an amino group, —NH2, and a carboxyl group, —COOH. The term “amino acid” includes both naturally occurring and unnatural amino acids. Nomenclature for the twenty common amino acids is as follows: alanine (ala or A); arginine (arg or R); asparagine (asn or N); aspartic acid (asp or D); cysteine (cys or C); glutamine (gln or Q); glutamic acid (glu or E); glycine (gly or G); histidine (his or H); isoleucine (ile or I); leucine (leu or L); lysine (lys or K); methionine (met or M); phenylalanine (phe or F); proline (pro or P); serine (ser or S); threonine (thr or T); tryptophan (trp or W); tyrosine (tyr or Y); and valine (val or V). Non-limiting examples of unnatural amino acids include homo-amino acids, proline and pyruvic acid derivatives, 3-substituted alanine derivatives, glycine derivatives, ring-substituted phenylalanine derivatives, ring-substituted tyrosine derivatives, linear core amino acids, amino acids with protecting groups including Fmoc, Boc, and Cbz, β-amino acids (β3 and β2), and N-methyl amino acids.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of, or a substituent that is, a straight-chain or branched saturated hydrocarbon group having from 1 to 20 carbon atoms (“C₁₋₂₀ alkyl”). In certain embodiments, the term “alkyl” refers to a radical of, or a substituent that is, a straight-chain or branched saturated hydrocarbon group having from 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 2 to 7 carbon atoms (“C₂₋₇alkyl”). In some embodiments, an alkyl group has 3 to 7 carbon atoms (“C₃₋₇ alkyl”). In some embodiments, an alkyl group has 1 to 6 carbon atoms (“C₁₋₆ alkyl”). In some embodiments, an alkyl group has 2 to 6 carbon atoms (“C₂₋₆ alkyl”). In some embodiments, an alkyl group has 3 to 5 carbon atoms (“C₃₋₅ alkyl”). In some embodiments, an alkyl group has 5 carbon atoms (“C₅ alkyl”). In some embodiments, the alkyl group has 3 carbon atoms (“C₃ alkyl”). In some embodiments, the alkyl group has 7 carbon atoms (“C₇ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”).

Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (C₅) (e.g., n-pentyl, 3-pentanyl, amyl, neopentyl, 3-methyl-2-butanyl, tertiary amyl), and hexyl (C₆) (e.g., n-hexyl). Additional examples of alkyl groups include n-heptyl (C₇), n-octyl (C₈), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₀ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu), unsubstituted isobutyl (i-Bu)). In certain embodiments, the alkyl group is a substituted C₁₋₁₀ alkyl (such as substituted C₁₋₆ alkyl, e.g., —CF₃, benzyl).

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described in this application that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

“Alkenyl” refers to a radical of, or a substituent that is, a straight-chain or branched hydrocarbon group having from 2 to 20 carbon atoms, one or more carbon-carbon double bonds, and no triple bonds (“C₂₋₂₀ alkenyl”). In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently optionally substituted, i.e., unsubstituted (an “unsubstituted alkenyl”) or substituted (a “substituted alkenyl”) with one or more substituents. In certain embodiments, the alkenyl group is unsubstituted C₂₋₁₀ alkenyl. In certain embodiments, the alkenyl group is substituted C₂₋₁₀ alkenyl.

“Alkynyl” refers to a radical of, or a substituent that is, a straight-chain or branched hydrocarbon group having from 2 to 20 carbon atoms, one or more carbon-carbon triple bonds, and optionally one or more double bonds (“C₂₋₂₀ alkynyl”). In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₅), and the like. Unless otherwise specified, each instance of an alkynyl group is independently optionally substituted, i.e., unsubstituted (an “unsubstituted alkynyl”) or substituted (a “substituted alkynyl”) with one or more substituents. In certain embodiments, the alkynyl group is unsubstituted C₂₋₁₀ alkynyl. In certain embodiments, the alkynyl group is substituted C₂₋₁₀ alkynyl.

“Carbocyclyl” or “carbocyclic” refers to a radical of a non-aromatic cyclic hydrocarbon group having from 3 to 10 ring carbon atoms (“C₃₋₁₀ carbocyclyl”) and zero heteroatoms in the non-aromatic ring system. In some embodiments, a carbocyclyl group has 3 to 8 ring carbon atoms (“C₃₋₈ carbocyclyl”). In some embodiments, a carbocyclyl group has 3 to 6 ring carbon atoms (“C₃₋₆ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or contain a fused, bridged or spiro ring system such as a bicyclic system (“bicyclic carbocyclyl”) and can be saturated or can be partially unsaturated. “Carbocyclyl” also includes ring systems wherein the carbocyclic ring, as defined above, is fused with one or more aryl or heteroaryl groups wherein the point of attachment is on the carbocyclic ring, and in such instances, the number of carbons continue to designate the number of carbons in the carbocyclic ring system. Unless otherwise specified, each instance of a carbocyclyl group is independently optionally substituted, i.e., unsubstituted (an “unsubstituted carbocyclyl”) or substituted (a “substituted carbocyclyl”) with one or more substituents. In certain embodiments, the carbocyclyl group is unsubstituted C₃₋₁₀ carbocyclyl. In certain embodiments, the carbocyclyl group is a substituted C₃₋₁₀ carbocyclyl.

In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl.

“Aryl” refers to a radical of a monocyclic or polycyclic (e.g., bicyclic or tricyclic) 4n+2 aromatic ring system (e.g., having 6, 10, or 14 pi electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

“Aralkyl” is a subset of alkyl and aryl and refers to an optionally substituted alkyl group substituted by an optionally substituted aryl group. In certain embodiments, the aralkyl is optionally substituted benzyl. In certain embodiments, the aralkyl is benzyl. In certain embodiments, the aralkyl is optionally substituted phenethyl. In certain embodiments, the aralkyl is phenethyl. In certain embodiments, the aralkyl is 7-phenylheptanyl. In certain embodiments, the aralkyl is C7 alkyl substituted by an optionally substituted aryl group (e.g., phenyl). In certain embodiments, the aralkyl is a C7-C10 alkyl group substituted by an optionally substituted aryl group (e.g., phenyl).

“Partially unsaturated” refers to a group that includes at least one double or triple bond. A “partially unsaturated” ring system is further intended to encompass rings having multiple sites of unsaturation but is not intended to include aromatic groups (e.g., aryl or heteroaryl groups) as defined in this application. Likewise. “saturated” refers to a group that does not contain a double or triple bond, i.e., contains all single bonds.

The term “optionally substituted” means substituted or unsubstituted.

Alkyl, alkenyl, alkynyl, carbocyclyl, heterocyclyl, aryl, and heteroaryl groups are optionally substituted (e.g., “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted,” whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described in this application that results in the formation of a stable compound. The present invention contemplates any and all such combinations in order to arrive at a stable compound. For purposes of this invention, heteroatoms such as nitrogen may have hydrogen substituents and/or any suitable substituent as described in this application which satisfy the valencies of the heteroatoms and results in the formation of a stable moiety.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —CO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃ —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl;
wherein:
   each instance of R^{aa} is, independently, selected from C¹⁻¹⁰ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
   each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O))R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion;
   each instance of R^{dd} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
   each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and
   each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl) ⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₄ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃ —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═OX(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion. Alternatively, two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc}; wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
wherein:
each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{cc}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion:
each instance of R^{cc} is, independently, selected from hydrogen. C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion;
each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups:
each instance of R^{ff} is, independently, selected from hydrogen, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃ —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(C₁₋₆ alkyl)₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

The term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describe pharmaceutically acceptable salts in detail in J. Pharmaceutical Sciences, 1977, 66, 1-19, incorporated by reference. Pharmaceutically acceptable salts of the compounds disclosed in this application include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of a compound that are associated with a solvent, usually by a solvolysis reaction. This physical association may include hydrogen bonding. Conventional solvents include water, methanol, ethanol, acetic acid, DMSO, THF, diethyl ether, and the like. The compounds of Formula (1), (9), (10), and (11) may be prepared. e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound and wherein x is a number greater than 0. A given compound may form more than one type of hydrates, including, e.g., monohydrates (x is 1), lower hydrates (x is a number greater than 0 and smaller than 1, e.g., hemihydrates (R·0.5 H₂O)), and polyhydrates (x is a number greater than 1, e.g., dihydrates (R·2 H₂O) and hexahydrates (R·6 H₂O)).

The term “tautomers” refer to compounds that are interchangeable forms of a particular compound structure, and that vary in the displacement of hydrogen atoms and electrons. Thus, two structures may be in equilibrium through the movement of a electrons and an atom (usually H). For example, enols and ketones are tautomers because they are rapidly interconverted by treatment with either acid or base. Another example of tautomerism is the aci- and nitro-forms of phenylnitromethane, which are likewise formed by treatment with acid or base. Tautomeric forms may be relevant to the attainment of the optimal chemical reactivity and biological activity of a compound of interest.

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.”

Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and described by the R- and S-sequencing rules of Cahn and Prelog. An enantiomer can also be characterized by the manner in which the molecule rotates the plane of polarized light, and designated as dextrorotatory or levorotatory (i.e., as (+) or (−)-isomers respectively). A chiral compound can exist as either an individual enantiomer or as a mixture of enantiomers. A mixture containing equal proportions of the enantiomers is called a “racemic mixture.”

The term “co-crystal” refers to a crystalline structure comprising at least two different components (e.g., a compound described in this application and an acid), wherein each of the components is independently an atom, ion, or molecule. In certain embodiments, none of the components is a solvent. In certain embodiments, at least one of the components is a solvent. A co-crystal of a compound and an acid is different from a salt formed from a compound and the acid. In the salt, a compound described in this application is complexed with the acid in a way that proton transfer (e.g., a complete proton transfer) from the acid to a compound described in this application easily occurs at room temperature. In the co-crystal, however, a compound described in this application is complexed with the acid in a way that proton transfer from the acid to a compound described in this application does not easily occur at room temperature. In certain embodiments, in the co-crystal, there is no proton transfer from the acid to a compound described in this application. In certain embodiments, in the co-crystal, there is partial proton transfer from the acid to a compound described in this application. Co-crystals may be useful to improve the properties (e.g., solubility, stability, and ease of formulation) of a compound described in this application.

The term “polymorphs” refers to a crystalline form of a compound (or a salt, hydrate, or solvate thereof) in a particular crystal packing arrangement. All polymorphs of the same compound have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “prodrug” refers to compounds, including derivatives of the compounds of Formula (X), (8), (9), (10), or (11), that have cleavable groups and become by solvolysis or under physiological conditions the compounds of Formula (X), (8), (9), (10), or (11) and that are pharmaceutically active in vivo. The prodrugs may have attributes such as, without limitation, solubility, bioavailability, tissue compatibility, or delayed release in a mammalian organism. Examples include, but are not limited to, derivatives of compounds described in this application, including derivatives formed from glycosylation of the compounds described in this application (e.g., glycoside derivatives), carrier-linked prodrugs (e.g., ester derivatives), bioprecursor prodrugs (a prodrug metabolized by molecular modification into the active compound), and the like. Non-limiting examples of glycoside derivatives are disclosed in and incorporated by reference from PCT Publication No. WO2018/208875 and U.S. Patent Publication No. 2019/0078168. Non-limiting examples of ester derivatives are disclosed in and incorporated by reference from U.S. Patent Publication No. US2017/0362195.

Other derivatives of the compounds of this invention have activity in both their acid and acid derivative forms, but the acid sensitive form often offers advantages of solubility, bioavailability, tissue compatibility, or delayed release in a mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds of this invention are particular prodrugs. In some cases it is desirable to prepare double ester type prodrugs such as (acyloxy)alkyl esters or ((alkoxycarbonyl)oxy)alkylesters. C₁-C₈ alkyl, C₂-C₈ alkenyl, C₂-C₈ alkynyl, aryl, C₇-C₁₂ substituted aryl, and C₇-C₁₂ arylalkyl esters of the compounds of Formula (X), (8), (9), (10), or (11) may be preferred.

### Cannabinoids

As used in this application, the term “cannabinoid” includes compounds of Formula (X):

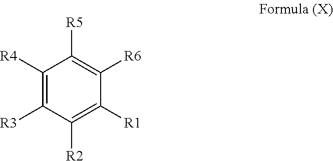

or a pharmaceutically acceptable salt, co-crystal, tautomer, stereoisomer, solvate, hydrate, polymorph, isotopically enriched derivative, or prodrug thereof, wherein R1 is hydrogen, optionally substituted acyl, optionally substituted alkyl, optionally substituted alkenyl, optionally substituted alkynyl, optionally substituted carbocyclyl, or optionally substituted aryl; R2 and R6 are, independently, hydrogen or carboxyl; R3 and R5 are, independently, hydroxyl, halogen, or alkoxy; and R4 is a hydrogen or an optionally substituted prenyl moiety; or optionally R4 and R3 are taken together with their intervening atoms to form a cyclic moiety, or optionally R4 and R5 are taken together with their intervening atoms to form a cyclic moiety, or optionally both 1) R4 and R3 are taken together with their intervening atoms to form a cyclic moiety and 2) R4 and R5 are taken together with their intervening atoms to form a cyclic moiety. In certain embodiments, R4 and R3 are taken together with their intervening atoms to form a cyclic moiety. In certain embodiments, R4 and R5 are taken together with their intervening atoms to form a cyclic moiety. In certain embodiments, “cannabinoid” refers to a compound of Formula (X), or a pharmaceutically acceptable salt thereof. In certain embodiments, both 1) R4 and R3 are taken together with their intervening atoms to form a cyclic moiety and 2) R4 and R5 are taken together with their intervening atoms to form a cyclic moiety.

In some embodiments, cannabinoids may be synthesized via the following steps: a) one or more reactions to incorporate three additional ketone moieties onto an acyl-CoA scaffold, where the acyl moiety in the acyl-CoA scaffold comprises between four and fourteen carbons; b) a reaction cyclizing the product of step (a); and c) a reaction to incorporate a prenyl moiety to the product of step (b) or a derivative of the product of step (b). In some embodiments, non-limiting examples of the acyl-CoA scaffold described in step (a) include hexanoyl-CoA and butyryl-CoA. In some embodiments, non-limiting examples of the product of step (b) or a derivative of the product of step (b) include olivetolic acid, divarinic acid, and sphaerophorolic acid.

In some embodiments, a cannabinoid compound of Formula (X) is of Formula (X-A), (X-B), or (X-C):

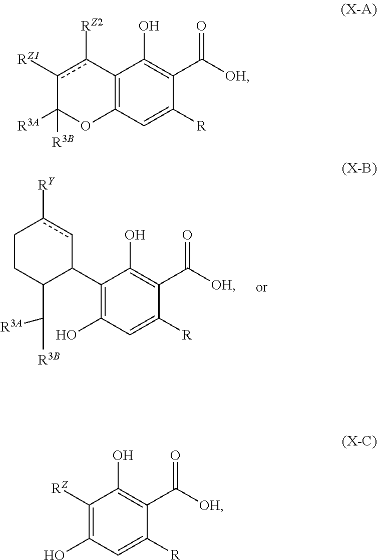

or a pharmaceutically acceptable salt, solvate, hydrate, polymorph, co-crystal, tautomer, stereoisomer, isotopically labeled derivative, or prodrug thereof:
wherein [symbol] is a double bond or a single bond, as valency permits,
R is hydrogen, optionally substituted acyl, optionally substituted alkyl, optionally substituted alkenyl, optionally substituted alkynyl, optionally substituted carbocyclyl, or optionally substituted aryl;
R^{Z1} is hydrogen, optionally substituted acyl, optionally substituted alkyl, optionally substituted alkenyl, optionally substituted alkynyl, optionally substituted carbocyclyl, or optionally substituted aryl;
R^{Z2} is hydrogen, optionally substituted acyl, optionally substituted alkyl, optionally substituted alkenyl, optionally substituted alkynyl, optionally substituted carbocyclyl, or optionally substituted aryl;
or optionally, R^{Z1} and R^{Z2} are taken together with their intervening atoms to form an optionally substituted carbocyclic ring;
R^{3A} is hydrogen, optionally substituted acyl, optionally substituted alkyl, optionally substituted alkenyl, or optionally substituted alkynyl;
R^{3B} is hydrogen, optionally substituted acyl, optionally substituted alkyl, optionally substituted alkenyl, or optionally substituted alkynyl;
R^{Y} is hydrogen, optionally substituted acyl, optionally substituted alkyl, optionally substituted alkenyl, or optionally substituted alkynyl;
R^{Z} is hydrogen, optionally substituted acyl, optionally substituted alkyl, optionally substituted alkenyl, or optionally substituted alkynyl.

In certain embodiments, a cannabinoid compound is of Formula (X-A):

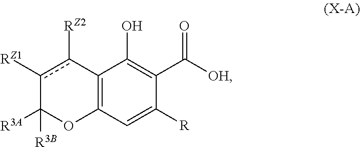

wherein [symbol] is a double bond, and each of R^{Z1} and R^{Z2} is hydrogen, one of R^{3A} and R^{3B} is optionally substituted C₂₋₆ alkenyl, and the other one of R^{3A} and R^{3B} is optionally substituted C₂₋₆ alkyl. In some embodiments, a cannabinoid compound of Formula (X) is of Formula (X-A), wherein each of R^{Z1} and R^{Z2} is hydrogen, one of R^{3A} and R^{3B} is a prenyl group, and the other one of R^{3A} and R^{3B} is optionally substituted methyl.

In certain embodiments, a cannabinoid compound of Formula (X) of Formula (X-A) is of Formula (11-z):

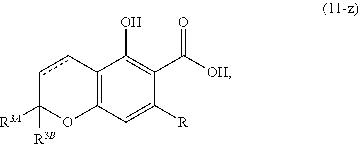

wherein [symbol] is a double bond or single bond, as valency permits; one of R^{3A} and R^{3B} is C₁₋₆ alkyl optionally substituted with alkenyl, and the other of R^{3A} and R^{3B} is optionally substituted C₁₋₆ alkyl. In certain embodiments, in a compound of Formula (11-z), [symbol] is a single bond; one of R^{3A} and R^{3B} is C₁₋₆ alkyl optionally substituted with prenyl; and the other of one of R^{3A} and R^{3B} is unsubstituted methyl; and R is as described in this application. In certain embodiments, in a compound of Formula (11-z), [symbol] is a single bond; one of R^{3A} and R^{3B} is

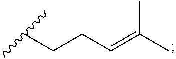 [SMILES: *CCC=C(C)C]

and the other of one of R^{3A} and R^{3B} is unsubstituted methyl; and R is as described in this application. In certain embodiments, a cannabinoid compound of Formula (11-z) is of Formula (11a):
om

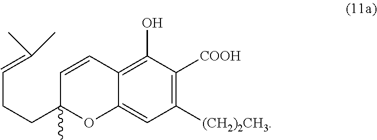

In certain embodiments, a cannabinoid compound of Formula (X) of Formula (X-A) is of Formula (11a):

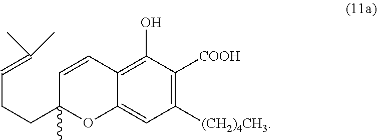

In certain embodiments, a cannabinoid compound of Formula (X-A) is of Formula (10-z):

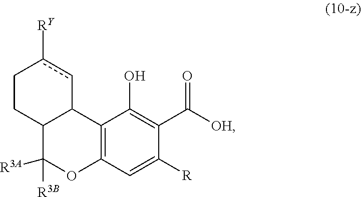

wherein [symbol] is a double bond or single bond, as valency permits; R^{Y} is hydrogen, optionally substituted acyl, optionally substituted alkyl, optionally substituted alkenyl, or optionally substituted alkynyl; and each of R^{3A} and R^{3B} is independently optionally substituted C₁₋₆ alkyl. In certain embodiments, in a compound of Formula (10-z) [symbol] is a single bond; each of R^{3A} and R^{3B} is unsubstituted methyl, and R is as described in this application. In certain embodiments, a cannabinoid compound of Formula (10-z) is of Formula (10a):

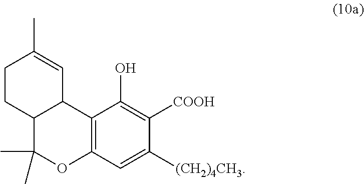

In certain embodiments, a compound of Formula (10a)

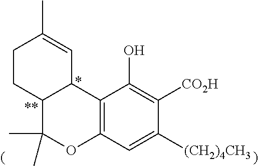

has a chiral atom labeled with * at carbon 10 and a chiral atom labeled with ** at carbon 6. In certain embodiments, in a compound of Formula (10a)

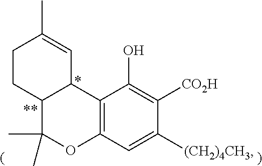

the chiral atom labeled with * at carbon 10 is of the R-configuration or S-configuration; and a chiral atom labeled with ** at carbon 6 is of the R-configuration. In certain embodiments, in a compound of Formula (10a)

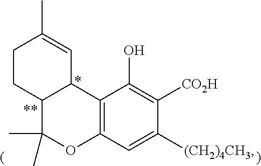

the chiral atom labeled with * at carbon 10 is of the S-configuration; and a chiral atom labeled with ** at carbon 6 is of the R-configuration or S-configuration. In certain embodiments, in a compound of Formula (10a)

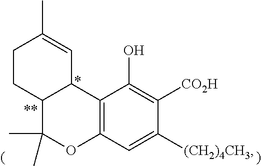

the chiral atom labeled with * at carbon 10 is of the R-configuration and a chiral atom labeled with ** at carbon 6 is of the R-configuration. In certain embodiments, a compound of Formula (10a)

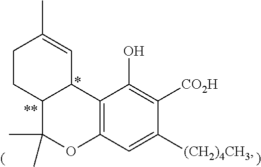

is of the formula:

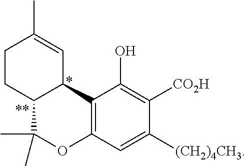

In certain embodiments, in a compound of Formula (10a)

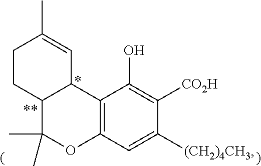

the chiral atom labeled with * at carbon 10 is of the S-configuration and a chiral atom labeled with ** at carbon 6 is of the S-configuration. In certain embodiments, a compound of Formula (10a)

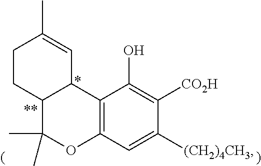

is of the formula:

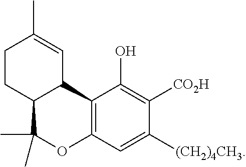

In certain embodiments, a cannabinoid compound is of Formula (X-B):

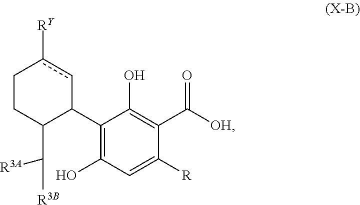

wherein [symbol] a double bond; R^{Y} is hydrogen, optionally substituted acyl, optionally substituted alkyl, optionally substituted alkenyl, or optionally substituted alkynyl; and each of R^{3A} and R^{3B} is independently optionally substituted C₁₋₆ alkyl. In certain embodiments, in a compound of Formula (X-B), R^{Y} is optionally substituted C₁₋₆ alkyl; one of R^{3A} and R^{3B} is

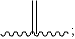

and the other one of R^{3A} and R^{3B} is unsubstituted methyl, and R is as described in this application. In certain embodiments, a compound of Formula (X-B) is of Formula (9a):

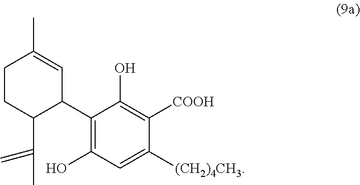

In certain embodiments, a compound of Formula (9a)

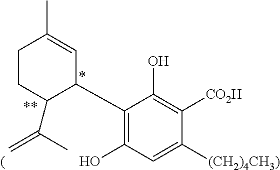

has a chiral atom labeled with * at carbon 3 and a chiral atom labeled with ** at carbon 4. In certain embodiments, in a compound of Formula (9a)

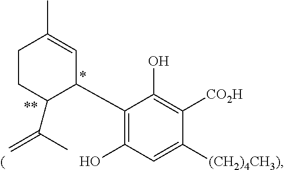

the chiral atom labeled with * at carbon 3 is of the R-configuration or S-configuration; and a chiral atom labeled with ** at carbon 4 is of the R-configuration. In certain embodiments, in a compound of Formula (9a)

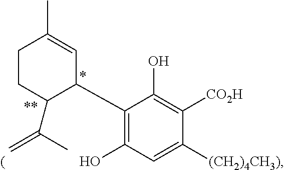

the chiral atom labeled with * at carbon 3 is of the S-configuration; and a chiral atom labeled with ** at carbon 4 is of the R-configuration or S-configuration. In certain embodiments, in a compound of Formula (9a)

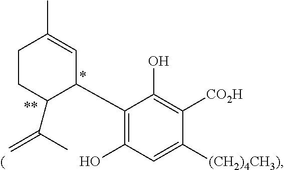

the chiral atom labeled with * at carbon 3 is of the R-configuration and a chiral atom labeled with ** at carbon 4 is of the R-configuration. In certain embodiments, a compound of Formula (9a)

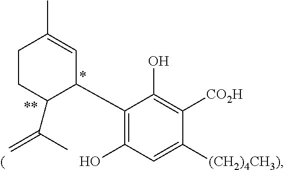

is of the formula:

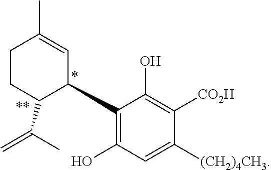

In certain embodiments, in a compound of Formula (9a)

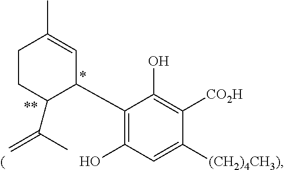

the chiral atom labeled with * at carbon 3 is of the S-configuration and a chiral atom labeled with ** at carbon 4 is of the S-configuration. In certain embodiments, a compound of Formula (9a)

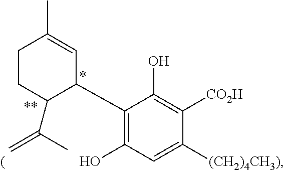

is of the formula:

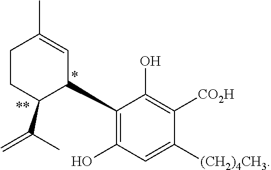

In certain embodiments, a cannabinoid compound is of Formula (X-C):

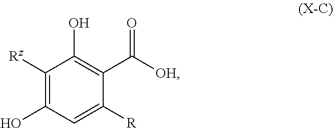

wherein R^{Z} is optionally substituted alkyl or optionally substituted alkenyl. In certain embodiments, a compound of Formula (X-C) is of formula:

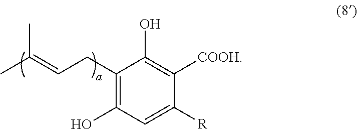

wherein a is 1, 2, 3, 4, 5, 6, 7, 8, 9, or 10. In certain embodiments, a is 1. In certain embodiments, a is 2. In certain embodiments, a is 3. In certain embodiments, a is 1, 2, or 3 for a compound of Formula (X-C). In certain embodiments, a cannabinoid compound is of Formula (X-C), and a is 1, 2, 3, 4, or 5. In certain embodiments, a compound of Formula (X-C) is of Formula (8a):

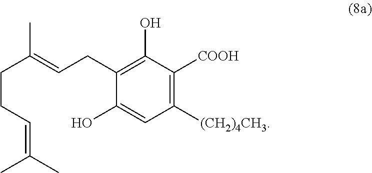

In some embodiments, cannabinoids of the present disclosure comprise cannabinoid receptor ligands. Cannabinoid receptors are a class of cell membrane receptors in the G protein-coupled receptor superfamily. Cannabinoid receptors include the CB₁ receptor and the CB₂ receptor. In some embodiments, cannabinoid receptors comprise GPR18, GPR55, and PPAR. (See Brain et al. “Activation of GPR18 by cannabinoid compounds: a tale of biased agonism” Br J Pharmcol v171 (16) (2014); Shi et al. “The novel cannabinoid receptor GPR55 mediates anxiolytic-like effects in the medial orbital cortex of mice with acute stress” Molecular Brain 10, No. 38 (2017); and O'Sullvan, Elizabeth. “An update on PPAR activation by cannabinoids” Br J Pharmcol v. 173(12) (2016)).

In some embodiments, cannabinoids comprise endocannabinoids, which are substances produced within the body, and phytocannabinoids, which are cannabinoids that are naturally produced by plants of genus Cannabis. In some embodiments, phytocannabinoids comprise the acidic and decarboxylated acid forms of the naturally-occurring plant-derived cannabinoids, and their synthetic and biosynthetic equivalents.

Over 94 phytocannabinoids have been identified to date (Berman, Paula, et al. “A new ESI-LC/MS approach for comprehensive metabolic profiling of phytocannabinoids in Cannabis.” Scientific reports 8.1 (2018): 14280; El-Alfy et al., 2010, “Antidepressant-like effect of delta-9-tetrahydrocannabinol and other cannabinoids isolated from Cannabis sativa L”, Pharmacology Biochemistry and Behavior 95 (4): 434-42; Rudolf Brenneisen, 2007, Chemistry and Analysis of Phytocannabinoids, Citti, Cinzia, et al. “A novel phytocannabinoid isolated from Cannabis sativa L. with an in vivo cannabimimetic activity higher than Δ9-tetrahydrocannabinol: Δ9-Tetrahydrocannabiphorol.” Sci Rep 9 (2019): 20335, each of which is incorporated by reference in this application in its entirety). In some embodiments, cannabinoids comprise Δ⁹-tetrahydrocannabinol (THC) type (e.g., (−)-trans-delta-9-tetrahydrocannabinol or dronabinol, (+)-trans-delta-9-tetrahydrocannabinol, (−)-cis-delta-9-tetrahydrocannabinol, or (+)-cis-delta-9-tetrahydrocannabinol), cannabidiol (CBD) type, cannabigerol (CBG) type, cannabichromene (CBC) type, cannabicyclol (CBL) type, cannabinodiol (CBND) type, or cannabitriol (CBT) type cannabinoids, or any combination thereof (see, e.g., R Pertwee, ed, Handbook of Cannabis (Oxford, UK: Oxford University Press, 2014)), which is incorporated by reference in this application in its entirety). A non-limiting list of cannabinoids comprises: cannabiorcol-C1 (CBNO), CBND-C1 (CBNDO), Δ⁹-trans-Tetrahydrocannabiorcolic acid-C1 (Δ⁹-THCO), Cannabidiorcol-C1 (CBDO), Cannabiorchromene-C1 (CBCO), (−)-Δ⁸-trans-(6aR,10aR)-Tetrahydrocannabiorcol-C1 (Δ⁸-THCO), Cannabiorcyclol C1 (CBLO). CBG-C1 (CBGO), Cannabinol-C2 (CBN-C2), CBND-C2, Δ⁹-THC-C2, CBD-C2, CBC-C2, Δ⁸-THC-C2, CBL-C2, Bisnor-cannabielsoin-C1 (CBEO), CBG-C2, Cannabivarin-C3 (CBNV), Cannabinodivarin-C3 (CBNDV), (−)-Δ⁹-trans-Tetrahydrocannabivarin-C3 (Δ⁹-THCV), (−)-Cannabidivarin-C3 (CBDV), (±)-Cannabichromevarin-C3 (CBCV), (−)-Δ⁸-trans-THC-C3 (Δ⁸-THCV), (+)-(1aS,3aR,8bR,8cR)-Cannabicyclovarin-C3 (CBLV), 2-Methyl-2-(4-methyl-2-pentenyl)-7-propyl-2H-1-benzopyran-5-ol, Δ⁷-tetrahydrocannabivarin-C3 (Δ⁷-THCV), CBE-C2. Cannabigerovarin-C3 (CBGV), Cannabitriol-C1 (CBTO), Cannabinol-C4 (CBN-C4), CBND-C4, (−)-Δ⁹-trans-Tetrahydrocannabinol-C4 (Δ⁹-THC-C4), Cannabidiol-C4 (CBD-C4), CBC-C4, (−)-trans-Δ⁸-THC-C4, CBL-C4, Cannabielsoin-C3 (CBEV), CBG-C4, CBT-C2, Cannabichromanone-C3, Cannabiglendol-C3 (OH-iso-HHCV-C3), Cannabioxepane-C5 (CBX), Dehydrocannabifuran-C5 (DCBF), Cannabinol-C5 (CBN), Cannabinodiol-C5 (CBND), (−)-Δ⁹-trans-Tetrahydrocannabinol-C5 (Δ⁹-THC), (−)-Δ⁸-trans-(6aR,10aR)-Tetrahydrocannabinol-C5 (Δ⁸-THC), (±)-Cannabichromene-C5 (CBC), (−)-Cannabidiol-C5 (CBD), (±)-(1aS,3aR,8bR,8cR)-CannabicyclolC5 (CBL), Cannabicitran-C5 (CBR), (−)-Δ⁹-(6aS,10aR-cis)-Tetrahydrocannabinol-C5 ((−)-cis-Δ⁹-THC), (−)-Δ⁷-trans-(1R,3R,6R)-Isotetrahydrocannabinol-C5 (trans-isoΔ⁷-THC), CBE-C4, Cannabigerol-C5 (CBG), Cannabitrol-C3 (CBTV), Cannabinol methyl ether-C5 (CBNM), CBNDM-C5, 8-OH-CBN-C5 (OH-CBN), OH-CBND-C5 (OH-CBND), 10-Oxo-Δ^{6a(10a)}-Tetrahydrocannabinol-C5 (OTHC), Cannabichromanone D-C5, Cannabicoumaronone-C5 (CBCON-C5), Cannabidiol monomethyl ether-C5 (CBDM), Δ⁹-THCM-C5, (±)-3″-hydroxy-Δ⁴″-cannabichromene-C5, (5aS,6S,9R,9aR)-Cannabielsoin-C5 (CBE), 2-geranyl-5-hydroxy-3-n-pentyl-1,4-benzoquinone-C5, 5-geranyl olivetolic acid, 5-geranyl olivetolate, 8α-Hydroxy-Δ⁹-Tetrahydrocannabinol-C5 (8α-OH-Δ⁹-THC), 8β-Hydroxy-Δ⁹-Tetrahydrocannabinol-C5 (8β-OH-Δ⁹-THC), 10α-Hydroxy-Δ⁸-Tetrahydrocannabinol-C5 (10α-OH-Δ⁸-THC), 10β-Hydroxy-Δ⁸-Tetrahydrocannabinol-C5 (10β-OH-Δ⁸-THC), 10α-hydroxy-Δ^{9,11}-hexahydrocannabinol-C5, 9β,10β-Epoxyhexahydrocannabinol-C5, OH-CBD-C5 (OH-CBD), Cannabigerol monomethyl ether-C5 (CBGM). Cannabichromanone-C5, CBT-C4, (±)-6,7-cis-epoxycannabigerol-C5, (±)-6,7-trans-epoxycannabigerol-C5, (−)-7-hydroxycannabichromane-C5, Cannabimovone-C5, (−)-trans-Cannabitriol-C5 ((−)-trans-CBT), (+)-trans-Cannabitriol-C5 ((+)-trans-CBT), (±)-cis-Cannabitriol-C5 ((±)-cis-CBT), (−)-trans-10-Ethoxy-9-hydroxy-Δ^{6a(10a)}-tetrahydrocannabivarin-C3 [(−)-trans-CBT-OEt], (−)-(6aR,9S,10S,10aR)-9,10-Dihydroxyhexahydrocannabinol-C5 [(−)-Cannabiripsol] (CBR), Cannabichromanone C-C5, (−)-6a,7,10a-Trihydroxy-Δ⁹-tetrahydrocannabinol-C5 [(−)-Cannabitetrol] (CBTT), Cannabichromanone B-C5, 8,9-Dihydroxy-Δ^{6a(10a)}-tetrahydrocannabinol-C5 (8,9-Di-OHCBT), (±)-4-acetoxycannabichromene-C5, 2-acetoxy-6-geranyl-3-n-pentyl-1,4-benzoquinone-C5, 11-Acetoxy-Δ 9-TetrahydrocannabinolC5 (11-OAc-Δ 9-THC), 5-acetyl-4-hydroxycannabigerol-C5, 4-acetoxy-2-geranyl-5-hydroxy-3-npentylphenol-C5, (−)-trans-10-Ethoxy-9-hydroxy-Δ^{6a(10a)}-tetrahydrocannabinol-C5 ((−)-trans-CBTOEt), sesquicannabigerol-C5 (SesquiCBG), carmagerol-C5, 4-terpenyl cannabinolate-C5, β-fenchyl-Δ⁹-tetrahydrocannabinolate-C5, α-fenchyl-Δ⁹-tetrahydrocannabinolate-C5, epi-bornyl-Δ⁹-tetrahydrocannabinolate-C5, bornyl-Δ⁹-tetrahydrocannabinolate-C5, α-terpenyl-Δ⁹-tetrahydrocannabinolate-C5, 4-terpenyl-Δ⁹-tetrahydrocannabinolate-C5, 6,6,9-trimethyl-3-pentyl-6H-dibenzo[b,d]pyran-1-ol, 3-(1,1-dimethylheptyl)-6,6a,7,8,10,10a-hexahydro-1-hydroxy-6,6-dimethyl-9H-dibenzo[b,d]pyran-9-one, (−)-(3S,4S)-7-hydroxy-Δ⁶-tetrahydrocannabinol-1,1-dimethylheptyl, (+)-(3S,4S)-7-hydroxy-Δ⁶-tetrahydrocannabinol-1,1-dimethylheptyl, 11-hydroxy-Δ⁹-tetrahydrocannabinol, and Δ⁸-tetrahydrocannabinol-11-oic acid)); certain piperidine analogs (e.g., (−)-(6S,6aR,9R,10aR)-5,6a,7,8,9,10,10a-octahydro-6-methyl-3-[(R)-1-methy-4-phenylbutoxy]-1,9-phenanthridinediol 1-acetate)), certain aminoalkylindole analogs (e.g., (R)-(+)-[2,3-dihydro-5-methyl-3-(4-morpholinymlethyl)-pyrrolo[1,2,3-de]-1,4-benzoxazin-6-yl]-1-naphthalenyl-methanone), certain open pyran ring analogs (e.g., 2-[3-methyl-6-(1-methylethenyl)-2-cyclohexen-1-yl]-5-pentyl-1,3-benzenediol and 4-(1,1-dimethylheptyl)-2,3′-dihydroxy-6′alpha-(3-hydroxypropyl)-1′,2′,3′,4′,5′6′-hexahydrobiphenyl, tetrahydrocannabiphorol (THCP), cannabidiphorol (CBDP), CBGP, CBCP, their acidic forms, salts of the acidic forms, dimers of any combination of the above, trimers of any combination of the above, polymers of any combination of the above, or any combination thereof.

A cannabinoid described in this application can be a rare cannabinoid. For example, in some embodiments, a cannabinoid described in this application corresponds to a cannabinoid that is naturally produced in conventional Cannabis varieties at concentrations of less than 10%, 9%, 8%, 7%, 6%, 5%, 4%, 3%, 2%, 1%, 0.9%, 0.8%, 0.7%, 0.6%, 0.5%, 0.25%, or 0.1% by dry weight of the female flower. In some embodiments, rare cannabinoids include CBGA, CBGVA, THCVA, CBDVA, CBCVA, and CBCA. In some embodiments, rare cannabinoids are cannabinoids that are not THCA, THC, CBDA or CBD.

A cannabinoid described in this application can also be a non-rare cannabinoid.

In some embodiments, the cannabinoid is selected from the cannabinoids listed in Table 1.

TABLE 1
Non-limiting examples of cannabinoids according to the present disclosure.

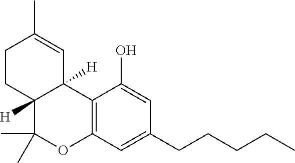 [SMILES: CCCCCc1cc(O)c2c(c1)OC(C)(C)[C@@H]1CCC(C)=C[C@@H]21]

Δ⁹-Tetrahydro-
cannabinol
Δ⁹-THC-C₅

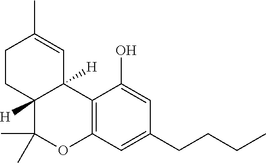 [SMILES: CCCCc1cc(O)c2c(c1)OC(C)(C)[C@@H]1CCC(C)=C[C@@H]21]

Δ⁹-Tetrahydro-
cannabinol-C₄
Δ⁹-THC-C₄

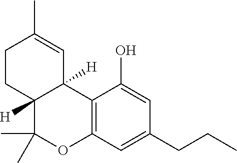 [SMILES: CCCc1cc(O)c2c(c1)OC(C)(C)[C@@H]1CCC(C)=C[C@@H]21]

Δ⁹-Tetrahydro-
cannabivarin
Δ⁹-THCV-C₃

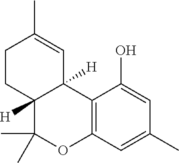

Δ⁹-Tetrahydro-
cannabiorcol
Δ⁹-THCO-C₁

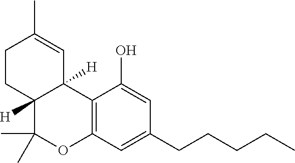 [SMILES: CCCCCc1cc(O)c2c(c1)OC(C)(C)[C@@H]1CCC(C)=C[C@@H]21]

(−)-(6aS,10aR)-Δ⁹-
Tetrahydro-
cannabinol
(−)-cis-Δ⁹-THC-C₅

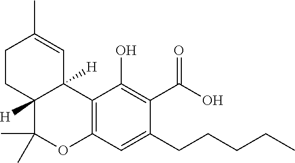 [SMILES: CCCCCc1cc2c(c(O)c1C(=O)O)[C@@H]1C=C(C)CC[C@H]1C(C)(C)O2]

Δ⁹-Tetrahydro-
cannabinolic acid A
Δ⁹-THCA-C₅ A

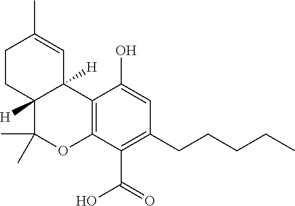 [SMILES: CCCCCc1cc(O)c2c(c1C(=O)O)OC(C)(C)[C@@H]1CCC(C)=C[C@@H]21]

Δ⁹-Tetrahydro-
cannabinolic acid B
Δ⁹-THCA-C₅ B

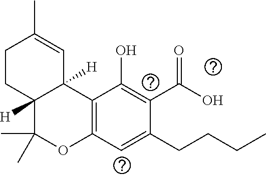 [SMILES: CCCCc1cc2c(c(O)c1C(=O)O)[C@@H]1C=C(C)CC[C@H]1C(C)(C)O2]

Δ⁹-Tetrahydro-
cannabinolic acid-C₄
A and/or B
Δ⁹-THCA-C₄ A and/or
B

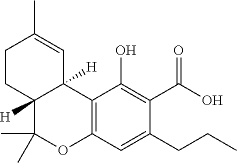 [SMILES: CCCc1cc2c(c(O)c1C(=O)O)[C@@H]1C=C(C)CC[C@H]1C(C)(C)O2]

Δ⁹-Tetrahydro-
cannabivarinic acid A
Δ⁹-THCVA-C₃ A

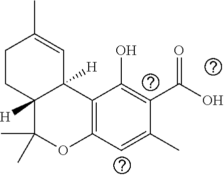 [SMILES: CC1=C[C@H]2c3c(cc(C)c(C(=O)O)c3O)OC(C)(C)[C@@H]2CC1]

Δ⁹-Tetrahydro-
cannabiorcolic acid
A and/or B
Δ⁹-THCOA-C₁ A
and/or B

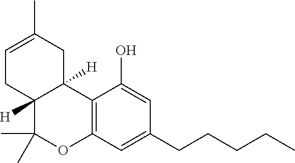 [SMILES: CCCCCc1cc(O)c2c(c1)OC(C)(C)[C@@H]1CC=C(C)C[C@@H]21]

(−)-Δ⁸-trans-
(6aR,10aR)-
Δ⁸-Tetrahydro-
cannabinol
Δ⁸-THC-C₅

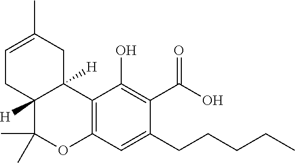 [SMILES: CCCCCc1cc2c(c(O)c1C(=O)O)[C@@H]1CC(C)=CC[C@H]1C(C)(C)O2]

(−)-Δ⁸-trans-
(6aR,10aR)-
Tetrahydro-
cannabinolic
acid A
Δ⁸-THCA-C₅ A

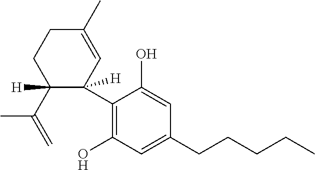 [SMILES: C=C(C)[C@@H]1CCC(C)=C[C@H]1c1c(O)cc(CCCCC)cc1O]

(−)-Cannabidiol
CBD-C5

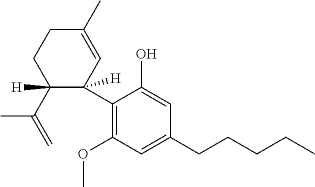 [SMILES: C=C(C)[C@@H]1CCC(C)=C[C@H]1c1c(O)cc(CCCCC)cc1OC]

Cannabidiol
momomethyl ether
CBDM-C5

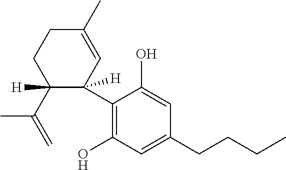 [SMILES: C=C(C)[C@@H]1CCC(C)=C[C@H]1c1c(O)cc(CCCC)cc1O]

Cannabidiol-C4
CBD-C4

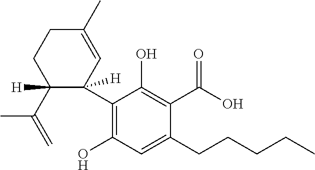 [SMILES: C=C(C)[C@@H]1CCC(C)=C[C@H]1c1c(O)cc(CCCCC)c(C(=O)O)c1O]

Cannabidiolic acid
CBDA-C5

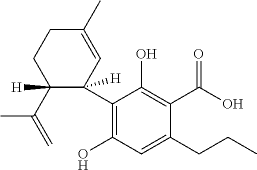 [SMILES: C=C(C)[C@@H]1CCC(C)=C[C@H]1c1c(O)cc(CCC)c(C(=O)O)c1O]

Cannabidivarinic acid
CBDVA-C3

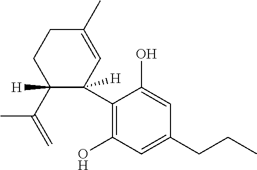 [SMILES: C=C(C)[C@@H]1CCC(C)=C[C@H]1c1c(O)cc(CCC)cc1O]

(−)-Cannabidivarin
CBDV-C3

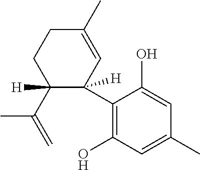 [SMILES: C=C(C)[C@@H]1CCC(C)=C[C@H]1c1c(O)cc(C)cc1O]

Cannabidiorcol
CBD-C1

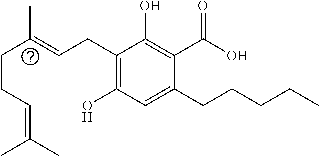 [SMILES: CCCCCc1cc(O)c(C/C=C(\C)CCC=C(C)C)c(O)c1C(=O)O]

Cannabigerolic acid A
(E)-CBGA-C₅ A

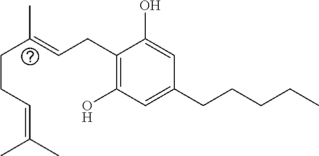 [SMILES: CCCCCc1cc(O)c(C/C=C(\C)CCC=C(C)C)c(O)c1]

Cannabigerol
(E)-CBG-C₅

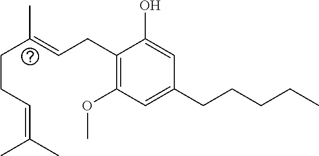 [SMILES: CCCCCc1cc(O)c(C/C=C(\C)CCC=C(C)C)c(OC)c1]

Cannabigerol
monomethyl ether
(E)-CBGM-C₅ A

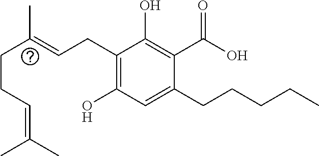 [SMILES: CCCCCc1cc(O)c(C/C=C(\C)CCC=C(C)C)c(O)c1C(=O)O]

Cannabinerolic acid A
(Z)-CBGA-C₅ A

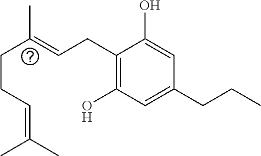 [SMILES: CCCc1cc(O)c(C/C=C(\C)CCC=C(C)C)c(O)c1]

Cannabigerovarin
(E)-CBGV-C₃

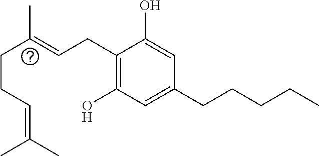 [SMILES: CCCCCc1cc(O)c(C/C=C(\C)CCC=C(C)C)c(O)c1]

Cannabigerol
(E)-CBG-C₅

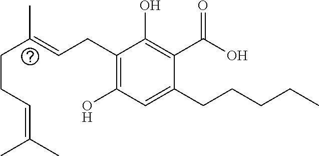 [SMILES: CCCCCc1cc(O)c(C/C=C(\C)CCC=C(C)C)c(O)c1C(=O)O]

Cannabigerolic acid A
(E)-CBGA-C₅ A

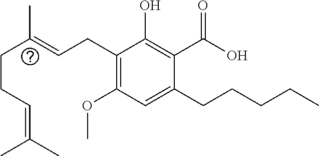 [SMILES: CCCCCc1cc(OC)c(C/C=C(\C)CCC=C(C)C)c(O)c1C(=O)O]

Cannabigerolic acid A
monomethyl ether
(E)-CBGAM-C₅ A

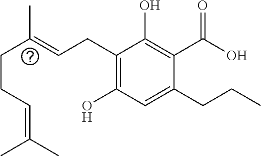 [SMILES: CCCc1cc(O)c(C/C=C(\C)CCC=C(C)C)c(O)c1C(=O)O]

Cannabigerovarinic
acid A
(E)-CBGVA-C₃ A

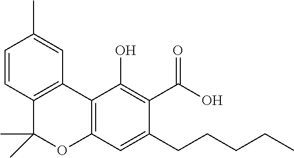 [SMILES: CCCCCc1cc2c(c(O)c1C(=O)O)-c1cc(C)ccc1C(C)(C)O2]

Cannabinolic acid A
CBNA-C5 A

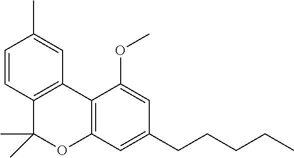 [SMILES: CCCCCc1cc(OC)c2c(c1)OC(C)(C)c1ccc(C)cc1-2]

Cannabinol methyl
ether
CBNM-C5

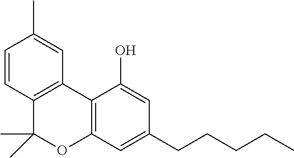 [SMILES: CCCCCc1cc(O)c2c(c1)OC(C)(C)c1ccc(C)cc1-2]

Cannabinol
CBN-C5

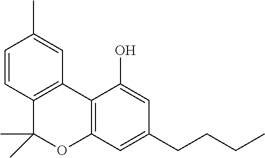 [SMILES: CCCCc1cc(O)c2c(c1)OC(C)(C)c1ccc(C)cc1-2]

Cannabinol-C4
CBN-C4

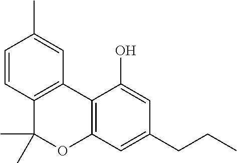 [SMILES: CCCc1cc(O)c2c(c1)OC(C)(C)c1ccc(C)cc1-2]

Cannabivarin
CBN-C3

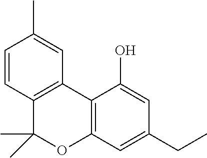 [SMILES: CCc1cc(O)c2c(c1)OC(C)(C)c1ccc(C)cc1-2]

Cannabinol-C2
CBN-C2

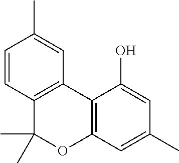

Cannabiorcol
CBN-C1

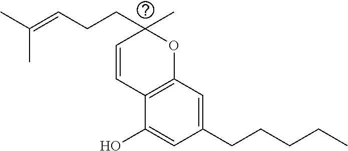 [SMILES: CCCCCc1cc(O)c2c(c1)OC(C)(CCC=C(C)C)C=C2]

(±)-Cannabichromene
CBC-C₅

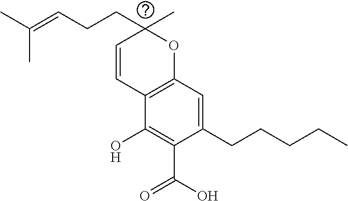 [SMILES: CCCCCc1cc2c(c(O)c1C(=O)O)C=CC(C)(CCC=C(C)C)O2]

(±)-Cannabichromenic
acid A
CBCA-C₅ A

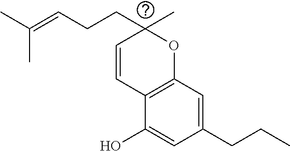 [SMILES: CCCc1cc(O)c2c(c1)OC(C)(CCC=C(C)C)C=C2]

(±)-
Cannabivarichromene,
(±)-
Cannabichromevarin
CBCV-C₃

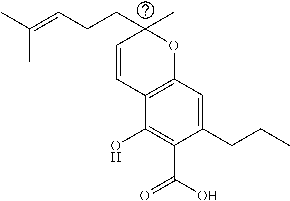 [SMILES: CCCc1cc2c(c(O)c1C(=O)O)C=CC(C)(CCC=C(C)C)O2]

(±)-Cannabichro-
mevarinic
acid A
CBCVA-C₃ A

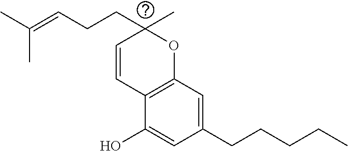 [SMILES: CCCCCc1cc(O)c2c(c1)OC(C)(CCC=C(C)C)C=C2]

(±)-Cannabichromene
CBC-C₅

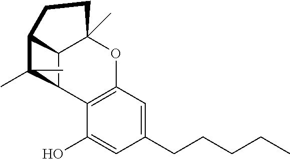

(±)-(1aS,3aR,8bR,8cR)-
Cannabicyclol
CBL-C₅

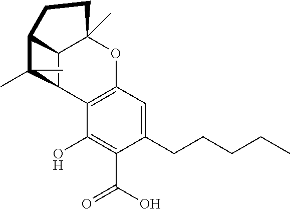

(±)-(1aS,3aR,8bR,8cR)-
Cannabicyclolic acid A
CBLA-C₅ A

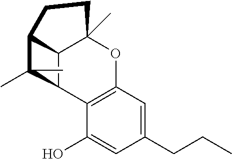

(±)-
(1aS,3aR,8bR,8cR)-
Cannabicyclovarin
CBLV-C₃

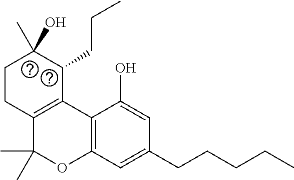 [SMILES: CCCCCc1cc(O)c2c(c1)OC(C)(C)C1=C2[C@@H](CCC)[C@](C)(O)CC1]

(−)-(9R,10R)-trans-
10-O-Ethyl-
cannabitriol
(−)-trans-CBT-OEt-C5

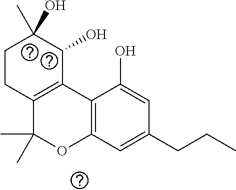 [SMILES: CCCc1cc(O)c2c(c1)OC(C)(C)C1=C2[C@@H](O)[C@](C)(O)CC1]

(±)-(9R,10R/9S,10S)-
Cannabitriol-C3
(±)-trans-CBT-C3

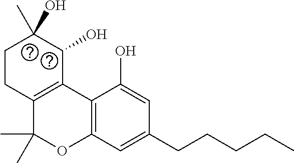 [SMILES: CCCCCc1cc(O)c2c(c1)OC(C)(C)C1=C2[C@@H](O)[C@](C)(O)CC1]

(−)-(9R,10R)-trans-
Cannabitriol
(−)-trans-CBT-C5

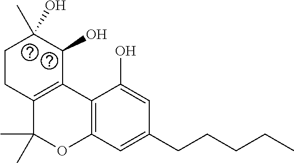 [SMILES: CCCCCc1cc(O)c2c(c1)OC(C)(C)C1=C2[C@H](O)[C@@](C)(O)CC1]

(+)-(9S,10S)-
Cannabitriol
(+)-trans-CBT-C5

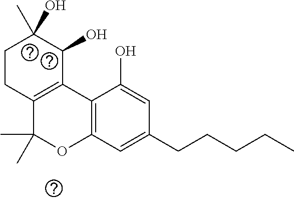 [SMILES: CCCCCc1cc(O)c2c(c1)OC(C)(C)C1=C2[C@H](O)[C@](C)(O)CC1]

(±)-(9R,10S/9S,10R)-
Cannabitriol
(±)-cis-CBT-C5

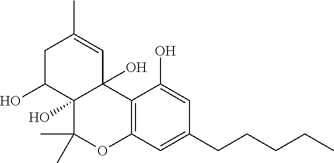 [SMILES: CCCCCc1cc(O)c2c(c1)OC(C)(C)[C@@]1(O)C(O)CC(C)=CC21O]

(−)-6a,7,10a-Trihydroxy-
Δ9-tetrahydro-
cannabinol
(−)-Cannabitetrol

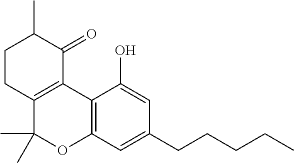 [SMILES: CCCCCc1cc(O)c2c(c1)OC(C)(C)C1=C2C(=O)C(C)CC1]

10-Oxo-Δ6a(10a)-
tetrahydro-
cannabinol
OTHC

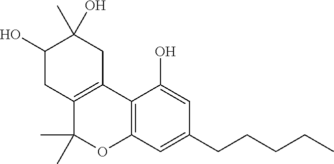 [SMILES: CCCCCc1cc(O)c2c(c1)OC(C)(C)C1=C2CC(C)(O)C(O)C1]

8,9-Dihydroxy-
Δ6a(10a)-
tetrahydro-
cannabinol
8,9-Di-OH-CBT-C5

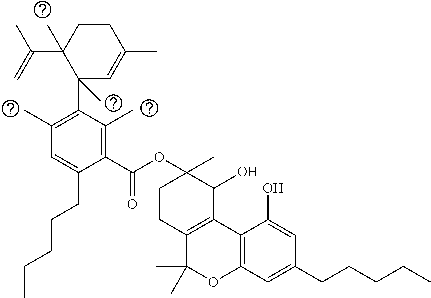 [SMILES: C=C(C)C1(C)CCC(C)=CC1(C)c1c(C)cc(CCCCC)c(C(=O)OC2(C)CCC3=C(c4c(O)cc(CCCCC)cc4OC3(C)C)C2O)c1C]

Cannabidiolic acid A
cannabitriol ester
CBDA-C5 9-OH-CBT-
C5 ester

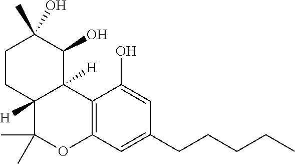 [SMILES: CCCCCc1cc(O)c2c(c1)OC(C)(C)[C@@H]1CC[C@](C)(O)[C@@H](O)[C@@H]21]

(−)-
(6aR,98,10S,10aR)-
9,10-Dihydroxy-
hexahydrocannabinol,
Cannabiripsol
Cannabiripsol-C5

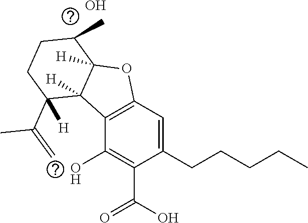

(5aS,6S,9R,9aR)-
Cannabielsoic acid B
CBEA-C5 B

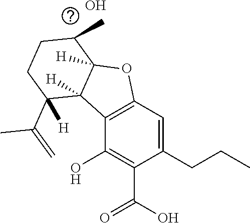

(5aS,68,9R,9aR)-
C3-Cannabielsoic
acid B
CBEA-C3 B

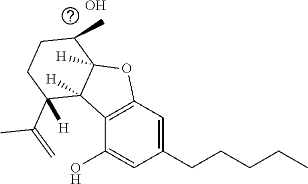

(5aS,6S,9R,9aR)-
Cannabielsoin
CBE-C5

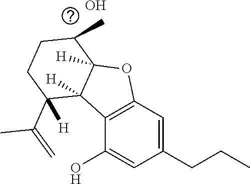

(5aS,6S,9R,9aR)-
C3-Cannabielsoin
CBB-C3

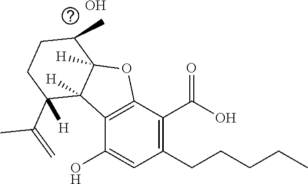

(5aS,68,9R,9aR)-
Cannabielsoic acid A.
CBEA-C5 A

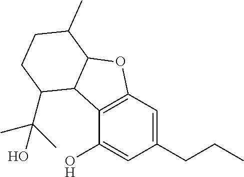 [SMILES: CCCc1cc(O)c2c(c1)OC1C(C)CCC(C(C)(C)O)C21]

Cannabiglendol-C3
OH-iso-HHCV-C3

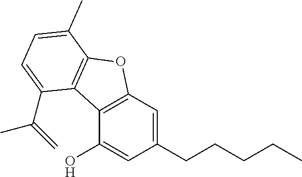 [SMILES: C=C(C)c1ccc(C)c2oc3cc(CCCCC)cc(O)c3c12]

Dehydro-
cannabifuran
DCBF-C5

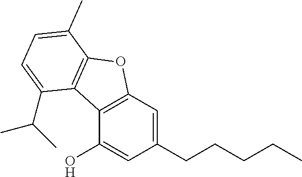 [SMILES: CCCCCc1cc(O)c2c(c1)oc1c(C)ccc(C(C)C)c12]

Cannabifuran
CBF-C5

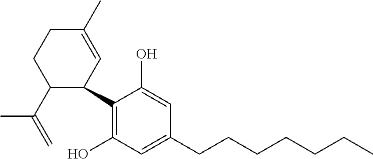 [SMILES: C=C(C)C1CCC(C)=C[C@H]1c1c(O)cc(CCCCCCC)cc1O]

Cannabidiphorol
(CBDP)

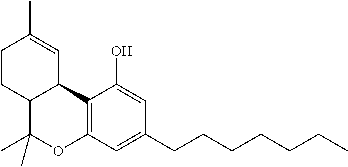

Tetrahydro-
cannabiphorol
(THCP)

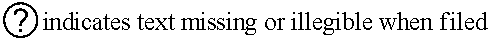

Cannabinoids are often classified by “type”, i.e., by the topological arrangement of their prenyl moieties (See, for example, M. A. Elsohly and D. Slade, Life Sci., 2005, 78, 539-548; and L. O. Hanus et al. Nat. Prod. Rep., 2016, 33, 1357). Generally, each “type” of cannabinoid includes the variations possible for ring substitutions of the resorcinol moiety at the position meta to the two hydroxyl moieties. As used herein, a “CBG-type” cannabinoid is a 3-[(2E)-3,7-dimethylocta-2,6-dienyl]-2,4-dihydroxybenzoic acid optionally substituted at the 6 position of the benzoic acid moiety. As used herein, “CBC-type” cannabinoids refer to 5-hydroxy-2-methyl-2-(4-methylpent-3-enyl)-chromene-6-carboxylic acid optionally substituted at the 7 position of the chromene moiety. As used herein, a “THC-type” cannabinoid is a (6aR,10aR)-1-hydroxy-6,6,9-trimethyl-6a,7,8,10a-tetrahydrobenzo[c]chromene-2-carboxylic acid optionally substituted at the 3 position of the benzo[c]chromene moiety. As used herein, a “CBD-type” cannabinoid is a 2,4-dihydroxy-3-[(1R,6R)-3-methyl-6-prop-1-en-2-ylcyclohex-2-en-1-yl]-benzoic acid optionally substituted at the 6 position of the benzoic acid moiety. In some embodiments, the optional ring substitution for each “type” is an optionally substituted C1-C11 alkyl, an optionally substituted C1-C11 alkenyl, an optionally substituted C1-C11 alkynyl, or an optionally substituted C1-C11 aralkyl.

### Biosynthesis of Cannabinoids and Cannabinoid Precursors

Aspects of the present disclosure provide tools, sequences, and methods for the biosynthetic production of cannabinoids in host cells. In some embodiments, the present disclosure teaches expression of enzymes that are capable of producing cannabinoids by biosynthesis.

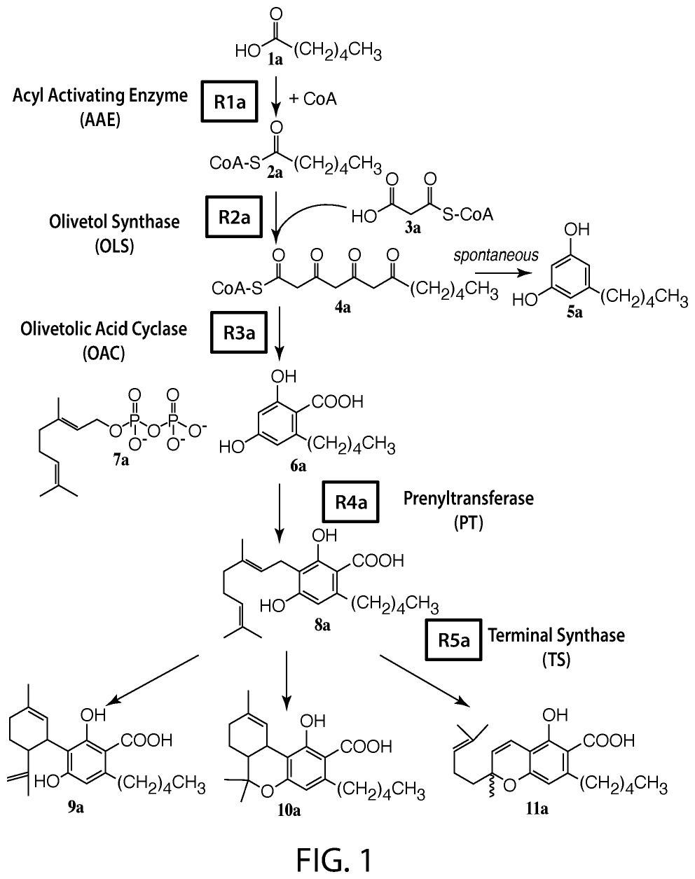

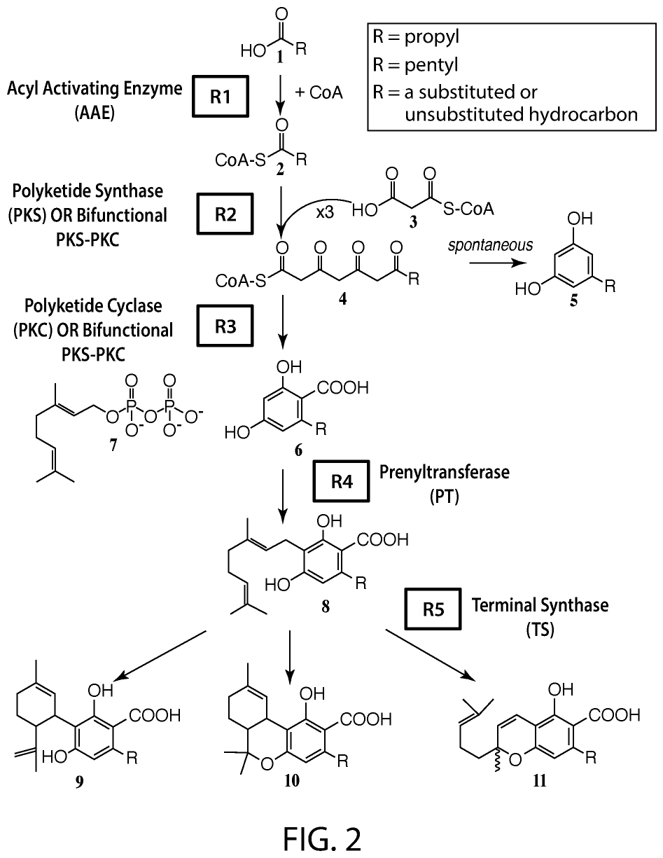

As a non-limiting example, one or more of the enzymes depicted in FIG. 2 may be used to produce a cannabinoid or cannabinoid precursor of interest. FIG. 1 shows a cannabinoid biosynthesis pathway for the most abundant phytocannabinoids found in Cannabis. See also, de Meijer et al. I, II, III, and IV (I: 2003, Genetics, 163:335-346; II: 2005, Euphytica, 145:189-198; III: 2009, Euphytica, 165:293-311; and IV: 2009, Euphytica, 168:95-112), and Carvalho et al. “Designing Microorganisms for Heterologous Biosynthesis of Cannabinoids” (2017) FEMS Yeast Research June 1:17(4), each of which is incorporated by reference in this application in its entirety for all purposes.

It should be appreciated that a precursor substrate for use in cannabinoid biosynthesis is generally selected based on the cannabinoid of interest. Non-limiting examples of cannabinoid precursors include compounds of Formulae (1)-(8) in FIG. 2. In some embodiments, polyketides, including compounds of Formula (5), could be prenylated. In certain embodiments, the precursor is a precursor compound shown in FIG. 1, 2, or 3. Substrates in which R contains 1-40 carbon atoms are preferred. In some embodiments, substrates in which R contains 3-8 carbon atoms are most preferred.

As used in this application, a cannabinoid or a cannabinoid precursor may comprise an R group. See, e.g., FIG. 2. In some embodiments, R may be a hydrogen. In certain embodiments, R is optionally substituted alkyl. In certain embodiments, R is optionally substituted C₁₋₄₀ alkyl. In certain embodiments, R is optionally substituted C2-40 alkyl. In certain embodiments, R is optionally substituted C2-40 alkyl, which is straight chain or branched alkyl. In certain embodiments, R is optionally substituted C3-8 alkyl. In certain embodiments, R is optionally substituted C1-C40 alkyl, C1-C20 alkyl, C1-C10 alkyl, C1-C8 alkyl, C1-C5 alkyl, C3-C5 alkyl, C3 alkyl, or C5 alkyl. In certain embodiments, R is optionally substituted C1-C20 alkyl. In certain embodiments, R is optionally substituted C1-C10 alkyl. In certain embodiments, R is optionally substituted C1-C8 alkyl. In certain embodiments, R is optionally substituted C1-C5 alkyl. In certain embodiments, R is optionally substituted C1-C7 alkyl. In certain embodiments, R is optionally substituted C3-C5 alkyl. In certain embodiments, R is optionally substituted C3 alkyl. In certain embodiments, R is unsubstituted C3 alkyl. In certain embodiments, R is n-C3 alkyl. In certain embodiments, R is n-propyl. In certain embodiments, R is n-butyl. In certain embodiments, R is n-pentyl. In certain embodiments, R is n-hexyl. In certain embodiments, R is n-heptyl. In certain embodiments, R is of formula:

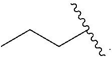 [SMILES: *CCC]

In certain embodiments, R is optionally substituted C4 alkyl. In certain embodiments, R is unsubstituted C4 alkyl. In certain embodiments, R is optionally substituted C5 alkyl. In certain embodiments, R is unsubstituted C5 alkyl. In certain embodiments, R is optionally substituted C6 alkyl. In certain embodiments, R is unsubstituted C6 alkyl. In certain embodiments, R is optionally substituted C7 alkyl. In certain embodiments, R is unsubstituted C7 alkyl. In certain embodiments, R is of formula:

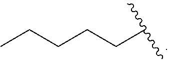 [SMILES: *CCCCC]

In certain embodiments, R is of formula:

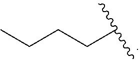 [SMILES: *CCCC]

In certain embodiments, R is of formula:

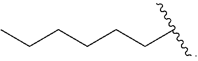 [SMILES: *CCCCCC]

In certain embodiments, R is of formula

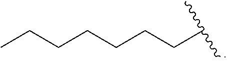 [SMILES: *CCCCCCC]

In certain embodiments, R is of formula:

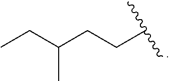 [SMILES: *CCC(C)CC]

In certain embodiments, R is optionally substituted n-propyl. In certain embodiments, R is n-propyl optionally substituted with optionally substituted aryl. In certain embodiments, R is n-propyl optionally substituted with optionally substituted phenyl. In certain embodiments, R is n-propyl substituted with unsubstituted phenyl. In certain embodiments, R is optionally substituted butyl. In certain embodiments, R is optionally substituted n-butyl. In certain embodiments, R is n-butyl optionally substituted with optionally substituted aryl. In certain embodiments, R is n-butyl optionally substituted with optionally substituted phenyl. In certain embodiments, R is n-butyl substituted with unsubstituted phenyl. In certain embodiments, R is optionally substituted pentyl. In certain embodiments, R is optionally substituted n-pentyl. In certain embodiments, R is n-pentyl optionally substituted with optionally substituted aryl. In certain embodiments, R is n-pentyl optionally substituted with optionally substituted phenyl. In certain embodiments, R is n-pentyl substituted with unsubstituted phenyl. In certain embodiments, R is optionally substituted hexyl. In certain embodiments, R is optionally substituted n-hexyl. In certain embodiments, R is optionally substituted n-heptyl. In certain embodiments, R is optionally substituted n-octyl. In certain embodiments, R is alkyl optionally substituted with aryl (e.g., phenyl). In certain embodiments. R is optionally substituted acyl (e.g., —C(═O)Me).

In certain embodiments, R is optionally substituted alkenyl (e.g., substituted or unsubstituted C₂₋₆ alkenyl). In certain embodiments, R is substituted or unsubstituted C₂₋₆ alkenyl. In certain embodiments, R is substituted or unsubstituted C₂₋₅ alkenyl. In certain embodiments, R is of formula:

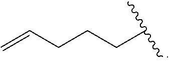 [SMILES: *CCCC=C]

In certain embodiments, R is optionally substituted alkynyl (e.g., substituted or unsubstituted C₂₋₆ alkynyl). In certain embodiments, R is substituted or unsubstituted C₂₋₆ alkynyl. In certain embodiments, R is of formula:

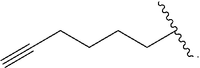 [SMILES: *CCCCC#C]

In certain embodiments, R is optionally substituted carbocyclyl. In certain embodiments, R is optionally substituted aryl (e.g., phenyl or napthyl).

The chain length of a precursor substrate can be from C1-C40. Those substrates can have any degree and any kind of branching or saturation or chain structure, including, without limitation, aliphatic, alicyclic, and aromatic. In addition, they may include any functional groups including hydroxy, halogens, carbohydrates, phosphates, methyl-containing or nitrogen-containing functional groups.

In some embodiments, R is H, an optionally substituted C1-C11 alkyl, an optionally substituted C1-C11 alkenyl, an optionally substituted C1-C11 alkynyl, or an optionally substituted C1-C11 aralkyl.

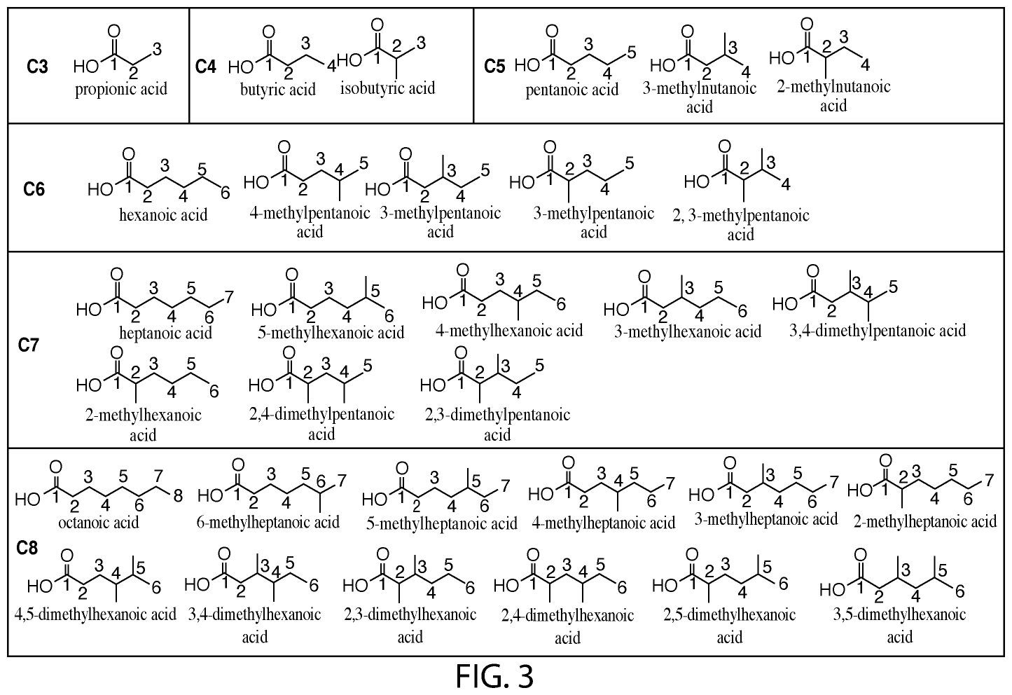

For example, FIG. 3 shows a non-exclusive set of putative precursors for the cannabinoid pathway. Aliphatic carboxylic acids including four to eight total carbons (“C4”-“C8” in FIG. 3) and up to 10-12 total carbons with either linear or branched chains may be used as precursors for the heterologous pathway. Non-limiting examples include methanoic acid, butyric acid, pentanoic acid, hexanoic acid, heptanoic acid, isovaleric acid, octanoic acid, and decanoic acid. Additional precursors may include ethanoic acid and propanoic acid. In some embodiments, in addition to acids, the ester, salt, and acid forms may all be used as substrates. Substrates may have any degree and any kind of branching, saturation, and chain structure, including, without limitation, aliphatic, alicyclic, and aromatic. In addition, they may include any functional modifications or combination of modifications including, without limitation, halogenation, hydroxylation, amination, acylation, alkylation, phenylation, and/or installation of pendant carbohydrates, phosphates, sulfates, heterocycles, or lipids, or any other functional groups.

Substrates for any of the enzymes disclosed in this application may be provided exogenously or may be produced endogenously by a host cell. In some embodiments, the cannabinoids are produced from a glucose substrate, so that compounds of Formula 1 shown in FIG. 2 and CoA precursors are synthesized by the cell. In other embodiments, a precursor is fed into the reaction. In some embodiments, a precursor is a compound selected from Formulae 1-8 in FIG. 2.

Cannabinoids produced by methods disclosed in this application include rare cannabinoids. Due to the low concentrations at which cannabinoids, including rare cannabinoids, occur in nature, producing industrially significant amounts of isolated or purified cannabinoids from the Cannabis plant may become prohibitive, especially in the case of rare cannabinoids, due to, e.g., the large volumes of Cannabis plants, and the large amounts of space, labor, time, and capital requirements to grow, harvest, and/or process the plant materials (see, for example, Crandall, K., 2016. A Chronic Problem: Taming Energy Costs and Impacts from Marijuana Cultivation. EQ Research; Mills, E., 2012. The carbon footprint of indoor Cannabis production. Energy Policy, 46, pp. 58-67; Jourabchi. M. and M. Lahet. 2014. Electrical Load Impacts of Indoor Commercial Cannabis Production. Presented to the Northwest Power and Conservation Council; O'Hare, M., D. Sanchez, and P. Alstone. 2013. Environmental Risks and Opportunities in Cannabis Cultivation. Washington State Liquor and Cannabis Board; 2018. Comparing Cannabis Cultivation Energy Consumption. New Frontier Data; and Madhusoodanan. J., 2019. Can Cannabis go green? Nature Outlook: Cannabis; all of which are incorporated by reference in this disclosure). The disclosure provided in this application represents a potentially efficient method for producing high yields of cannabinoids, including rare cannabinoids. The disclosure provided in this application also represents a potential method for addressing concerns related to agricultural practices and water usage associated with traditional methods of cannabinoid production (Dillis et al. “Water storage and irrigation practices for Cannabis drive seasonal patterns of water extraction and use in Northern California.” Journal of Environmental Management 272 (2020): 110955, incorporated by reference in this disclosure).

Cannabinoids produced by the disclosed methods also include non-rare cannabinoids. Without being bound by a particular theory, the methods described in this application may be advantageous compared with traditional plant-based methods for producing non-rare cannabinoids. For example, methods provided in this application represent potentially efficient means for producing consistent and high yields of non-rare cannabinoids. With traditional methods of cannabinoid production, in which cannabinoids are harvested from plants, maintaining consistent and uniform conditions, including airflow, nutrients, lighting, temperature, and humidity, can be difficult. For example, with plant-based methods, there can be microclimates created by branching, which can lead to inconsistent yields and by-product formation. In some embodiments, the methods described in this application are more efficient at producing a cannabinoid of interest as compared to harvesting cannabinoids from plants. For example, with plant-based methods, seed-to-harvest can take up to half a year, while cutting-to-harvest usually takes about 4 months. Additional steps including drying, curing, and extraction are also usually needed with plant-based methods. In contrast, in some embodiments, the fermentation-based methods described in this application only take about 1, 2, 3, 4, 5, 6, 7, 8, 9, or 10 days. In some embodiments, the fermentation-based methods described in this application only take about 3-5 days. In some embodiments, the fermentation-based methods described in this application only take about 5 days. In some embodiments, the methods provided in this application reduce the amount of security needed to comply with regulatory standards. For example, a smaller secured area may be needed to be monitored and secured to practice the methods described in this application as compared to the cultivation of plants. In some embodiments, the methods described in this application are advantageous over plant-sourced cannabinoids.

### Penyltransferase (PT)

Aspects of the disclosure relate to prenyltransferase (PT) enzymes. As used in this disclosure, a “PT” refers to an enzyme that is capable of transferring prenyl groups to acceptor molecule substrates. Non-limiting examples of prenyltransferases are described in U.S. Pat. No. 7,544,498 and Kumano et al., Bioorg Med Chem. 2008 Sep. 1; 16(17): 8117-8126 (e.g., NphB), PCT Publication No. WO 2018/200888 (e.g., CsPT4), U.S. Pat. No. 8,884,100 (e.g., CsPT1); CA2718469; Valliere et al., Nat Commun. 2019 Feb. 4; 10(1):565 (e.g., NphB variants); PCT Publication Nos: WO2019/173770, WO2019/183152, and WO2020/210810 (e.g., NphB variants); Luo et al., Nature 2019 March; 567(7746):123-126 (e.g., CsPT4); and WO2021/034848. In some embodiments, a PT is capable of producing cannabigerolic acid (CBGA), cannabigerophorolic acid (CBGPA), cannabigerovarinic acid (CBGVA), a CBG-type cannabinoid, or other cannabinoids or cannabinoid-like substances. In some embodiments, a PT is a cannabigerolic acid synthase (CBGAS). In some embodiments, a PT is cannabigerovarinic acid synthase (CBGVAS).

In some embodiments, the PT is a NphB prenyltransferase. See, e.g., U.S. Pat. No. 7,544,498; and Kumano et al., Bioorg Med Chem. 2008 Sep. 1; 16(17): 8117-8126, which are incorporated by reference in this application in their entireties. In some embodiments, a PT corresponds to NphB from Streptomyces sp. (see, e.g., UniprotKB Accession No. Q4R2T2, see also SEQ ID NO: 2 of U.S. Pat. No. 7,361,483). The protein sequence corresponding to UniprotKB Accession No. Q4R2T2 is provided by SEQ ID NO: 1:

(SEQ ID NO: 1)
MSEAADVERVYAAMEEAAGLLGVACARDKIYPLLSTFQDT
LVEGGSVVVFSMASGRHSTELDFSISVPTSHGDPYATVVE
KGLFPATGHPVDDLLADTQKHLPVSMFAIDGEVTGGFKKT
YAFFPTDNMPGVAELSAIPSMPPAVAENAELFARYGLDKV
QMTSMDYKKRQVNLYFSELSAQTLEAESVLALVRELGLHV
PNELGLKFCKRSFSVYPTLNWETGKIDRLCFAVISNDPTL
VPSSDEGDIEKFHNYATKAPYAYVGEKRTLVYGLTLSPKE
EYYKLGAYYHITDVQRGLLKAFDSLED.

A non-limiting example of a nucleic acid sequence encoding NphB is:

(SEQ ID NO: 2)
atgtcagaagccgcagatgtcgaaagagtttacgccgcta
tggaagaagccgccggittgttaggtgttgcctgtgccag
agataagatctacccattgttgtctacttttcaagataca
ttagttgaaggggttcagttgttgttttctctatggcttc
aggtagacattctacagaattggatttctctatctcagtt
ccaacatcacatggtgatccatacgctactgttgttgaaa
aaggtttatttccagcaacaggtcatccagttgatgattt
gttggctgatactcaaaagcatttgccagittctatgtit
gcaattgatggtgaagttactggtggtttcaagaaaactt
acgctttctttccaactgataacatgccaggtgttgcaga
attatctgctattccatcaatgccaccagctgttgcagaa
aatgcagaattatttgctagatacggtttggataaggttc
aaatgacatctatggattacaagaaaagacaagttaattt
gtactittctgaattatcagcacaaactitggaagctgaa
tcagttttggcattagttagagaattgggtitacatgttc
caaacgaattgggtttgaagttttgtaaaagatctttctc
agtttatccaactttaaactgggaaacaggcaagatcgat
agattatgtttcgcagttatctctaacgatccaacattgg
ttccatcttcagatgaaggtgatatcgaaaagtttcataa
ctacgctactaaagcaccatatgcttacgttggtgaaaag
agaacattagtttatggittgactttatcaccaaaggaag
aatactacaagttgggtgcttactaccacattaccgacgt
acaaagaggtttattgaaagcattcgatagtttagaagac
taa.

In other embodiments, a PT is CsPT1, which is disclosed as SEQ ID NO:2 in U.S. Pat. No. 8,884,100, corresponding to SEQ ID NO: 3 in this application:

(SEQ ID NO: 3)
MGLSSVCTFSFQTNYHTLLNPHNNNPKTSLLCYRHPKTPI
KYSYNNFPSKHCSTKSFHLQNKCSESLSIAKNSIRAATTN
QTEPPESDNHSVATKILNFGKACWKLQRPYTIIAFTSCAC
GLFGKELLHNTNLISWSLMFKAFFFLVAILCIASFTTTIN
QIYDLHIDRINKPDLPLASGEISVNTAWIMSIIVALFGLI
ITIKMKGGPLYIFGYCFGIFGGIVYSVPPFRWKQNPSTAF
LLNFLAHIITNFTFYYASRAALGLPFELRPSFTFLLAFMK
SMGSALALIKDASDVEGDTKFGISTLASKYGSRNLTLFCS
GIVLLSYVAAILAGIIWPQAFNSNVMLLSHAILAFWLILQ
TRDFALTNYDPEAGRRFYEFMWKLYYAEYLVYVFI.

In some embodiments, a PT is a truncated CsPT1. In some embodiments, a truncated CsPT1 corresponds to SEQ ID NO: 1185:

(SEQ ID NO: 1185)
MAATTNQTEPPESDNHSVATKILNFGKACWKLQRPYTIIAFTSCACGLF
GKELLHNTNLISWSLMFKAFFFLVAILCIASFTTTINQIYDLHIDRINK
PDLPLASGEISVNTAWIMSIIVALFGLIITIKMKGGPLYIFGYCFGIFG
GIVYSVPPFRWKQNPSTAFLLNFLAHIITNFTFYYASRAALGLPFELRP
SFTFLLAFMKSMGSALALIKDASDVEGDTKFGISTLASKYGSRNLTLFc
SGIVLLSYVAAILAGIIWPQAFNSNVMLLSHAILAFWLILQTRDFALTN
YDPEAGRRFYEFMWKLYYAEYLVYVFI.

In some embodiments, a PT is CsPT4, which is disclosed as SEQ ID NO:1 in WO 2019/071000, corresponding to SEQ ID NO: 4 in this application;

(SEQ ID NO: 4)
MGLSLVCTFSFQTNYHTLLNPHNKNPKNSLLSYQHPKTPIIKSSYDNFP
SKYCLTKNFHLLGLNSHNRISSQSRSIRAGSDQIEGSPHHESDNSIATK
ILNFGHTCWKLQRPYVVKGMISIACGLFGRELFNNRHLFSWGLMWKAFF
ALVPILSFNFFAAIMNQIYDVDIDRINKPDLPLVSGEMSIETAWILSII
VALTGLIVTIKLKSAPLFVFIYIFGIFAGFAYSVPPIRWKQYPFTNFLI
TISSHVGLAFTSYSATTSALGLPFVWRPAFSFIIAFMTVMGMTIAFAKD
ISDIEGDAKYGVSTVATKLGARNMTFVVSGVLLLNYLVSISIGIIWPQV
FKSNIMILSHAILAFCLIFQTRELALANYASAPSRQFFEFIWLLYYAEY
FVYVFI.

In some embodiments, a PT is a truncated CsPT4. In some embodiments, a truncated CsPT4 is provided by SEQ ID NO: 5;

(SEQ ID NO: 5)
MSAGSDQIEGSPHHESDNSIATKILNFGHTCWKLQRPYVVKGMISIACG
LFGRELFNNRHLFSWGLMWKAFFALVPILSFNFFAAIMNQIYDVDIDRI
NKPDLPLVSGEMSIETAWILSIIVALTGLIVTIKLKSAPLFVFIYIFGI
FAGFAYSVPPIRWKQYPFTNFLITISSHVGLAFTSYSATTSALGLPFVW
RPAFSFIIAFMTVMGMTIAFAKDISDIEGDAKYGVSTVATKLGARNMTF
VVSGVLLLNYLVSISIGIIWPQVFKSNIMILSHAILAFCLIFQTRELAL
ANYASAPSRQFFEFIWLLYYAEYFVYVFI.

In some embodiments, a truncated CsPT4 is provided by SEQ ID NO: 6.

(SEQ ID NO: 6)
SAGSDQIEGSPHHESDNSIATKILNFGHTCWKLQRPYVVKGMISIACGL
FGRELFNNRHLFSWGLMWKAFFALVPILSFNFFAAIMNQIYDVDIDRIN
KPDLPLVSGEMSIETAWILSIIVALTGLIVTIKLKSAPLFVFIYIFGIF
AGFAYSVPPIRWKQYPFTNFLITISSHVGLAFTSYSATTSALGLPFVWR
PAFSFIIAFMTVMGMTIAFAKDISDIEGDAKYGVSTVATKLGARNMTFV
VSGVLLLNYLVSISIGIIWPQVFKSNIMILSHAILAFCLIFQTRELALA
NYASAPSRQFFEFIWLLYYAEYFVYVFI.

In some embodiments, a truncated CsPT4 is provided by SEQ ID NO: 7.

(SEQ ID NO: 7)
IEGSPHHESDNSIATKILNFGHTCWKLQRPYVVKGMISIACGLFGRELF
NNRHLFSWGLMWKAFFALVPILSFNFFAAIMNQIYDVDIDRINKPDLPL
VSGEMSIETAWILSIIVALTGLIVTIKLKSAPLFVFIYIFGIFAGFAYS
VPPIRWKQYPFTNFLITISSHVGLAFTSYSATTSALGLPFVWRPAFSFI
IAFMTVMGMTIAFAKDISDIEGDAKYGVSTVATKLGARNMTFVVSGVLL
LNYLVSISIGIIWPQVFKSNIMILSHAILAFCLIFQTRELALANYASAP
SRQFFEFIWLLYYAEYFVYVFI.

In some embodiments, a truncated CsPT4 is provided by SEQ ID NO: 8.

(SEQ ID NO: 8)
HHESDNSIATKILNFGHTCWKLQRPYVVKGMISIACGLFGRELFNNRHL
FSWGLMWKAFFALVPILSFNFFAAIMNQIYDVDIDRINKPDLPLVSGEM
SIETAWILSIIVALTGLIVTIKLKSAPLFVFIYIFGIFAGFAYSVPPIR
WKQYPFTNFLITISSHVGLAFTSYSATTSALGLPFVWRPAFSFIIAFMT
VMGMTIAFAKDISDIEGDAKYGVSTVATKLGARNMTFVVSGVLLLNYLV
SISIGIIWPQVFKSNIMILSHAILAFCLIFQTRELALANYASAPSRQFF
EFIWLLYYAEYFVYVFI.

In some embodiments, a PT is CsPT6, which is provided by SEQ ID NO: 9, corresponding to UniProt Accession No. A0A455ZIL7.

(SEQ ID NO: 9)
SSDLPSVLSKGGSNWRRCNLKNVEFSGSYVAYNVSRLRVWKVREKPCSA
VFQPSSLKHCAKGSETFVFYQRPNERFLVKAAGGQPLESEPKNDMNSAK
DALDAFYRFSRPHTVIGTALSIVSVSLLAIEKLSDFSPLFFVGMLEAIV
AALLMNIYIVGLNQLYDIDIDKVNKPYLPLASGEYSIQTGVMIVASFSI
LSFGVGWLVGSWPLFWALFISFVLGTAYSINVPLLRWKRFALVAAMCIL
AVRAVIVQLAFFLHIQTHVFKRPAVFSRPLIFATAFMSFFSVVIALFKD
IPDIDGDRIYGIRSFTVRLGQKRVFWICISLLEIAYTVALLVGASSGFL
WSKVVTVLGHTILASILWTNAKSVDLSSKAAITSFYMFIWKLFYAEYLL
IPLVR.

In other embodiments, a PT is a truncated CsPT6. In some embodiments, a truncated CsPT6 is provided by SEQ ID NO: 701.

(SEQ ID NO: 701)
MSYVAYNVSRLRVWKVREKPCSAVFQPSSLKHCAKGSETFVFYQRPNER
FLVKAAGGQPLESEPKNDMNSAKDALDAFYRFSRPHTVIGTALSIVSVS
LLAIEKLSDFSPLFFVGMLEAIVAALLMNIYIVGLNQLYDIDIDKVNKP
YLPLASGEYSIQTGVMIVASFSILSFGVGWLVGSWPLFWALFISFVLGT
AYSINVPLLRWKRFALVAAMCILAVRAVIVQLAFFLHIQTHVFKRPAVF
SRPLIFATAFMSFFSVVIALFKDIPDIDGDRIYGIRSFTVRLGQKRVFW
ICISLLEIAYTVALLVGASSGFLWSKVVTVLGHTILASILWTNAKSVDL
SSKAAITSFYMFIWKLFYAEYLLIPLVR.

In some embodiments, a PT is CsPT7, which is provided by SEQ ID NO: 10, corresponding to UniProt Accession No. A0A455ZJ77.

MELSSICNFSFQTNYHTLLNPHNKNPKSSLLSHQHPKTPIITSSYNNFP
SNYCSNKNFHLQNRCSKSLLIAKNSIRTDTANQTEPPESNTKYSVVTKI
LSFGHTCWKLQRPYTFIGVISCACGLFGRELFHNTNLLSWSLMLKAFSS
LMVILSVNLCTNIINQITDLDIDRINKPDLPLASGEMSIETAWIMSIIV
ALTGLILTIKLNCGPLFISLYCVSILVGALYSVPPFRWKQNPNTAFSSY
FMGLVIVNFTCYYASRAAFGLPFEMSPPFTFILAFVKSMGSALFLCKDV
SDIEGDSKHGISTLATRYGAKNITFLCSGIVLLTYVSAILAAIIWPQAF
KSNVMLLSHATLAFWLIFQTREFALTNYNPEAGRKFYEFMWKLHYAEYL
VYVFI.

In other embodiments, a CsPT is a truncated CsPT7. In some embodiments, a truncated CsPT7 is provided by SEQ ID NO: 702

(SEQ ID NO: 702)
MSTDTANQTEPPESNTKYSVVTKILSFGHTCWKLQRPYTFIGVISCACG
LFGRELFHNTNLLSWSLMLKAFSSLMVILSVNLCTNIINQITDLDIDRI
NKPDLPLASGEMSIETAWIMSIIVALTGLILTIKLNCGPLFISLYCVSI
LVGALYSVPPFRWKQNPNTAFSSYFMGLVIVNFTCYYASRAAFGLPFEM
SPPFTFILAFVKSMGSALFLCKDVSDIEGDSKHGISTLATRYGAKNITF
LCSGIVLLTYVSAILAAIIWPQAFKSNVMLLSHATLAFWLIFQTREFAL
TNYNPEAGRKFYEFMWKLHYABYLVYVFI.

a. Chimeric Prenyltransferase

Examples 1-8 describe identification of synthetic PTs that can be functionally expressed in host cells such as S. cerevisiae. Nucleic acid and protein sequences for PTs identified in this application are provided in Tables 13-16 and 19-20.

PTs provided in this disclosure include chimeric PTs. As used in this disclosure, a “chimeric PT” refers to a PT that includes one or more portions of at least two different PT proteins. It has previously been reported that it is difficult to express C. sativa PTs in S. cerevisiae; for example, out of CsPT1-7, only CsPT4 was reported to produce CBGA when expressed heterologously in S. cerevisiae, and only at low titers (Luo et al., Nature 2019 March; 567(7746):123-126). It was surprisingly shown in Examples 1-8 of this disclosure that chimeric PTs, such as PTs that included portions of at least two of CsPT1, CsPT4, CsPT6, and CsPT7, were able to produce CBGA and/or CBGVA.

In some embodiments, chimeric PTs comprise one or more portions of CsPT1 and one or more portions of a non-CsPT1 PT. A portion can include, for example, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, or more than 390 amino acids. In some embodiments, a non-CsPT1 PT is a PT from C. sativa. In some embodiments, a non-CsPT1 PT is CsPT4, CsPT6, or CsPT7.

In some embodiments, chimeric PTs comprise one or more portions of CsPT4 and one or more portions of a non-CsPT4 PT. A portion can include, for example, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, or more than 390 amino acids. In some embodiments, a non-CsPT4 PT is a PT from C. sativa. In some embodiments, a non-CsPT4 PT is CsPT1, CsPT6, or CsPT7.

In some embodiments, chimeric PTs comprise one or more portions of CsPT6 and one or more portions of a non-CsPT6 PT. A portion can include, for example, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, or more than 390 amino acids. In some embodiments, a non-CsPT6 PT is a PT from C. sativa. In some embodiments, anon-CsPT6 PT is CsPT1, CsPT4, or CsPT7.

In some embodiments, chimeric PTs comprise one or more portions of CsPT7 and one or more portions of a non-CsPT7 PT. A portion can include, for example, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, or more than 390 amino acids. In some embodiments, a non-CsPT7 PT is a PT from C. sativa. In some embodiments, a non-CsPT7 PT is CsPT1, CsPT4, or CsPT6.

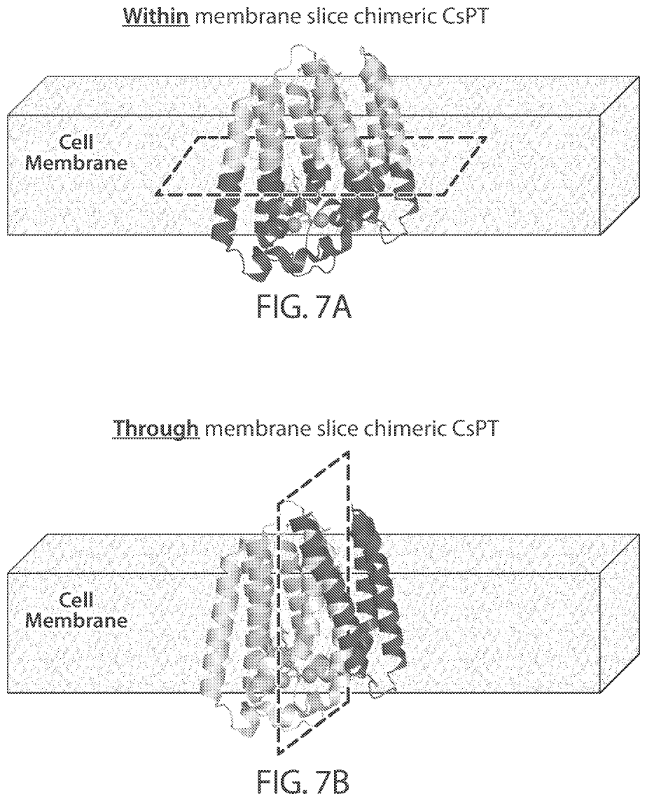

As described in Example 1 and FIG. 7, two different approaches were pursued for developing chimeric PTs based on where the cross-over points between different PT proteins occurred. As used in this disclosure, a “cross-over point” for a chimeric PT that contains portions of proteins “A” and “B” refers to the position where the sequence of the chimeric PT changes from protein A to B or vice versa. As discussed in Example 1 and as shown in FIG. 7, chimeric PTs can be generated using a “within membrane” approach or a “through membrane” approach. An example of a chimeric PT generated using a “within membrane” approach is shown in FIG. 7A. In this approach, the one or more cross-over points in the chimeric PT occur within the transmembrane helices of the chimeric PT. A “through membrane approach” is shown in FIG. 7B. In this approach, the one or more cross-over points in the chimeric PT occur outside of the transmembrane helices of the chimeric PT. For example, in FIG. 7B one single cross-over point is shown between helices 6&7 of the chimeric PT protein. Cross-over points can also occur between other helices, such as between helices 7&8 or 8&9.

Chimeric PTs associated with the disclosure include multiple transmembrane helices. As used in this disclosure, “multiple” transmembrane helices refers to more than one transmembrane helix. In some embodiments, chimeric PTs include 6, 7, 8, 9, 10, 11, 12, 13, 14, 15 or more than 15 transmembrane helices. In some embodiments, chimeric PTs include 9 transmembrane helices.

In some embodiments, at least one transmembrane helix includes both a portion of CsPT1 and a portion of a non-CsPT1 PT. In some embodiments, the non-CsPT1 PT is a PT from C. sativa. In some embodiments, the non-CsPT1 PT is CsPT4, CsPT6 or CsPT7. In some embodiments, all the transmembrane helices comprise both a portion of CsPT1 and a portion of a non-CsPT1 PT. In some embodiments, all the transmembrane helices comprise both a portion of CsPT1 and a portion of CsPT4, CsPT6 or CsPT7.

In some embodiments, at least one transmembrane helix includes both a portion of CsPT4 and a portion of a non-CsPT4 PT. In some embodiments, the non-CsPT4 PT is a PT from C. sativa. In some embodiments, the non-CsPT4 PT is CsPT1, CsPT6 or CsPT7. In some embodiments, all the transmembrane helices comprise both a portion of CsPT4 and a portion of a non-CsPT4 PT. In some embodiments, all the transmembrane helices comprise both a portion of CsPT4 and a portion of CsPT1, CsPT6 or CsPT7.

In some embodiments, at least one transmembrane helix includes both a portion of CsPT6 and a portion of a non-CsPT6 PT. In some embodiments, the non-CsPT6 PT is a PT from C. sativa. In some embodiments, the non-CsPT6 PT is CsPT1, CsPT4 or CsPT7. In some embodiments, all the transmembrane helices comprise both a portion of CsPT6 and a portion of a non-CsPT6 PT. In some embodiments, all the transmembrane helices comprise both a portion of CsPT6 and a portion of CsPT1, CsPT4 or CsPT7.

In some embodiments, at least one transmembrane helix includes both a portion of CsPT7 and a portion of a non-CsPT7 PT. In some embodiments, the non-CsPT7 PT is a PT from C. sativa. In some embodiments, the non-CsPT7 PT is CsPT1, CsPT4 or CsPT6. In some embodiments, all the transmembrane helices comprise both a portion of CsPT7 and a portion of a non-CsPT7 PT. In some embodiments, all the transmembrane helices comprise both a portion of CsPT7 and a portion of CsPT1, CsPT4 or CsPT6.

As one of ordinary skill in the art would appreciate, multiple different computational analysis programs may be used to determine secondary structures in proteins, such as CsPT proteins. Different computational analysis programs may define the boundaries of the secondary structures differently. For example, the Uniprot entry AOA455ZJC3 (corresponding to CsPT4) uses Phobius to predict that there are 8 sequences therewithin that are highly probable to be transmembrane helices. There is also a portion of the sequence with lower probability to be a transmembrane domain that is not listed on the Uniprot entry. As a comparison, for Uniprot entry 028625, which is a protein with the highest sequence identity to CsPT4 for which there is a crystal structure (ex. pdbID: 4tq3), the Uniprot entry similarly indicates that there are 8 transmembrane helices, while the structure itself shows 9 transmembrane helices. Without being bound by any theory, the lower probability transmembrane domain helix of CsPTs may be an actual transmembrane domain helix that did not meet an arbitrary probability threshold for annotation on UniProt based on the software prediction.

Table 2 provides a non-limiting example of predicted domains within CsPT1-CsPT7. “Inner” means inside the cell, “membrane” means in the cell membrane, and “outer” means outside the cell.

TABLE 2
Predicted domains within CsPT1-CsPT7

| Domain | CsPT1 | CsPT2 | CsPT3 | CsPT4 | CsPT5 | CsPT6 | CsPT7 |
|---|---|---|---|---|---|---|---|
| 1 | Inner, | Inner, | Inner, | Inner, | Inner, | Inner, | Inner, |
| | 1-35 | 1-34 | 1-94 | 1-37 | 1-34 | 1-85 | 1-37 |
| 2 | Membrane, | Membrane, | Membrane, | Membrane, | Membrane, | Membrane, | Membrane, |
| | 36-53 | 35-51 | 95-111 | 38-55 | 35-54 | 86-102 | 38-55 |
| 3 | Outer, | Outer, | Outer, | Outer, | Outer, | Outer, | Outer, |
| | 54-57 | 52-59 | 112-125 | 56-69 | 55-62 | 103-110 | 56-69 |
| 4 | Membrane, | Membrane, | Membrane, | Membrane, | Membrane, | Membrane, | Membrane, |
| | 68-84 | 60-82 | 126-143 | 70-87 | 63-80 | 111-133 | 70-87 |
| 5 | Inner, | Inner, | Inner, | Inner, | Inner, | Inner, | Inner, |
| | 86-111 | 83-108 | 144-169 | 88-113 | 81-106 | 134-159 | 88-113 |
| 6 | Membrane, | Membrane, | Membrane, | Membrane, | Membrane, | Membrane, | Membrane, |
| | 112-129 | 109-128 | 170-188 | 114-131 | 107-125 | 160-179 | 114-131 |
| 7 | Outer, | Outer, | Outer, | Outer, | Outer, | Outer, | Outer, |
| | 130-135 | 129-132 | 189-196 | 132-137 | 126-129 | 180-183 | 132-137 |
| 8 | Membrane, | Membrane, | Membrane, | Membrane, | Membrane, | Membrane, | Membrane, |
| | 136-153 | 133-150 | 197-214 | 138-155 | 130-149 | 184-201 | 138-155 |
| 9 | Inner, | Inner, | Inner, | Inner, | Inner, | Inner, | Inner, |
| | 154-165 | 151-158 | 215-226 | 156-167 | 150-157 | 202-209 | 156-167 |
| 10 | Membrane, | Membrane, | Membrane, | Membrane, | Membrane, | Membrane, | Membrane, |
| | 166-183 | 159-181 | 227-246 | 168-192 | 158-177 | 210-232 | 168-185 |
| 11 | Outer, | Outer, | Outer, | Outer, | Outer, | Outer, | Outer, |
| | 184-197 | 182-197 | 247-254 | 193-198 | 178-189 | 233-248 | 186-199 |
| 12 | Membrane, | Membrane, | Membrane, | Membrane. | Membrane, | Membrane, | Membrane, |
| | 200-215 | 198-215 | 255-272 | 199-216 | 190-209 | 249-266 | 200-217 |
| 13 | Inner, | Inner, | Inner, | Inner, | Inner, | Inner, | Inner, |
| | 216-241 | 216-241 | 273-298 | 217-244 | 210-237 | 267-292 | 218-243 |
| 14 | Membrane, | Membrane, | Membrane, | Membrane, | Membrane, | Membrane, | Membrane, |
| | 242-259 | 242-261 | 299-320 | 245-264 | 238-257 | 293-312 | 244-263 |
| 15 | Outer, | Outer, | Outer, | Outer, | Outer, | Outer, | Outer, |
| | 260-265 | 262-269 | 321-328 | 265-270 | 258-265 | 313-320 | 264-267 |
| 16 | Membrane, | Membrane, | Membrane, | Membrane, | Membrane, | Membrane, | Membrane, |
| | 266-284 | 270-287 | 329-348 | 271-288 | 266-285 | 321-338 | 268-287 |
| 17 | Inner, | Inner, | Inner, | Inner, | Inner, | Inner, | Inner, |
| | 285-302 | 288-299 | 349-360 | 289-304 | 286-293 | 339-350 | 288-304 |
| 18 | Membrane, | Membrane, | Membrane, | Membrane, | Membrane, | Membrane, | Membrane, |
| | 303-320 | 300-319 | 361-378 | 305-322 | 294-313 | 351-370 | 305-322 |
| 19 | Outer, | Outer, | Outer, | Outer, | Outer, | Outer, | Outer, |
| | 321 | 320 | 379 | 323 | 314-316 | 371 | 323 |

In some embodiments, a chimeric PT comprises portions of 1, 2, 3, 4, 5, 6, 7, or more than 7 different PTs. In some embodiments, the chimeric PT comprises one or more portions of CsPT1 and one or more portions of CsPT2, CsPT3, CsPT4, CsPT5, CsPT6, or CsPT7. In some embodiments, the chimeric PT comprises one or more portions of CsPT1 and one or more portions of CsPT4. In some embodiments, the chimeric PT comprises one or more portions of CsPT1 and one or more portions of CsPT6. In some embodiments, the chimeric PT comprises one or more portions of CsPT1 and one or more portions of CsPT7. In some embodiments, the chimeric PT comprises one or more portions of CsPT1, one or more portions of CsPT4, one or more portions of CsPT6, and/or one or more portions of CsPT7.

In some embodiments, the chimeric PT comprises one or more portions of CsPT4 and one or more portions of CsPT1, CsPT2, CsPT3, CsPT5, CsPT6 or CsPT7. In some embodiments, the chimeric PT comprises one or more portions of CsPT4 and one or more portions of CsPT1. In some embodiments, the chimeric PT comprises one or more portions of CsPT4 and one or more portions of CsPT6. In some embodiments, the chimeric PT comprises one or more portions of CsPT4 and one or more portions of CsPT7. In some embodiments, the chimeric PT comprises one or more portions of CsPT4, one or more portions of CsPT1, one or more portions of CsPT6, and/or one or more portions of CsPT7.

In some embodiments, the chimeric PT comprises one or more portions of CsPT6 and one or more portions of CsPT1, CsPT2, CsPT3, CsPT4, CsPT5 or CsPT7. In some embodiments, the chimeric PT comprises one or more portions of CsPT6 and one or more portions of CsPT1. In some embodiments, the chimeric PT comprises one or more portions of CsPT6 and one or more portions of CsPT4. In some embodiments, the chimeric PT comprises one or more portions of CsPT6 and one or more portions of CsPT7. In some embodiments, the chimeric PT comprises one or more portions of CsPT6, one or more portions of CsPT1, one or more portions of CsPT4, and/or one or more portions of CsPT7.

In some embodiments, the chimeric PT comprises one or more portions of CsPT7 and one or more portions of CsPT1, CsPT2, CsPT3, CsPT4, CsPT5 or CsPT6. In some embodiments, the chimeric PT comprises one or more portions of CsPT7 and one or more portions of CsPT1. In some embodiments, the chimeric PT comprises one or more portions of CsPT7 and one or more portions of CsPT4. In some embodiments, the chimeric PT comprises one or more portions of CsPT7 and one or more portions of CsPT6. In some embodiments, the chimeric PT comprises one or more portions of CsPT7, one or more portions of CsPT1, one or more portions of CsPT4, and/or one or more portions of CsPT6.

In some embodiments, at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, or 95% of a chimeric PT is derived from CsPT1. In some embodiments, at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, or 95% of a transmembrane helix of a chimeric PT is derived from CsPT1.

In some embodiments, at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39% 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, or 95% of a chimeric PT is derived from CsPT2. In some embodiments, at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, or 95% of a transmembrane helix of a chimeric PT is derived from CsPT2.

In some embodiments, at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, or 95% of a chimeric PT is derived from CsPT3. In some embodiments, at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, or 95% of a transmembrane helix of a chimeric PT is derived from CsPT3.

In some embodiments, at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, or 95% of a chimeric PT is derived from CsPT4. In some embodiments, at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, or 95% of a transmembrane helix of a chimeric PT is derived from CsPT4.

In some embodiments, at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44% 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, or 95% of a chimeric PT is derived from CsPT5. In some embodiments, at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, or 95% of a transmembrane helix of a chimeric PT is derived from CsPT5.

In some embodiments, at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, or 95% of a chimeric PT is derived from CsPT6. In some embodiments, at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, or 95% of a transmembrane helix of a chimeric PT is derived from CsPT6.

In some embodiments, at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, or 95% of a chimeric PT is derived from CsPT7. In some embodiments, at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, or 95% of a transmembrane helix of a chimeric PT is derived from CsPT7.

In some embodiments, a chimeric PT comprises all or part of the active site of CsPT1. In some embodiments, a chimeric PT comprises all or part of the active site of CsPT2. In some embodiments, a chimeric PT comprises all or part of the active site of CsPT3. In some embodiments, a chimeric PT comprises all or part of the active site of CsPT4. In some embodiments, a chimeric PT comprises all or part of the active site of CsPT5. In some embodiments, a chimeric PT comprises all or part of the active site of CsPT6. In some embodiments, a chimeric PT comprises all or part of the active site of CsPT7.

In some embodiments, a chimeric PT includes one or more of the following motifs: MTVMGMT (SEQ ID NO; 11); [EV][LMW][RS]P[SAP]F[ST]F[IL][IL]AF (SEQ ID NO: 12); QFFEFIW (SEQ ID NO: 13); HNTNL (SEQ ID NO: 14); TCWKL (SEQ ID NO: 15); M[IL]LSHAILAFC (SEQ ID NO: 16); HVG[LV][AN]FT[SCF]Y[YS]A[ST][RT][AS]A[LF] (SEQ ID NO; 17); GLIVT (SEQ ID NO: 18); L[YH]YAEY[LF]V (SEQ ID NO: 19); KAFFAL (SEQ ID NO: 20); KLGARNMT (SEQ ID NO: 21); QAF[NK]SN (SEQ ID NO: 22); LIFQT (SEQ ID NO: 23); SIIVALT (SEQ ID NO: 24); MSIETAW (SEQ ID NO: 25); VVSGV (SEQ ID NO: 26); RPYVV (SEQ ID NO: 27); KPDLP (SEQ ID NO: 28); RWKQY (SEQ ID NO: 29); FLITI (SEQ ID NO: 30); DIEGD (SEQ ID NO: 31); and KYGVST (SEQ ID NO: 32).

In some embodiments, motifs identified in this disclosure are located at chimeric junctions. Chimeric junctions refer to crossover points in a chimeric sequence. For example, in a chimeric PT that includes portions of CsPT4 and portions of CsPT7, a chimeric junction occurs at a region where a sequence derived from CsPT4 is joined to a sequence derived from CsPT7. A motif located at a chimeric junction therefore includes sequences derived from two or more CsPT proteins.

In some embodiments, a chimeric PT includes the motif MTVMGMT (SEQ ID NO: 11) at or near a chimeric junction. In some embodiments, a chimeric PT includes the motif MTVMGMT (SEQ ID NO: 11) at residues corresponding to residues 207-213 in SEQ ID NO: 5.

In some embodiments, a chimeric PT includes the motif [EV][LMW][RS]P[SAP]F[ST]F[IL][IL]AF (SEQ ID NO: 12) at or near a chimeric junction. In some embodiments, a chimeric PT includes the motif MTVMGMT (SEQ ID NO: 11) at residues corresponding to residues 195-206 of SEQ ID NO: 5.

In some embodiments, a chimeric PT includes the motif QFFEFIW (SEQ ID NO: 13) at or near a chimeric junction. In some embodiments, a chimeric PT includes the motif QFFEFIW (SEQ ID NO: 13) at residues corresponding to residues 304-310 of SEQ ID NO: 5.

In some embodiments, a chimeric PT includes the motif HNTNL (SEQ ID NO: 14) at residues corresponding to residues 57-61 of SEQ ID NO: 5.

In some embodiments, a chimeric PT includes the motif TCWKL (SEQ ID NO: 15) at residues corresponding to residues 30-34 of SEQ ID NO: 5.

In some embodiments, a chimeric PT includes the motif M[IL]LSHAILAFC (SEQ ID NO: 16) at or near a chimeric junction. In some embodiments, a chimeric PT includes the motif M[IL]LSHAILAFC (SEQ ID NO: 16) at residues corresponding to residues 274-284 of SEQ ID NO: 5.

In some embodiments, a chimeric PT includes the motif HVG[LV][AN]FT[SCF]Y[YS]A[ST][RT][AS]A[LF] (SEQ ID NO: 17) at or near a chimeric junction. In some embodiments, a chimeric PT includes the motif HVG[LV][AN]FT[SCF]Y[YS]A[ST][RT][AS]A[LF] (SEQ ID NO: 17) at residues corresponding to residues 175-190 of SEQ ID NO: 5.

In some embodiments, a chimeric PT includes the motif GLIVT (SEQ ID NO: 18) at or near a chimeric junction. In some embodiments, a chimeric PT includes the motif GLIVT (SEQ ID NO: 18) at residues corresponding to residues 126-130 of SEQ ID NO: 5.

In some embodiments, a chimeric PT includes the motif L[YH]YAEY[LF]V (SEQ ID NO: 19) at or near a chimeric junction. In some embodiments, a chimeric PT includes the motif L[YH]YAEY[LF]V (SEQ ID NO: 19) at residues corresponding to residues 312-319 of SEQ ID NO: 5.

In some embodiments, a chimeric PT includes the motif KAFFAL (SEQ ID NO: 20) at or near a chimeric junction. In some embodiments, a chimeric PT includes the motif KAFFAL (SEQ ID NO: 20 at residues corresponding to residues 69-74 of SEQ ID NO: 5.

In some embodiments, a chimeric PT includes the motif KLGARNMT (SEQ ID NO: 21) at residues corresponding to residues 237-244 of SEQ ID NO: 5.

In some embodiments, a chimeric PT includes the motif QAF[NK]SN (SEQ ID NO: 22) at or near a chimeric junction. In some embodiments, a chimeric PT includes the motif QAF[NK]SN (SEQ ID NO: 22) at residues corresponding to residues 267-272 of SEQ ID NO: 5.

In some embodiments, a chimeric PT includes the motif LIFQT (SEQ ID NO: 23) at or near a chimeric junction. In some embodiments, a chimeric PT includes the motif LIFQT (SEQ ID NO: 23) at residues corresponding to residues 285-289 of SEQ ID NO: 5.

In some embodiments, a chimeric PT includes the motif SIIVALT (SEQ ID NO: 24) at or near a chimeric junction. In some embodiments, a chimeric PT includes the motif SIIVALT (SEQ ID NO: 24) at residues corresponding to residues 119-125 of SEQ ID NO: 5.

In some embodiments, a chimeric PT includes the motif MSIETAW (SEQ ID NO: 25) at or near a chimeric junction. In some embodiments, a chimeric PT includes the motif MSIETAW (SEQ ID NO: 25) at residues corresponding to residues 110-116 of SEQ ID NO: 5.

In some embodiments, a chimeric PT includes the motif VVSGV (SEQ ID NO: 26) at or near a chimeric junction. In some embodiments, a chimeric PT includes the motif VVSGV (SEQ ID NO: 26) at residues corresponding to residues 246-250 of SEQ ID NO: 5.

In some embodiments, a chimeric PT includes the motif RPYVV (SEQ ID NO: 27) at or near a chimeric junction. In some embodiments, a chimeric PT includes the motif RPYVV (SEQ ID NO: 27) at residues corresponding to residues 36-40 of SEQ ID NO: 5.

In some embodiments, a chimeric PT includes the motif KPDLP (SEQ ID NO: 28) at residues corresponding to residues 100-104 of SEQ ID NO: 5.

In some embodiments, a chimeric PT includes the motif RWKQY (SEQ ID NO: 29) at residues corresponding to residues 100-104 of SEQ ID NO: 5.

In some embodiments, a chimeric PT includes the motif FLITI (SEQ ID NO: 30) at or near a chimeric junction. In some embodiments, a chimeric PT includes the motif FLITI (SEQ ID NO: 30) at residues corresponding to residues 168-172 of SEQ ID NO: 5.

In some embodiments, a chimeric PT includes the motif DIEGD (SEQ ID NO: 31) at residues corresponding to residues 222-226 of SEQ ID NO: 5.

In some embodiments, a chimeric PT includes the motif KYGVST (SEQ ID NO: 32) at residues corresponding to residues 228-233 of SEQ ID NO: 5.

The sequence of a chimeric PT associated with the disclosure can comprise the structure: X1-X2-X3-X4-X5-X6-X7-X8-X9-X10. In some embodiments, any one of X1, X2, X3, X4, X5, X6, X7, X8, X9, and X10 can comprise portions of CsPT1, CsPT2, CsPT3, CsPT4, CsPT5, CsPT6 or CsPT7. In some embodiments, X1, X2, X3, X4, X5, X6, X7, X8, X9 and/or X10 comprise portions of CsPT1. In some embodiments, X1, X2, X3, X4, X5, X6, X7, X8, X9 and/or X10 comprise portions of CsPT4. In some embodiments, X1, X2, X3, X4, X5, X6, X7, X8, X9 and/or X10 comprise portions of CsPT6. In some embodiments, X1, X2, X3, X4, X5, X6, X7, X8, X9 and/or X10 comprise portions of CsPT7. In some embodiments, X1, X3, X5, X7, and X9 comprise portions of CsPT4. In some embodiments, X2, X4, X6, X8, and X10 comprise portions of CsPT1, CsPT6 or CsPT7. In some embodiments, one or more of X1, X2, X3, X4, X5, X6, X7, X8, X9 and X10 includes a portion of a transmembrane helix. In some embodiments, each of X1, X2, X3, X4, X5, X6, X7, X8, X9 and X10 includes a portion of a transmembrane helix.

In some embodiments, the sequence of X1 comprises any of SEQ ID NOs: 33-39 or a sequence that comprises no more than 2 amino acid substitutions, insertions, additions or deletions relative to any one of SEQ ID NOs: 33-39. In some embodiments, the sequence of X2 comprises any of SEQ ID NOs: 40-46 or a sequence that comprises no more than 2 amino acid substitutions, insertions, additions or deletions relative to any one of SEQ ID NOs: 40-46. In some embodiments, the sequence of X3 comprises any of SEQ ID NOs: 47-53 or a sequence that comprises no more than 2 amino acid substitutions, insertions, additions or deletions relative to any one of SEQ ID NOs: 47-53. In some embodiments, the sequence of X4 comprises any of SEQ ID NOs: 54-60 or a sequence that comprises no more than 2 amino acid substitutions, insertions, additions or deletions relative to any one of SEQ ID NOs: 54-60. In some embodiments, the sequence of X5 comprises any of SEQ ID NOs: 61-67 or a sequence that comprises no more than 2 amino acid substitutions, insertions, additions or deletions relative to any one of SEQ ID NOs: 61-67. In some embodiments, the sequence of X6 comprises any of SEQ ID NOs: 68-74 or a sequence that comprises no more than 2 amino acid substitutions, insertions, additions or deletions relative to any one of SEQ ID NOs: 68-74. In some embodiments, the sequence of X7 comprises any of SEQ ID NOs: 75-81 or a sequence that comprises no more than 2 amino acid substitutions, insertions, additions or deletions relative to any one of SEQ ID NOs: 75-81. In some embodiments, the sequence of X8 comprises any of SEQ ID NOs: 82-88 or a sequence that comprises no more than 2 amino acid substitutions, insertions, additions or deletions relative to any one of SEQ ID NOs: 82-88. In some embodiments, the sequence of X9 comprises any of SEQ ID NOs: 89-95 or a sequence that comprises no more than 2 amino acid substitutions, insertions, additions or deletions relative to any one of SEQ ID NOs: 89-95. In some embodiments, the sequence of X10 comprises any of SEQ ID NOs: 96-102 or a sequence that comprises no more than 2 amino acid substitutions, insertions, additions or deletions relative to any one of SEQ ID NOs: 96-102.

In some embodiments, a chimeric PT comprises a sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical, including all values in between, to any one of SEQ ID NOs: 110-121, 133-144, 757-868, 869-980, 982-1081 or 1083-1182, to any chimeric PT disclosed in Tables 13-16 and 19-20, or to any chimeric PT disclosed in this application.

b. Prenyltransferase Fusions

Further aspects of the disclosure relate to fusion proteins comprising PTs associated with the disclosure, including chimeric PTs. Chimeric PTs that are components of fusion proteins may in some instances be referred to within this disclosure as “chimeric fusions.”

For example, a PT may be linked to one or more genes in the cannabinoid biosynthesis pathway or a metabolic pathway of a host cell. In some embodiments, the one or more genes linked to the PT includes a gene that encodes a polypeptide having enzymatic activity such that its product is a substrate for the PT. In some embodiments, the one or more genes linked to the PT includes a gene that encodes a polypeptide having enzymatic activity such that the product of the PT is a substrate for the downstream polypeptide. In certain embodiments, a PT may be linked to a mutant form of one or more genes in the metabolic pathway of a host cell. In certain embodiments, a PT may be linked to a farnesyl pyrophosphate synthase. The farnesyl pyrophosphate synthase can be linked to the amino terminus or the carboxy terminus of a PT. In some embodiments, the farnesyl pyrophosphate synthase is linked to the amino terminus of the PT, with or without a linker sequence separating the farnesyl pyrophosphate synthase and the PT sequence.

Farnesyl pyrophosphate synthase enzymes convert isopentenyl diphosphate (IPP) and dimethylallyl diphosphate (DMAP) to geranyl pyrophosphate (GPP) and farnesyl pyrophosphate (FPP) in yeast cells. In some embodiments, a farnesyl pyrophosphate synthase enzyme may produce neryl pyrophosphate (NPP). In some embodiments, the farnesyl pyrophosphate synthase component of a PT fusion protein is the S. cerevisiae ERG20 protein. In some embodiments, the farnesyl pyrophosphate synthase comprises one or more mutations relative to a wild-type farnesyl pyrophosphate synthase. Mutations in a farnesyl pyrophosphate synthase may modulate the ratio of GPP and FPP produced by the enzyme. In some embodiments, the farnesyl pyrophosphate synthase comprises a mutation that increases the production of GPP relative to FPP. In some embodiments, the farnesyl pyrophosphate synthase comprises one or more mutations that reduce the levels of production of FPP and/or increase production of GPP. See, Ignea et al. ACS Synth. Biol. (2014) 3: 298-306.

In some embodiments, the farnesyl pyrophosphate synthase is ERG20, corresponding to UniProt Accession No. P08524, provided by SEQ ID NO: 424:

(SEQ ID NO: 424)
MASEKEIRRERFLNVFPKLVEELNASLLAYGMPKEACDWYAHSLNYNTP
GGKLNRGLSVVDTYAILSNKTVEQLGQEEYEKVAILGWCIELLQAYFLV
ADDMMDKSITRRGQPCWYKVPEVGEIAINDAFMLEAAIYKLLKSHFRNE
KYYIDITELFHEVTFQTELGQLMDLITAPEDKVDLSKFSLKKHSFIVTF
KTAYYSFYLPVALAMYVAGITDEKDLKQARDVLIPLGEYFQIQDDYLDC
FGTPEQIGKIGTDIQDNKCSWVINKALELASAEQRKTLDENYGKKDSVA
EAKCKKIFNDLKIEQLYHEYEESIAKDLKAKISQVDESRGFKADVLTAF
LNKVYKRSK.

In some embodiments, the farnesyl pyrophosphate synthase is ERG20 comprising F96W and/or N127W substitutions relative to the wildtype ERG20 sequence. The sequence of ERG20 F96W N127W is provided by SEQ ID NO: 103.

(SEQ ID NO: 103)
MASEKEIRRERFLNVFPKLVEELNASLLAYGMPKEACDWYAHSLNYNTP
GGKLNRGLSVVDTYAILSNKTVEQLGQEEYEKVAILGWCIELLQAYWLV
ADDMMDKSITRRGQPCWYKVPEVGEIAIWDAFMLEAAIYKLLKSHFRNE
KYYIDITELFHEVTFQTELGQLMDLITAPEDKVDLSKFSLKKHSFIVTF
KTAYYSFYLPVALAMYVAGITDEKDLKQARDVLIPLGEYFQIQDDYLDC
FGTPEQIGKIGTDIQDNKCSWVINKALELASAEQRKTLDENYGKKDSVA
EAKCKKIFNDLKIEQLYHEYEESIAKDLKAKISQVDESRGFKADVLTAF
LNKVYKRSK.

In some embodiments, the farnesyl pyrophosphate synthase comprises a mutation at position K197 of ERG20.

In some embodiments, the farnesyl pyrophosphate synthase comprises a protein sequence that is at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical, to SEQ ID NO: 424 or 103. In some embodiments, a farnesyl pyrophosphate synthase does not comprise SEQ ID NO: 103 or SEQ ID NO: 424.

Example 6 describes the identification of ERG20 homologs. In some embodiments, the farnesyl pyrophosphate synthase component of a fusion protein is an ERG20 homolog identified in Example 6, the sequences of which are provided in Table 17. In some embodiments, an ERG20 homolog comprises a tryptophan residue at a residue corresponding to amino acid positions F96 and/or N127 in S. cerevisiae ERG20. In some embodiments, an ERG20 homolog comprises a substitution at a residue corresponding to amino acid position K197 in S. cerevisiae ERG20.

In some embodiments, the farnesyl pyrophosphate synthase comprises a protein or nucleic acid sequence that is at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% identical, or is 100% identical, to any one of SEQ ID NOs: 426-476, 479-529, 753 or 754, to any sequence provided in Table 17, or to any other ERG20 homolog sequence provided in this disclosure.

Example 6 describes the identification of putative farnesyl pyrophosphate synthases that were effective in producing CBGA when fused with a prenyltransferase. Table 10 provides non-limiting examples of motifs that were identified in the sequences of the putative farnesyl pyrophosphate synthases that were effective in producing CBGA. In some embodiments, a farnesyl pyrophosphate synthase includes one or more of the following motifs, provided in Table 10: NVPGGKLNR (SEQ ID NO: 647), FYLPVALA[LM]H (SEQ ID NO: 648), A[EH]D[IV]LIPLG (SEQ ID NO: 651), LGW[CL][ITV]ELLQA[FY]FL (SEQ ID NO: 655), KKEV[FL][ET][SA]FL[AGN]KIYK (SEQ ID NO: 663), QRK[VI]L[DE]ENYG (SEQ ID NO: 667), VGMIAIWD (SEQ ID NO: 672), TDI[QK]DNKCSW (SEQ ID NO; 673), TAYYSFYLP (SEQ ID NO; 676), GKIGTDI[QK]DNKCSW (SEQ ID NO: 677), ILIP[LM]GEYFQ (SEQ ID NO: 680), IL[VM][EP][ML]G[ET][YF]FQ (SEQ ID NO: 683), AKIYKRSK (SEQ ID NO: 685), DPEVIGKI (SEQ ID NO: 686), RGQPCW[YF]RVP[EQ] (SEQ ID NO: 687), IVKYKTA[YF]Y[ST]FYLP (SEQ ID NO: 689), WC[IV]E[LW]LQA[YF][WF]LV[ALW]D (SEQ ID NO: 692), CSWLV[VN]Q[AC]L[AQ][RI][AC][ST]P[ED]Q (SEQ ID NO: 699).

In some embodiments, a farnesyl pyrophosphate synthase includes the motif NVPGGKLNR (SEQ ID NO. 647) at residues corresponding to residues 47-55 in SEQ ID NO: 424.

In some embodiments, a farnesyl pyrophosphate synthase includes the motif FYLPVALA[LM]H (SEQ ID NO: 648) at residues corresponding to residues 203-212 in SEQ ID NO: 424. In some embodiments, the motif FYLPVALA[LM]H is FYLPVALALH (SEQ ID NO: 649) or FYLPVALAMH (SEQ ID NO: 650).

In some embodiments, a farnesyl pyrophosphate synthase includes the motif A[EH]D[IV]LIPLG (SEQ ID NO: 651) at residues corresponding to residues 225-233 of SEQ ID NO: 424. In some embodiments, the motif A[EH]D[IV]LIPLG (SEQ ID NO: 651) is AEDILIPLG (SEQ ID NO: 652), AHDILIPLG (SEQ ID NO: 653), or AHDVLIPLG (SEQ ID NO: 654).

In some embodiments, a farnesyl pyrophosphate synthase includes the motif LGW[CL][ITV]ELLQA[FY]FL (SEQ ID NO: 655) at residues corresponding to residues 85-97 of SEQ ID NO: 424. In some embodiments, the motif LGW[CL][ITV]ELLQA[FY]FL (SEQ ID NO: 655) is LGWLTELLQAYFL (SEQ ID NO: 656), LGWLTELLQAFFL (SEQ ID NO: 657), LGWCIELLQAYFL (SEQ ID NO: 658), LGWCVELLQAYFL (SEQ ID NO: 659), LGWCVELLQAFFL (SEQ ID NO: 660), LGWCIELLQAFFL (SEQ ID NO: 661), or LGWCTELLQAFFL (SEQ ID NO: 662).

In some embodiments, a farnesyl pyrophosphate synthase includes the motif KKEV[FL][ET][SA]FL[AGN]KIYK (SEQ ID NO: 663) at residues corresponding to residues 336-349 of SEQ ID NO: 424. In some embodiments, the motif KKEV[FL][ET][SA]FL[AGN]KIYK (SEQ ID NO: 663) is KKEVFESFLAKIYK (SEQ ID NO: 664), KKEVFEAFLGKIYK (SEQ ID NO: 665), or KKEVLTSFLNKIYK (SEQ ID NO: 666).

In some embodiments, a farnesyl pyrophosphate synthase includes the motif QRK[VI]L[DE]ENYG (SEQ ID NO: 667) at residues corresponding to residues 279-288 of SEQ ID NO: 424. In some embodiments, the motif QRK[VI]L[DE]ENYG (SEQ ID NO: 667) is QRKVLDENYG (SEQ ID NO: 668), QRKILDENYG (SEQ ID NO: 669), QRKILEENYG (SEQ ID NO: 670), or QRKVLEENYG (SEQ ID NO: 671).

In some embodiments, a farnesyl pyrophosphate synthase includes the motif VGMIAIWD at residues corresponding to residues 121-128 of SEQ ID NO: 424.

In some embodiments, a farnesyl pyrophosphate synthase includes the motif TDI[QK]DNKCSW (SEQ ID NO: 673) at residues corresponding to residues 217-226 of SEQ ID NO: 424. In some embodiments, the motif TDI[QK]DNKCSW (SEQ ID NO: 673) is TDIQDNKCSW (SEQ ID NO: 674) or TDIKDNKCSW (SEQ ID NO: 675).

In some embodiments, a farnesyl pyrophosphate synthase includes the motif TAYYSFYLP (SEQ ID NO: 676) at residues corresponding to residues 198-206 of SEQ ID NO: 424.

In some embodiments, a farnesyl pyrophosphate synthase includes the motif GKIGTDI[QK]DNKCSW (SEQ ID NO: 677) at residues corresponding to residues 253-266 of SEQ ID NO: 424. In some embodiments, the motif GKIGTDI[QK]DNKCSW (SEQ ID NO: 677) is GKIGTDIQDNKCSW (SEQ ID NO: 678) or GKIGTDIKDNKCSW (SEQ ID NO: 679).

In some embodiments, a farnesyl pyrophosphate synthase includes the motif ILIP[LM]GEYFQ (SEQ ID NO: 680) at residues corresponding to residues 228-237 of SEQ ID NO: 424. In some embodiments, the motif ILIP[LM]GEYFQ (SEQ ID NO: 680) is ILIPLGEYFQ (SEQ ID NO: 681) or ILIPMGEYFQ (SEQ ID NO: 682).

In some embodiments, a farnesyl pyrophosphate synthase includes the motif IL[VM][EP][ML]G[ET][YF]FQ (SEQ ID NO: 683) at residues corresponding to residues 228-237 of SEQ ID NO: 424. In some embodiments, the motif IL[VM][EP][ML]G[ET][YF]FQ (SEQ ID NO: 683) is ILVPMGEYFQ (SEQ ID NO: 684).

In some embodiments, a farnesyl pyrophosphate synthase includes the motif AKIYKRSK (SEQ ID NO: 685) at residues corresponding to residues 345-352 of SEQ ID NO: 424.

In some embodiments, a farnesyl pyrophosphate synthase includes the motif DPEVIGKI (SEQ ID NO: 248) at residues corresponding to residues 248-255 of SEQ ID NO: 424.

In some embodiments, a farnesyl pyrophosphate synthase includes the motif RGQPCW[YF]RVP[EQ] (SEQ ID NO: 687) at residues corresponding to residues 110-120 of SEQ ID NO: 424. In some embodiments, the motif RGQPCW[YF]RVP[EQ] (SEQ ID NO: 687) is RGQPCWYRVPE (SEQ ID NO: 688).

In some embodiments, a farnesyl pyrophosphate synthase includes the motif IVKYKTA[YF]Y[ST]FYLP (SEQ ID NO: 689) at residues corresponding to residues 193-206 of SEQ ID NO: 424. In some embodiments, the motif IVKYKTA[YF]Y[ST]FYLP (SEQ ID NO: 689) is IVKYKTAFYSFYLP (SEQ ID NO: 690) or IVKYKTAYYSFYLP (SEQ ID NO: 691).

In some embodiments, a farnesyl pyrophosphate synthase includes the motif WC[IV]E[LW]LQA[YF][WF]LV[ALW]D (SEQ ID NO: 692) at residues corresponding to residues 87-100 of SEQ ID NO: 424. In some embodiments, the motif WC[IV]E[LW]LQA[YF][WF]LV[ALW]D (SEQ ID NO: 692) is WCIELLQAFFLVAD (SEQ ID NO: 693), WCIELLQAFWLVAD (SEQ ID NO: 694), WCIELLQAYFLVAD (SEQ ID NO: 695), WCIELLQAYWLVAD (SEQ ID NO: 696), WCIEWLQAFFLVAD (SEQ ID NO: 697) or WCVELLQAYFLVAD (SEQ ID NO: 698).

In some embodiments, a farnesyl pyrophosphate synthase includes the motif CSWLV[VN]Q[AC]L[AQ][RI][AC][ST]P[ED]Q (SEQ ID NO: 699) at residues corresponding to residues 264-279 of SEQ ID NO: 424. In some embodiments, the motif CSWLV[VN]Q[AC]L[AQ][RI][AC][ST]P[ED]Q (SEQ ID NO: 699) is CSWLVVQALARATPEQ (SEQ ID NO: 700).

In some embodiments of fusion proteins associated with the disclosure, a farnesyl pyrophosphate synthase and a chimeric PT are separated by a linker sequence. In some embodiments, the linker joins a C-terminal residue of the farnesyl pyrophosphate synthase and an N-terminal residue of the PT enzyme. In some embodiments, the linker is a peptide linker. Examples of peptide linkers include, for example SG, GGGS (SEQ ID NO: 104), SGSGSGSGS (SEQ ID NO: 105), GGGSGGGGSGGGGS (SEQ ID NO: 106), GGGSGGGGSGGGGSGGGGSGGGGS (SEQ ID NO: 107), GGGSGGGGSGGGGSGGGGSGGGGSGGGGSGGGGS (SEQ ID NO: 108), and GGGSGGGGSGGGGSGGGGSGGGGSGGGGSGGGGSGGGGSGGGGS (SEQ ID NO: 109).

Any of the PTs provided in this disclosure, including truncated PTs and/or chimeric PTs can be expressed as fusion proteins with any farnesyl pyrophosphate synthase provided in this disclosure.

In some embodiments, fusion proteins associated with the disclosure comprise, from N-terminus to C-terminus, a farnesyl pyrophosphate synthase, a linker, and a chimeric PT enzyme, or truncation thereof. In some embodiments, a fusion protein comprises, from N-terminus to C-terminus, ERG20 F96W N127W provided by SEQ ID NO: 103, a linker, and any of the chimeric PTs described in this disclosure, including truncations thereof. In other embodiments, a fusion protein comprises, from N-terminus to C-terminus, an ERG20 homolog provided by any one of SEQ ID NOs: 426476, a linker, and any of the chimeric PTs described in this disclosure, including truncations thereof.

In some embodiments, a fusion protein that includes a farnesyl pyrophosphate synthase and a PT comprises a protein or nucleic acid sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical, including all values in between, to any one of SEQ ID NOs: 122-132, 145-155, 156-225, 226-423, 532-582, 585-635, 704, 710, 724, 729, 735, 749, 755 or 756, or any fusion protein disclosed in Tables 13-14, 16 and 18, or any fusion protein disclosed in this application.

c. Prenyltransferase Mutations

PTs associated with the disclosure, including chimeric PTs and chimeric fusions, may include one or more amino acid substitutions, additions, deletions or insertions corresponding to a reference sequence. In some embodiments, a PT comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, or 323 amino acid substitutions, additions, deletions or insertions relative to a reference sequence. In some embodiments, the reference sequence is SEQ ID NO: 5.

In some embodiments, a PT comprises an amino acid substitution, addition, deletion or insertion at a residue corresponding to position 29, 31, 39, 41, 43, 46, 47, 48, 52, 56, 59, 60, 67, 68, 72, 80, 82, 83, 86, 87, 91, 94, 110, 113, 136, 140, 141, 142, 145, 147, 148, 149, 151, 162, 163, 167, 170, 173, 174, 182, 184, 187, 197, 199, 210, 215, 216, 223, 231, 232, 243, 244, 245, 258, 260, 261, 263, 267, 272, 273, 277, 284, 288, 289, 298, 301, 302, 311, and/or 318 in SEQ ID NO: 5.

In some embodiments, the PT comprises the amino acid D at a residue corresponding to position 29 in SEQ ID NO: 5; the amino acid A at a residue corresponding to position 30 in SEQ ID NO: 5; the amino acid F at a residue corresponding to position 31 in SEQ ID NO: 5; the amino acid F at a residue corresponding to position 34 in SEQ ID NO: 5: the amino acid T at a residue corresponding to position 35 in SEQ ID NO; 5; the amino acid M, T, or A at a residue corresponding to position 39 in SEQ ID NO: 5; the amino acid I at a residue corresponding to position 40 in SEQ ID NO: 5; the amino acid V or I at a residue corresponding to position 41 in SEQ ID NO: 5; the amino acid V, A, or L at a residue corresponding to position 43 in SEQ ID NO: 5; the amino acid L, F, or I at a residue corresponding to position 45 in SEQ ID NO: 5; the amino acid G, C, or A at a residue corresponding to position 46 in SEQ ID NO: 5; the amino acid V or S at a residue corresponding to position 47 in SEQ ID NO: 5; the amino acid T at a residue corresponding to position 48 in SEQ ID NO: 5, the amino acid S or A at a residue corresponding to position 49 in SEQ ID NO: 5; the amino acid L or A at a residue corresponding to position 52 in SEQ ID NO: 5; the amino acid L, T, I at a residue corresponding to position 56 in SEQ ID NO: 5; the amino acid P at a residue corresponding to position 59 in SEQ ID NO: 5; the amino acid E. D, or N at a residue corresponding to position 60 in SEQ ID NO: 5; the amino acid I or F at a residue corresponding to position 62 in SEQ ID NO: 5; the amino acid L or I at a residue corresponding to position 67 in SEQ ID NO: 5; the amino acid G or F at a residue corresponding to position 68 in SEQ ID NO: 5; the amino acid E at a residue corresponding to position 72 in SEQ ID NO: 5; the amino acid G at a residue corresponding to position 73 in SEQ ID NO: 5; the amino acid V, L, F, or I at a residue corresponding to position 75 in SEQ ID NO: 5; the amino acid L or C at a residue corresponding to position 79 in SEQ ID NO: 5; the amino acid W at a residue corresponding to position 80 in SEQ ID NO: 5; the amino acid G at a residue corresponding to position 82 in SEQ ID NO: 5; the amino acid Y at a residue corresponding to position 83 in SEQ ID NO: 5; the amino acid N at a residue corresponding to position 85 in SEQ ID NO: 5; the amino acid S, T, A, G, F, V, or C at a residue corresponding to position 86 in SEQ ID NO: 5; the amino acid T, I, C, Q, V, or L at a residue corresponding to position 87 in SEQ ID NO: 5; the amino acid L or F at a residue corresponding to position 91 in SEQ ID NO: 5; the amino acid E at a residue corresponding to position 94 in SEQ ID NO: 5; the amino acid Y at a residue corresponding to position 102 in SEQ ID NO: 5; the amino acid I at a residue corresponding to position 105 in SEQ ID NO: 5; the amino acid A at a residue corresponding to position 106 in SEQ ID NO: 5; the amino acid I or L at a residue corresponding to position 110 in SEQ ID NO: 5; the amino acid R at a residue corresponding to position 113 in SEQ ID NO: 5; the amino acid L at a residue corresponding to position 117 in SEQ ID NO: 5; the amino acid I at a residue corresponding to position 118 in SEQ ID NO: 5; the amino acid A at a residue corresponding to position 119 in SEQ ID NO: 5; the amino acid S at a residue corresponding to position 121 in SEQ ID NO: 5; the amino acid S or F at a residue corresponding to position 122 in SEQ ID NO: 5; the amino acid I or L at a residue corresponding to position 129 in SEQ ID NO: 5; the amino acid G at a residue corresponding to position 134 in SEQ ID NO: 5; the amino acid P or S at a residue corresponding to position 136 in SEQ ID NO: 5; the amino acid L or I at a residue corresponding to position 139 in SEQ ID NO: 5; the amino acid L, I, T, or F at a residue corresponding to position 140 in SEQ ID NO: 5; the amino acid L, S, V, A, C, or I at a residue corresponding to position 141 in SEQ ID NO: 5; the amino acid A, L, M, or T at a residue corresponding to position 142 in SEQ ID NO: 5, the amino acid S, I, C, V, L, M, T, or F at a residue corresponding to position 145 in SEQ ID NO: 5; the amino acid L at a residue corresponding to position 147 in SEQ ID NO: 5; the amino acid S, A or L at a residue corresponding to position 148 in SEQ ID NO: 5; the amino acid E, W, C, I, Q, S, T or L at a residue corresponding to position 149 in SEQ ID NO: 5; the amino acid M, G, H, T, I, A, or C at a residue corresponding to position 151 in SEQ ID NO: 5; the amino acid I or L at a residue corresponding to position 152 in SEQ ID NO: 5; the amino acid R at a residue corresponding to position 162 in SEQ ID NO: 5; the amino acid F at a residue corresponding to position 163 in SEQ ID NO: 5; the amino acid A at a residue corresponding to position 167 in SEQ ID NO: 5; the amino acid A at a residue corresponding to position 169 in SEQ ID NO: 5; the amino acid T or C at a residue corresponding to position 170 in SEQ ID NO: 5; the amino acid I at a residue corresponding to position 171 in SEQ ID NO: 5; the amino acid F, L, or V at a residue corresponding to position 172 in SEQ ID NO: 5; the amino acid W, G, L, or T at a residue corresponding to position 173 in SEQ ID NO: 5; the amino acid T at a residue corresponding to position 174 in SEQ ID NO: 5; the amino acid F at a residue corresponding to position 176 in SEQ ID NO: 5; the amino acid T, L, A, I, or V at a residue corresponding to position 177 in SEQ ID NO: 5; the amino acid P or N at a residue corresponding to position 179 in SEQ ID NO: 5; the amino acid L, V, F, or S at a residue corresponding to position 182 in SEQ ID NO: 5; the amino acid Y or L at a residue corresponding to position 184 in SEQ ID NO: 5; the amino acid R at a residue corresponding to position 187 in SEQ ID NO: 5; the amino acid L or V at a residue corresponding to position 190 in SEQ ID NO: 5; the amino acid L, I, F, or W at a residue corresponding to position 196 in SEQ ID NO: 5; the amino acid I, A, V, or S at a residue corresponding to position 197 in SEQ ID NO: 5; the amino acid S or A at a residue corresponding to position 199 in SEQ ID NO: 5; the amino acid L at a residue corresponding to position 200 in SEQ ID NO: 5; the amino acid I or T at a residue corresponding to position 204 in SEQ ID NO: 5; the amino acid V at a residue corresponding to position 207 in SEQ ID NO: 5; the amino acid L at a residue corresponding to position 209 in SEQ ID NO: 5; the amino acid Y or F at a residue corresponding to position 210 in SEQ ID NO: 5; the amino acid S, T. or A at a residue corresponding to position 211 in SEQ ID NO: 5, the amino acid I or L at a residue corresponding to position 212 in SEQ ID NO: 5; the amino acid V, A, I, or G at a residue corresponding to position 213 in SEQ ID NO: 5; the amino acid Y at a residue corresponding to position 215 in SEQ ID NO: 5; the amino acid I at a residue corresponding to position 216 in SEQ ID NO: 5; the amino acid L at a residue corresponding to position 220 in SEQ ID NO: 5; the amino acid V at a residue corresponding to position 223 in SEQ ID NO: 5: the amino acid R or K at a residue corresponding to position 227 in SEQ ID NO: 5; the amino acid E or A at a residue corresponding to position 228 in SEQ ID NO: 5; the amino acid H or F at a residue corresponding to position 229 in SEQ ID NO: 5; the amino acid N at a residue corresponding to position 230 in SEQ ID NO: 5; the amino acid M, L, or I at a residue corresponding to position 231 in SEQ ID NO: 5; the amino acid R or K at a residue corresponding to position 232 in SEQ ID NO: 5, the amino acid L, F, or M at a residue corresponding to position 234 in SEQ ID NO: 5; the amino acid V at a residue corresponding to position 236 in SEQ ID NO: 5; the amino acid K at a residue corresponding to position 241 in SEQ ID NO: 5; the amino acid T at a residue corresponding to position 242 in SEQ ID NO: 5; the amino acid I, T, L, or A at a residue corresponding to position 243 in SEQ ID NO: 5; the amino acid A at a residue corresponding to position 244 in SEQ ID NO: 5; the amino acid W or R at a residue corresponding to position 245 in SEQ ID NO: 5; the amino acid L, I, M, or F at a residue corresponding to position 246 in SEQ ID NO: 5; the amino acid C, S. G, or A at a residue corresponding to position 247 in SEQ ID NO: 5; the amino acid L, T, I, A, or F at a residue corresponding to position 250 in SEQ ID NO: 5; the amino acid N, L, A, or C at a residue corresponding to position 254 in SEQ ID NO: 5, the amino acid V at a residue corresponding to position 256 in SEQ ID NO: 5; the amino acid G or L at a residue corresponding to position 257 in SEQ ID NO: 5; the amino acid A at a residue corresponding to position 258 in SEQ ID NO: 5; the amino acid L, V, A, 1, or F at a residue corresponding to position 260 in SEQ ID NO: 5; the amino acid G at a residue corresponding to position 262 in SEQ ID NO: 5; the amino acid A at a residue corresponding to position 261 in SEQ ID NO: 5: the amino acid A at a residue corresponding to position 263 in SEQ ID NO: 5; the amino acid G at a residue corresponding to position 262 in SEQ ID NO: 5; the amino acid N or F at a residue corresponding to position 264 in SEQ ID NO: 5; the amino acid F at a residue corresponding to position 267 in SEQ ID NO: 5; the amino acid K or L at a residue corresponding to position 271 in SEQ ID NO: 5; the amino acid S at a residue corresponding to position 272 in SEQ ID NO: 5; the amino acid F at a residue corresponding to position 273 in SEQ ID NO: 5; the amino acid I at a residue corresponding to position 275 in SEQ ID NO: 5; the amino acid F at a residue corresponding to position 276 in SEQ ID NO: 5; the amino acid S at a residue corresponding to position 277 in SEQ ID NO: 5; the amino acid L, W, or 1 at a residue corresponding to position 284 in SEQ ID NO: 5; the amino acid S at a residue corresponding to position 283 in SEQ ID NO: 5; the amino acid I or W at a residue corresponding to position 284 in SEQ ID NO: 5; the amino acid F at a residue corresponding to position 286 in SEQ ID NO: 5; the amino acid R at a residue corresponding to position 288 in SEQ ID NO: 5; the amino acid A at a residue corresponding to position 289 in SEQ ID NO: 5; the amino acid D at a residue corresponding to position 298 in SEQ ID NO: 5; the amino acid D, G, or T at a residue corresponding to position 301 in SEQ ID NO: 5; the amino acid T at a residue corresponding to position 302 in SEQ ID NO: 5 the amino acid R, N, or K at a residue corresponding to position 311 in SEQ ID NO: 5; and/or the amino acid L at a residue corresponding to position 318 in SEQ ID NO: 5.

In some embodiments, one or more substitution mutations are located at residues at or near the active site of a PT protein. The active site of a PT may be defined by generating the three-dimensional structure of the PT and identifying the residues within a particular distance of the GPP substrate binding site and/or the Mg binding site. As a non-limiting example, the structure of a PT may be generated using ROSETTA software. See, e.g., Kaufmann et al., Biochemistry 2010, 49, 2987-2998. As used in this disclosure, a residue is within the active site of a PT enzyme if it is within about 8 angstroms from the GPP substrate binding site and/or the Mg binding site. As used in this disclosure, a residue is near the active site of a PT enzyme if it is within about 8-12 angstroms from the GPP substrate binding site and/or the Mg binding site. In some embodiments, a substitution mutation is present in a residue corresponding to residue M43, F82, F83, I86, M87, S119, V122, F145, I147, or F151 in SEQ ID NO: 5.

In some embodiments, one or more substitution mutations are located in an apposing face of a helix that forms part of the active site of a CsPT. For example, in some embodiments, a substitution mutation is present in a residue corresponding to residue 186. F83, or M87 of SEQ ID NO: 5. In some embodiments, one or more substitution mutations are located in residues that are predicted to interact with a residue corresponding to residue 186 of SEQ ID NO: 5. For example, in some embodiments, a substitution mutation is present in a residue corresponding to residue F82, F83, M87, S119, or V122.

Without wishing to be bound by any theory, substitution mutations at a residue corresponding to position 86 in SEQ ID NO: 5 (e.g., I86S, I86G, I86A) may increase activity of the PT enzyme due to the decreased residue size relative to the corresponding residue in the wildtype protein. Reduction in side-chain volume at this position may lead to a slight shift in the helix, which could increase the volume of the olivetolic/divarinic acid binding pocket. Without wishing to be bound by any theory, substitution mutations at a residue corresponding to position 82 (e.g., F82G), 83 (e.g., F83Y), 87 (e.g., M87T, M87I, M87C, M87Q or M87V), 119 (e.g., S119A) and/or 122 (e.g., V122F or V122S) of SEQ ID NO: 5, may impact the olivetolic/divarinic acid binding pocket in a similar manner to that discussed above for position 86 in SEQ ID NO: 5. Without wishing to be bound by any theory, substitution mutations at a residue corresponding to position 82 (e.g., F82G), 94 (e.g., D94E), 147 (e.g., I147L), 227 (e.g., A227K), and/or 254 (e.g., T254N) of SEQ ID NO: 5, may increase CBGA production.

It should be appreciated that any of the PTs provided in this disclosure, including chimeric PTs and fusion proteins, can comprise any of the point mutations provided in this disclosure.

A PT described in this disclosure, including a chimeric PT and/or a chimeric fusion, may be capable of producing more CBGA and/or CBGVA relative to a control PT. In some embodiments, a control PT comprises any of SEQ ID NOs: 1-5.

In some embodiments, a PT described in this disclosure, including a chimeric PT and/or a chimeric fusion, that produces more CBGA and/or CBGVA relative to a control PT may be capable of producing at least 1% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 300%, at least 400%, at least 500%, at least 600%, at least 700%, at least 800%, at least 900%, or at least 1,000%) more CBGA and/or CBGVA than a control PT. In some embodiments, a control PT comprises any of SEQ ID NOs: 1-5.

In some embodiments, a PT described in this disclosure, including a chimeric PT and/or a fusion protein, that produces more CBGA and/or CBGVA relative to a control PT may be capable of producing at least 1% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 300%, at least 400%, at least 500%, at least 600%, at least 700%, at least 800%, at least 900%, or at least 1,000%) more CBGA and/or OGOA than a control PT. In some embodiments, a control PT comprises any of SEQ ID NOs: 1-5.

A recombinant host cell that expresses a heterologous gene encoding a PT described in this disclosure, including a chimeric PT and/or a chimeric fusion, may be capable of producing more CBGA and/or CBGVA relative to a host cell that expresses a control PT. In some embodiments, a control PT comprises any of SEQ ID NOs: 1-5.

In some embodiments, a recombinant host cell that expresses a heterologous gene encoding a PT described in this disclosure, including a chimeric PT and/or a chimeric fusion, that produces more CBGA and/or CBGVA relative to a control PT may be capable of producing at least 1% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 300%, at least 400%, at least 500%, at least 600%, at least 700%, at least 800%, at least 900%, or at least 1,000%) more CBGA and/or CBGVA relative to a host cell that expresses a control PT. In some embodiments, a control PT comprises any of SEQ ID NOs: 1-5.

In some embodiments, a recombinant host cell that expresses a heterologous gene encoding a PT described in this disclosure, including a chimeric PT and/or a fusion protein, that produces more CBGA and/or CBGVA relative to a control PT may be capable of producing at least 1% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 300%, at least 400%, at least 500%, at least 600%, at least 700%, at least 800%, at least 900%, or at least 1,000%) more CBGA and/or OGOA relative to a host cell that expresses a control PT. In some embodiments, a control PT comprises any of SEQ ID NOs: 1-5.

PTs for use in producing cannabinoids may be selected based on any one or more desired features, such as substrate selectivity, potential products formed, yield/titer of a product of interest, solubility, and/or localization (e.g. cytosolic localization, intramembrane localization) of the enzyme.

d. Substrate Selectivity

Many prenyltransferases are known to have promiscuity in regard to prenyl donors and acceptors, which may result in a broad spectrum of potential products formed using a particular enzyme (Chen et al. Nat. Chem. Biol. (2017): 13(2): 226-234). Without being bound by a particular theory, promiscuous enzymes may be useful in some embodiments because different products may be produced by the enzyme by varying the substrate. In some embodiments, a promiscuous enzyme may be useful in producing different products from a composition of heterogenous substrates.

As a non-limiting example, the PT from Streptomyces sp., NphB, has been previously shown to prenylate both olivetol and olivetolic acid (Kuzuyama et al. Nature, 2005). Wild-type NphB has also been reported to display a high degree of both substrate and product promiscuity. Similarly, C. sativa CsPT4 has been previously shown to prenylate both olivetol and olivetolic acid (Luo et al. Nature, 2019).

In some instances, it may be preferable for the prenyltransferase to have high specificity and not be promiscuous. For example, it may be preferable for the prenyltransferase to be specific for a particular substrate, so that the prenyltransferase produces a more homogenous product mix (i.e., greater product purity). Without being bound by a particular theory, an enzyme that has high specificity for a particular substrate may be useful because it may reduce possible by-products due to impurities in the substrate composition. For instance, when an enzyme is used with a host cell, the host cell may have intracellular mechanisms to convert a particular feed substrate into an undesirable substrate. In such instances, an enzyme that is highly specific for the non-converted substrate may be used to produce a product that has a higher purity of a compound of interest. In some instances, a highly specific enzyme may be useful for simplifying downstream processing, e.g., removing the need for further product purification.

In certain embodiments, prenyltransferases may use a resorcinol optionally substituted at the 5-position, a compound of Formula (5), a β-resorcylic acid optionally substituted at the 6-position, or a compound of Formula (6):

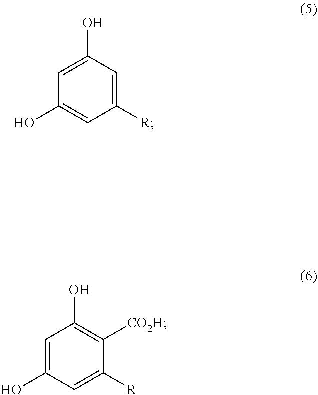

wherein R is hydrogen, optionally substituted acyl, optionally substituted alkyl, optionally substituted alkenyl, optionally substituted alkynyl, optionally substituted carbocyclyl, or optionally substituted aryl; and a compound comprising a prenyl group (e.g., geranyl diphosphate (GPP), isopentenyl diphosphate (IPP), neryl diphosphate (NPP), farnesyl diphosphate (FPP), and geranylgeranyl diphosphate (GGPP)) as substrates. R is as defined in this disclosure. In some embodiments, R is H, an optionally substituted C1-C11 alkyl, an optionally substituted C1-C11 alkenyl, an optionally substituted C1-C11 alkynyl, or an optionally substituted C1-C11 aralkyl.

In certain embodiments, prenyltransferases may use a compound of Formula (6):

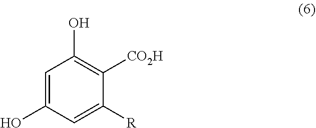

wherein R is hydrogen, optionally substituted acyl, optionally substituted alkyl, optionally substituted alkenyl, optionally substituted alkynyl, optionally substituted carbocyclyl, or optionally substituted aryl; and a compound comprising a prenyl group (e.g., geranyl diphosphate (GPP), isopentenyl diphosphate (IPP), farnesyl diphosphate (FPP), and geranylgeranyl diphosphate (GGPP)) as substrates. R is as defined in this disclosure.

A prenyltransferase may have different affinities for a particular substrate based on the R group on the substrate (e.g., the R group on a compound of Formula (5) and/or the R group on a compound of Formula (6)) and/or based on the presence or absence of a carboxylic acid on the substrate. In some embodiments, a particular R group may confer particular physiological effects to a compound. In some embodiments, a prenyltransferase may be chosen based on the ability of the prenyltransferase to use a substrate with a particular R group to produce a cannabinoid or cannabinoid precursor with a particular physiological effect.

In certain embodiments, a compound of Formula (6) is olivetolic acid (OA) (compound 6a of formula:

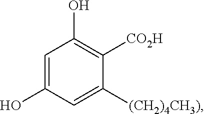

divarinic acid, a 6-acyl-resorcinolic acid derivative, 6-alkyl-resorcinolic acid derivative, or a 2,4 dihydroxy-6-acylbenzoic acid. In certain embodiments, a compound of Formula (6) is olivetolic acid (OA). In certain embodiments, a compound of Formula (6) is of the formula:

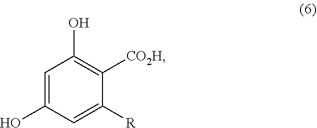

wherein R is optionally substituted C₁₋₆ alkyl. In certain embodiments, a compound of Formula (6) is of the formula:

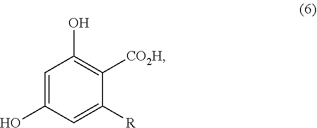

wherein R is unsubstituted C₁₋₆ alkyl. In certain embodiments, a compound of Formula (6) is divarinic acid. In certain embodiments, a compound of Formula (6) is a 6-acyl-resorcinolic acid derivative. In certain embodiments, a compound of Formula (6) is a 6-alkyl-resorcinolic acid derivative. In certain embodiments, a compound of Formula (6) is a 2,4 dihydroxy-6-acylbenzoic acid. In certain embodiments, in a compound of Formula (6). R is optionally substituted acyl. In some embodiments, orcinol, orsellinic acid, divarinol, divaric acid, olivetol, olivetolic acid, sphaerophorol, sphaeropholic acid, phlorisovalerophenone, naringenin, resveratrol, or a combination thereof are substrates.

In some embodiments, a substrate of the prenyltransferase is a compound of Formula (7′):

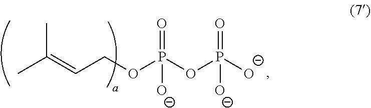

wherein a is 1, 2, 3, 4, 5, 6, 7, 8, 9, or 10, where examples include, but are not limited to, geranyl diphosphate or geranyl pyrophosphate (GPP), neryl pyrophosphate (NPP) or farnesyl pyrophosphate. In certain embodiments, a prenyltransferase substrate is a compound of Formula (7′):

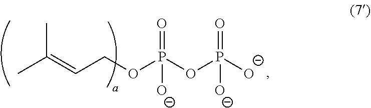

wherein a is 1, 2, 3, 4, 5, 6, 7, 8, 9, or 10. In certain embodiments, a prenyltransferase substrate is a compound of Formula (7′):

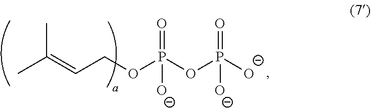

wherein a is 1, 2, 3, 4, or 5. In certain embodiments, a prenyltransferase substrate is geranyl diphosphate or geranyl pyrophosphate (GPP).

In some embodiments, a is 1. In some embodiments, a is 2. In some embodiments, a is 3. In some embodiments, a is 4. In some embodiments, a is 5. In some embodiments, a is 6. In some embodiments, a is 7. In some embodiments, a is 8. In some embodiments, a is 9. In some embodiments, a is 10. In some embodiments, a is 1, 2, 3, 4, or 5. In some embodiments, a is 1, 2, 3, or 4. In some embodiments, a is 6, 7, 8, 9, or 10.

In some embodiments, a substrate of the prenyltransferase is a compound of Formula (7a):

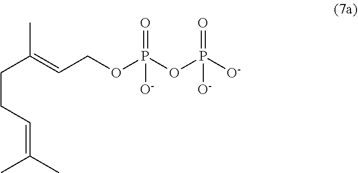 [SMILES: CC(C)=CCC/C(C)=C/COP(=O)([O-])OP(=O)([O-])[O-]]

In some embodiments, PT catalyzes the formation of a compound one or more of Formula (8a), Formula (8w), Formula (8x), Formula (8′), Formula (8y), and/or Formula (8z):

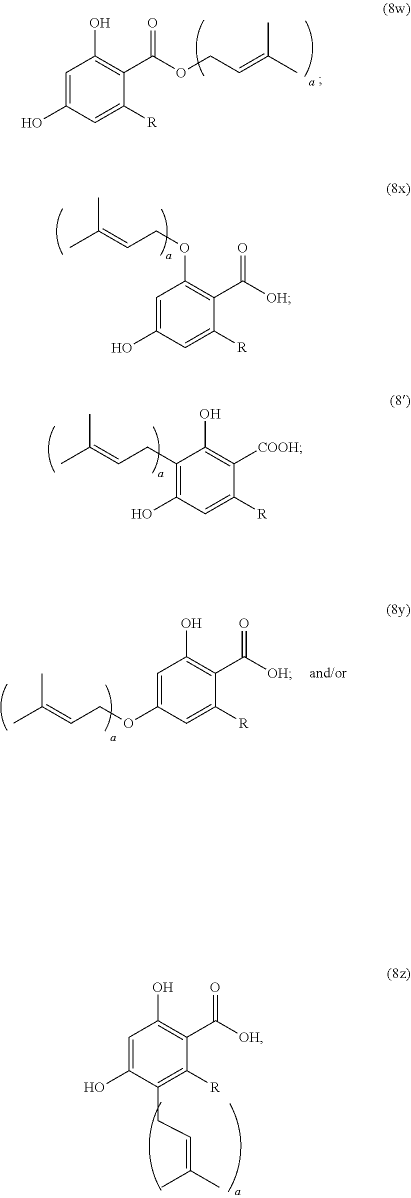

wherein a is 1, 2, 3, 4, 5, 6, 7, 8, 9, or 10.

In some embodiments, PT catalyzes the formation of a compound of Formula (8′);

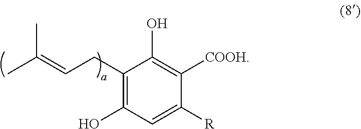

wherein a is 1, 2, 3, 4, 5, 6, 7, 8, 9, or 10.

In some embodiments, a is 1. In some embodiments, a is 2. In some embodiments, a is 3. In some embodiments, a is 4. In some embodiments, a is 5. In some embodiments, a is 6. In some embodiments, a is 7. In some embodiments, a is 8. In some embodiments, a is 9. In some embodiments, a is 10. In some embodiments, a is 1, 2, 3, 4, or 5. In some embodiments, a is 1, 2, 3, or 4. In some embodiments, a is 6, 7, 8, 9, or 10.

In some embodiments, PT catalyzes the formation of a compound of Formula (8):

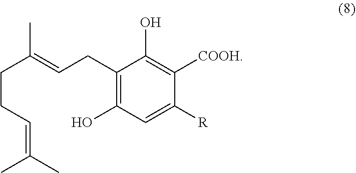

In some embodiments, a compound of Formula (8) is a compound of Formula (8a):

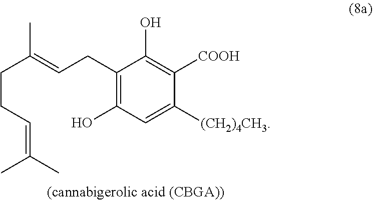

In some embodiments, PT catalyzes the formation of a compound of Formula (8x):

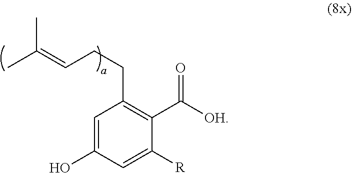

In some embodiments, a compound of Formula (8x) is of Formula (13):

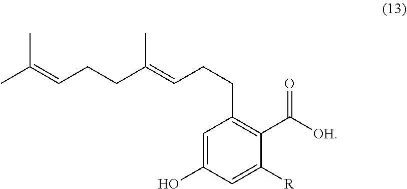

In some embodiments, PT catalyzes the formation of a compound of Formula (13):

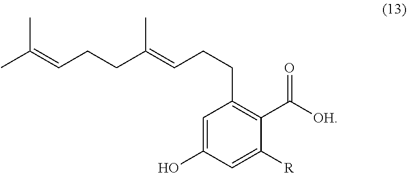

In some embodiments, a compound of Formula (13) is a compound of Formula (8b):

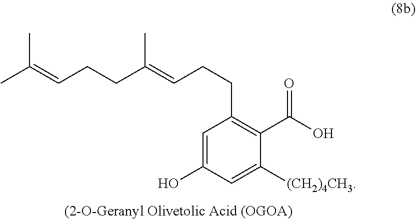

In some embodiments, the PT is a cannabigerolic acid synthase (CBGAS). CBGAS catalyzes the formation of CBGA from OA and GPP.

In some embodiments, a PT is a cannabigerovarinic acid synthase (CBGVAS). CBGVAS catalyze the formation of CBGVA from divarinic acid (DVA) and geranyl pyroshosphate (GPP).

In some embodiments, a PT may be capable of consuming a substrate of a compound of Formula 6 in FIG. 2 at a rate that is at least 1% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 300%, at least 400%, at least 500%, at least 600%, at least 700%, at least 800%, at least 900%, or at least 1,000%) faster or slower relative to a control.

In some embodiments, a control is a wild-type reference PT. A wild-type reference PT can be full-length or truncated. A wild-type reference PT can be part of a fusion protein. In some embodiments, a control is any one of SEQ ID NOs: 1-10. In some embodiments, a control is a fusion protein comprising any one of SEQ ID NOs: 1-10.

e. Prenylation

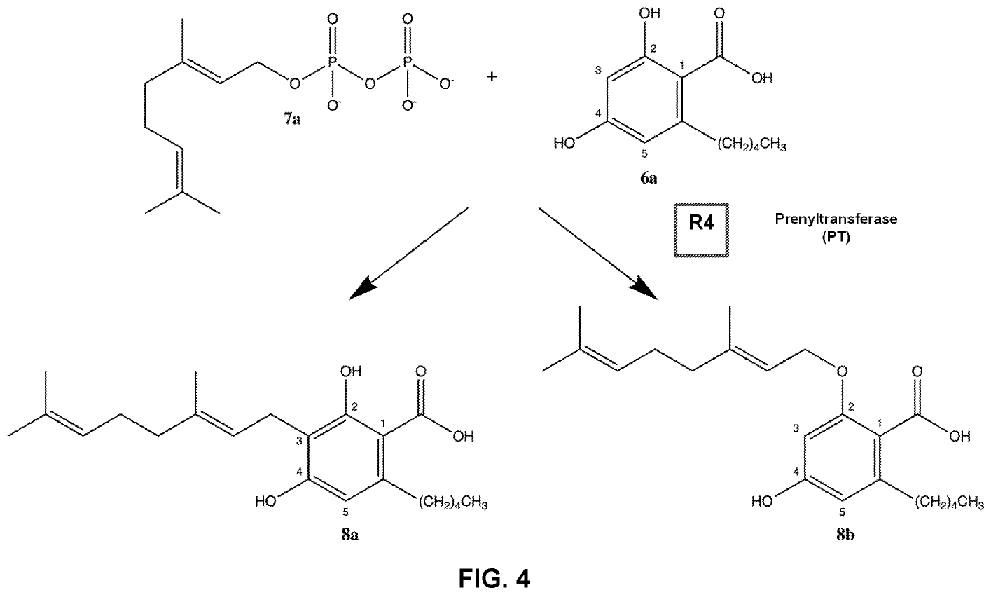

In addition to promiscuity in regard to potential substrates utilized, many prenyltransferases are known to also be promiscuous as to the products formed due to the ability to prenylate a prenyl acceptor at different sites, further resulting in a broad spectrum of potential products formed using a particular enzyme (Chen et al. Nat. Chem. Biol. (2017): 13(2): 226-234). When tested for activity using geranyl pyrophosphate (GPP) and olivetolic acid (OA) as substrates, NphB and CsPT4 produce multiple prenylation products (Kumano et al. Bioorganic Medicinal Chemistry, 2008; Luo et al. Nature, 2019). In particular, on OA at carbon positions labeled 3 and 5 and oxygen positions labeled 2 and 4 in Structure 6a (FIG. 4). Zirpel et al. reported the major prenylation product of wild-type NphB to be 2-O-Geranyl Olivetolic Acid (OGOA, Formula (8b) in FIG. 4)), with CBGA produced as the minor product (Formula (8a) in FIG. 1 and FIG. 4, Zirpel et al. Journal of Biotechnology, 2017). Functional expression of NphB and production of CBGA in S. cerevisiae was detected (Zirpel et al. Journal of Biotechnology, 2017).

In some instances, it may be preferable to prenylate at a particular position in Formula (6) or Formula (5). For example, it may be preferable to use a prenyltransferase (e.g., in combination with a terminal synthase) to produce phytocannabinoids, which are commonly prenylated at the C3 position of Formula (6).

In some instances, prenylation at a particular position in Formula (6) or Formula (5) may be used to alter the pharmacokinetic profile of cannabinoid products. For example, prenylation at a particular position in Formula (6) or Formula (5) may allow for the development of a cannabinoid product that crosses the blood brain barrier.

In some embodiments, a PT described in this disclosure transfers one or more prenyl groups to any of positions 2, 3, 4, or 5 in a compound of Formula (5), shown below:

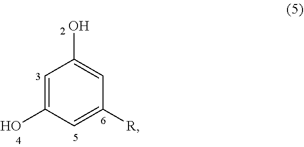

In some embodiments, a PT described in this disclosure transfers one or more prenyl groups to position 3 in a compound of Formula (5), shown below:

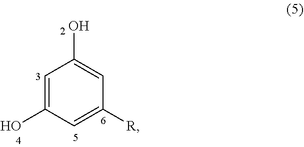

In some embodiments, a PT described in this disclosure transfers one or more prenyl groups to any of positions 1, 2, 3, 4, or 5 in a compound of Formula (6), shown below:

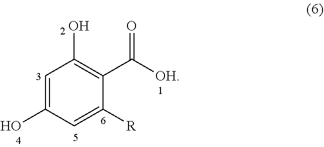

In some embodiments, the PT transfers a prenyl group to any of positions 1, 2, 3, 4, or 5 in a compound of Formula (6), shown below:

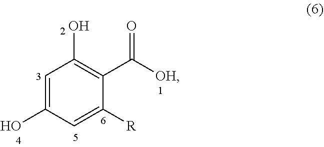

to form a compound of one or more of Formula (8w), Formula (8x), Formula (8′), Formula (8y), Formula (8z):

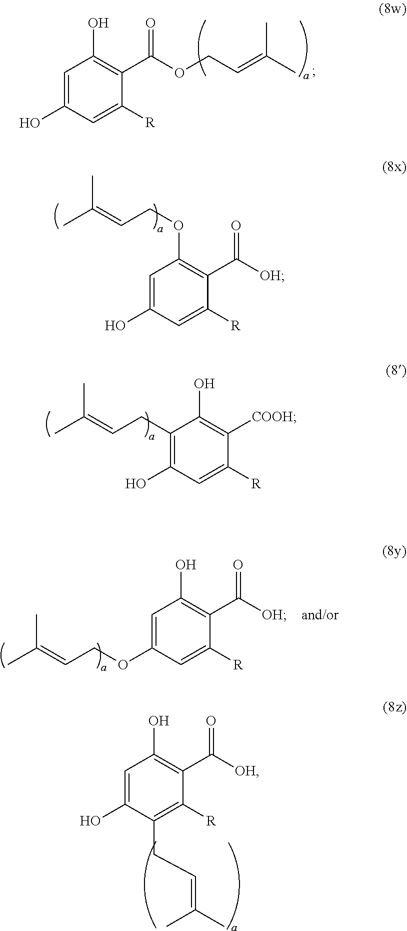

or a pharmaceutically acceptable salt, solvate, hydrate, polymorph, co-crystal, tautomer, stereoisomer, isotopically labeled derivative, or prodrug thereof, wherein a is 1, 2, 3, 4, 5, 6, 7, 8, 9, or 10. In some embodiments, the PT transfers a prenyl group to any of positions 1, 2, 3, 4, or 5 in a compound of Formula (6), shown below:

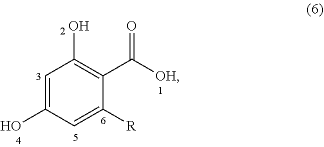

to form a compound of one or more of Formula (8w), Formula (8x), Formula (8′), Formula (8y), Formula (8z), wherein a is 1, 2, 3, 4, or 5. In some embodiments, the PT transfers a prenyl group to any of positions 1, 2, 3, 4, or 5 in a compound of Formula (6), shown below:

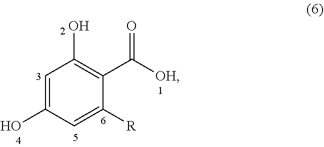

to form a compound of one or more of Formula (8w), Formula (8x), Formula (8′), Formula (8y), Formula (8z), or a pharmaceutically acceptable salt thereof, wherein a is 1, 2, 3, 4, 5, 6, 7, 8, 9, or 10.

In some embodiments, provided is a host cell where the PT is capable of producing a compound using a substrate of Formula (6):

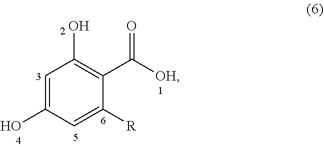

by transferring one or more prenyl groups to any of positions 1, 2, 3, 4, or 5 in the substrate of Formula (6).

In some embodiments, provided is a host cell where the PT is capable of producing a compound using a substrate of Formula (6):

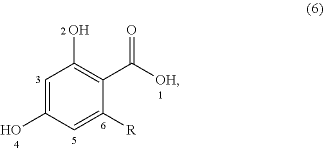

by transferring a prenyl group to any of positions 1, 2, 3, 4, or 5 in the substrate of Formula (6),
to form a compound of one or more of Formula (8w), Formula (8x). Formula (8′), Formula (8y), and/or Formula (8z):

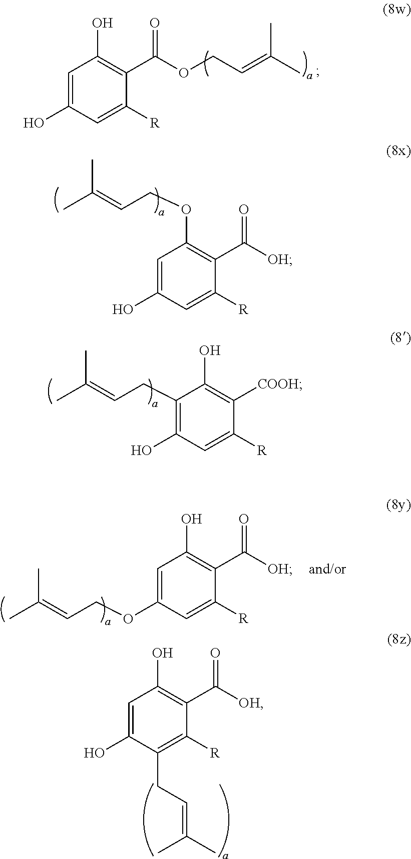

wherein a is 1, 2, 3, 4, 5, 6, 7, 8, 9, or 10.

In some embodiments, provided is a host cell where the PT is capable of producing a compound using a substrate of Formula (6):

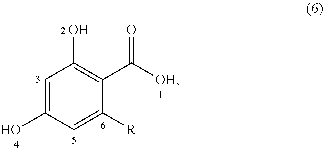

by transferring aprenyl group to position 1 in the substrate of Formula (6), to form a compound of Formula (8w):

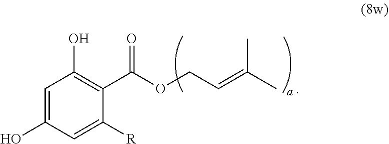

In some embodiments, provided is a host cell where the PT is capable of producing a compound using a substrate of Formula (6):

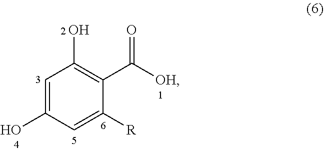

by transferring a prenyl group to position 2 in the substrate of Formula (6), to form a compound of Formula (8x):

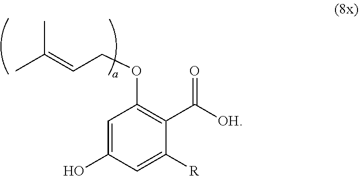

In some embodiments, provided is a host cell where the PT is capable of producing a compound using a substrate of Formula (6):

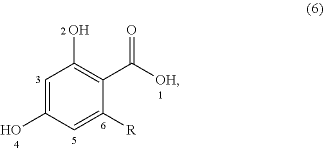

by transferring a prenyl group to position 2 in the substrate of Formula (6), to form a compound of Formula (13):

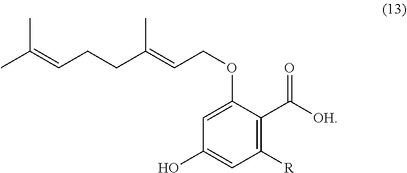

In some embodiments, provided is a host cell where the PT is capable of producing a compound using a substrate of Formula (6):

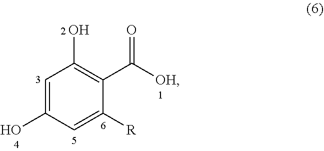

by transferring a prenyl group to position 3 in the substrate of Formula (6), to form a compound of Formula (8′):

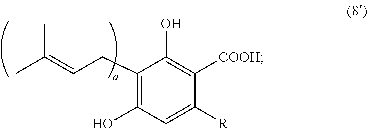

In some embodiments, provided is a host cell where the PT is capable of producing a compound using a substrate of Formula (6):

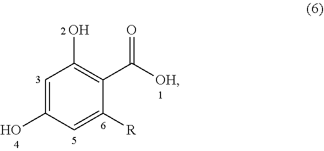

by transferring a prenyl group to position 3 in the substrate of Formula (6), to form a compound of Formula (8):

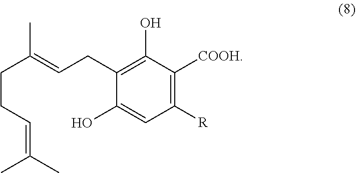

In some embodiments, provided is a host cell where the PT is capable of producing a compound using a substrate of Formula (6):

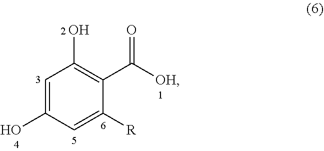

by transferring a prenyl group to position 4 in the substrate of Formula (6), to form a compound of Formula (8y):

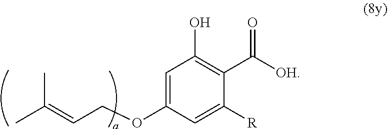

In some embodiments, provided is a host cell where the PT is capable of producing a compound using a substrate of Formula (6):

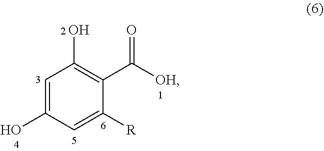

by transferring a prenyl group to position 5 in the substrate of Formula (6), to form a compound of Formula (8z):

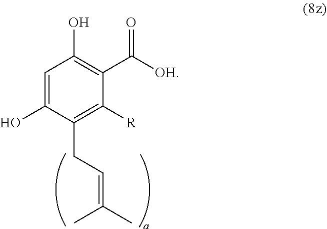

In some embodiments, provided is a method for producing a prenylated product of a compound of Formula (6);

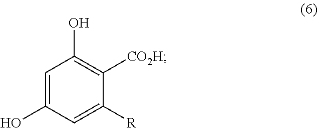

comprising contacting:
(a) a compound of Formula (6):

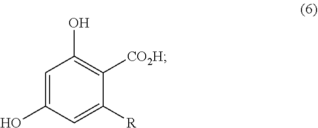

and
   (b) a compound of Formula (7′):

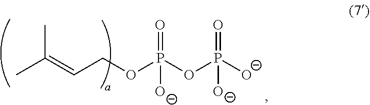

wherein a is 1, 2, 3, 4, 5, 6, 7, 8, 9, or 10;
in the presence of (c) a PT comprising a sequence that is at least 90% identical to a PT sequence disclosed in this application, including chimeric PTs and fusions comprising chimeric PTs.

In some embodiments, provided is a method for producing a prenylated product of a compound of Formula (6):

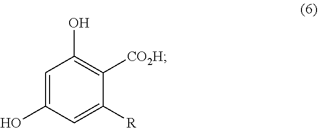

comprising contacting:
(a) a compound of Formula (6): 198

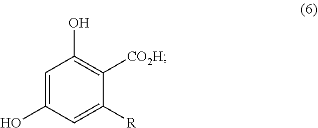

and
(b) a compound of Formula (7a):

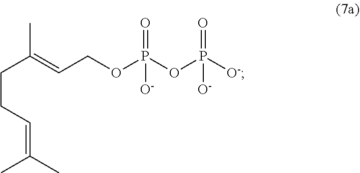

in the presence of (c) a PT comprising a sequence that is at least 90% identical to a PT sequence disclosed in this application, including chimeric PTs and fusions comprising chimeric PTs.

In some embodiments, the prenylated product of a compound of Formula (6) is a compound of Formula (8w), Formula (8x), Formula (8′), Formula (8y), or Formula (8z):

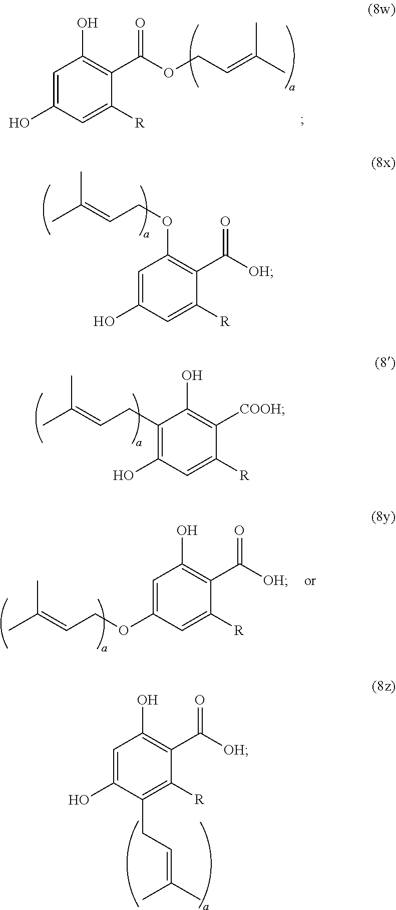

wherein a is 1, 2, 3, 4, 5, 6, 7, 8, 9, or 10. In some embodiments, the prenylated product of a compound of Formula (6) is a compound of Formula (8w), Formula (8x), Formula (8′), Formula (8y), or Formula (8z); wherein a is 1, 2, 3, 4, or 5. In some embodiments, the prenylated product of a compound of Formula (6) is a compound of Formula (8w), Formula (8x), Formula (8′), Formula (8y), or Formula (8z); wherein a is 6, 7, 8, 9, or 10.

In some embodiments, one or more mutations may be introduced into a prenyltransferase to change the enzyme's preferred prenylation site on a substrate. In some embodiments, the mutations are located at one or more residues corresponding to Y288, F213, Y288, G286, F213, Y288, and A232 in wild-type NphB. For example, in some embodiments, the mutations correspond to one or more of Y288A, F213H, Y288N, G286S, F213N, Y288V, and A232S in wild-type NphB. See, e.g., the NphB mutations disclosed in Valliere et al. Nat Commun. 2019 Feb. 4; 10(1):565, which is incorporated by reference in this disclosure in its entirety.

Any of the enzymes, host cells, and methods described in this application may be used for the production of cannabinoids and cannabinoid precursors, such as those provided in Table 1. In general, the term “production” is used to refer to the generation of one or more products (e.g., products of interest and/or by-products/off-products), for example, from a particular substrate or reactant. The amount of production may be evaluated at any one or more steps of a pathway, such as a final product or an intermediate product, using metrics familiar to one of ordinary skill in the art. For example, the amount of production may be assessed for a single enzymatic reaction (e.g., conversion of OA to CBGAS by a PT). Alternatively or in addition, the amount of production may be assessed for a series of enzymatic reactions (e.g., the biosynthetic pathway shown in FIG. 1 and/or FIG. 2). Production may be assessed by any metrics known in the art, for example, by assessing volumetric productivity, enzyme kinetics/reaction rate, specific productivity biomass-specific productivity, titer, yield, and total titer of one or more products (e.g. products of interest and/or by-products/off-products).

In some embodiments, the metric used to measure production may depend on whether a continuous process is being monitored (e.g., several cannabinoid biosynthesis steps are used in combination) or whether a particular end product is being measured. For example, in some embodiments, metrics used to monitor production by a continuous process may include volumetric productivity, enzyme kinetics and reaction rate. In some embodiments, metrics used to monitor production of a particular product may include specific productivity biomass-specific productivity, titer, yield, and total titer of one or more products (e.g., products of interest and/or by-products/off-products).

Production of one or more products (e.g., products of interest and/or by-products/off-products) may be assessed indirectly, for example by determining the amount of a substrate remaining following termination of the reaction/fermentation. For example, for a CBGAS that catalyzes the formation of products (e.g., CBGA and OGOA) from OA and GPP, production of the products may be assessed by quantifying the CBGA (or OGOA) directly or by quantifying the amount of substrate remaining following the reaction (e.g. amount of OA or GPP).

In instances in which prenylation at a particular position in a compound is desired, it may be preferable to monitor production of products directly. For example, if one or more mutations are introduced into a reference prenyltransferase to alter the preferred prenylation site on a substrate, the reference prenyltransferase and its mutated counterpart may consume the same amount of a particular substrate, but may produce a different ratio of products. In some embodiments, a PT that exhibits high production of by-products but low production of a desired product may still be used, for example if one or more mutations are introduced that shift production to a preferred product.

In some embodiments, the production of a product (e.g., products of interest and/or by-products/off-products) may be assessed as relative production, for example relative to a control. In some embodiments, the production of CBGA by a particular PT may be assessed relative to a control. The control PT may be, e.g., a wild-type enzyme, or an enzyme containing one or more mutations. In some embodiments, the production of CBGA by a particular PT in a host cell may be assessed relative to a PT in another host cell. In some embodiments, the production of CBGA from a particular substrate may be assessed relative to a control using a different substrate.

In some embodiments, a PT may be capable of producing at least 1% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 300%, at least 40%, at least 500%, at least 6000%, at least 700%, at least 800%, at least 900%, or at least 1,000%) the amount of one or more products relative to a control.

In some embodiments, a PT may be capable of producing a product at a higher titer or yield relative to a control. In some embodiments, a PT may be capable of producing a product at a faster rate (e.g., higher productivity) relative to a control. In some embodiments, a PT may have preferential binding and/or activity towards one substrate relative to another substrate. In some embodiments, a PT may preferentially produce one product relative to another product.

In some embodiments, a PT may produce at least 0.0001 μg/L, at least 0.001 μg/L, at least 0.01 μg/L, at least 0.02 μg/L, at least 0.03 μg/L, at least 0.04 μg/L, at least 0.05 μg/L, at least 0.06 μg/L, at least 0.07 μg/L, at least 0.08 μg/L, at least 0.09 μg/L, at least 0.1 μg/L, at least 0.11 μg/L, at least 0.12 μg/L, at least 0.13 μg/L, at least 0.14 μg/L, at least 0.15 μg/L, at least 0.16 μg/L, at least 0.17 μg/L, at least 0.18 μg/L, at least 0.19 μg/L, at least 0.2 μg/L, at least 0.21 μg/L, at least 0.22 μg/L, at least 0.23 μg/L, at least 0.24 μg/L, at least 0.25 μg/L, at least 0.26 μg/L, at least 0.27 μg/L, at least 0.28 μg/L, at least 0.29 μg/L, at least 0.3 μg/L, at least 0.31 μg/L, at least 0.32 μg/L, at least 0.33 μg/L, at least 0.34 μg/L, at least 0.35 μg/L, at least 0.36 μg/L, at least 0.37 μg/L, at least 0.38 μg/L, at least 0.39 μg/L, at least 0.4 μg/L, at least 0.41 μg/L, at least 0.42 μg/L, at least 0.43 μg/L, at least 0.44 μg/L, at least 0.45 μg/L, at least 0.46 μg/L, at least 0.47 μg/L, at least 0.48 μg/L, at least 0.49 μg/L, at least 0.5 μg/L, at least 0.51 μg/L, at least 0.52 μg/L, at least 0.53 μg/L, at least 0.54 μg/L, at least 0.55 μg/L, at least 0.56 μg/L, at least 0.57 μg/L, at least 0.58 μg/L, at least 0.59 μg/L, at least 0.6 μg/L, at least 0.61 μg/L, at least 0.62 μg/L, at least 0.63 μg/L, at least 0.64 μg/L, at least 0.65 μg/L, at least 0.66 μg/L, at least 0.67 μg/L, at least 0.68 μg/L, at least 0.69 μg/L, at least 0.7 μg/L, at least 0.71 μg/L, at least 0.72 μg/L, at least 0.73 μg/L, at least 0.74 μg/L, at least 0.75 μg/L, at least 0.76 μg/L, at least 0.77 μg/L, at least 0.78 μg/L, at least 0.79 μg/L, at least 0.8 μg/L, at least 0.81 μg/L, at least 0.82 μg/L, at least 0.83 μg/L, at least 0.84 μg/L, at least 0.85 μg/L, at least 0.86 μg/L, at least 0.87 μg/L, at least 0.88 μg/L, at least 0.89 μg/L, at least 0.9 μg/L, at least 0.91 μg/L, at least 0.92 μg/L, at least 0.93 μg/L, at least 0.94 μg/L, at least 0.95 μg/L, at least 0.96 μg/L, at least 0.97 μg/L, at least 0.98 μg/L, at least 0.99 μg/L, at least 1 μg/L, at least 1.1 μg/L, at least 1.2 μg/L, at least 1.3 μg/L, at least 1.4 μg/L, at least 1.5 μg/L, at least 1.6 μg/L, at least 1.7 μg/L, at least 1.8 μg/L, at least 1.9 μg/L, at least 2 μg/L, at least 2.1 μg/L, at least 2.2 μg/L, at least 2.3 μg/L, at least 2.4 μg/L, at least 2.5 μg/L, at least 2.6 μg/L, at least 2.7 μg/L, at least 2.8 μg/L, at least 2.9 μg/L, at least 3 μg/L, at least 3.1 μg/L, at least 3.2 μg/L, at least 3.3 μg/L, at least 3.4 μg/L, at least 3.5 μg/L, at least 3.6 μg/L, at least 3.7 μg/L, at least 3.8 μg/L, at least 3.9 μg/L, at least 4 μg/L, at least 4.1 μg/L, at least 4.2 μg/L, at least 4.3 μg/L, at least 4.4 μg/L, at least 4.5 μg/L, at least 4.6 μg/L, at least 4.7 μg/L, at least 4.8 μg/L, at least 4.9 μg/L, at least 5 μg/L, at least 5.1 μg/L, at least 5.2 μg/L, at least 5.3 μg/L, at least 5.4 μg/L, at least 5.5 μg/L, at least 5.6 μg/L, at least 5.7 μg/L, at least 5.8 μg/L, at least 5.9 μg/L, at least 6 μg/L, at least 6.1 μg/L, at least 6.2 μg/L, at least 6.3 μg/L, at least 6.4 μg/L, at least 6.5 μg/L, at least 6.6 μg/L, at least 6.7 μg/L, at least 6.8 μg/L, at least 6.9 μg/L, at least 7 μg/L, at least 7.1 μg/L, at least 7.2 μg/L, at least 7.3 μg/L, at least 7.4 μg/L, at least 7.5 μg/L, at least 7.6 μg/L, at least 7.7 μg/L, at least 7.8 μg/L, at least 7.9 μg/L, at least 8 μg/L, at least 8.1 μg/L, at least 8.2 μg/L, at least 8.3 μg/L, at least 8.4 μg/L, at least 8.5 μg/L, at least 8.6 μg/L, at least 8.7 μg/L, at least 8.8 μg/L, at least 8.9 μg/L, at least 9 μg/L, at least 9.1 μg/L, at least 9.2 μg/L, at least 9.3 μg/L, at least 9.4 μg/L, at least 9.5 μg/L, at least 9.6 μg/L, at least 9.7 μg/L, at least 9.8 μg/L, at least 9.9 μg/L, at least 10 μg/L, at least 10.1 μg/L, at least 10.2 μg/L, at least 10.3 μg/L, at least 10.4 μg/L, at least 10.5 μg/L, at least 10.6 μg/L, at least 10.7 μg/L, at least 10.8 μg/L, at least 10.9 μg/L, at least 11 μg/L, at least 11.1 μg/L, at least 11.2 μg/L, at least 11.3 μg/L, at least 11.4 μg/L, at least 11.5 μg/L, at least 11.6 μg/L, at least 11.7 μg/L, at least 11.8 μg/L, at least 11.9 μg/L, at least 12 μg/L, at least 12.1 μg/L, at least 12.2 μg/L, at least 12.3 μg/L, at least 12.4 μg/L, at least 12.5 μg/L, at least 12.6 μg/L, at least 12.7 μg/L, at least 12.8 μg/L, at least 12.9 μg/L, at least 13 μg/L, at least 13.1 μg/L, at least 13.2 μg/L, at least 13.3 μg/L, at least 13.4 μg/L, at least 13.5 μg/L, at least 13.6 μg/L, at least 13.7 μg/L, at least 13.8 μg/L, at least 13.9 μg/L, at least 14 μg/L, at least 14.1 μg/L, at least 14.2 μg/L, at least 14.3 μg/L, at least 14.4 μg/L, at least 14.5 μg/L, at least 14.6 μg/L, at least 14.7 μg/L, at least 14.8 μg/L, at least 14.9 μg/L, at least 15 μg/L, at least 15.1 μg/L, at least 15.2 μg/L, at least 15.3 μg/L, at least 15.4 μg/L, at least 15.5 μg/L, at least 15.6 μg/L, at least 15.7 μg/L, at least 15.8 μg/L, at least 15.9 μg/L, at least 16 μg/L, at least 16.1 μg/L, at least 16.2 μg/L, at least 16.3 μg/L, at least 16.4 μg/L, at least 16.5 μg/L, at least 16.6 μg/L, at least 16.7 μg/L, at least 16.8 μg/L, at least 16.9 μg/L, at least 17 μg/L, at least 17.1 μg/L, at least 17.2 μg/L, at least 17.3 μg/L, at least 17.4 μg/L, at least 17.5 μg/L, at least 17.6 μg/L, at least 17.7 μg/L, at least 17.8 μg/L, at least 17.9 μg/L, at least 18 μg/L, at least 18.1 μg/L, at least 18.2 μg/L, at least 18.3 μg/L, at least 18.4 μg/L, at least 18.5 μg/L, at least 18.6 μg/L, at least 18.7 μg/L, at least 18.8 μg/L, at least 18.9 μg/L, at least 19 μg/L, at least 19.1 μg/L, at least 19.2 μg/L, at least 19.3 μg/L, at least 19.4 μg/L, at least 19.5 μg/L, at least 19.6 μg/L, at least 19.7 μg/L, at least 19.8 μg/L, at least 19.9 μg/L, at least 20 μg/L, at least 25 μg/L, at least 30 μg/L, at least 35 μg/L, at least 40 μg/L, at least 45 μg/L, at least 50 μg/L, at least 55 μg/L, at least 60 μg/L, at least 65 μg/L, at least 70 μg/L, at least 75 μg/L, at least 80 μg/L, at least 85 μg/L, at least 90 μg/L, at least 95 μg/L, at least 100 μg/L, at least 105 μg/L, at least 110 μg/L, at least 115 μg/L, at least 120 μg/L, at least 125 μg/L, at least 130 μg/L, at least 135 μg/L, at least 140 μg/L, at least 145 μg/L, at least 150 μg/L, at least 155 μg/L, at least 160 μg/L, at least 165 μg/L, at least 170 μg/L, at least 175 μg/L, at least 180 μg/L, at least 185 μg/L, at least 190 μg/L, at least 195 μg/L, at least 200 μg/L, at least 205 μg/L, at least 210 μg/L, at least 215 μg/L, at least 220 μg/L, at least 225 μg/L, at least 230 μg/L, at least 235 μg/L, at least 240 μg/L, at least 245 μg/L, at least 250 μg/L, at least 255 μg/L, at least 260 μg/L, at least 265 μg/L, at least 270 μg/L, at least 275 μg/L, at least 280 μg/L, at least 285 μg/L, at least 290 μg/L, at least 295 μg/L, at least 300 μg/L, at least 305 μg/L, at least 310 μg/L, at least 315 μg/L, at least 320 μg/L, at least 325 μg/L, at least 330 μg/L, at least 335 μg/L, at least 340 μg/L, at least 345 μg/L, at least 350 μg/L, at least 355 μg/L, at least 360 μg/L, at least 365 μg/L, at least 370 μg/L, at least 375 μg/L, at least 380 μg/L, at least 385 μg/L, at least 390 μg/L, at least 395 μg/L, at least 400 μg/L, at least 405 μg/L, at least 410 μg/L, at least 415 μg/L, at least 420 μg/L, at least 425 μg/L, at least 430 μg/L, at least 435 μg/L, at least 440 μg/L, at least 445 μg/L, at least 450 μg/L, at least 455 μg/L, at least 460 μg/L, at least 465 μg/L, at least 470 μg/L, at least 475 μg/L, at least 480 μg/L, at least 485 μg/L, at least 490 μg/L, at least 495 μg/L, at least 500 μg/L, at least 600 μg/L, at least 700 μg/L, at least 800 μg/L, at least 900 μg/L, at least or 1000 μg/L of one or more compounds selected from those listed in Table 3. In Table 3, for each compound, a may independently be 1, 2, 3, 4, 5, 6, 7, 8, 9, or 10. In some embodiments, the compound is CBGA. In some embodiments, the compound is CBGVA. In some embodiments, the compound is OGOA.

In some embodiments, a PT may be capable of producing at least 1% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 300%, at least 400%, at least 500%, at least 600%, at least 700%, at least 800%, at least 900%, or at least 1,000%) more of one or more compounds selected from those listed in Table 3 relative to a control. In Table 3, for each compound, a may independently be 1, 2, 3, 4, 5, 6, 7, 8, 9, or 10.

In some embodiments, a PT may be capable of producing at least 1% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 800%, at least 85%, at least 90%, at least 95%, at least 100%, at least 125%, at least 150%, at least 175%, at least 200%, at least 300%, at least 400%, at least 500%, at least 600%, at least 700%, at least 800%, at least 900%, or at least 1,000%) higher titer or yield of one or more compounds selected from those listed in Table 3 relative to a control. In Table 3, for each compound, a may independently be 1, 2, 3, 4, 5, 6, 7, 8, 9, or 10.

In some embodiments, a PT may be capable of producing one or more compounds selected from Table 3 at a rate that is at least 1% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 300%, at least 400%, at least 500%, at least 600%, at least 700%, at least 800%, at least 90)%, or at least 1,000%) faster relative to a control. In Table 3, for each compound, a may independently be 1, 2, 3, 4, 5, 6, 7, 8, 9, or 10.

In some embodiments, a PT may be capable of producing at least 1% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 300%, at least 400%, at least 500%, at least 600%, at least 700%, at least 800%, at least 900%, or at least 1,000%) more of a compound of Formula (8):

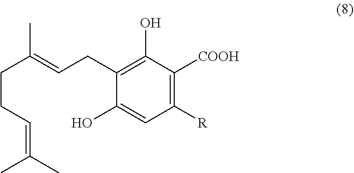

relative to a control.

In some embodiments, a PT may be capable of producing at least 1% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 300%, at least 400%, at least 500%, at least 600%, at least 700%, at least 800%, at least 900%, or at least 1,000%) more of a compound of Formula (8a):

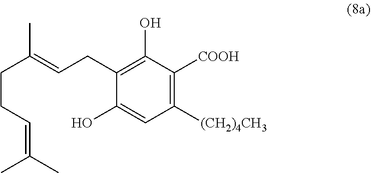

(cannabigerolic Acid (CBGA)) relative to a control.

In some embodiments, a PT may be capable of producing at least 1% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 300%, at least 400%, at least 500%, at least 600%, at least 700%, at least 800%, at least 900%, or at least 1,000%) more of a compound of Formula (8c):

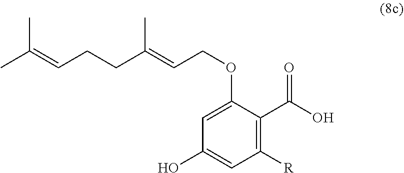

relative to a control.

In some embodiments, a PT may be capable of producing at least 1% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 300%, at least 400%, at least 500%, at least 600%, at least 700%, at least 800%, at least 900%, or at least 1,000%) more of a compound of Formula (8b):

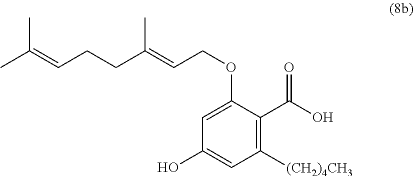

(2-O-Geranyl Olivetolic Acid (OGOA) relative to a control.

In some embodiments, a PT may be capable of producing at least 1% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 300%, at least 400%, at least 500%, at least 600%, at least 700%, at least 800%, at least 900%, or at least 1,000%) more of a compound of Formula (13):

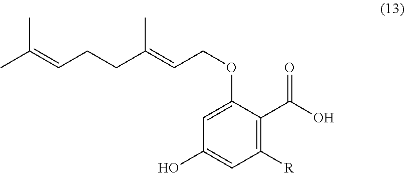

relative to a control.

In some embodiments, a PT may be capable of producing at least 1% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 300%, at least 400%, at least 500%, at least 600%, at least 700%, at least 800%, at least 900%, or at least 1,000%) more of a compound of Formula (8w), Formula (8x), Formula (8′), Formula (8y), or Formula (8z):

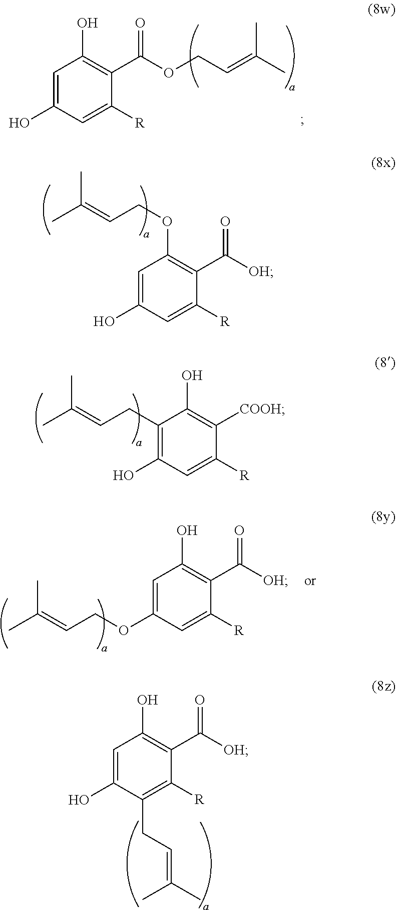

wherein a is 1, 2, 3, 4, 5, 6, 7, 8, 9, or 10, relative to a control. In some embodiments, a PT may be capable of producing at least 1% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 300%, at least 400%, at least 500%, at least 600%, at least 700%, at least 800%, at least 900%, or at least 1,000%) more of a compound of Formula (8w). Formula (8x), Formula (8′), Formula (8y), or Formula (8z), wherein a is 1, 2, 3, 4, or 5, relative to a control. In certain embodiments, a is 2, 3, 4, or 5.

In some embodiments, a PT may be capable of producing at least 1% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 300%, at least 400%, at least 500%, at least 600%, at least 700%, at least 800%, at least 900%, or at least 1,000%) more of a compound of Formula (8′):

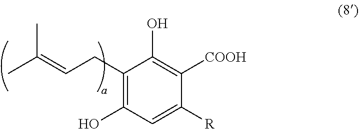

wherein a is 1, 2, 3, 4, 5, 6, 7, 8, 9, or 10, relative to a control.

In some embodiments, a PT may be capable of producing at least 1% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 300%, at least 400%, at least 500%, at least 600%, at least 700%, at least 800%, at least 900%, or at least 1.000%) less of one or more compounds selected from those listed in Table 3 relative to a control. In Table 3, for each compound, a may independently be 1, 2, 3, 4, 5, 6, 7, 8, 9, or 10.

In some embodiments, a PT may be capable of producing at least 1% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 300%, at least 400%, at least 500%, at least 600%, at least 700%, at least 800%, at least 900%, or at least 1,000%) less of a compound of Formula (8):

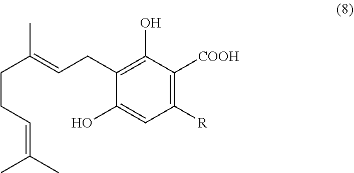

relative to a control.

In some embodiments, a PT may be capable of producing at least 1% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 300%, at least 400%, at least 500%, at least 600%, at least 700%, at least 800%, at least 900%, or at least 1,000%) less of a compound of Formula (8a): (cannabigerolic Acid (CBGA)) relative to a control.

In some embodiments, a PT may be capable of producing at least 1% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 300%, at least 400%, at least 500%, at least 600%, at least 700%, at least 800%, at least 900%, or at least 1,000%) less of a compound of Formula (8c):

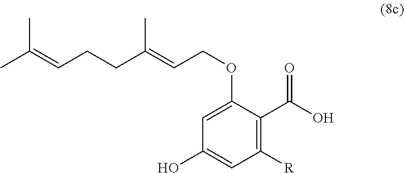

relative to a control.

In some embodiments, a PT may be capable of producing at least 1% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 300%, at least 400%, at least 500%, at least 600%, at least 700%, at least 800%, at least 900%, or at least 1,000%) less of a compound of Formula (8b) CBGA relative to a control.

In some embodiments, a PT may be capable of producing at least 1% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 300%, at least 400%, at least 500%, at least 600%, at least 700%, at least 800%, at least 900%, or at least 1,000%) less of a compound of Formula (13):

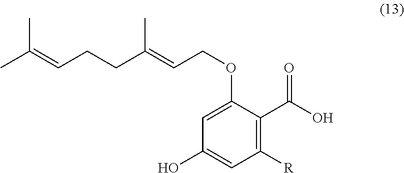

relative to a control.

In some embodiments, a PT may be capable of producing at least 1% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 300%, at least 400%, at least 500%, at least 600%, at least 700%, at least 800%, at least 900%, or at least 1,000%) less of a compound of Formula (8w), Formula (8x), Formula (8′), Formula (8y), or Formula (8z):

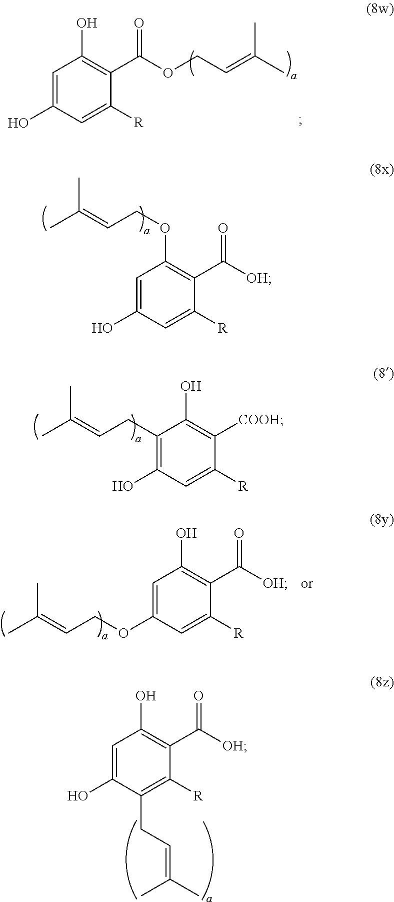

wherein a is 1, 2, 3, 4, 5, 6, 7, 8, 9, or 10, relative to a control.

In some embodiments, a PT may be capable of producing at least 1% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 300%, at least 400%, at least 500%, at least 600%, at least 700%, at least 800%, at least 900%, or at least 1,000%) less of a compound of Formula (8′):

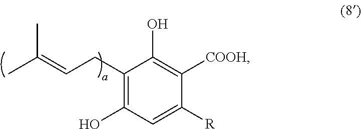

wherein a is 1, 2, 3, 4, 5, 6, 7, 8, 9, or 10, relative to a control.

In some embodiments, a PT may be capable of producing at least 1% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 10W %, at least 125%, at least 150%, at least 175%, at least 200%, at least 300%, at least 400%, at least 500%, at least 600%, at least 700%, at least 800%, at least 900%, or at least 1,000%) lower titer or yield of one or more compounds selected from those listed in Table 3 relative to a control. In Table 3, for each compound, a may independently be 1, 2, 3, 4, 5, 6, 7, 8, 9, or 10.

In some embodiments, a PT may be capable of producing one or more compounds selected from Table 3 at a rate that is at least 1% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 300%, at least 400%, at least 500%, at least 600%, at least 700%, at least 800%, at least 900%, or at least 1,000%) slower relative to a control. In Table 3, for each compound, a may independently be 1, 2, 3, 4, 5, 6, 7, 8, 9, or 10.

TABLE 3
Non-limiting examples of PT products.

| 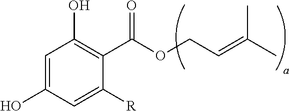 | (8w) |
|---|---|
| 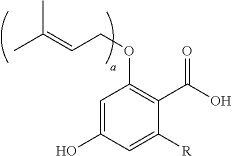 | (8x) |
| 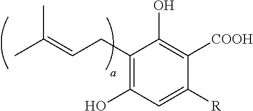 | (8′) |
| 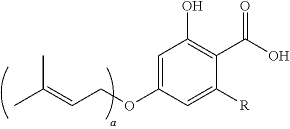 | (8y) |
| 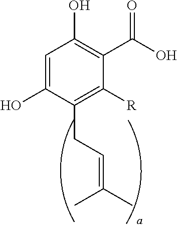 | (8z) |
| 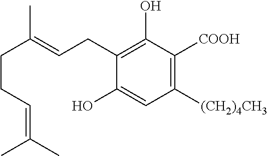 | (8a) |
| (cannabigerolic Acid (CBGA)) | |
| 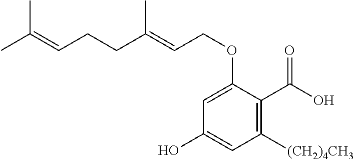 | (8b) |
| (2-O-Geranyl Olivetolic Acid | |
| (OGOA) | |
| 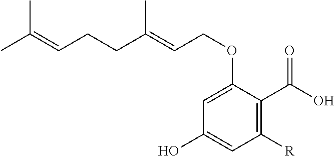 | (13) |

In some embodiments, a control is a wild-type reference PT. A wild-type reference PT can be full-length or truncated. A wild-type reference PT can be part of a fusion protein. In some embodiments, a control is any one of SEQ ID NOs: 1-10. In some embodiments, a control is a fusion protein comprising any one of SEQ ID NOs: 1-10.

In some embodiments, a PT is capable of producing a product mixture comprising one or more of Formula (8w), Formula (8x), Formula (8′), Formula (8y), and/or Formula (8z):

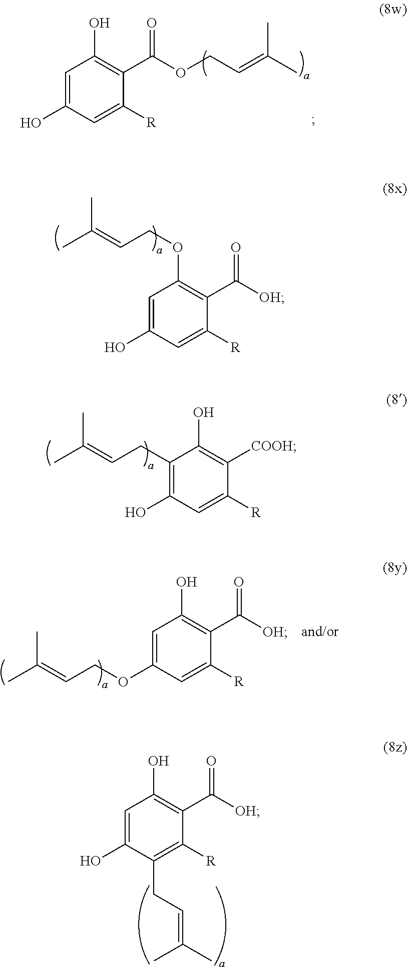

resulting from the prenylation of a compound of Formula (6), shown below:

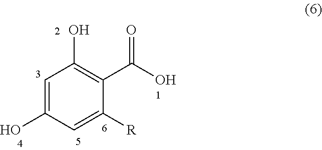

In some embodiments, at least approximately 50-100%, at least approximately 50-60%, at least approximately 60-70%, at least approximately 70-80%, at least approximately 80-90%, at least approximately 90-100%, of compounds within the product mixture are compounds of Formula (8′),

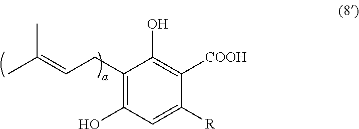

wherein a is 1, 2, 3, 4, 5, 6, 7, 8, 9, or 10.

In some embodiments, a PT is capable of producing a product mixture of prenylated products resulting from the prenylation of a compound of Formula (6), shown below:

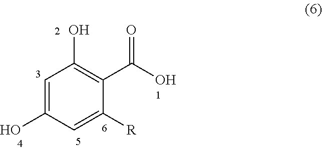

wherein at least approximately 50-100%, at least approximately 50-60%, at least approximately 60-70%, at least approximately 70-80%, at least approximately 80-90%, or at least approximately 90-100%, of the products are compounds of Formula (8′),

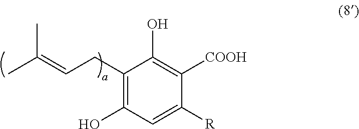

wherein a is 1, 2, 3, 4, 5, 6, 7, 8, 9, or 10.

In some embodiments, a PT is capable of producing a product mixture of prenylated products resulting from the prenylation of a compound of Formula (6), shown below:

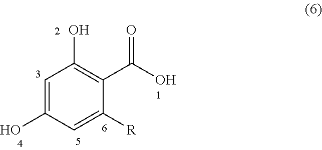

wherein at least approximately 50-100%, at least approximately 50-60%, at least approximately 60-70%, at least approximately 70-80%, at least approximately 80-90%, at least approximately 90-100%, of the products are compounds of Formula (8),

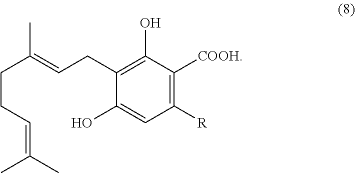

In some embodiments, a PT is capable of producing at least 1.1 times, 1.2 times, 1.3 times, 1.4 times, 1.5 times, 1.6 times, 1.7 times, 1.8 times, 1.9 times, 2 times, 2.1 times, 2.2 times, 2.3 times, 2.4 times, 2.5 times, 2.6 times, 2.7 times, 2.8 times, 2.9 times, 3 times, 3.1 times, 3.2 times, 3.3 times, 3.4 times, 3.5 times, 3.6 times, 3.7 times, 3.8 times, 3.9 times, 4 times, 5 times, 6 times, 8 times, 9 times, 10 times, 20 times, 30 times, 40 times, 50 times, 60 times, 70 times, 80 times, 90 times, 100 times, 200 times, 300 times, 400 times, 500 times, 600 times, 700 times, 800 times or 1,000 times more of a compound of Formula (8);

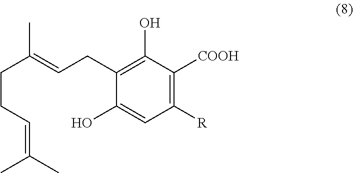

than a compound of Formula (13):

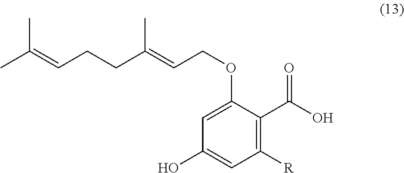

In some embodiments, a PT is capable of producing at least 1.1 times, 1.2 times, 1.3 times, 1.4 times, 1.5 times, 1.6 times, 1.7 times, 1.8 times, 1.9 times, 2 times, 2.1 times, 2.2 times, 2.3 times, 2.4 times, 2.5 times, 2.6 times, 2.7 times, 2.8 times, 2.9 times, 3 times, 3.1 times, 3.2 times, 3.3 times, 3.4 times, 3.5 times, 3.6 times, 3.7 times, 3.8 times, 3.9 times, 4 times, 5 times, 6 times, 8 times, 9 times, 10 times, 20 times, 30 times, 40 times, 50 times, 60 times, 70 times, 80 times, 90 times, 100 times, 200 times, 300 times, 400 times, 500 times, 600 times, 700 times, 800 times or 1,000 times more of a compound of Formula (8a):

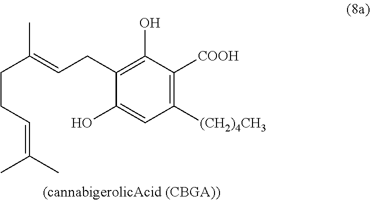

than a compound of Formula (8b):

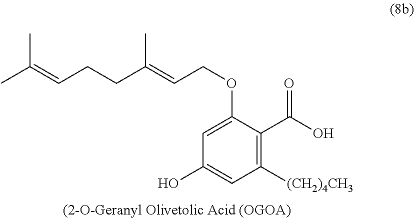

In some embodiments, a PT is capable of producing at least 1.1 times, 1.2 times, 1.3 times, 1.4 times, 1.5 times, 1.6 times, 1.7 times, 1.8 times, 1.9 times, 2 times, 2.1 times, 2.2 times, 2.3 times, 2.4 times, 2.5 times, 2.6 times, 2.7 times, 2.8 times, 2.9 times, 3 times, 3.1 times, 3.2 times, 3.3 times, 3.4 times, 3.5 times, 3.6 times, 3.7 times, 3.8 times, 3.9 times, 4 times, 5 times, 6 times, 8 times, 9 times, 10 times, 20 times, 30 times, 40 times, 50 times, 60 times, 70 times, 80 times, 90 times, 100 times, 200 times, 300 times, 400 times, 500 times, 600 times, 700 times, 800 times or 1,000 times more of a compound of Formula (13):

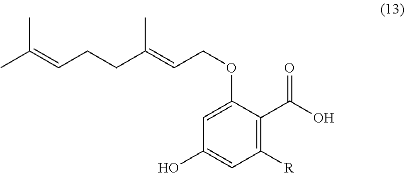

than a compound of Formula (8):

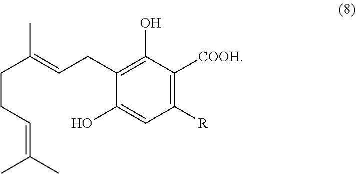

In some embodiments, a PT is capable of producing at least 1.1 times, 1.2 times, 1.3 times, 1.4 times, 1.5 times, 1.6 times, 1.7 times, 1.8 times, 1.9 times, 2 times, 2.1 times, 2.2 times, 2.3 times, 2.4 times, 2.5 times, 2.6 times, 2.7 times, 2.8 times, 2.9 times, 3 times, 3.1 times, 3.2 times, 3.3 times, 3.4 times, 3.5 times, 3.6 times, 3.7 times, 3.8 times, 3.9 times, 4 times, 5 times, 6 times, 8 times, 9 times, 10 times, 20 times, 30 times, 40 times, 50 times, 60 times, 70 times, 80 times, 90 times, 100 times, 200 times, 300 times, 400 times, 500 times, 600 times, 700 times, 800 times or 1.000 times more of a compound of Formula (8b):

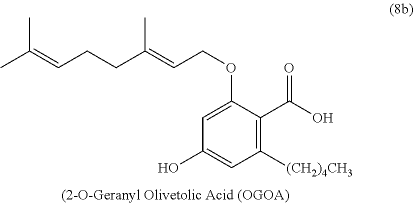

than a compound of Formula (8a):

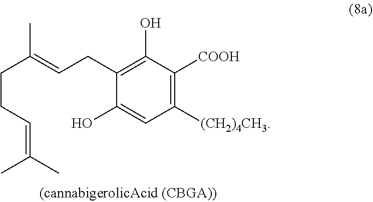

f. Solubility

The C. sativa Cannabigerolic Acid Synthase (CBGAS) enzyme is an integral membrane enzyme that converts olivetolic acid (OA) and geranyl pyrophosphate (GPP) to Cannabigerolic Acid (CBGA) (R4a in FIG. 1, Fellermeier and Zenk FEBS Letters, 1998, Page and Boubakir US 20120144523, 2012, and Luo et al. Nature, 2019). Expression of heterologous membrane proteins can be challenging due to, for example, failure of the protein to refold into a functional protein, accumulation in the cytoplasmic membrane or cytoplasmic inclusion bodies, saturation of the protein sorting and translocation machineries, integrity of the cellular membrane, and/or cellular toxicity (e.g., Wagner et al. Molecular & Cellular Proteomics (2007) 6(9): 1527-1550).

Functional expression of paralog C. sativa CBGAS enzymes in S. cerevisiae and production of the major cannabinoid CBGA has been reported (Page and Boubakir US 20120144523, 2012, and Luo et al. Nature, 2019). Luo et al. reported the production of CBGA in S. cerevisiae by expressing a truncated version of a C. sativa CBGAS, CsPT4, with its native signal peptide removed (Luo et al. Nature, 2019). Without being bound by a particular theory, the integral-membrane nature of C. sativa CBGAS enzymes may render functional expression of C. sativa CBGAS enzymes in heterologous hosts challenging. Removal of transmembrane domain(s) or signal sequences or use of prenyltransferases that are not associated with the membrane and are not integral membrane proteins may facilitate increased interaction between the enzyme and available substrate, for example in the cellular cytosol and/or in organelles that may be targeted using peptides that confer localization.

In some embodiments, the PT is a soluble PT. In some embodiments, the PT is a cytosolic PT. In some embodiments, the PT is a secreted protein. In some embodiments, the PT is not a membrane-associated protein. In some embodiments, the PT is not an integral membrane protein. In some embodiments, the PT does not comprise a transmembrane domain or a predicted transmembrane domain. In some embodiments, the PT may be primarily detected in the cytosol (e.g., detected in the cytosol to a greater extent than detected associated with the cell membrane). In some embodiments, the PT is a protein from which one or more transmembrane domains have been removed and/or mutated (e.g., by truncation, deletions, substitutions, insertions, and/or additions) so that the PT localizes or is predicted to localize in the cytosol of the host cell, or to cytosolic organelles within the host cell, or, in the case of bacterial hosts, in the periplasm. In some embodiments, the PT is a protein from which one or more transmembrane domains have been removed or mutated (e.g., by truncation, deletions, substitutions, insertions, and/or additions) so that the PT has increased localization to the cytosol, organelles, or periplasm of the host cell, as compared to membrane localization.

Within the scope of the term “transmembrane domains” are predicted or putative transmembrane domains in addition to transmembrane domains that have been empirically determined. In general, transmembrane domains are characterized by a region of hydrophobicity that facilitates integration into the cell membrane. Methods of predicting whether a protein is a membrane protein or a membrane-associated protein are known in the art and may include, for example amino acid sequence analysis, hydropathy plots, and/or protein localization assays.

In some embodiments, the PT is a protein from which a signal sequence has been removed and/or mutated such that the PT is not directed to the cellular secretory pathway. In some embodiments, the PT is a protein from which a signal sequence has been removed and/or mutated such that the PT is localized to the cytosol or has increased localization to the cytosol (e.g., as compared to the secretory pathway).

In general, signal sequences, also referred to, for example, as “signal peptides,” are comprised of about 15-30 amino acid and direct a newly translated protein to the cellular secretory pathway. Within the scope of the term “signal sequences” are predicted or putative signal sequences in addition to signal sequences that have been empirically determined.

In some embodiments, the PT is a secreted protein. In some embodiments, the PT contains a signal sequence.

### Additional Cannabinoid Pathway Enzymes

Methods for production of cannabinoids and cannabinoid precursors can further include expression of one or more of: an Acyl Activating Enzyme (AAE): a polyketide synthase (PKS) (e.g., OLS); an Olivetolic acid cyclase (OAC); and a terminal synthase (TS).

### Acyl Activating Enzyme (AAE)

A host cell described in this disclosure may comprise an acyl activating enzyme (AAE). As used in this disclosure, an acyl activating enzyme (AAE) refers to an enzyme that is capable of catalyzing the esterification between a thiol and a substrate (e.g., optionally substituted aliphatic or aryl group) that has a carboxylic acid moiety. In some embodiments, an AAE is capable of using Formula (1):

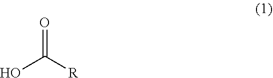

or a salt, solvate, hydrate, polymorph, co-crystal, tautomer, stereoisomer, isotopically labeled derivative thereof to produce a product of Formula (2):

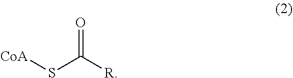

R is as defined in this application. In certain embodiments, R is hydrogen. In certain embodiments, R is optionally substituted alkyl. In certain embodiments, R is optionally substituted C1-40 alkyl. In certain embodiments, R is optionally substituted C2-40 alkyl. In certain embodiments, R is optionally substituted C2-40 alkyl, which is straight chain or branched alkyl. In certain embodiments, R is optionally substituted C2-10 alkyl, optionally substituted C10-C20 alkyl, optionally substituted C20-C30 alkyl, optionally substituted C30-C40 alkyl, or optionally substituted C40-C50 alkyl, which is straight chain or branched alkyl. In certain embodiments, R is optionally substituted C3-8 alkyl. In certain embodiments, R is optionally substituted C1-C40 alkyl, C1-C20 alkyl, C1-C110 alkyl, C1-C8 alkyl, C1-C5 alkyl, C3-C5 alkyl, C3 alkyl, or C5 alkyl. In certain embodiments, R is optionally substituted C1-C20 alkyl. In certain embodiments, R is optionally substituted C1-C20 branched alkyl. In certain embodiments, R is optionally substituted C1-C20 alkyl, optionally substituted C1-C10 alkyl, optionally substituted C10-C20 alkyl, optionally substituted C20-C30 alkyl, optionally substituted C30-C40 alkyl, or optionally substituted C40-C50 alkyl. In certain embodiments, R is optionally substituted C1-C10 alkyl. In certain embodiments, R is optionally substituted C3 alkyl. In certain embodiments, R is optionally substituted n-propyl. In certain embodiments, R is unsubstituted n-propyl. In certain embodiments, R is optionally substituted C1-C8 alkyl. In some embodiments, R is a C2-C6 alkyl. In certain embodiments, R is optionally substituted C1-C5 alkyl. In certain embodiments, R is optionally substituted C3-C5 alkyl. In certain embodiments, R is optionally substituted C3 alkyl. In certain embodiments, R is optionally substituted C5 alkyl. In certain embodiments, R is of formula:

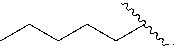 [SMILES: *CCCCC]

In certain embodiments, R is of formula:

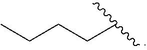 [SMILES: *CCCC]

In certain embodiments, R is of formula:

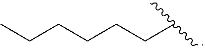 [SMILES: *CCCCCC]

In certain embodiments, R is of formula:

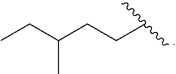 [SMILES: *CCC(C)CC]

In certain embodiments, R is optionally substituted propyl. In certain embodiments, R is optionally substituted n-propyl. In certain embodiments, R is n-propyl optionally substituted with optionally substituted aryl. In certain embodiments, R is n-propyl optionally substituted with optionally substituted phenyl. In certain embodiments, R is n-propyl substituted with unsubstituted phenyl. In certain embodiments, R is optionally substituted butyl. In certain embodiments, R is optionally substituted n-butyl. In certain embodiments, R is n-butyl optionally substituted with optionally substituted aryl. In certain embodiments, R is n-butyl optionally substituted with optionally substituted phenyl. In certain embodiments, R is n-butyl substituted with unsubstituted phenyl. In certain embodiments, R is optionally substituted pentyl. In certain embodiments, R is optionally substituted n-pentyl. In certain embodiments, R is n-pentyl optionally substituted with optionally substituted aryl. In certain embodiments, R is n-pentyl optionally substituted with optionally substituted phenyl. In certain embodiments, R is n-pentyl substituted with unsubstituted phenyl. In certain embodiments, R is optionally substituted hexyl. In certain embodiments, R is optionally substituted n-hexyl. In certain embodiments, R is optionally substituted n-heptyl. In certain embodiments, R is optionally substituted n-octyl. In certain embodiments, R is alkyl optionally substituted with aryl (e.g., phenyl). In certain embodiments, R is optionally substituted acyl (e.g., —C(═O)Me).

In certain embodiments, R is optionally substituted alkenyl (e.g., substituted or unsubstituted C₂₋₆ alkenyl). In certain embodiments, R is substituted or unsubstituted C₂₋₆ alkenyl. In certain embodiments, R is substituted or unsubstituted C₂₋₅ alkenyl. In certain embodiments, R is of formula:

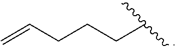 [SMILES: *CCCC=C]

In certain embodiments, R is optionally substituted alkynyl (e.g., substituted or unsubstituted C₂₋₆ alkynyl). In certain embodiments, R is substituted or unsubstituted C₂₋₆ alkynyl. In certain embodiments, R is of formula:

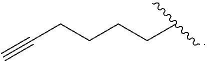 [SMILES: *CCCCC#C]

In certain embodiments, R is optionally substituted carbocyclyl. In certain embodiments, R is optionally substituted aryl (e.g., phenyl or napthyl).

In some embodiments, a substrate for an AAE is produced by fatty acid metabolism within a host cell. In some embodiments, a substrate for an AAE is provided exogenously.

In some embodiments, an AAE is capable of catalyzing the formation of hexanoyl-coenzyme A (hexanoyl-CoA) from hexanoic acid and coenzyme A (CoA). In some embodiments, an AAE is capable of catalyzing the formation of butanoyl-coenzyme A (butanoyl-CoA) from butanoic acid and coenzyme A (CoA).

As one of ordinary skill in the art would appreciate, an AAE could be obtained from any source, including naturally occurring sources and synthetic sources (e.g., a non-naturally occurring AAE). In some embodiments, an AAE is a Cannabis enzyme. Non-limiting examples of AAEs include C. sativa hexanoyl-CoA synthetase 1 (CsHCS1) and C. sativa hexanoyl-CoA synthetase 2 (CsHCS2) as disclosed in U.S. Pat. No. 9,546,362, which is incorporated by reference in this application in its entirety.

CsHCS1 has the sequence:

(SEQ ID NO: 636)
MGKNYKSLDSVVASDFIALGITSEVAETLHGRLAEIVCNYGAATPQTWI
NIANHILSPDLPFSLHQMLFYGCYKDFGPAPPAWIPDPEKVKSTNLGAL
LEKRGKEFLGVKYKDPISSFSHFQEFSVRNPEVYWRTVLMDEMKISFSK
DPECILRRDDINNPGGSEWLPGGYLNSAKNCLNVNSNKKLNDTMIVWRD
EGNDDLPLNKLTLDQLRKRVWLVGYALEEMGLEKGCAIAIDMPMHVDAV
VIYLAIVLAGYVVVSIADSFSAPEISTRLRLSKAKAIFTQDHIIRGKKR
IPLYSRVVEAKSPMAIVIPCSGSNIGAELRDGDISWDYFLERAKEFKNC
EFTAREQPVDAYTNILFSSGTTGEPKAIPWTQATPLKAAADGWSHLDIR
KGDVIVWPTNLGWMMGPWLVYASLLNGASIALYNGSPLVSGFAKFVQDA
KVTMLGVVPSIVRSWKSTNCVSGYDWSTIRCFSSSGEASNVDEYLWLMG
RANYKPVIEMCGGTEIGGAFSAGSFLQAQSLSSFSSQCMGCTLYILDKN
GYPMPKNKPGIGELALGPVMFGASKTLLNGNHHDVYFKGMPTLNGEVLR
RHGDIFELTSNGYYHAHGRADDTMNIGGIKISSIEIERVCNEVDDRVFE
TTAIGVPPLGGGPEQLVIFFVLKDSNDTTIDLNQLRLSFNLGLQKKLNP
LFKVTRVVPLSSLPRTATNKIMRRVLRQFSHFE.

CsHCS2 has the sequence:

(SEQ ID NO: 637)
MEKSGYGRDGIYRSLRPPLHLPNNNNLSMVSFLFRNSSSYPQKPALIDS
ETNQILSFSHFKSTVIKVSHGFLNLGIKKNDVVLIYAPNSIHFPVCFLG
IIASGAIATTSNPLYTVSELSKQVKDSNPKLIITVPQLLEKVKGFNLPT
ILIGPDSEQESSSDKVMTENDLVNLGGSSGSEFPIVDDFKQSDTAALLY
SSGTTGMSKGVVLTHKNFIASSLMVTMEQDLVGEMDNVFLCFLPMFHVF
GLAIITYAQLQRGNTVISMARFDLEKMLKDVEKYKVTHLWVVPPVILAL
SKNSMVKKFNLSSIKYIGSGAAPLGKDLMEECSKVVPYGIVAQGYGMTE
TCGIVSMEDIRGGKRNSGSAGMLASGVEAQIVSVDTLKPLPPNQLGEIW
VKGPNMMQGYFNNPQATKLTIDKKGWVHTGDLGYFDEDGHLYVVDRIKE
LIKYKGFQVAPAELEGLLVSHPEILDAVVIPFPDAEAGEVPVAYVVRSP
NSSLTENDVKKFIAGQVASFKRLRKVTFINSVPKSASGKILRRELIQKV
RSNM.

Additional AAE enzymes are disclosed in, and incorporated by reference from, PCT Publication No. WO2020/176547 and U.S. Patent Publication No. 2021/0071209, both of which are entitled “BIOSYNTHESIS OF CANNABINOIDS AND CANNABINOID PRECURSORS, and each of which is incorporated by reference in its entirety.

### Polyketide Synthases (PKS)

A host cell described in this application may comprise a PKS. As used in this application, a “PKS” refers to an enzyme that is capable of producing a polyketide. In certain embodiments, a PKS converts a compound of Formula (2) to a compound of Formula (4), (5), and/or (6). In certain embodiments, a PKS converts a compound of Formula (2) to a compound of Formula (4). In certain embodiments, a PKS converts a compound of Formula (2) to a compound of Formula (5). In certain embodiments, a PKS converts a compound of Formula (2) to a compound of Formula (4) and/or (5). In certain embodiments, a PKS converts a compound of Formula (2) to a compound of Formula (5) and/or (6).

In some embodiments, a PKS is a tetraketide synthase (TKS). In certain embodiments, a PKS is an olivetol synthase (OLS). As used in this application, an “OLS” refers to an enzyme that is capable of using a substrate of Formula (2a) to form a compound of Formula (4a), (5a) or (6a) as shown in FIG. 1.

In certain embodiments, a PKS is a divarinic acid synthase (DVS).

In certain embodiments, polyketide synthases can use hexanoyl-CoA or any acyl-CoA (or a product of Formula (2):

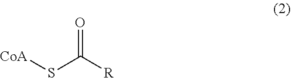

and three malonyl-CoAs as substrates to form 3,5,7-trioxododecanoyl-CoA or other 3,5,7-trioxo-acyl-CoA derivatives; or to form a compound of Formula (4):

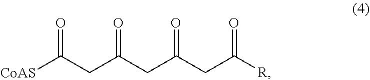

wherein R is hydrogen, optionally substituted acyl, optionally substituted alkyl, optionally substituted alkenyl, optionally substituted alkynyl, optionally substituted carbocyclyl, or optionally substituted aryl; depending on substrate. R is as defined in this application. In some embodiments, R is a C2-C6 optionally substituted alkyl. In some embodiments, R is a propyl or pentyl. In some embodiments, R is pentyl. In some embodiments, R is propyl. A PKS may also bind isovaleryl-CoA, octanoyl-CoA, hexanoyl-CoA, and butyryl-CoA. In some embodiments, PKS is capable of catalyzing the formation of a 3,5,7-trioxoalkanoyl-CoA (e.g. 3,5,7-trioxododecanoyl-CoA). In some embodiments, an OLS is capable of catalyzing the formation of a 3,5,7-trioxoalkanoyl-CoA (e.g. 3,5,7-trioxododecanoyl-CoA).

In some embodiments, a PKS uses a substrate of Formula (2) to form a compound of Formula (4):

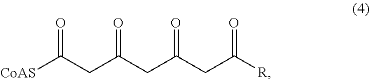

wherein R is unsubstituted pentyl.

As one of ordinary skill in the art would appreciate a PKS, such as an OLS, could be obtained from any source, including naturally occurring sources and synthetic sources (e.g., a non-naturally occurring PKS). In some embodiments a PKS is from Cannabis. In some embodiments a PKS is from Dictyostelium. Non-limiting examples of PKS enzymes may be found in U.S. Pat. No. 6,265,633; PCT Publication No. WO2018/148848 A1; PCT Publication No. WO2018/148849 A1; and U.S. Patent Publication No. 2018/155748, which are incorporated by reference in this application in their entireties.

A non-limiting example of an OLS is provided by UniProtKB—B1Q2B6 from C. sativa. In C. sativa, this OLS uses hexanoyl-CoA and malonyl-CoA as substrates to form 3,5,7-trioxododecanoyl-CoA. OLS (e.g., UniProtKB—B1Q2B6) in combination with olivetolic acid cyclase (OAC) produces olivetolic acid (OA) in C. sativa.

The amino acid sequence of UniProtKB—B1Q2B6 is:

(SEQ ID NO: 638)
MNHLRAEGPASVLAIGTANPENILLQDEFPDYYFRVTKSEHMTQLKEKF
RKICDKSMIRKRNCFLNEEHLKQNPRLVEHEMQTLDARQDMLVVEVPKL
GKDACAKAIKEWGQPKSKITHLIFTSASTTDMPGADYHCAKLLGLSPSV
KRVMMYQLGCYGGGTVLRIAKDIAENNKGARVLAVCCDIMACLFRGPSE
SDLELLVGQAIFGDGAAAVIVGAEPDESVGERPIFELVSTGQTILPNSE
GTIGGHIREAGLIFDLHKDVPMLISNNIEKCLIEAFTPIGISDWNSIFW
ITHPGGKAILDKVEEKLHLKSDKFVDSRHVLSEHGNMSSSTVLFVMDEL
RKRSLEEGKSTTGDGFEWGVLFGFGPGLTVERVVVRSVPIKY.

Additional PKS enzymes are disclosed in, and incorporated by reference from, PCT Publication No. WO2020/176547 and U.S. Patent Publication No. 2021/0071209, both of which are entitled “BIOSYNTHESIS OF CANNABINOIDS AND CANNABINOID PRECURSORS, and each of which is incorporated by reference in its entirety.

In some embodiments, the PKS comprises the sequence of SEQ ID NO: 1183:

(SEQ ID NO: 1183)
MPSLESVKKSNRADGFASILAIGRANPENFIEQSTYPDFFFRVINSEHLVN
LKKKFQRICDKTAIRKRHFVWNEELLNANPCLGTFMDNSLNVRQEFAIREI
PKLGAEAATKAIQEWGQPKSRITHLIFCTTSGMDLPGADYQLTQILGLNPN
IERVMLYQQGCFAGGTTLRLAKCLAESRKGARVLVVCAETTAVLFRAPSEE
HQDDLVTQALFADGASALIVGADPDETAHERASFVIVSTSQVLLPDSAGAI
GGHVSEGGLIATLHRDVPQIVSKNVGKCLEEAFTPLGISDWNSIFWVPHPG
GRAILDQVEERVGLKPEKLIVSRHVLAEYGNMSSVCVHFALDEMRKRSKKE
GKATTGEGLDWGVLFGFGPGLTVETVVLHSVPI.

In some embodiments, the PKS is encoded by a nucleic acid sequence comprising the sequence of SEQ ID NO: 1184:

(SEQ ID NO: 1184)
atgcccagtttagagtcagttaagaaatccaatcgtgccgacggcttcgca
tcgattctggctataggtagagctaaccctgaaaactttatcgaacagtct
acatatccagatttctttttcagagtcaccaatagcgaacaccttgtaaac
ctaaagaaaaagttccaaagaatttgcgacaagactgctatcaggaagcgt
cattttgtgtggaacgaagaattgttgaatgccaacccatgtttgggtacg
tttatggataactcattaaacgtcagacaagaatttgctattagagagatt
ccaaaactaggtgctgaagctgccactaaggcaatccaagaatggggtcaa
ccaaagtccagaataacccacttgatcttctgtactacctctggaatggat
ttgccaggtgctgactaccaattgacccaaattctgggtttgaatcctaat
attgagagggttatgttataccagcaaggttgtttcgctggtggtactact
ttgagattggccaaatgtttagccgaatctcgtaagggagctagagttttg
gttgtctgtgctgaaacaaccgctgttctattcagagcaccttccgaagaa
catcaagatgatttagtaactcaagctttgttcgccgacggtgcttctgct
cttatcgttggtgcagacccagacgagactgcccacgaaagagctagtttt
gttattgtctctacatctcaagtcttgttaccagatagcgctggtgctatc
ggcggtcatgtgtccgaaggtggtttgatcgccactttgcacagagatgtt
ccacagatagttagcaaaaatgtcggtaagtgcttggaagaagcattcacc
cccttgggtattagtgattggaacagtattttttgggttccacacccagga
ggtagagctattcttgaccaagtggaagaaagagtcggtttaaagcctgag
aagttgatcgtatccagacatgtgttagccgaatatggcaacatgtcttct
gtttgtgttcactttgctctggatgaaatgaggaagagatctaaaaaagaa
ggtaaggctacaaccggtgagggtttagactggggtgttttgttcggcttc
ggtccaggattaactgtcgaaaccgtcgttttgcactctgttccaatata
a.

In some embodiments, a PKS comprises a protein or nucleic acid sequence that is at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or is 100% identical, including all values in between, to SEQ ID NO: 1183 or 1184.

PKS enzymes described in this application may or may not have cyclase activity. In some embodiments where the PKS enzyme does not have cyclase activity, one or more exogenous polynucleotides that encode a polyketide cyclase (PKC) enzyme may also be co-expressed in the same host cells to enable conversion of hexanoic acid or butyric acid or other fatty acid conversion into olivetolic acid or divarinolic acid or other precursors of cannabinoids. In some embodiments, the PKS enzyme and a PKC enzyme are expressed as separate distinct enzymes. In some embodiments, a PKS enzyme that lacks cyclase activity and a PKC are linked as part of a fusion polypeptide that is a bifunctional PKS. In some embodiments, a bifunctional PKC is referred to as a bifunctional PKS-PKC. In some embodiments, a bifunctional PKC is a bifunctional tetraketide synthase (TKS-TKC). As used in this application, a bifunctional PKS is an enzyme that is capable of producing a compound of Formula (6):

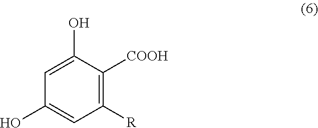

from a compound of Formula (2):

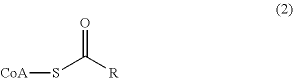

and a compound of Formula (3):

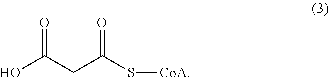

In some embodiments, a PKS produces more of a compound of Formula (6):

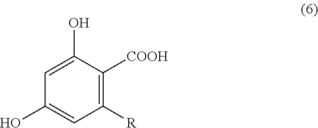

as compared to a compound of Formula (5):

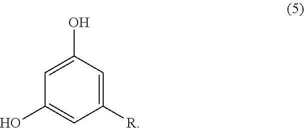

As a non-limiting example, a compound of Formula (6):

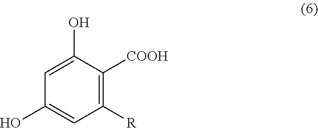

is olivetolic acid (Formula (6a)):

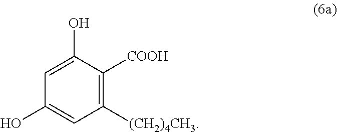

As a non-limiting example, a compound of Formula (5):

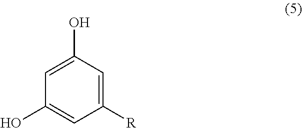

is olivetol (Formula (5a)):

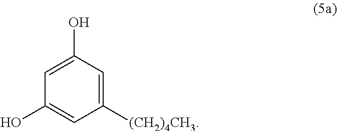

In some embodiments, a polyketide synthase of the present disclosure is capable of catalyzing a compound of Formula (2):

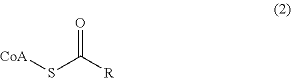

and a compound of Formula (3):

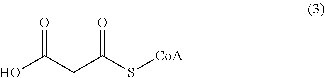

to produce a compound of Formula (4):

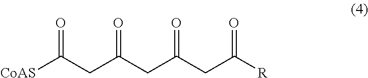

and also further catalyzes a compound of Formula (4):

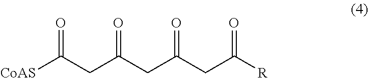

to produce a compound of Formula (6):

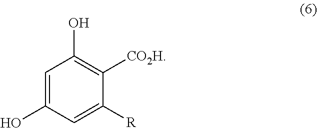

In some embodiments, the PKS is not a fusion protein. In some embodiments, a PKS is capable of catalyzing a compound of Formula (2):

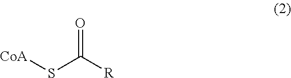

and a compound of Formula (3):

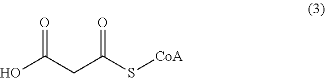

to produce a compound of Formula (4):

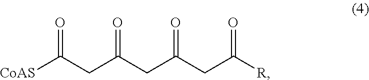

and is also capable of further catalyzing the production of a compound of Formula (6):

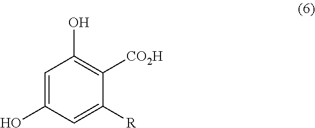

from the compound of Formula (4):

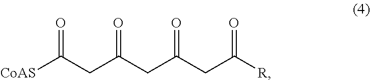

is preferred because it avoids the need for an additional polyketide cyclase to produce a compound of Formula (6):

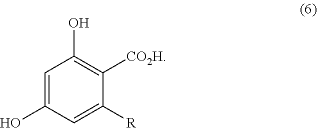

In some embodiments, such an enzyme that is a bifunctional PKS eliminates the transport considerations needed with addition of a polyketide cyclase, whereby the compound of Formula (4), being the product of the PKS, must be transported to the PKS for use as a substrate to be converted into the compound of Formula (6).

In some embodiments, a PKS is capable of producing olivetolic acid in the presence of a compound of Formula (2a):

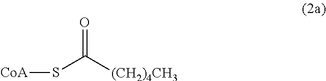

### and Formula (3a):

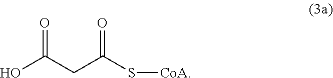

In some embodiments, an OLS is capable of producing olivetolic acid in the presence of a compound of Formula (2a):

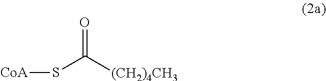

### and Formula (3a):

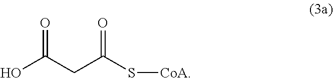

### Polyketide Cyclase (PKC)

A host cell described in this disclosure may comprise a PKC. As used in this application, a “PKC” refers to an enzyme that is capable of cyclizing a polyketide.

In certain embodiments, a polyketide cyclase (PKC) catalyzes the cyclization of an oxo fatty acyl-CoA (e.g., a compound of Formula (4):

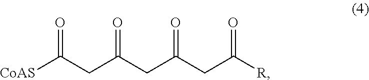

or 3,5,7-trioxododecanoyl-COA, 3,5,7-trioxodecanoyl-COA) to the corresponding intramolecular cyclization product (e.g., compound of Formula (6), including olivetolic acid and divarinic acid). In some embodiments, a PKC catalyzes the formation of a compound which occurs in the presence of a PKS. PKC substrates include trioxoalkanol-CoA, such as 3,5,7-Trioxododecanoyl-CoA, or a compound of Formula (4):

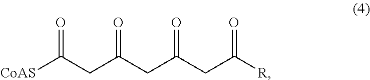

wherein R is hydrogen, optionally substituted acyl, optionally substituted alkyl, optionally substituted alkenyl, optionally substituted alkynyl, optionally substituted carbocyclyl, or optionally substituted aryl. In certain embodiments, a PKC catalyzes a compound of Formula (4):

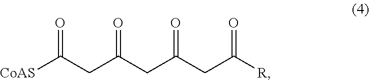

wherein R is hydrogen, optionally substituted acyl, optionally substituted alkyl, optionally substituted alkenyl, optionally substituted alkynyl, optionally substituted carbocyclyl, or optionally substituted aryl; to form a compound of Formula (6):

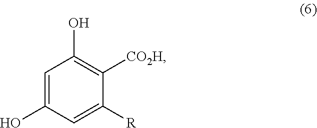

wherein R is hydrogen, optionally substituted acyl, optionally substituted alkyl, optionally substituted alkenyl, optionally substituted alkynyl, optionally substituted carbocyclyl, or optionally substituted aryl; as substrates. R is as defined in this application. In some embodiments, R is a C2-C6 optionally substituted alkyl. In some embodiments, R is a propyl or pentyl. In some embodiments, R is pentyl. In some embodiments, R is propyl. In certain embodiments, a PKC is an olivetolic acid cyclase (OAC).

In some embodiments, a PKC is an OAC. As used in this application, an “OAC” refers to an enzyme that is capable of catalyzing the formation of olivetolic acid (OA). In some embodiments, an OAC is an enzyme that is capable of using a substrate of Formula (4a) (3,5,7-trioxododecanoyl-CoA):

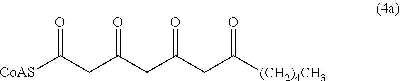

to form a compound of Formula (6a) (olivetolic acid):

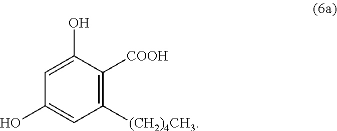

Olivetolic acid cyclase from C. sativa (CsOAC) is a 101 amino acid enzyme that performs non-decaboxylative cyclization of the tetraketide product of olivetol synthase (FIG. 4 Structure 4a) via aldol condensation to form olivetolic acid (FIG. 4 Structure 6a). CsOAC was identified and characterized by Gagne et al. (PNAS 2012) via transcriptome mining, and its cyclization function was recapitulated in vitro to demonstrate that CsOAC is required for formation of olivetolic acid in C. sativa. A crystal structure of the enzyme was published by Yang et al. (FEBS J. 2016 March; 283(6):1088-106), which revealed that the enzyme is a homodimer and belongs to the α+β barrel (DABB) superfamily of protein folds. CsOAC is the only known plant polyketide cyclase. Multiple fungal Type III polyketide synthases have been identified that perform both polyketide synthase and cyclization functions (Funa et al., J Biol Chem. 2007 May 11; 282(19):14476-81); however, in plants such a dual function enzyme has not yet been discovered.

A non-limiting example of an amino acid sequence of an OAC in C. sativa is provided by UniProtKB—I6WU39 (SEQ ID NO: 639), which catalyzes the formation of olivetolic acid (OA) from 3,5,7-Trioxododecanoyl-CoA.

The sequence of UniProtKB—I6WU39 (SEQ ID NO: 639) is:

MAVKHLIVLKFKDEITEAQKEEFFKTYVNLVNIIPAMKDVYWGKDVTQKNK
EEGYTHIVEVTFESVETIQDYIIHPAHVGFGDVYRSFWEKLLIFDYTPRK.

A non-limiting example of a nucleic acid sequence encoding C saliva OAC is:

(SEQ ID NO: 640)
atggcagtgaagcatttgattgtattgaagttcaaagatgaaatcacagaa
gcccaaaaggaagaatttttcaagacgtatgtgaatcttgtgaatatcatc
ccagccatgaaagatgtatactggggtaaagatgtgactcaaaagaataag
gaagaagggtacactcacatagttgaggtaacatttgagagtgtggagact
attcaggactacattattcatcctgcccatgttggatttggagatgtctat
cgttctttctgggaaaaacttctcatttttgactacacaccacgaaag.

In certain embodiments, a PKC is a divarinic acid cyclase (DAC).

As one of ordinary skill in the art would appreciate a PKC could be obtained from any source including naturally occurring sources and synthetic sources (e.g., a non-naturally occurring PKC). In some embodiments, a PKC is from Cannabis. Non-limiting examples of PKCs include those disclosed in U.S. Pat. Nos. 9,611,460; 10,059,971; and U.S. Patent Publication No. 2019/0169661, which are incorporated by reference in this application in their entireties.

### Terminal Synthases (TS)

A host cell described in this application may comprise a terminal synthase (TS). As used in this application, a “TS” refers to an enzyme that is capable of catalyzing oxidative cyclization of a prenyl moiety (e.g., terpene) to produce a ring-containing product (e.g., heterocyclic ring-containing product). In certain embodiments, a TS is capable of catalyzing oxidative cyclization of a prenyl moiety (e.g., terpene) to produce a carbocyclic-ring containing product (e.g., cannabinoid). In certain embodiments, a TS is capable of catalyzing oxidative cyclization of a prenyl moiety (e.g., terpene) to produce a heterocyclic-ring containing product (e.g., cannabinoid). In certain embodiments, a TS is capable of catalyzing oxidative cyclization of a prenyl moiety (e.g., terpene) to produce a cannabinoid. In some embodiments, a terminal synthase is a terpene cyclase that uses a terpenophenolic compound as a substrate.

In some embodiments, a TS is a tetrahydrocannabinolic acid synthase (THCAS), a cannabidiolic acid synthase (CBDAS), and/or a cannabichromenic acid synthase (CBCAS). As one of ordinary skill in the art would appreciate a TS could be obtained from any source, including naturally occurring sources and synthetic sources (e.g., a non-naturally occurring TS).

### A. Substrates

A TS may be capable of using one or more substrates. In some instances, the location of the prenyl group and/or the R group differs between TS substrates. For example, a TS may be capable of using as a substrate one or more compounds of Formula (8w), Formula (8x), Formula (8′), Formula (8y), and/or Formula (8z):

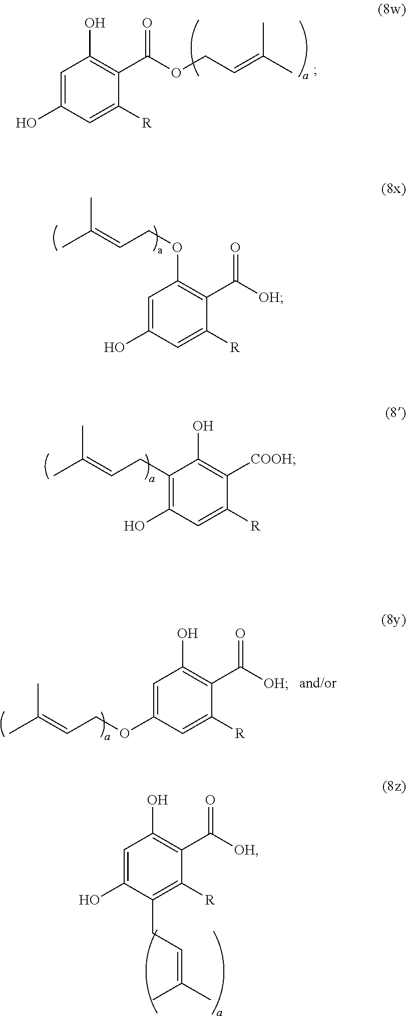

or a pharmaceutically acceptable salt, solvate, hydrate, polymorph, co-crystal, tautomer, stereoisomer, isotopically labeled derivative, or prodrug thereof, wherein a is 1, 2, 3, 4, 5, 6, 7, 8, 9, or 10.

In certain embodiments, a compound of Formula (8′) is a compound of Formula (8):

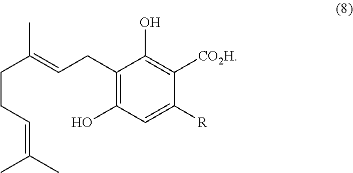

In some embodiments, a TS catalyzes oxidative cyclization of the prenyl moiety (e.g., terpene) of a compound of Formula (8) described in this application and shown in FIG. 2. In certain embodiments, a compound of Formula (8) is a compound of Formula (8a):

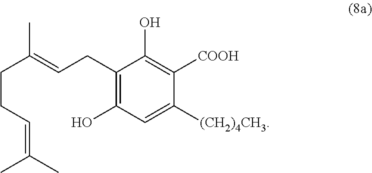

### B. Products

In embodiments wherein CBGA is the substrate, the TS enzymes CBDAS, THCAS and CBCAS would generally catalyze the formation of cannabidiolic acid (CBDA), Δ9-tetrahydrocannabinolic acid (THCA) and cannabichromenic acid (CBCA), respectively. However, in some embodiments, a TS can produce more than one different product depending on reaction conditions. For example, the pH of the reaction environment may cause a THCAS or a CBDAS to produce CBCA in greater proportions than THCA or CBDAS, respectively (see, for example, U.S. Pat. No. 9,359,625 to Winnicki and Donsky, incorporated by reference in its entirety). In some embodiments, a TS has a predetermined product specificity in intracellular conditions, such as cytosolic conditions or organelle conditions. By expressing a TS with a predetermined product specificity based on intracellular conditions, in vivo products produced by a cell expressing the TS may be more predictably produced. In some embodiments, a TS produces a desired product at a pH of 5.5. In some embodiments, a TS produces a desired product at a pH of 1, 2, 3, 4, 5, 6, 7, 8, 9, 10, 11, 12, 13 or 14. In some embodiments, a TS produces a desired product at a pH that is between 4.5 and 8.0. In some embodiments, a TS produces a desired product at a pH that is between 5 and 6. In some embodiments, a TS produces a desired product at a pH that is around 4.5, 4.6, 4.7, 4.8, 4.9, 5.0, 5,1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, or 8.0, including all values in between. In some embodiments, the product profile of a TS is dependent on the TS's signal peptide because the signal peptide targets the TS to a particular intracellular location having particular intracellular conditions (e.g. a particular organelle) that regulate the type of product produced by the TS.

A TS may be capable of using one or more substrates described in this application to produce one or more products. Non-limiting example of TS products are shown in Table 1. In some instances, a TS is capable of using one substrate to produce 1, 2, 3, 4, 5, 6, 7, 8, 9, or 10 different products. In some embodiments, a TS is capable of using more than one substrate to produce 1, 2, 3, 4, 5, 6, 7, 8, 9, or 10 different products.

In some embodiments, a TS is capable of producing a compound of Formula (X-A) and/or a compound of Formula (X-B):

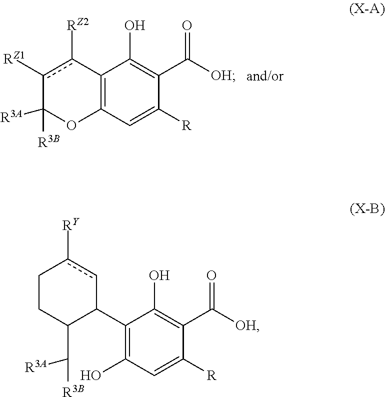

or a pharmaceutically acceptable salt, solvate, hydrate, polymorph, co-crystal, tautomer, stereoisomer, isotopically labeled derivative, or prodrug thereof;
wherein [symbol] is a double bond or a single bond, as valency permits;
R is hydrogen, optionally substituted acyl, optionally substituted alkyl, optionally substituted alkenyl, optionally substituted alkynyl, optionally substituted carbocyclyl, or optionally substituted aryl;
R^{Z1} is hydrogen, optionally substituted acyl, optionally substituted alkyl, optionally substituted alkenyl, optionally substituted alkynyl, optionally substituted carbocyclyl, or optionally substituted aryl;
R^{Z2} is hydrogen, optionally substituted acyl, optionally substituted alkyl, optionally substituted alkenyl, optionally substituted alkynyl, optionally substituted carbocyclyl, or optionally substituted aryl;
or optionally, R^{Z1} and R^{Z2} are taken together with their intervening atoms to form an optionally substituted carbocyclic ring;
R^{3A} is hydrogen, optionally substituted acyl, optionally substituted alkyl, optionally substituted alkenyl, or optionally substituted alkynyl;
R^{3B} is hydrogen, optionally substituted acyl, optionally substituted alkyl, optionally substituted alkenyl, or optionally substituted alkynyl; and/or
R^{Y} is hydrogen, optionally substituted acyl, optionally substituted alkyl, optionally substituted alkenyl, or optionally substituted alkynyl.

In some embodiments, a compound of Formula (X-A) is:

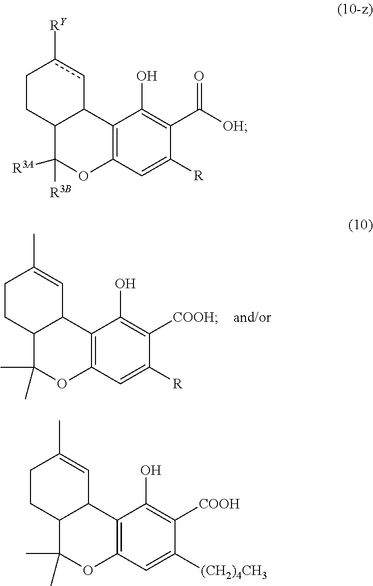

(Tetrahydrocannabinolic acid (THCA) (10a)).

In certain embodiments, a compound of Formula (10)

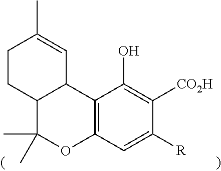

has a chiral atom labeled with * at carbon 10 and a chiral atom labeled with ** at carbon 6. In certain embodiments, in a compound of Formula (10)

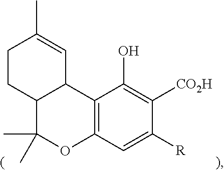

the chiral atom labeled with * at carbon 10 is of the R-configuration or S-configuration; and a chiral atom labeled with ** at carbon 6 is of the R-configuration. In certain embodiments, in a compound of Formula (10)

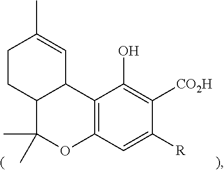

the chiral atom labeled with * at carbon 10 is of the S-configuration; and a chiral atom labeled with ** at carbon 6 is of the R-configuration or S-configuration. In certain embodiments, in a compound of Formula (10)

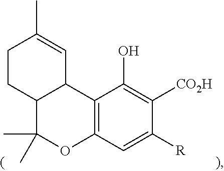

the chiral atom labeled with * at carbon 10 is of the R-configuration and a chiral atom labeled with ** at carbon 6 is of the R-configuration. In certain embodiments, a compound of Formula (10)

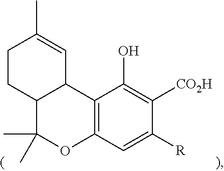

is of the formula:

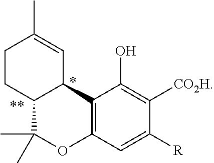

In certain embodiments, in a compound of Formula (10)

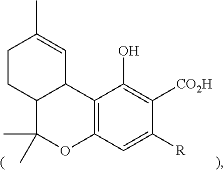

the chiral atom labeled with * at carbon 10 is of the S-configuration and a chiral atom labeled with ** at carbon 6 is of the S-configuration. In certain embodiments, a compound of Formula (10)

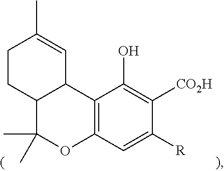

is of the formula:

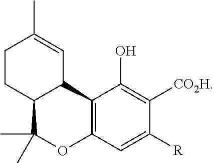

In certain embodiments, a compound of Formula (10a)

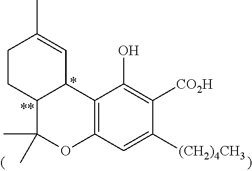

has a chiral atom labeled with * at carbon 10 and a chiral atom labeled with ** at carbon 6. In certain embodiments, in a compound of Formula (10a)

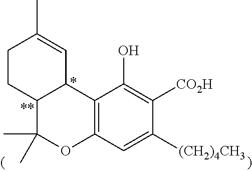

the chiral atom labeled with * at carbon 10 is of the R-configuration or S-configuration; and a chiral atom labeled with ** at carbon 6 is of the R-configuration. In certain embodiments, in a compound of Formula (10a)

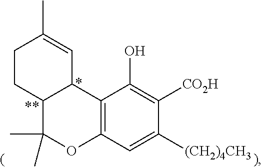

the chiral atom labeled with * at carbon 10 is of the S-configuration; and a chiral atom labeled with ** at carbon 6 is of the R-configuration or S-configuration. In certain embodiments, in a compound of Formula (10a)

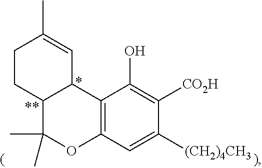

the chiral atom labeled with * at carbon 10 is of the R-configuration and a chiral atom labeled with ** at carbon 6 is of the R-configuration. In certain embodiments, a compound of Formula (10a)

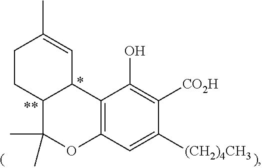

is of the formula:

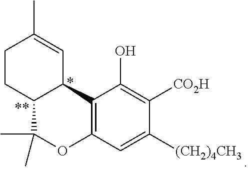

In certain embodiments, in a compound of Formula (10a)

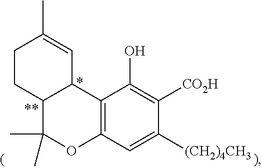

the chiral atom labeled with * at carbon 10 is of the S-configuration and a chiral atom labeled with ** at carbon 6 is of the S-configuration. In certain embodiments, a compound of Formula (10a)

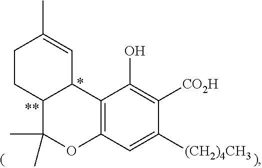

is of the formula:

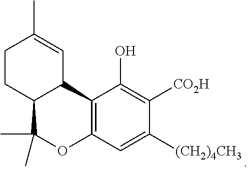

In some embodiments, a compound of Formula (X-A) is:

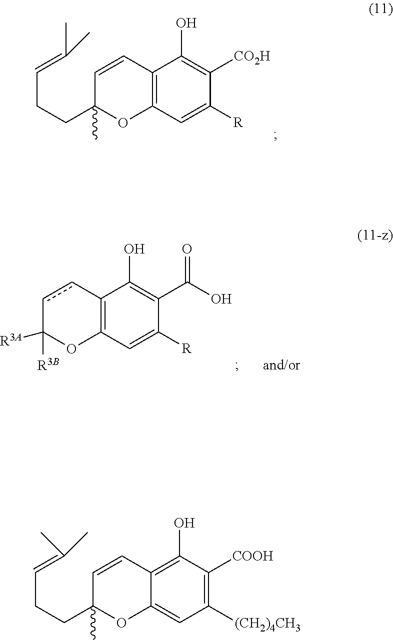

(cannabichromenic acid (CBCA) (11a)).

In some embodiments, a compound of Formula (X-A) is:

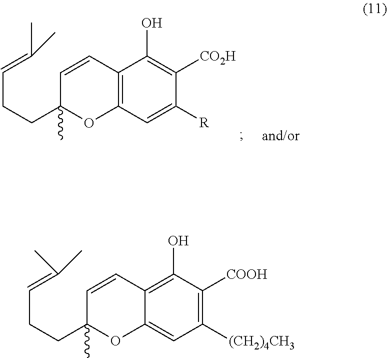

(cannabichromenic acid (CBCA) (11a)).

In some embodiments, a compound of Formula (X-B) is:

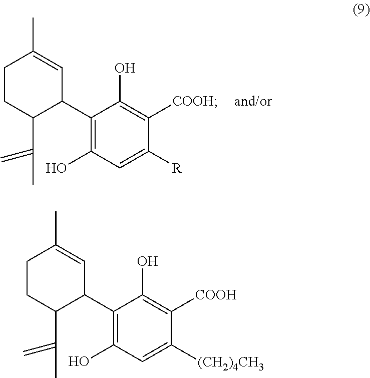

(cannabidiolic acid (CBDA) (9a)).

In certain embodiments, a compound of Formula (9)

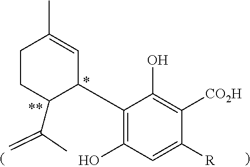

has a chiral atom labeled with * at carbon 3 and a chiral atom labeled with ** at carbon 4. In certain embodiments, in a compound of Formula (9)

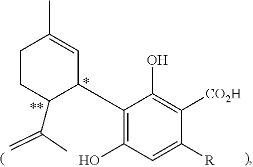

the chiral atom labeled with * at carbon 3 is of the R-configuration or S-configuration; and a chiral atom labeled with ** at carbon 4 is of the R-configuration. In certain embodiments, in a compound of Formula (9)

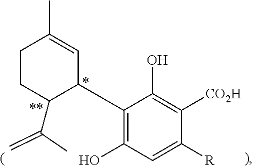

the chiral atom labeled with * at carbon 3 is of the S-configuration; and a chiral atom labeled with ** at carbon 4 is of the R-configuration or S-configuration. In certain embodiments, in a compound of Formula (9)

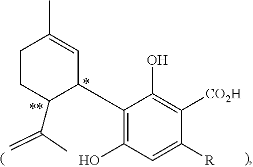

the chiral atom labeled with * at carbon 3 is of the R-configuration and a chiral atom labeled with ** at carbon 4 is of the R-configuration. In certain embodiments, a compound of Formula (9)

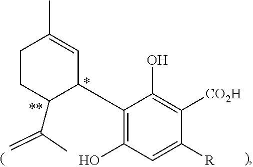

is of the formula:

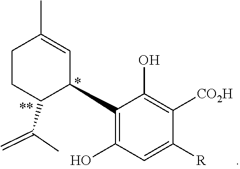

In certain embodiments, in a compound of Formula (9)

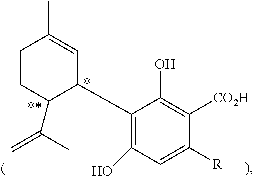

the chiral atom labeled with * at carbon 3 is of the S-configuration and a chiral atom labeled with ** at carbon 4 is of the S-configuration. In certain embodiments, a compound of Formula (9)

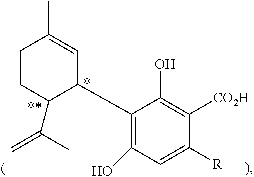

is of the formula:

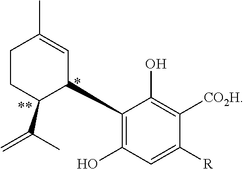

In certain embodiments, a compound of Formula (9a) (CBDA)

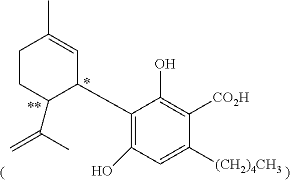

has a chiral atom labeled with * at carbon 3 and a chiral atom labeled with ** at carbon 4. In certain embodiments, in a compound of Formula (9a)

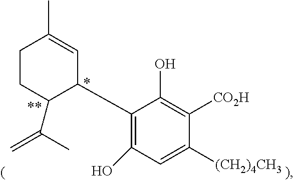

the chiral atom labeled with * at carbon 3 is of the R-configuration or S-configuration; and a chiral atom labeled with ** at carbon 4 is of the R-configuration. In certain embodiments, in a compound of Formula (9a)

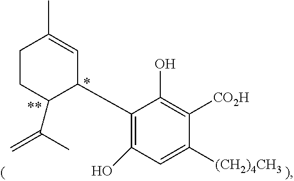

the chiral atom labeled with * at carbon 3 is of the S-configuration; and a chiral atom labeled with ** at carbon 4 is of the R-configuration or S-configuration. In certain embodiments, in a compound of Formula (9a)

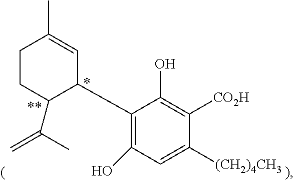

the chiral atom labeled with * at carbon 3 is of the R-configuration and a chiral atom labeled with ** at carbon 4 is of the R-configuration. In certain embodiments, a compound of Formula (9a)

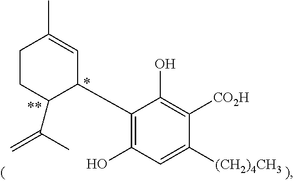

is of the formula:

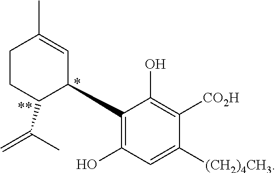

In certain embodiments, in a compound of Formula (9a)

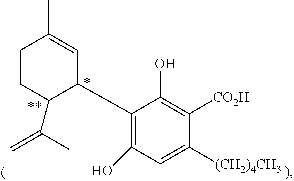

the chiral atom labeled with * at carbon 3 is of the S-configuration and a chiral atom labeled with ** at carbon 4 is of the S-configuration. In certain embodiments, a compound of Formula (9a)

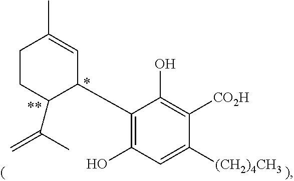

is of the formula:

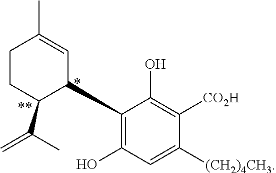

In some embodiments, as shown in FIG. 2, a TS is capable of producing a cannabinoid from the product of a PT, including, without limitation, an enzyme capable of producing a compound of Formula (9), (10), or (11):

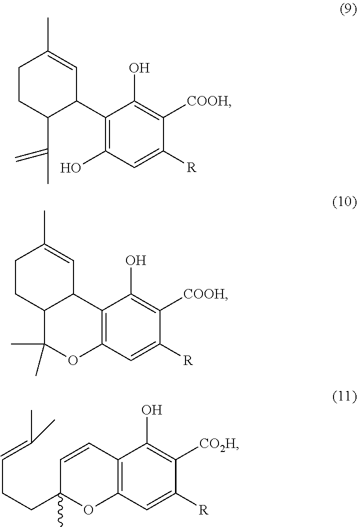

or a pharmaceutically acceptable salt, solvate, hydrate, polymorph, co-crystal, tautomer, stereoisomer, isotopically labeled derivative, or prodrug thereof, wherein R is hydrogen, optionally substituted acyl, optionally substituted alkyl, optionally substituted alkenyl, optionally substituted alkynyl, optionally substituted carbocyclyl, or optionally substituted aryl; produced from a compound of Formula (8′):

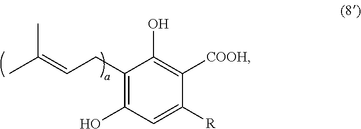

wherein a is 1, 2, 3, 4, 5, 6, 7, 8, 9, or 10; and R is hydrogen, optionally substituted acyl, optionally substituted alkyl, optionally substituted alkenyl, optionally substituted alkynyl, optionally substituted carbocyclyl, or optionally substituted aryl; or using any other substrate. In certain embodiments, a compound of Formula (8′) is a compound of Formula (8):

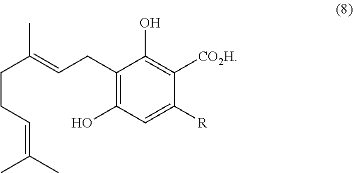

In certain embodiments, a compound of Formula (9), (10), or (11) is produced using a TS from a substrate compound of Formula (8′) (e.g., compound of Formula (8)), for example. Non-limiting examples of substrate compounds of Formula (8′) include but are not limited to cannabigerolic acid (CBGA), cannabigerovarinic acid (CBGVA), or cannabinerolic acid. In certain embodiments, at least one of the hydroxyl groups of the product compounds of Formula (9), (10), or (11) is further methylated. In certain embodiments, a compound of Formula (9) is methylated to form a compound of Formula (12):

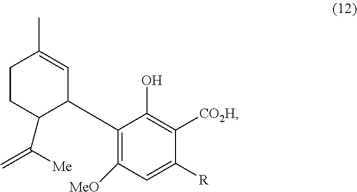

or a pharmaceutically acceptable salt, solvate, hydrate, polymorph, co-crystal, tautomer, stereoisomer, isotopically labeled derivative, or prodrug thereof.

### Tetrahydrocannabinolic Acid Synthase (THCAS)

A host cell described in this application may comprise a TS that is a tetrahydrocannabinolic acid synthase (THCAS). As used in this application “tetrahydrocannabinolic acid synthase (THCAS)” or “Δ¹-tetrahydrocannabinolic acid (THCA) synthase” refers to an enzyme that is capable of catalyzing oxidative cyclization of a prenyl moiety (e.g., terpene) of a compound of Formula (8) to produce a ring-containing product (e.g., heterocyclic ring-containing product, carbocyclic-ring containing product) of Formula (10). In certain embodiments, a THCAS refers to an enzyme that is capable of producing Δ9-tetrahydrocannabinolic acid (Δ9-THCA, THCA, Δ9-Tetrahydro-cannabivarinic acid A (Δ9-THCVA-C3 A), THCVA, THCP, or a compound of Formula 10(a), from a compound of Formula (8). In certain embodiments, a THCAS is capable of producing Δ⁹-tetrahydrocannabinolic acid (Δ⁹-THCA, THCA, or a compound of Formula 10(a)). In certain embodiments, a THCAS is capable of producing Δ9-tetrahydrocannabivarinic acid (Δ9-THCVA, THCVA, or a compound of Formula 10 where R is n-propyl).

In some embodiments, a THCAS may catalyze the oxidative cyclization of substrates, such as 3-prenyl-2,4-dihydroxy-6-alkylbenzoic acids. In some embodiments, a THCAS may use cannabigerolic acid (CBGA) as a substrate. In some embodiments, the THCAS produces Δ9-THCA from CBGA. In some embodiments, a THCAS may catalyze the oxidative cyclization of cannabigerovarinic acid (CBGVA). In some embodiments, a THCAS exhibits specificity for CBGA substrates as compared to other substrates. In some embodiments, a THCAS may use a compound of Formula (8) of FIG. 2 where R is C4 alkyl (e.g., n-butyl) or R is C7 alkyl (e.g., n-heptyl) as a substrate. In some embodiments, a THCAS may use a compound of Formula (8) where R is C4 alkyl (e.g., n-butyl) as a substrate. In some embodiments, a THCAS may use a compound of Formula (8) of FIG. 2 where R is C7 alkyl (e.g., n-heptyl) as a substrate. In some embodiments, the THCAS exhibits specificity for substrates that can result in THCP as a product.

In some embodiments, a THCAS is from C. sativa. C, sativa THCAS performs the oxidative cyclization of the geranyl moiety of Cannabigerolic Acid (CBGA) (FIG. 4 Structure 8a) to form Tetrahydrocannabinolic Acid (FIG. 4 Structure 10a) using covalently bound flavin adenine dinucleotide (FAD) as a cofactor and molecular oxygen as the final electron acceptor. THCAS was first discovered and characterized by Taura et al. (JACS. 1995) following extraction of the enzyme from the leaf buds of C. sativa and confirmation of its THCA synthase activity in vitro upon the addition of CBGA as a substrate. Additional analysis indicated that the enzyme is a monomer and possesses FAD binding and Berberine Bridge Enzyme (BBE) sequence motifs. A crystal structure of the enzyme published by Shoyama et al. (J Mol Biol. 2012 Oct. 12, 423(1):96-105) revealed that the enzyme covalently binds to a molecule of the cofactor FAD. See also, e.g., Sirikantarams et al., J. Biol. Chem. 2004 Sep. 17; 279(38):39767-39774. There are several THCAS isozymes in Cannabis sativa.

In some embodiments, a C. sativa THCAS (Uniprot KB Accession No.: I1V0C5) comprises the amino acid sequence shown below, in which the signal peptide is underlined and bolded:

(SEQ ID NO: 641)
MNCSAFSFWFVCKIIFFFLSFNIQISIANPQENFLKCFSEYIPNNPANPKF
IYTQHDQLYMSVLNSTIQNLRFTSDTTPKPLVIVTPSNVSHIQASILCSKK
VGLQIRTRSGGHDAEGMSYISQVPFVVVDLRNMHSIKIDVHSQTAWVEAGA
TLGEVYYWINEKNENFSFPGGYCPTVGVGGHFSGGGYGALMRNYGLAADNI
IDAHLVNVDGKVLDRKSMGEDLFWAIRGGGGENFGIIAAWKIKLVAVPSKS
TIFSVKKNMEIHGLVKLFNKWQNIAYKYDKDLVLMTHFITKNITDNHGKNK
TTVHGYFSSIFHGGVDSLVDLMNKSFPELGIKKTDCKEFSWIDTTIFYSGV
VNFNTANFKKEILLDRSAGKKTAFSIKLDYVKKPIPETAMVKILEKLYEED
VGVGMYVLYPYGGIMEEISESAIPFPHRAGIMYELWYTASWEKQEDNEKHI
NWVRSVYNFTTPYVSQNPRLAYLNYRDLDLGKTNPESPNNYTQARIWGEKY
FGKNFNRLVKVKTKADPNNFFRNEQSIPPLPPHHH.

In some embodiments, a THCAS comprises the sequence shown below:

(SEQ ID NO: 642)
NPQENFLKCFSEYIPNNPANPKFIYTQHDQLYMSVLNSTIQNLRFTSDTTP
KPLVIVTPSNVSHIQASILCSKKVGLQIRTRSGGHDAEGMSYISQVPFVVV
DLRNMHSIKIDVHSQTAWVEAGATLGEVYYWINEKNENFSFPGGYCPTVGV
GGHFSGGGYGALMRNYGLAADNIIDAHLVNVDGKVLDRKSMGEDLFWAIRG
GGGENFGIIAAWKIKLVAVPSKSTIFSVKKNMEIHGLVKLFNKWQNIAYKY
DKDLVLMTHFITKNITDNHGKNKTTVHGYFSSIFHGGVDSLVDLMNKSFPE
LGIKKTDCKEFSWIDTTIFYSGVVNFNTANFKKEILLDRSAGKKTAFSIKL
DYVKKPIPETAMVKILEKLYEEDVGVGMYVLYPYGGIMEEISESAIPFPHR
AGIMYELWYTASWEKQEDNEKHINWVRSVYNFTTPYVSQNPRLAYLNYRDL
DLGKTNPESPNNYTQARIWGEKYFGKNFNRLVKVKTKADPNNFFRNEQSIP
PLPPHHH.

A non-limiting example of a nucleotide sequence encoding SEQ ID NO: 641 is:

(SEQ ID NO: 643)
aacccgcaagaaaactttctaaaatgcttttctgaatacattcctaacaac
cctgccaacccgaagtttatctacacacaacacgatcaattgtatatgagc
gtgttgaatagtacaatacagaacctgaggtttacatccgacacaacgccg
aaaccgctagtgatcgtcacaccctccaacgtaagccacattcaggcaagc
attttatgcagcaagaaagtcggactgcagataaggacgaggtccggagga
cacgacgccgaagggatgagctatatctcccaggtaccttttgtggtggta
gacttgagaaatatgcactctatcaagatagacgttcactcccaaaccgct
tgggttgaggcgggagccacccttggtgaggtctactactggatcaacgaa
aagaatgaaaattttagctttcctgggggatattgcccaactgtaggtgtt
ggcggccacttctcaggaggcggttatggggccttgatgcgtaactacgga
cttgcggccgacaacattatagacgcacatctagtgaatgtagacggcaaa
gttttagacaggaagagcatgggtgaggatcttttttgggcaattagaggc
ggagggggagaaaattttggaattatcgctgcttggaaaattaagctagtt
gcggtaccgagcaaaagcactatattctctgtaaaaaagaacatggagata
catggtttggtgaagctttttaataagtggcaaaacatcgcgtacaagtac
gacaaagatctggttctgatgacgcattttataacgaaaaatatcaccgac
aaccacggaaaaaacaaaaccacagtacatggctacttctctagtatattt
catgggggagtcgattctctggttgatttaatgaacaaatcattcccagag
ttgggtataaagaagacagactgtaaggagttctcttggattgacacaact
atattctattcaggcgtagtcaactttaacacggcgaatttcaaaaaagag
atccttctggacagatccgcaggtaagaaaactgcgttctctatcaaattg
gactatgtgaagaagcctattcccgaaaccgcgatggtcaagatacttgag
aaattatacgaggaagatgtgggagttggaatgtacgtactttatccctat
ggtgggataatggaagaaatcagcgagagcgccattccatttccccatcgt
gccggcatcatgtacgagctgtggtatactgcgagttgggagaagcaagaa
gacaacgaaaagcacattaactgggtcagatcagtttacaatttcaccacc
ccatacgtgtcccagaatccgcgtctggcttacttgaactaccgtgatctt
gacctgggtaaaacgaacccggagtcacccaacaattacactcaagctaga
atctggggagagaaatactttgggaagaacttcaacaggttagtaaaggtt
aaaaccaaggcagatccaaacaacttttttagaaatgaacaatccattccc
ccgctacccccgcaccatcac.

In some embodiments, a C. sativa THCAS comprises the amino acid sequence set forth in UniProtKB—Q8GTB6 (SEQ ID NO: 644):

MNCSAFSFWFVCKIIFFFLSFHIQISIANPRENFLKCFSKHIPNNVANPKL
VYTQHDQLYMSILNSTIQNLRFISDTTPKPLVIVTPSNNSHIQATILCSKK
VGLQIRTRSGGHDAEGMSYISQVPFVVVDLRNMHSIKIDVHSQTAWVEAGA
TLGEVYYWINEKNENLSFPGGYCPTVGVGGHFSGGGYGALMRNYGLAADNI
IDAHLVNVDGKVLDRKSMGEDLFWAIRGGGGENFGIIAAWKIKLVAVPSKS
TIFSVKKNMEIHGLVKLFNKWQNIAYKYDKDLVLMTHFITKNITDNHGKNK
TTVHGYFSSIFHGGVDSLVDLMNKSFPELGIKKTDCKEFSWIDTTIFYSGV
VNFNTANFKKEILLDRSAGKKTAFSIKLDYVKKPIPETAMVKILEKLYEED
VGAGMYVLYPYGGIMEEISESAIPFPHRAGIMYELWYTASWEKQEDNEKHI
NWVRSVYNFTTPYVSQNPRLAYLNYRDLDLGKTNHASPNNYTQARIWGEKY
FGKNFNRLVKVKTKVDPNNFFRNEQSIPPLPPHHH.

Additional non-limiting examples of THCAS enzymes may also be found in U.S. Pat. No. 9,512,391, U.S. Patent Application Publication No. 2018/0179564 and PCT Application No. PCT/US21/40941, which are incorporated by reference in this application in their entireties.

### Cannabidiolic Acid Synthase (CBDAS)

A host cell described in this application may comprise a TS that is a cannabidiolic acid synthase (CBDAS). As used in this application, a “CBDAS” refers to an enzyme that is capable of catalyzing oxidative cyclization of a prenyl moiety (e.g., terpene) of a compound of Formula (8) to produce a compound of Formula (9). In some embodiments, a compound of Formula 9 is a compound of Formula (9a) (cannabidiolic acid (CBDA)), CBDVA, or CBDP. A CBDAS may use cannabigerolic acid (CBGA) or cannabinerolic acid as a substrate. In some embodiments, a cannabidiolic acid synthase is capable of oxidative cyclization of cannabigerolic acid (CBGA) to produce cannabidiolic acid (CBDA). In some embodiments, the CBDAS may catalyze the oxidative cyclization of other substrates, such as 3-geranyl-2,4-dihydro-6-alkylbenzoic acids like cannabigerovarinic acid (CBGVA) or a substrate of Formula (8) with R as a C7 alkyl (heptyl) group (cannabigerophorolic acid (CBGPA)). In some embodiments, the CBDAS exhibits specificity for CBGA substrates.

In some embodiments, a CBDAS is from Cannabis. In C. sativa, CBDAS is encoded by the CBDAS gene and is a flavoenzyme. A non-limiting example of a CBDAS is provided by UniProtKB—A6P6V9 (SEQ ID NO: 645) from C. sativa:

MKCSTFSFWFVCKIIFFFFSFNIQTSIANPRENFLKCFSQYIPNNATNLKL
VYTONNPLYMSVLNSTIHNLRFTSDTTPKPLVIVTPSHVSHIQGTILCSKK
VGLQIRTRSGGHDSEGMSYISQVPFVIVDLRNMRSIKIDVHSQTAWVEAGA
TLGEVYYWVNEKNENLSLAAGYCPTVCAGGHFGGGGYGPLMRNYGLAADNI
IDAHLVNVHGKVLDRKSMGEDLFWALRGGGAESFGIIVAWKIRLVAVPKST
MFSVKKIMEIHELVKLVNKWQNIAYKYDKDLLLMTHFITRNITDNQGKNKT
AIHTYFSSVFLGGVDSLVDLMNKSFPELGIKKTDCRQLSWIDTHIFYSGVV
NYDTDNFNKEILLDRSAGONGAFKIKLDYVKKPIPESVFVQILEKLYEEDI
GAGMYALYPYGGIMDEISESAIPFPHRAGILYELWYICSWEKQEDNEKHLN
WIRNIYNFMTPYVSKNPRLAYLNYRDLDIGINDPKNPNNYTQARIWGEKYF
GKNFDRLVKVKTLVDPNNFFRNEQSIPPLPRHRH.

Additional non-limiting examples of CBDAS enzymes may also be found in U.S. Pat. No. 9,512,391, U.S. Patent Application Publication No. 2018/0179564 and PCT Application No. PCT/US21/40941 w % which are incorporated by reference in this application in their entireties.

### Cannabichromenic Acid Synthase (CBCAS)

A host cell described in this application may comprise a TS that is a cannabichromenic acid synthase (CBCAS). As used in this application, a “CBCAS” refers to an enzyme that is capable of catalyzing oxidative cyclization of a prenyl moiety (e.g., terpene) of a compound of Formula (8) to produce a compound of Formula (11). In some embodiments, a compound of Formula (11) is a compound of Formula (11a) (cannabichromenic acid (CBCA)), CBCVA, or a compound of Formula (8) with R as a C7 alkyl (heptyl) group. A CBCAS may use cannabigerolic acid (CBGA) as a substrate. In some embodiments, a CBCAS produces cannabichromenic acid (CBCA) from cannabigerolic acid (CBGA). In some embodiments, the CBCAS may catalyze the oxidative cyclization of other substrates, such as 3-geranyl-2,4-dihydro-6-alkylbenzoic acids like cannabigerovarinic acid (CBGVA), or a substrate of Formula (8) with R as a C7 alkyl (heptyl) group. In some embodiments, the CBCAS exhibits specificity for CBGA substrates.

In some embodiments, a CBCAS is from Cannabis. In C. sativa, an amino acid sequence encoding CBCAS is provided by, and incorporated by reference from, SEQ ID NO:2 disclosed in U.S. Patent Publication No. 2017/0211049. In other embodiments, a CBCAS may be a THCAS described in and incorporated by reference from U.S. Pat. No. 9,359,625. SEQ ID NO:2 disclosed in U.S. Patent Application Publication No. 2017/0211049 (corresponding to SEQ ID NO: 646 in this application) has the amino acid sequence:

MNCSTFSFWFVCKIIFFFLSFNIQISIANPQENFLKCFSEYIPNNPANPKF
IYTQHDQLYMSVLNSTIQNLRFTSDTTPKPLVIVTPSNVSHIQASILCSKK
VGLQIRTRSGGHDAEGLSYISQVPFAIVDLRNMHTVKVDIHSQTAWVEAGA
TLGEVYYWINEMNENFSFPGGYCPTVGVGGHFSGGGYGALMRNYGLAADNI
IDAHLVNVDGKVLDRKSMGEDLFWAIRGGGGENFGIIAACKIKLVVVPSKA
TIFSVKKNMEIHGLVKLENKWQNIAYKYDKDLMLTTHFRTRNITDNHGKNK
TTVHGYFSSIFLGGVDSLVDLMNKSFPELGIKKTDCKELSWIDTTIFYSGV
VNYNTANFKKEILLDRSAGKKTAFSIKLDYVKKLIPETAMVKILEKLYEEE
VGVGMYVLYPYGGIMDEISESAIPFPHRAGIMYELWYTATWEKQEDNEKHI
NWVRSVYNFTTPYVSQNPRLAYLNYRDLDLGKTNPESPNNYTQARIWGEKY
FGKNFNRLVKVKTKADPNNFFRNEQSIPPLPPRHH.

Additional non-limiting examples of CBCAS enzymes may also be found in PCT Publication No. WO/2021/195520 and PCT Application No. PCT/US21/40941, which are incorporated by reference in this application in their entireties.

### Variants

Aspects of the disclosure relate to nucleic acids encoding any of the polypeptides (e.g., AAE, PKS, PKC, PT, or TS) described in this application. In some embodiments, a nucleic acid encompassed by the disclosure is a nucleic acid that hybridizes under high or medium stringency conditions to a nucleic acid encoding an AAE, PKS, PKC. PT, or TS and is biologically active. For example, high stringency conditions of 0.2 to 1×SSC at 65° C. followed by a wash at 0.2×SSC at 65° C. can be used. In some embodiments, a nucleic acid encompassed by the disclosure is a nucleic acid that hybridizes under low stringency conditions to a nucleic acid encoding an AAE, PKS, PKC, PT, or TS and is biologically active. For example, low stringency conditions of 6×SSC at room temperature followed by a wash at 2×SSC at room temperature can be used. Other hybridization conditions include 3×SSC at 40 or 50° C., followed by a wash in 1 or 2×SSC at 20, 30, 40, 50, 60, or 65° C.

Hybridizations can be conducted in the presence of formaldehyde, e.g., 10%, 20%, 30% 40% or 50%, which further increases the stringency of hybridization. Theory and practice of nucleic acid hybridization is described, e.g., in S. Agrawal (ed.) Methods in Molecular Biology, volume 20; and Tijssen (1993) Laboratory Techniques in biochemistry and molecular biology-hybridization with nucleic acid probes, e.g., part 1 chapter 2 “Overview of principles of hybridization and the strategy of nucleic acid probe assays,” Elsevier, New York provide a basic guide to nucleic acid hybridization.

Variants of enzyme sequences described in this application (e.g., AAE, PKS. PKC, PT, or TS, including nucleic acid or amino acid sequences) are also encompassed by the present disclosure. A variant may share at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with a reference sequence, including all values in between.

Unless otherwise noted, the term “sequence identity,” which is used interchangeably in this disclosure with the term “percent identity,” as known in the art, refers to a relationship between the sequences of two polypeptides or polynucleotides, as determined by sequence comparison (alignment). In some embodiments, sequence identity is determined across the entire length of a sequence (e.g., AAE, PKS, PKC, PT, or TS sequence). In some embodiments, sequence identity is determined over a region (e.g., a stretch of amino acids or nucleic acids, e.g., the sequence spanning an active site) of a sequence (e.g., AAE, PKS, PKC, PT, or TS sequence). For example, in some embodiments, sequence identity is determined over a region corresponding to at least 30%, at least 40%, at least 50%, at least 60%, at least 70%, at least 80%, at least 90%, at least 95%, or over 100% of the length of the reference sequence.

Identity measures the percent of identical matches between the smaller of two or more sequences with gap alignments (if any) addressed by a particular mathematical model, algorithm, or computer program.

Identity of related polypeptides or nucleic acid sequences can be readily calculated by any of the methods known to one of ordinary skill in the art. The percent identity of two sequences (e.g., nucleic acid or amino acid sequences) may, for example, be determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST® and XBLAST® programs (version 2.0) of Altschul et al., J. Mol. Biol. 215:403-10, 1990. BLAST® protein searches can be performed, for example, with the XBLAST program, score=50, wordlength=3 to obtain amino acid sequences homologous to the proteins described in this application. Where gaps exist between two sequences, Gapped BLAST can be utilized, for example, as described in Altschul et al., Nucleic Acids Res. 25(17):3389-3402, 1997. When utilizing BLAST® and Gapped BLAST® programs, the default parameters of the respective programs (e.g., XBLAST® and NBLAST®) can be used, or the parameters can be adjusted appropriately as would be understood by one of ordinary skill in the art.

Another local alignment technique which may be used, for example, is based on the Smith-Waterman algorithm (Smith, T. F. & Waterman, M. S. (1981) “Identification of common molecular subsequences.” J. Mol. Biol. 147:195-197). A general global alignment technique which may be used, for example, is the Needleman-Wunsch algorithm (Needleman, S. B. & Wunsch, C. D. (1970) “A general method applicable to the search for similarities in the amino acid sequences of two proteins.” J. Mol. Biol. 48:443-453), which is based on dynamic programming.

More recently, a Fast Optimal Global Sequence Alignment Algorithm (FOGSAA) was developed that purportedly produces global alignment of nucleic acid and amino acid sequences faster than other optimal global alignment methods, including the Needleman-Wunsch algorithm. In some embodiments, the identity of two polypeptides is determined by aligning the two amino acid sequences, calculating the number of identical amino acids, and dividing by the length of one of the amino acid sequences. In some embodiments, the identity of two nucleic acids is determined by aligning the two nucleotide sequences and calculating the number of identical nucleotide and dividing by the length of one of the nucleic acids.

For multiple sequence alignments, computer programs including Clustal Omega (Sievers et al., Mol Syst Biol. 2011 Oct. 11; 7:539) may be used.

In preferred embodiments, a sequence, including a nucleic acid or amino acid sequence, is found to have a specified percent identity to a reference sequence, such as a sequence disclosed in this application and/or recited in the claims when sequence identity is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993 (e.g., BLAST®, NBLAST®, XBLAST® or Gapped BLAST programs, using default parameters of the respective programs).

In some embodiments, a sequence, including a nucleic acid or amino acid sequence, is found to have a specified percent identity to a reference sequence, such as a sequence disclosed in this application and/or recited in the claims when sequence identity is determined using the Smith-Waterman algorithm (Smith, T. F. & Waterman, M. S. (1981) “Identification of common molecular subsequences.” J. Mol. Biol. 147:195-197) or the Needleman-Wunsch algorithm (Needleman, S. B. & Wunsch, C. D. (1970) “A general method applicable to the search for similarities in the amino acid sequences of two proteins.” J. Mol. Biol. 48:443453) using default parameters.

In some embodiments, a sequence, including a nucleic acid or amino acid sequence, is found to have a specified percent identity to a reference sequence, such as a sequence disclosed in this application and/or recited in the claims when sequence identity is determined using a Fast Optimal Global Sequence Alignment Algorithm (FOGSAA) using default parameters.

In some embodiments, a sequence, including a nucleic acid or amino acid sequence, is found to have a specified percent identity to a reference sequence, such as a sequence disclosed in this application and/or recited in the claims when sequence identity is determined using Clustal Omega (Sievers et al., Mol Syst Biol. 2011 Oct. 11; 7:539) using default parameters.

As used in this application, a residue (such as a nucleic acid residue or an amino acid residue) in sequence “X” is referred to as corresponding to a position or residue (such as a nucleic acid residue or an amino acid residue) “Z” in a different sequence “Y” when the residue in sequence “X” is at the counterpart position of “Z” in sequence “Y” when sequences X and Y are aligned using amino acid sequence alignment tools known in the art.

As used in this application, variant sequences may be homologous sequences. As used in this application, homologous sequences are sequences (e.g., nucleic acid or amino acid sequences) that share a certain percent identity (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% percent identity, including all values in between). Homologous sequences include but are not limited to paralogous or orthologous sequences. Paralogous sequences arise from duplication of a gene within a genome of a species, while orthologous sequences diverge after a speciation event.

In some embodiments, a polypeptide variant (e.g., AAE, PKS, PKC, PT, or TS enzyme variant) comprises a domain that shares a secondary structure (e.g., alpha helix, beta sheet) with a reference polypeptide (e.g., a reference AAE, PKS, PKC, PT, or TS enzyme). In some embodiments, a polypeptide variant (e.g., AAE, PKS, PKC, PT, or TS enzyme variant) shares a tertiary structure with a reference polypeptide (e.g., a reference AAE, PKS, PKC, PT, or TS enzyme). As a non-limiting example, a polypeptide variant (e.g., AAE, PKS. PKC, PT, or TS enzyme) may have low primary sequence identity (e.g., less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, or less than 5% sequence identity) compared to a reference polypeptide, but share one or more secondary structures (e.g., including but not limited to loops, alpha helices, or beta sheets), or have the same tertiary structure as a reference polypeptide. For example, a loop may be located between a beta sheet and an alpha helix, between two alpha helices, or between two beta sheets. Homology modeling may be used to compare two or more tertiary structures.

Functional variants of the recombinant AAE, PKS, PKC, PT, or TS enzyme disclosed in this application are encompassed by the present disclosure. For example, functional variants may bind one or more of the same substrates or produce one or more of the same products. Functional variants may be identified using any method known in the art. For example, the algorithm of Karlin and Altschul Proc. Natl. Acad. Sci. USA 87:2264-68, 1990 described above may be used to identify homologous proteins with known functions.

Putative functional variants may also be identified by searching for polypeptides with functionally annotated domains. Databases including Pfam (Sonnhammer et al., Proteins. 1997 July; 28(3):405-20) may be used to identify polypeptides with a particular domain.

Homology modeling may also be used to identify amino acid residues that are amenable to mutation (e.g., substitution, deletion, and/or insertion) without affecting function. A non-limiting example of such a method may include use of position-specific scoring matrix (PSSM) and an energy minimization protocol.

Position-specific scoring matrix (PSSM) uses a position weight matrix to identify, consensus sequences (e.g., motifs). PSSM can be conducted on nucleic acid or amino acid sequences. Sequences are aligned and the method takes into account the observed frequency of a particular residue (e.g., an amino acid or a nucleotide) at a particular position and the number of sequences analyzed. See, e.g., Stormo et al., Nucleic Acids Res. 1982 May 11:10(9):2997-3011. The likelihood of observing a particular residue at a given position can be calculated. Without being bound by a particular theory, positions in sequences with high variability may be amenable to mutation (e.g., substitution, deletion, and/or insertion; e.g., PSSM score≥0) to produce functional homologs.

PSSM may be paired with calculation of a Rosetta energy function, which determines the difference between the wild-type and the single-point mutant. The Rosetta energy function calculates this difference as (ΔΔG_{calc}). With the Rosetta function, the bonding interactions between a mutated residue and the surrounding atoms are used to determine whether an amino acid substitution, deletion, or insertion increases or decreases protein stability. For example, an amino acid substitution, deletion, or insertion that is designated as favorable by the PSSM score (e.g. PSSM score≥0), can then be analyzed using the Rosetta energy function to determine the potential impact of the mutation on protein stability. Without being bound by a particular theory, potentially stabilizing mutations are desirable for protein engineering (e.g., production of functional homologs). In some embodiments, a potentially stabilizing mutation has a ΔΔG_{calc} value of less than −0.1 (e.g., less than −0.2, less than −0.3, less than −0.35, less than −0.4, less than −0.45, less than −0.5, less than −0.55, less than −0.6, less than −0.65, less than −0.7, less than −0.75, less than −0.8, less than −0.85, less than −0.9, less than −0.95, or less than −1.0) Rosetta energy units (R.e.u.). See, e.g., Goldenzweig et al., Mol Cell. 2016 Jul. 21; 63(2):337-346. Doi: 10.1016/j.molcel.2016.06.012.

In some embodiments, an AAE, PKS, PKC, PT, or TS coding sequence comprises a mutation at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more than 100 positions relative to a reference (e.g., AAE, PKS, PKC, PT, or TS) coding sequence. In some embodiments, the AAE, PKS, PKC, PT, or TS coding sequence comprises a mutation in 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more codons of the coding sequence relative to a reference (e.g., AAE, PKS, PKC, PT, or TS) coding sequence. As will be understood by one of ordinary skill in the art, a mutation within a codon may or may not change the amino acid that is encoded by the codon due to degeneracy of the genetic code. In some embodiments, the one or more mutations in the coding sequence do not alter the amino acid sequence of the coding sequence (e.g., AAE. PKS, PKC, PT, or TS) relative to the amino acid sequence of a reference polypeptide (e.g., AAE, PKS, PKC, PT, or TS).

In some embodiments, the one or more mutations in a recombinant coding sequence (e.g., AAE, PKS, PKC, PT, or TS coding sequence) do alter the amino acid sequence of the corresponding polypeptide (e.g., AAE, PKS, PKC, PT, or TS) relative to the amino acid sequence of a reference polypeptide (e.g., AAE, PKS, PKC, PT, or TS). In some embodiments, the one or more mutations alters the amino acid sequence of the polypeptide (e.g., AAE, PKS, PKC, PT, or TS) relative to the amino acid sequence of a reference polypeptide (e.g., AAE. PKS, PKC, PT, or TS) and alters (enhances or reduces) an activity of the polypeptide relative to the reference polypeptide.

The activity (e.g., specific activity) of any of the recombinant polypeptides described in this application (e.g., AAE, PKS, PKC, PT, or TS) may be measured using routine methods. As a non-limiting example, a recombinant polypeptide's activity may be determined by measuring its substrate specificity, product(s) produced, the concentration of product(s) produced, or any combination thereof. As used in this application, “specific activity” of a recombinant polypeptide refers to the amount (e.g., concentration) of a particular product produced for a given amount (e.g., concentration) of the recombinant polypeptide per unit time.

The skilled artisan will also realize that mutations in a recombinant polypeptide (e.g., AAE, PKS, PKC, PT, or TS) coding sequence may result in conservative amino acid substitutions to provide functionally equivalent variants of the foregoing polypeptides, e.g., variants that retain the activities of the polypeptides. As used in this application, a “conservative amino acid substitution” refers to an amino acid substitution that does not alter the relative charge or size characteristics or functional activity of the protein in which the amino acid substitution is made.

In some instances, an amino acid is characterized by its R group (see, e.g., Table 4). For example, an amino acid may comprise a nonpolar aliphatic R group, a positively charged R group, a negatively charged R group, a nonpolar aromatic R group, or a polar uncharged R group. Non-limiting examples of an amino acid comprising a nonpolar aliphatic R group include alanine, glycine, valine, leucine, methionine, and isoleucine. Non-limiting examples of an amino acid comprising a positively charged R group includes lysine, arginine, and histidine. Non-limiting examples of an amino acid comprising a negatively charged R group include aspartate and glutamate. Non-limiting examples of an amino acid comprising a nonpolar, aromatic R group include phenylalanine, tyrosine, and tryptophan. Non-limiting examples of an amino acid comprising a polar uncharged R group include serine, threonine, cysteine, proline, asparagine, and glutamine.

Non-limiting examples of functionally equivalent variants of polypeptides may include conservative amino acid substitutions in the amino acid sequences of proteins disclosed in this application. As used in this application “conservative substitution” is used interchangeably with “conservative amino acid substitution” and refers to any one of the amino acid substitutions provided in Table 4.

In some embodiments, 1, 2, 3, 4, 5, 6, 7, 8, 9, 10, 11, 12, 13, 14, 15, 16, 17, 18, 19, 20 or more than 20 residues can be changed when preparing variant polypeptides. In some embodiments, amino acids are replaced by conservative amino acid substitutions. In some embodiments, amino acids are replaced by non-conservative amino acid substitutions.

TABLE 4
Conservative Amino Acid Substitutions

| Original | | Conservative Amino |
|---|---|---|
| Residue | R Group Type | Acid Substitutions |
| Ala | nonpolar aliphatic R group | Cys, Gly, Ser |
| Arg | positively charged R group | His, Lys |
| Asn | polar uncharged R group | Asp, Gln, Glu |
| Asp | negatively charged R group | Asn, Gln, Glu |
| Cys | polar uncharged R group | Ala, Ser |
| Gln | polar uncharged R group | Asn, Asp, Glu |
| Glu | negatively charged R group | Asn, Asp, Gln |
| Gly | nonpolar aliphatic R group | Ala, Ser |
| His | positively charged R group | Arg, Tyr, Trp |
| Ile | nonpolar aliphatic R group | Leu, Met, Val |
| Leu | nonpolar aliphatic R group | Ile, Met, Val |
| Lys | positively charged R group | Arg, His |
| Met | nonpolar aliphatic R group | Ile, Leu, Phe, Val |
| Pro | polar uncharged R group | |
| Phe | nonpolar aromatic R group | Met, Trp, Tyr |
| Ser | polar uncharged R group | Ala, Gly, Thr |
| Thr | polar uncharged R group | Ala, Asn, Ser |
| Trp | nonpolar aromatic R group | His, Phe, Tyr, Met |
| Tyr | nonpolar aromatic R group | His, Phe, Trp |
| Val | nonpolar aliphatic R group | Ile, Leu, Met, Thr |

Amino acid substitutions in the amino acid sequence of a polypeptide to produce a recombinant polypeptide (e.g., AAE, PKS, PKC, PT, or TS) variant having a desired property and/or activity can be made by alteration of the coding sequence of the polypeptide (e.g., AAE. PKS, PKC, PT, or TS). Similarly, conservative amino acid substitutions in the amino acid sequence of a polypeptide to produce functionally equivalent variants of the polypeptide typically are made by alteration of the coding sequence of the recombinant polypeptide (e.g., AAE, PKS, PKC. PT, or TS).

Mutations (e.g., substitutions, insertions, additions, or deletions) can be made in a nucleic acid sequence by a variety of methods known to one of ordinary skill in the art. For example, mutations (e.g., substitutions, insertions, additions, or deletions) can be made by PCR-directed mutation, site-directed mutagenesis according to the method of Kunkel (Kunkel, Proc. Nat. Acad. Sci. U.S.A. 82: 488-492, 1985), by chemical synthesis of a gene encoding a polypeptide, by CRISPR, or by insertions, such as insertion of a tag (e.g., a HIS tag or a GFP tag). Mutations can include, for example, substitutions, insertions, additions, deletions, and translocations, generated by any method known in the art. Methods for producing mutations may be found in in references such as Molecular Cloning: A Laboratory Manual, J. Sambrook, et al., eds., Fourth Edition, Cold Spring Harbor Laboratory Press, Cold Spring Harbor, New York, 2012, or Current Protocols in Molecular Biology, F. M. Ausubel, et al., eds., John Wiley & Sons, Inc., New York, 2010.

In some embodiments, methods for producing variants include circular permutation (Yu and Lutz, Trends Biotechnol. 2011 January; 29(1):18-25). In circular permutation, the linear primary sequence of a polypeptide can be circularized (e.g., by joining the N-terminal and C-terminal ends of the sequence) and the polypeptide can be severed (“broken”) at a different location. Thus, the linear primary sequence of the new polypeptide may have low sequence identity (e.g., less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less or less than 5%, including all values in between) as determined by linear sequence alignment methods (e.g., Clustal Omega or BLAST). Topological analysis of the two proteins, however, may reveal that the tertiary structure of the two polypeptides is similar or dissimilar. Without being bound by a particular theory, a variant polypeptide created through circular permutation of a reference polypeptide and with a similar tertiary structure as the reference polypeptide can share similar functional characteristics (e.g., enzymatic activity, enzyme kinetics, substrate specificity or product specificity). In some instances, circular permutation may alter the secondary structure, tertiary structure or quaternary structure and produce an enzyme with different functional characteristics (e.g., increased or decreased enzymatic activity, different substrate specificity, or different product specificity). See, e.g., Yu and Lutz, Trends Biotechnol. 2011 January; 29(1):18-25.

It should be appreciated that in a protein that has undergone circular permutation, the linear amino acid sequence of the protein would differ from a reference protein that has not undergone circular permutation. However, one of ordinary skill in the art would be able to determine which residues in the protein that has undergone circular permutation correspond to residues in the reference protein that has not undergone circular permutation by, for example, aligning the sequences and detecting conserved motifs, and/or by comparing the structures or predicted structures of the proteins, e.g., by homology modeling.

In some embodiments, an algorithm that determines the percent identity between a sequence of interest and a reference sequence described in this application accounts for the presence of circular permutation between the sequences. The presence of circular permutation may be detected using any method known in the art, including, for example, RASPODOM (Weiner et al., Bioinformatics. 2005 Apr. 1; 21(7):932-7). In some embodiments, the presence of circulation permutation is corrected for (e.g., the domains in at least one sequence are rearranged) prior to calculation of the percent identity between a sequence of interest and a sequence described in this application. The claims of this application should be understood to encompass sequences for which percent identity to a reference sequence is calculated after taking into account potential circular permutation of the sequence.

### Expression of Nucleic Acids in Host Cells

Aspects of the present disclosure relate to recombinant enzymes, functional modifications and variants thereof, as well as their uses. For example, the methods described in this application may be used to produce cannabinoids and/or cannabinoid precursors. The methods may comprise using a host cell comprising an enzyme disclosed in this application, cell lysate, isolated enzymes, or any combination thereof. Methods comprising recombinant expression of genes encoding an enzyme disclosed in this application in a host cell are encompassed by the present disclosure. In vitro methods comprising reacting one or more cannabinoid precursors or cannabinoids in a reaction mixture with an enzyme disclosed in this application are also encompassed by the present disclosure. In some embodiments, the enzyme is a PT.

A nucleic acid encoding any of the recombinant polypeptides (e.g., AAE, PKS, PKC, PT, or TS enzyme) described in this application may be incorporated into any appropriate vector through any method known in the art. For example, the vector may be an expression vector, including but not limited to a viral vector (e.g., a lentiviral, retroviral, adenoviral, or adeno-associated viral vector), any vector suitable for transient expression, any vector suitable for constitutive expression, or any vector suitable for inducible expression (e.g., a galactose-inducible or doxycycline-inducible vector).

A vector encoding any of the recombinant polypeptides (e.g., AAE, PKS, PKC, PT, or TS enzyme) described in this application may be introduced into a suitable host cell using any method known in the art. Non-limiting examples of yeast transformation protocols are described in Gietz el al., Yeast transformation can be conducted by the LiAc/SS Carrier DNA/PEG method. Methods Mol Biol. 2006; 313:107-20, which is hereby incorporated by reference in its entirety. Host cells may be cultured under any conditions suitable as would be understood by one of ordinary skill in the art. For example, any media, temperature, and incubation conditions known in the art may be used. For host cells carrying an inducible vector, cells may be cultured with an appropriate inducible agent to promote expression.

In some embodiments, a vector replicates autonomously in the cell. In some embodiments, a vector integrates into a chromosome within a cell. A vector can contain one or more endonuclease restriction sites that are cut by a restriction endonuclease to insert and ligate a nucleic acid containing a gene described in this application to produce a recombinant vector that is able to replicate in a cell. Vectors are typically composed of DNA, although RNA vectors are also available. Cloning vectors include, but are not limited to: plasmids, fosmids, phagemids, virus genomes and artificial chromosomes. As used in this application, the terms “expression vector” or “expression construct” refer to a nucleic acid construct, generated recombinantly or synthetically, with a series of specified nucleic acid elements that permit transcription of a particular nucleic acid in a host cell (e.g., microbe), such as a yeast cell. In some embodiments, the nucleic acid sequence of a gene described in this application is inserted into a cloning vector so that it is operably joined to regulatory sequences and, in some embodiments, expressed as an RNA transcript. In some embodiments, the vector contains one or more markers, such as a selectable marker as described in this application, to identify cells transformed or transfected with the recombinant vector. In some embodiments, a host cell has already been transformed with one or more vectors. In some embodiments, a host cell that has been transformed with one or more vectors is subsequently transformed with one or more vectors. In some embodiments, a host cell is transformed simultaneously with more than one vector. In some embodiments, a cell that has been transformed with a vector or an expression cassette incorporates all or part of the vector or expression cassette into its genome. In some embodiments, the nucleic acid sequence of a gene described in this application is recoded. Recoding may increase production of the gene product by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%, including all values in between) relative to a reference sequence that is not recoded.

In some embodiments, the nucleic acid encoding any of the proteins described in this application is under the control of regulatory sequences (e.g., enhancer sequences). In some embodiments, a nucleic acid is expressed under the control of a promoter. The promoter can be a native promoter, e.g., the promoter of the gene in its endogenous context, which provides normal regulation of expression of the gene. Alternatively, a promoter can be a promoter that is different from the native promoter of the gene, e.g., the promoter is different from the promoter of the gene in its endogenous context.

In some embodiments, the promoter is a eukaryotic promoter. Non-limiting examples of eukaryotic promoters include TDH3, PGK1, PKC1, PDC1, TEF1, TEF2, RPL18B, SSA1, TDH2, PYK1, TPI1, GAL1, GAL10, GAL7, GAL3, GAL2, MET3, MET25, HXT3, HXT7, ACT1, ADH1, ADH2, CUPI-1, ENO2, and SOD1, as would be known to one of ordinary skill in the art (see, e.g., Addgene website: blog.addgene.org/plasmids-101-the-promoter-region). In some embodiments, the promoter is a prokaryotic promoter (e.g., bacteriophage or bacterial promoter). Non-limiting examples of bacteriophage promoters include Pls1con, T3, T7, SP6, and PL. Non-limiting examples of bacterial promoters include Pbad, PmgrB, Ptrc2, Plac/ara, Ptac, and Pm.

In some embodiments, the promoter is an inducible promoter. As used in this application, an “inducible promoter” is a promoter controlled by the presence or absence of a molecule. This may be used, for example, to controllably induce the expression of an enzyme. In some embodiments, an inducible promoter linked to an enzyme may be used to regulate expression of the enzyme(s), for example to reduce cannabinoid production in certain scenarios (e.g., during transport of the genetically modified organism to satisfy regulatory restrictions in certain jurisdictions, or between jurisdictions, where cannabinoids may not be shipped). In some embodiments, an inducible promoter linked to an enzyme may be used to regulate expression of the enzyme(s), for example to reduce cannabinoid production in certain scenarios (e.g., during transport of the genetically modified organism to satisfy regulatory restrictions in certain jurisdictions, or between jurisdictions, where cannabinoids may not be shipped). Non-limiting examples of inducible promoters include chemically regulated promoters and physically regulated promoters. For chemically regulated promoters, the transcriptional activity can be regulated by one or more compounds, such as alcohol, tetracycline, galactose, a steroid, a metal, an amino acid, or other compounds. For physically regulated promoters, transcriptional activity can be regulated by a phenomenon such as light or temperature. Non-limiting examples of tetracycline-regulated promoters include anhydrotetracycline (aTc)-responsive promoters and other tetracycline-responsive promoter systems (e.g., a tetracycline repressor protein (tetR), a tetracycline operator sequence (tetO) and a tetracycline transactivator fusion protein (tTA)). Non-limiting examples of steroid-regulated promoters include promoters based on the rat glucocorticoid receptor, human estrogen receptor, moth ecdysone receptors, and promoters from the steroid/retinoid/thyroid receptor superfamily. Non-limiting examples of metal-regulated promoters include promoters derived from metallothionein (proteins that bind and sequester metal ions) genes. Non-limiting examples of pathogenesis-regulated promoters include promoters induced by salicylic acid, ethylene or benzothiadiazole (BTH). Non-limiting examples of temperature/heat-inducible promoters include heat shock promoters. Non-limiting examples of light-regulated promoters include light responsive promoters from plant cells. In certain embodiments, the inducible promoter is a galactose-inducible promoter. In some embodiments, the inducible promoter is induced by one or more physiological conditions (e.g., pH, temperature, radiation, osmotic pressure, saline gradients, cell surface binding, or concentration of one or more extrinsic or intrinsic inducing agents). Non-limiting examples of an extrinsic inducer or inducing agent include amino acids and amino acid analogs, saccharides and polysaccharides, nucleic acids, protein transcriptional activators and repressors, cytokines, toxins, petroleum-based compounds, metal containing compounds, salts, ions, enzyme substrate analogs, hormones or any combination.

In some embodiments, the promoter is a constitutive promoter. As used in this application, a “constitutive promoter” refers to an unregulated promoter that allows continuous transcription of a gene. Non-limiting examples of a constitutive promoter include TDH3, PGK1, PKC1, PDC1, TEF1, TEF2, RPL18B, SSA1, TDH2, PYK1, TPI1, HXT3, HXT7, ACT1. ADH1, ADH2, ENO2, and SOD1.

Other inducible promoters or constitutive promoters, including synthetic promoters, that may be known to one of ordinary skill in the art are also contemplated.

The precise nature of the regulatory sequences needed for gene expression may vary between species or cell types, but generally include, as necessary, 5′ non-transcribed and 5′ non-translated sequences involved with the initiation of transcription and translation respectively, such as a TATA box, capping sequence, CAAT sequence, and the like. In particular, such 5′ non-transcribed regulatory sequences will include a promoter region which includes a promoter sequence for transcriptional control of the operably joined gene. Regulatory sequences may also include enhancer sequences or upstream activator sequences. The vectors disclosed may include 5′ leader or signal sequences. The regulatory sequence may also include a terminator sequence. In some embodiments, a terminator sequence marks the end of a gene in DNA during transcription. The choice and design of one or more appropriate vectors suitable for inducing expression of one or more genes described in this application in a heterologous organism is within the ability and discretion of one of ordinary skill in the art.

Expression vectors containing the necessary elements for expression are commercially available and known to one of ordinary skill in the art (see, e.g., Sambrook et al., Molecular Cloning: A Laboratory Manual, Fourth Edition, Cold Spring Harbor Laboratory Press, 2012).

### Host Cells

The disclosed cannabinoid biosynthetic methods and host cells are exemplified with S. cerevisiae, but are also applicable to other host cells, as would be understood by one of ordinary skill in the art.

Suitable host cells include, but are not limited to: yeast cells, bacterial cells, algal cells, plant cells, fungal cells, insect cells, and animal cells, including mammalian cells. In one illustrative embodiment, suitable host cells include E. coli (e.g., Shuffle™ competent E. coli available from New England BioLabs in Ipswich, Mass.).

Other suitable host cells of the present disclosure include microorganisms of the genus Corynebacterium. In some embodiments, preferred Corynebacterium strains/species include: C. efficiens, with the deposited type strain being DSM44549, C. glutamicum, with the deposited type strain being ATCC13032, and C. ammoniagenes, with the deposited type strain being ATCC6871. In some embodiments the preferred host cell of the present disclosure is C. glutamicum.

Suitable host cells of the genus Corynebacterium, in particular of the species Corynebacterium glutamicum, are in particular the known wild-type strains: Corynebacterium glutamicum ATCC13032, Corynebacterium acetoglutamicum ATCC15806, Corynebacterium acetoacidophilum ATCC13870, Corynebacterium melassecola ATCC17965, Corynebacterium thermoaminogenes FERM BP-1539, Brevibacterium flavum ATCC14067, Brevibacterium lactofermentum ATCC13869, and Brevibacterium divaricatum ATCC14020; and L-amino acid-producing mutants, or strains, prepared therefrom, such as, for example, the L-lysine-producing strains: Corynebacterium glutamicum FERM-P 1709, Brevibacterium flavum FERM-P 1708, Brevibacterium lactofermentum FERM-P 1712, Corynebacterium glutamicum FERM-P 6463, Corynebacterium glutamicum FERM-P 6464, Corynebacterium glutamicum DM58-1, Corynebacterium glutamicum DG52-5, Corynebacterium glutamicum DSM5714, and Corynebacterium glutamicum DSM12866.

Suitable yeast host cells include, but are not limited to: Candida, Hansenula, Saccharomyces, Schizosaccharomyces, Pichia, Kluyveromyces, and Yarrowia. In some embodiments, the yeast cell is Hansenula polymorpha, Saccharomyces cerevisiae, Saccharomyces carlsbergensis, Saccharomyces diastaticus, Saccharomyces norbensis, Saccharomyces kluyveri, Schizosaccharomyces pombe, Komagataella phaffii, formerly known as Pichia pastoris, Pichia finlandica, Pichia trehalophila, Pichia kodamae, Pichia membranaefaciens, Pichia opuntiae, Pichia thermotolerans, Pichia salictaria, Pichia quercuum. Pichia pijperi, Pichia stipitis, Pichia methanolica, Pichia angusta, Kluyveromyces lactis, Candida albicans, or Yarrowia lipolytica.

In some embodiments, the yeast strain is an industrial polyploid yeast strain. Other non-limiting examples of fungal cells include cells obtained from Aspergillus spp., Penicillium spp., Fusarium spp., Rhizopus spp., Acremonium spp., Neurospora spp., Sordaria spp., Magnaporthe spp., Allomyces spp., Ustilago spp., Botrytis spp., and Trichoderma spp.

In certain embodiments, the host cell is an algal cell such as, Chlamydomonas (e.g., C. reinhardtii) and Phormidium (P. sp. ATCC29409).

In other embodiments, the host cell is a prokaryotic cell. Suitable prokaryotic cells include gram positive, gram negative, and gram-variable bacterial cells. The host cell may be a species of, but not limited to: Agrobacterium, Alicyclobacillus, Anabaena, Anacyslis, Acinetobacter, Acidothermus, Arthrobacter, Azobacter, Bacillus, Bifidobacterium, Brevibacterium, Butyrivibrio, Buchnera, Campestris, Camplyobacter, Clostridium, Corynebacterium, Chromatium, Coprococcus, Escherichia, Enterococcus, Enterobacter, Erwinia, Fusobacterium, Faecalibacterium, Francisella, Flavobacterium, Geobacillus, Haemophilus, Helicobacter, Klebsiella, Lactobacillus, Lactococcus, Ilyobacter, Micrococcus, Microbacterium, Mesorhizobium, Methylobacterium, Methylobacterium, Mycobacterium, Neisseria, Pantoea, Pseudomonas, Prochlorococcus, Rhodobacter, Rhodopseudomonas, Rhodopseudomonas, Roseburia, Rhodospirillum, Rhodococcus, Scenedesmus, Streptomyces, Streptococcus, Synechococcus, Saccharomonospora, Saccharopolyspora, Staphylococcus Serratia, Salmonella, Shugella, Thermoanaerobacterium, Tropheryma, Tularensis, Temecula, Thermosynechococcus, Thermococcus, Ureaplasma, Xanthomonas, Xylella, Yersinia, and Zymomonas.

In some embodiments, the bacterial host strain is an industrial strain. Numerous bacterial industrial strains are known and suitable for the methods and compositions described in this application.

In some embodiments, the bacterial host cell is of the Agrobacterium species (e.g., A. radiobacter, A. rhizogenes. A. rubi), the Arthrobacterspecies (e.g., A. aurescens, A. citreus, A. globformis, A. hydrocarboglutamicus, A. mysorens, A. nicotianae, A. paraffineus, A. protophonniae, A. roseoparaffinus, A. sulfureus, A. ureafaciens), the Bacillus species (e.g., B. thuringiensis. B. anthracis. B. megaterium, B. subtilis. B. lentus, B. circulars. B. pumilus, B. lautus, B. coagulans, B. brevis, B. firmus, B. alkaophius, B. licheniformis, B. clausii, B. stearothermophilus, B. halodurans and B. amyloliquefaciens. In particular embodiments, the host cell will be an industrial Bacillus strain including but not limited to B. subtilis. B. pumilus, B. licheniformis, B. megaterium, B. clausii, B. stearothermophilus and B. amyloliquefaciens. In some embodiments, the host cell will be an industrial Clostridium species (e.g., C. acetobutylicum, C. tetani E88, C. lituseburense, C. saccharobutylicum, C. perfringens, C. beijerinckii). In some embodiments, the host cell will be an industrial Corynebacterium species (e.g., C. glutamicum, C. acetoacidophilum). In some embodiments, the host cell will be an industrial Escherichia species (e.g., E. coli). In some embodiments, the host cell will be an industrial Erwinia species (e.g., E. uredovora, E. carotovora, E. ananas, E. herbicola, E. punctata, E. terreus). In some embodiments, the host cell will be an industrial Pantoea species (e.g., P. citrea, P. agglomerans). In some embodiments, the host cell will be an industrial Pseudomonas species, (e.g., P. putida, P. aeruginosa, P. mevalonii). In some embodiments, the host cell will be an industrial Streptococcus species (e.g., S. equisimiles, S. pyogenes, S. uberis). In some embodiments, the host cell will be an industrial Streptomyces species (e.g., S. ambofaciens, S. achromogenes, S. avermitilis, S. coelicolor, S. aureofaciens, S. aureus, S. fungicidicus, S. griseus, S. lividans). In some embodiments, the host cell will be an industrial Zymomonas species (e.g., Z. mobilis, Z. lipolytica), and the like.

The present disclosure is also suitable for use with a variety of animal cell types, including mammalian cells, for example, human (including 293, HeLa, W138. PER.C6 and Bowes melanoma cells), mouse (including 3T3, NS0, NS1, Sp2/0), hamster (CHO, BHK), monkey (COS. FRhL, Vero), insect cells, for example fall armyworm (including Sf9 and Sf21), silkmoth (including BmN), cabbage looper (including BTI-Tn-5B1-4) and common fruit fly (including Schneider 2), and hybridoma cell lines.

In various embodiments, strains that may be used in the practice of the disclosure including both prokaryotic and eukaryotic strains, and are readily accessible to the public from a number of culture collections such as American Type Culture Collection (ATCC), Deutsche Sammlung von Mikroorganismen and Zellkulturen GmbH (DSM), Centraalbureau Voor Schimmelcultures (CBS), and Agricultural Research Service Patent Culture Collection, Northern Regional Research Center (NRRL). The present disclosure is also suitable for use with a variety of plant cell types. In some embodiments, the plant is of the Cannabis genus in the family Cannabaceae. In certain embodiments, the plant is of the species Cannabis sativa, Cannabis indica, or Cannabis ruderalis. In other embodiments, the plant is of the genus Nicotiana in the family Solanaceae. In certain embodiments, the plant is of the species Nicotiana rustica.

The term “cell,” as used in this application, may refer to a single cell or a population of cells, such as a population of cells belonging to the same cell line or strain. Use of the singular term “cell” should not be construed to refer explicitly to a single cell rather than a population of cells. The host cell may comprise genetic modifications relative to a wild-type counterpart. Reduction of gene expression and/or gene inactivation in a host cell may be achieved through any suitable method, including but not limited to, deletion of the gene, introduction of a point mutation into the gene, selective editing of the gene and/or truncation of the gene. For example, polymerase chain reaction (PCR)-based methods may be used (see, e.g., Gardner et al., Methods Mol Biol. 2014; 1205:45-78). As a non-limiting example, genes may be deleted through gene replacement (e.g., with a marker, including a selection marker). A gene may also be truncated through the use of a transposon system (see, e.g., Poussu et al., Nucleic Acids Res. 2005; 33(12): e104). A gene may also be edited through of the use of gene editing technologies known in the art, such as CRISPR-based technologies.

### Culturing of Host Cells

Any of the cells disclosed in this application can be cultured in media of any type (rich or minimal) and any composition prior to, during, and/or after contact and/or integration of a nucleic acid. The conditions of the culture or culturing process can be optimized through routine experimentation as would be understood by one of ordinary skill in the art. In some embodiments, the selected media is supplemented with various components. In some embodiments, the concentration and amount of a supplemental component is optimized. In some embodiments, other aspects of the media and growth conditions (e.g., pH, temperature, etc.) are optimized through routine experimentation. In some embodiments, the frequency that the media is supplemented with one or more supplemental components, and the amount of time that the cell is cultured, is optimized.

Culturing of the cells described in this application can be performed in culture vessels known and used in the art. In some embodiments, an aerated reaction vessel (e.g., a stirred tank reactor) is used to culture the cells. In some embodiments, a bioreactor or fermenter is used to culture the cell. Thus, in some embodiments, the cells are used in fermentation. As used in this application, the terms “bioreactor” and “fermenter” are interchangeably used and refer to an enclosure, or partial enclosure, in which a biological, biochemical and/or chemical reaction takes place that involves a living organism or part of a living organism. A “large-scale bioreactor” or “industrial-scale bioreactor” is a bioreactor that is used to generate a product on a commercial or quasi-commercial scale. Large scale bioreactors typically have volumes in the range of liters, hundreds of liters, thousands of liters, or more.

Non-limiting examples of bioreactors include: stirred tank fermenters, bioreactors agitated by rotating mixing devices, chemostats, bioreactors agitated by shaking devices, airlift fermenters, packed-bed reactors, fixed-bed reactors, fluidized bed bioreactors, bioreactors employing wave induced agitation, centrifugal bioreactors, roller bottles, and hollow fiber bioreactors, roller apparatuses (for example benchtop, cart-mounted, and/or automated varieties), vertically-stacked plates, spinner flasks, stirring or rocking flasks, shaken multi-well plates, MD bottles, T-flasks. Roux bottles, multiple-surface tissue culture propagators, modified fermenters, and coated beads (e.g. beads coated with serum proteins, nitrocellulose, or carboxymethyl cellulose to prevent cell attachment).

In some embodiments, the bioreactor includes a cell culture system where the cell (e.g., yeast cell) is in contact with moving liquids and/or gas bubbles. In some embodiments, the cell or cell culture is grown in suspension. In other embodiments, the cell or cell culture is attached to a solid phase carrier. Non-limiting examples of a carrier system includes microcarriers (e.g., polymer spheres, microbeads, and microdisks that can be porous or non-porous), cross-linked beads (e.g., dextran) charged with specific chemical groups (e.g., tertiary amine groups), 2D microcarriers including cells trapped in nonporous polymer fibers, 3D carriers (e.g., carrier fibers, hollow fibers, multicartridge reactors, and semi-permeable membranes that can comprising porous fibers), microcarriers having reduced ion exchange capacity, encapsulation cells, capillaries, and aggregates. In some embodiments, carriers are fabricated from materials such as dextran, gelatin, glass, or cellulose.

In some embodiments, industrial-scale processes are operated in continuous, semi-continuous or non-continuous modes. Non-limiting examples of operation modes are batch, fed batch, extended batch, repetitive batch, draw/fill, rotating-wall, spinning flask, and/or perfusion mode of operation. In some embodiments, a bioreactor allows continuous or semi-continuous replenishment of the substrate stock, for example a carbohydrate source and/or continuous or semi-continuous separation of the product, from the bioreactor.

In some embodiments, the bioreactor or fermenter includes a sensor and/or a control system to measure and/or adjust reaction parameters. Non-limiting examples of reaction parameters include biological parameters (e.g., growth rate, cell size, cell number, cell density, cell type, or cell state, etc.), chemical parameters (e.g., pH, redox-potential, concentration of reaction substrate and/or product, concentration of dissolved gases, such as oxygen concentration and CO₂ concentration, nutrient concentrations, metabolite concentrations, concentration of an oligopeptide, concentration of an amino acid, concentration of a vitamin, concentration of a hormone, concentration of an additive, serum concentration, ionic strength, concentration of an ion, relative humidity, molarity, osmolarity, concentration of other chemicals, for example buffering agents, adjuvants, or reaction by-products), physical/mechanical parameters (e.g., density, conductivity, degree of agitation, pressure, and flow rate, shear stress, shear rate, viscosity, color, turbidity, light absorption, mixing rate, conversion rate, as well as thermodynamic parameters, such as temperature, light intensity/quality, etc.). Sensors to measure the parameters described in this application are well known to one of ordinary skill in the relevant mechanical and electronic arts. Control systems to adjust the parameters in a bioreactor based on the inputs from a sensor described in this application are well known to one of ordinary skill in the art in bioreactor engineering.

In some embodiments, the method involves batch fermentation (e.g., shake flask fermentation). General considerations for batch fermentation (e.g., shake flask fermentation) include the level of oxygen and glucose. For example, batch fermentation (e.g., shake flask fermentation) may be oxygen and glucose limited, so in some embodiments, the capability of a strain to perform in a well-designed fed-batch fermentation is underestimated. Also, the final product (e.g., cannabinoid or cannabinoid precursor) may display some differences from the substrate in terms of solubility, toxicity, cellular accumulation and secretion and in some embodiments can have different fermentation kinetics.

In some embodiments, the cells of the present disclosure are adapted to produce cannabinoids or cannabinoid precursors in vivo. In some embodiments, the cells are adapted to secrete one or more enzymes for cannabinoid synthesis (e.g., AAE, PKS, PKC, PT, or TS). In some embodiments, the cells of the present disclosure are lysed, and the lysate is recovered for subsequent use. In such embodiments, the secreted or lysed enzyme can catalyze reactions for the production of a cannabinoid or precursor by bioconversion in an in vitro or ex vivo process. In some embodiments, any and all conversions described in this application can be conducted chemically or enzymatically, in vitro or in vivo.

In some embodiments, the host cells of the present disclosure are adapted to produce cannabinoids or cannabinoid precursors in vivo. In some embodiments, the host cells are adapted to secrete one or more cannabinoid pathway substrates, intermediates, and/or terminal products (e.g., olivetol, THCA, THC, CBDA, CBD, CBGA, CBGVA, THCVA, CBDVA, CBCVA, or CBCA). In some embodiments, the host cells of the present disclosure are lysed, and the lysate is recovered for subsequent use. In such embodiments, the secreted substrates, intermediates, and/or terminal products may be recovered from the culture media.

### Purification and Further Processing

In some embodiments, any of the methods described in this application may include isolation and/or purification of the cannabinoids and/or cannabinoid precursors produced (e.g., produced in a bioreactor). For example, the isolation and/or purification can involve one or more of cell lysis, centrifugation, extraction, column chromatography, distillation, crystallization, and lyophilization.

The methods described in this application encompass production of any cannabinoid or cannabinoid precursor known in the art. Cannabinoids or cannabinoid precursors produced by any of the recombinant cells disclosed in this application or any of the in vitro methods described in this application may be identified and extracted using any method known in the art. Mass spectrometry (e.g., LC-MS, GC-MS) is a non-limiting example of a method for identification and may be used to extract a compound of interest.

In some embodiments, any of the methods described in this application further comprise decarboxylation of a cannabinoid or cannabinoid precursor. As a non-limiting example, the acid form of a cannabinoid or cannabinoid precursor may be heated (e.g., at least 90° C.) to decarboxylate the cannabinoid or cannabinoid precursor. See, e.g., U.S. Pat. Nos. 10,159,908, 10,143,706, 9,908,832 and 7,344,736. See also, e.g., Wang et al., Cannabis Cannabinoid Res. 2016; 1(1): 262-271.

### Compositions, Kits, and Administration

The present disclosure provides compositions, including pharmaceutical compositions, comprising a cannabinoid or a cannabinoid precursor, or pharmaceutically acceptable salt thereof, produced by any of the methods described in this application, and optionally a pharmaceutically acceptable excipient.

In certain embodiments, a cannabinoid or cannabinoid precursor described in this application is provided in an effective amount in a composition, such as a pharmaceutical composition. In certain embodiments, the effective amount is a therapeutically effective amount. In certain embodiments, the effective amount is a prophylactically effective amount.

Compositions, such as pharmaceutical compositions, described in this application can be prepared by any method known in the art. In general, such preparatory methods include bringing a compound described in this application (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described in this application will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition. Exemplary excipients include diluents, dispersing and/or granulating agents, surface active agents and/or emulsifiers, disintegrating agents, binding agents, preservatives, buffering agents, lubricating agents, and/or oils (e.g., synthetic oils, semi-synthetic oils) as disclosed in this application.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate. Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic or semi-synthetic oils include, but are not limited to, butyl stearate, medium chain triglycerides (such as caprylic triglyceride and capric triglyceride), cyclomethicone, diethyl sebacate, dimethicone 360, isopropyl myristate, mineral oil, octyldodecanol, oleyl alcohol, silicone oil, and mixtures thereof. In certain embodiments, exemplary synthetic oils comprise medium chain triglycerides (such as caprylic triglyceride and capric triglyceride).

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described in this application are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose, any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described in this application with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a compound described in this application may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described in this application include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described in this application.

A pharmaceutical composition described in this application can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally, the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Although the descriptions of pharmaceutical compositions provided in this application are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided in this application are typically formulated in dosage unit form for ease of administration and uniformity of dosage. It will be understood, however, that the total daily usage of the compositions described in this application will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The compounds and compositions provided in this application can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation, and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration).

In some embodiments, compounds or compositions disclosed in this application are formulated and/or administered in nanoparticles. Nanoparticles are particles in the nanoscale. In some embodiments, nanoparticles are less than 1 μm in diameter. In some embodiments, nanoparticles are between about 1 and 100 nm in diameter. Nanoparticles include organic nanoparticles, such as dendrimers, liposomes, or polymeric nanoparticles. Nanoparticles also include inorganic nanoparticles, such as fullerenes, quantum dots, and gold nanoparticles. Compositions may comprise an aggregate of nanoparticles. In some embodiments, the aggregate of nanoparticles is homogeneous, while in other embodiments the aggregate of nanoparticles is heterogeneous.

The exact amount of a compound required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of a compound described in this application. In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described in this application includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described in this application. In certain embodiments, a dose described in this application includes independently between 1 mg and 3 mg, inclusive, of a compound described in this application. In certain embodiments, a dose described in this application includes independently between 3 mg and 10 mg, inclusive, of a compound described in this application. In certain embodiments, a dose described in this application includes independently between 10 mg and 30 mg, inclusive, of a compound described in this application. In certain embodiments, a dose described in this application includes independently between 30 mg and 100 mg, inclusive, of a compound described in this application.

Dose ranges as described in this application provide guidance for the administration of provided pharmaceutical compositions to an adult. The amount to be administered to, for example, a child or an adolescent can be determined by a medical practitioner or person skilled in the art and can be lower or the same as that administered to an adult.

A compound or composition, as described in this application, can be administered in combination with one or more additional pharmaceutical agents (e.g., therapeutically and/or prophylactically active agents). The compounds or compositions can be administered in combination with additional pharmaceutical agents that improve their activity, improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described in this application including a compound described in this application and an additional pharmaceutical agent shows a synergistic effect that is absent in a pharmaceutical composition including one of the compound and the additional pharmaceutical agent, but not both.

The compound or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., proliferative disease, neurological disease, painful condition, psychiatric disorder, or metabolic disorder). Each additional pharmaceutical agent may be administered at a dose and/or on a time schedule determined for that pharmaceutical agent. The additional pharmaceutical agents may also be administered together with each other and/or with the compound or composition described in this application in a single dose or administered separately in different doses. The particular combination to employ in a regimen will take into account compatibility of the compound described in this application with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

In some embodiments, one or more of the compositions described in this application are administered to a subject. In certain embodiments, the subject is an animal. The animal may be of either sex and may be at any stage of development. In certain embodiments, the subject is a human. In other embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate.

Also encompassed by the disclosure are kits (e.g., pharmaceutical packs). The kits provided may comprise a composition, such as a pharmaceutical composition, or a compound described in this application and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described in this application. In some embodiments, the pharmaceutical composition or compound described in this application provided in the first container and the second container a combined to form one unit dosage form.

Thus, in one aspect, provided are kits including a first container comprising a compound or composition described in this application. In certain embodiments, the kits are useful for treating a disease in a subject in need thereof. In certain embodiments, the kits are useful for preventing a disease in a subject in need thereof. In certain embodiments, the kits are useful for reducing the risk of developing a disease in a subject in need thereof.

In certain embodiments, a kit described in this application further includes instructions for using the kit. A kit described in this application may also include information as required by a regulatory agency such as the U.S. Food and Drug Administration (FDA). In certain embodiments, the information included in the kits is prescribing information. In certain embodiments, the kits and instructions provide for treating a disease in a subject in need thereof. In certain embodiments, the kits and instructions provide for preventing a disease in a subject in need thereof. In certain embodiments, the kits and instructions provide for reducing the risk of developing a disease in a subject in need thereof. A kit described in this application may include one or more additional pharmaceutical agents described in this application as a separate composition.

In some embodiments, the compositions include consumer product, such as comestible, cosmetic, toiletry, potable, inhalable, and wellness products. Exemplary consumer products include salves, waxes, powdered concentrates, pastes, extracts, tinctures, powders, oils, capsules, skin patches, sublingual oral dose drops, mucous membrane oral spray doses, makeup, perfume, shampoos, cosmetic soaps, cosmetic creams, skin lotions, aromatic essential oils, massage oils, shaving preparations, oils for toiletry purposes, lip balm, cosmetic oils, facial washes, moisturizing creams, moisturizing body lotions, moisturizing face lotions, bath salts, bath gels, bath soaps in liquid form, shower gels, bath bombs, hair care preparations, shampoos, conditioner, chocolate bars, brownies, chocolates, cookies, crackers, cakes, cupcakes, puddings, honey, chocolate confections, frozen confections, fruit-based confectionery, sugar confectionery, gummy candies, dragées, pastries, cereal bars, chocolate, cereal based energy bars, candy, ice cream, tea-based beverages, coffee-based beverages, and herbal infusions.

The present invention is further illustrated by the following Examples, which in no way should be construed as limiting. The entire contents of all of the references (including literature references, issued patents, published patent applications, and co-pending patent applications) cited throughout this application are hereby expressly incorporated by reference. If a reference incorporated in this application contains a term whose definition is incongruous or incompatible with the definition of same term as defined in the present disclosure, the meaning ascribed to the term in this disclosure shall govern. However, mention of any reference, article, publication, patent, patent publication, and patent application cited in this disclosure is not, and should not be taken as an acknowledgment or any form of suggestion that they constitute valid prior art or form part of the common general knowledge in any country in the world.

### EXAMPLES

### Example 1: Primary Screen to Identify Functional Expression of Aromatic Prenyltransferases

Seven cannabigerolic acid synthase (CBGAS) genes have previously been identified in C. sativa: the prenyltransferases (PTs) CsPT1-7. These enzymes catalyze the C-alkylation by geranyl pyrophosphate of olivetolic acid (OA) to cannabigerolic acid (CBGA). It has previously been reported that it is difficult to express C. sativa PTs in S. cerevisiae; for example, out of CsPT1-7, only CsPT4 was reported to produce CBGA when expressed heterologously in S. cerevisiae, and only at low titers (Luo et al. Nature, 2019).

To identify additional PT proteins that could be functionally expressed in host cells, a protein engineering library of approximately 1074 proteins was designed using four different strategies: (1) point mutations based on bioinformatics analysis of CsPT sequences; (2) CsPT active-site saturation mutagenesis; (3) CsPT chimeras comprising portions of different CsPT sequences; and (4) protein fusions involving CsPTs and the farnesyl pyrophosphate synthase encoded by ERG20.

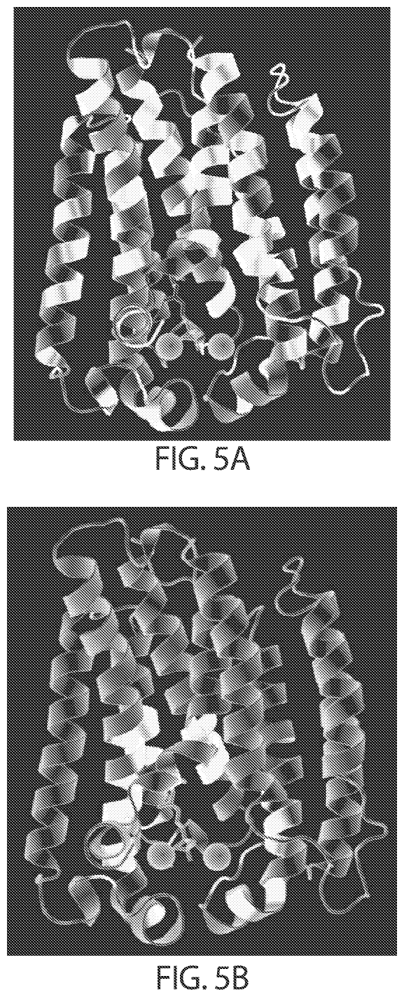

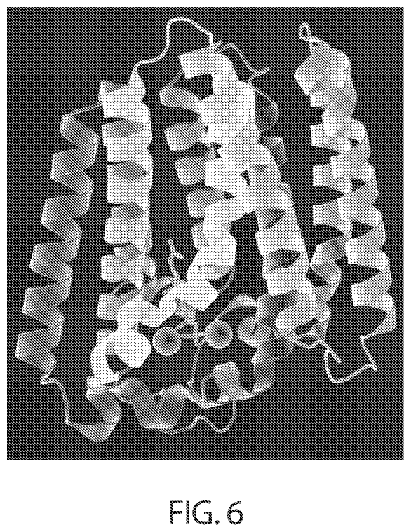

(1) Bioinformatics: bioinformatics analysis was used to predict the fitness of the native amino acid at every position in a CsPT4 protein sequence (SEQ ID NO; 5) and to suggest favorable alternatives if the native amino acid was suboptimal. This analysis produced a total of 281 protein sequences with single amino acid mutations. FIG. 5A depicts the structure of CsPT4 with regions spread throughout the sequence (shown in black) where point mutations were generated based on bioinformatics analysis.

(2) Active-site saturation mutagenesis: Based on structural modeling, 34 non-essential residue positions within 7 angstroms of the two Mg2+ ion positions or the non-hydrogen GPP substrate atom positions were identified and selected for saturation mutagenesis. This resulted in a total of 646 point mutations, including mutations at the following positions relative to SEQ ID NO: 5: V39, M43, F80, N81, F83, A84, A85, I86, M87, Q89, Y91, I95, L103, F145, G146, I147, F148, A149, F151, S154, R159, I170, T171, I172, S173, S174, H175, A215, K218, D219, I223, G225, V231, T233. FIG. 5B depicts the structure of CsPT4 with regions near the active site (shown in black) where point mutations were focused using this approach.

(3) Chimeras: chimeric proteins were generated from CsPT1-7 using cross-over points identified from sequence alignments between the CsPT proteins. The chimeras generated had nine presumed transmembrane helices and utilized two different cross-over design strategies: (A) “within membrane” CsPT chimeras with 9 cross-over points on each of nine presumed transmembrane helices (FIG. 7A); and (B) “through membrane chimeras” with a single cross-over point between helices 6&7 or 7&8 (FIG. 7B). In total, 54 “within membrane” and 36 “through membrane” chimeras were generated.

(4) CsPT fusion proteins: fusion proteins were constructed in which truncated versions of CsPTs were fused at their amino terminus to either ERG20 containing the point mutations F96W and N127W (ERG20ww; SEQ ID NO: 103), or to a GFP control protein. 7 different linkers of varying lengths and sequences were used in combination with 3 truncated versions of CsPTs. 42 proteins were generated using this design strategy.

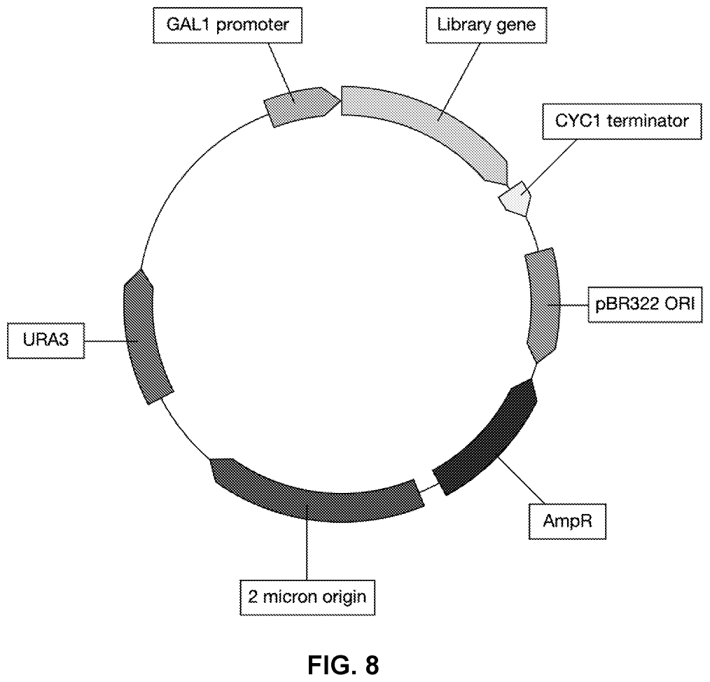

Protein sequences were recoded in silico for expression in S. cerevisiae and synthesized in the replicative yeast expression vector shown in FIG. 8. Each enzyme expression construct was transformed into an S. cerevisiae CEN.PK strain that was engineered to overproduce GPP. Transformants were selected based on ability to grow on media lacking uracil. Strain t459830, comprising a fluorescent protein (GFP), was included in the library screen as a negative control for enzyme activity. Strain t460439, comprising a truncated C saliva CsPT4 protein (SEQ ID NO:5) was included in the library as a positive control and was used to establish hit ranking.

The full set of PT enzymes was assayed for activity in a primary screen using a prenyltransferase assay which was conducted as follows: each thawed glycerol stock of PT transformants was stamped into a well of synthetic complete media minus uracil (SC-URA)+4% dextrose media. Samples were incubated at 30° C. in a shaking incubator for 2 days. A portion of each of the resulting cultures was stamped into a well of SC-URA+2% raffinose+2% galactose+1 mM olivetolic acid (C6). Samples were incubated at 30° C. and shaken in a shaking incubator for 4 days. A portion of each of the resulting production cultures was stamped into a well of phosphate buffered saline (PBS). Optical measurements were taken on a plate reader, with absorbance measured at 600 nm and fluorescence at 528 nm with 485 nm excitation. A portion of each of the production cultures was stamped into a well of 100% methanol in half-height deepwell plates. Plates were heat sealed and frozen. Samples were then thawed for 30 min and spun down at 4° C. A portion of the supernatant was stamped into half-area 96 well plates. CBGA production in the samples was measured via liquid chromatography-mass spectrometry (LC-MS) by measuring relative peak areas. CBGA production was quantified in μg/L by comparing LC/MS peak areas to a standard curve for CBGA.

The strains were tested for CBGA production by feeding OA to clonal expression cultures. LC-MS analysis revealed that 612 (57%) of library PTs produced measurable amounts of CBGA, and 138 (12.8%) of PTs produced CBGA concentrations comparable to or greater than the positive control strain. Importantly, 5% of the library PTs generated at least 30% more CBGA than the positive control strain, representing a significant improvement of CBGA production.

### Example 2: Secondary Screen to Confirm Functional Expression of Aromatic Prenyltransferases

To confirm the activity of the candidate PTs identified in Example 1, a secondary screen was performed. One hundred fifty of the candidate PTs from the primary screen described in Example 1 were subjected to the secondary screen to verify and further quantify cannabinoid production.

In addition to screening for activity on olivetolic acid (C6), a parallel experiment was performed to screen the set of enzymes tested in the secondary screen on the C4 substrate divaric acid (DA), by substituting 1 mM divaric acid for the 1 mM olivetolic acid (OA) in the prenyltransferase assay described in Example 1. The resulting products, CBGA and cannabigerovarinic acid (CBGVA), were quantified in μg/L by comparing LC-MS peak areas to the respective standard curve for CBGA and CBGVA. See, Example 1. The experimental protocols for the secondary screen were the same as the assays used in primary screen described in Example 1 except that both CBGA and CBGVA production were measured using LC-MS on four biological replicates incubated with OA or DA, respectively.

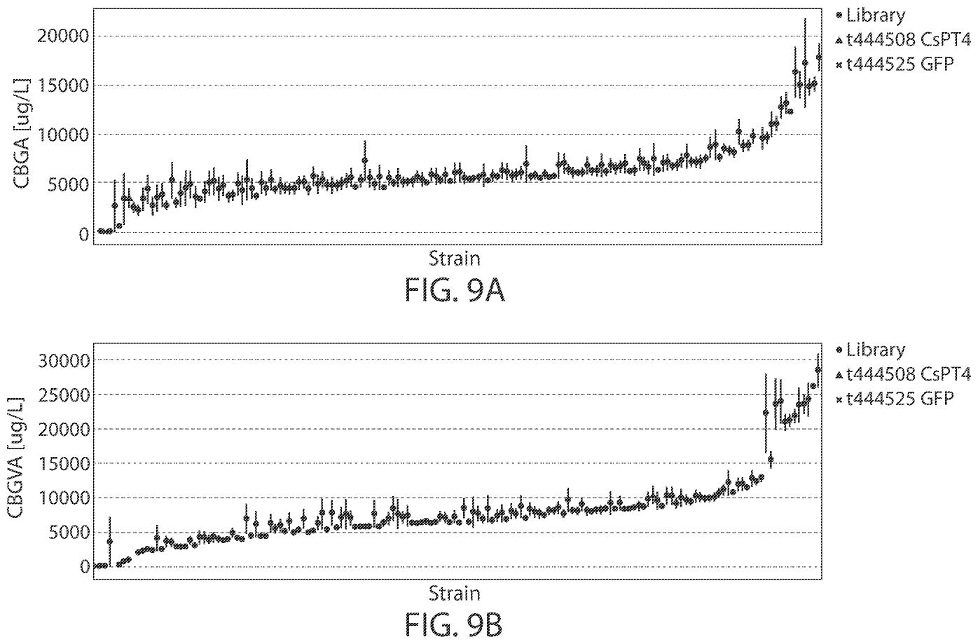

Strain t444525, comprising a fluorescent protein (GFP), was included in the library screen as a negative control for enzyme activity. Strain t444508, comprising a truncated C. sativa CsPT4 protein (SEQ ID NO: 5), was included in the library as a positive control and was used to establish hit ranking. Table 5 and FIGS. 9A-9d show the results of the secondary screen, in which enhanced cannabinoid production by PT variants was confirmed. The overall trends in PT activity observed in the secondary screen were consistent with the primary screen results. The distribution of CBGA and CBGVA production by library PTs was 1×-3× the activity of the CsPT4 positive control. Sequence information for strains in Table 5 comprising CsPT chimeras or fusions is provided in Table 13.

TABLE 5
Secondary screening activity data in S. cerevisiae
of PT library members described in Example 2

| | | | Standard | | Standard | | |
|---|---|---|---|---|---|---|---|
| | | Average | Deviation | Average | Deviation | | |
| | Strain | CBGA | CBGA | CBGVA | CBGVA | PT | |
| Strain | type | [μg/L] | [μg/L] | [μg/L] | [μg/L] | type | Mutation |
| t444508 | Positive | 3461.9 | 983.5 | 10321 | 1292.2 | Truncated | N/A |
| | control | | | | | CsPT4 | |
| t444525 | Negative | 30 | 64.1 | 6.5 | 14 | N/A | N/A |
| | control | | | | | | |
| t523578 | Library | 2665.5 | 484.7 | 3012.7 | 217.9 | chimera | N/A |
| t523602 | Library | 2501 | 676.2 | 2474.3 | 301.1 | chimera | N/A |
| t523722 | Library | 36.4 | 51.5 | 0 | 0 | chimera | N/A |
| t523777 | Library | 4684.7 | 635.3 | 680 | 83.9 | chimera | N/A |
| t523834 | Library | 4947.9 | 673.7 | 6087.2 | 2171.4 | chimera | N/A |
| t524736 | Library | 4397.6 | 678.9 | 6153.8 | 266.7 | chimera | N/A |
| t524816 | Library | 5149.2 | 699.4 | 2787.8 | 224.9 | chimera | N/A |
| t524866 | Library | 5286.2 | 2344.9 | 4084.8 | 2094.3 | chimera | N/A |
| t525864 | Library | 5654.6 | 291.6 | 8074 | 786 | chimera | N/A |
| t526650 | Library | 8824 | 1709.4 | 2748.5 | 227.6 | chimera | N/A |
| t526890 | Library | 3638.9 | 383.4 | 2809.4 | 175 | chimera | N/A |
| t526897 | Library | 4436.8 | 754 | 892.8 | 88 | chimera | N/A |
| t524521 | Library | 12191 | 321.3 | 26078.6 | 430.2 | fusion | N/A |
| t524649 | Library | 13070.7 | 1343.3 | 28480.3 | 2785.4 | fusion | N/A |
| t524722 | Library | 12636.2 | 1186.7 | 23994.2 | 3433.9 | fusion | N/A |
| t524730 | Library | 10994.5 | 753.9 | 21835 | 1015.3 | fusion | N/A |
| t524834 | Library | 14752.4 | 966 | 24313.7 | 1597.1 | fusion | N/A |
| t524842 | Library | 16251.5 | 3014.7 | 20941 | 1394.5 | fusion | N/A |
| t526009 | Library | 10114.8 | 1484 | 22259.7 | 6608.3 | fusion | N/A |
| t526811 | Library | 17124.9 | 4810.4 | 23525.9 | 4022.2 | fusion | N/A |
| t526843 | Library | 15033.7 | 749.6 | 23436.1 | 2612.2 | fusion | N/A |
| t526923 | Library | 17738.8 | 1649.1 | 23641.1 | 1574.2 | fusion | N/A |
| t526955 | Library | 14907.8 | 1537.6 | 21203.8 | 1082.6 | fusion | N/A |
| t523658 | Library | 581.4 | 164.3 | 128.2 | 43.9 | Point mutant | Y91L |
| | | | | | | (active site | |
| | | | | | | saturation | |
| | | | | | | mutation) | |
| t523737 | Library | 5014.2 | 1348.8 | 6322.3 | 559.8 | Point mutant | I263A |
| | | | | | | (bioinformatics) | |
| t523745 | Library | 3575.7 | 622.1 | 9887.9 | 547.7 | Point mutant | I86T |
| | | | | | | (active site | |
| | | | | | | saturation | |
| | | | | | | mutation) | |
| t523776 | Library | 6761.4 | 1376.4 | 8723.9 | 461.7 | Point mutant | E113R |
| | | | | | | (bioinformatics) | |
| t523786 | Library | 7552.2 | 499.1 | 5122.3 | 405.8 | Point mutant | F148S |
| | | | | | | (active site | |
| | | | | | | saturation | |
| | | | | | | mutation) | |
| t523810 | Library | 5041.5 | 594.6 | 6293.5 | 367 | Point mutant | F151M |
| | | | | | | (active site | |
| | | | | | | saturation | |
| | | | | | | mutation) | |
| t523817 | Library | 9706.6 | 822.9 | 12770.2 | 1301.3 | Point mutant | I86A |
| | | | | | | (active site | |
| | | | | | | saturation | |
| | | | | | | mutation) | |
| t523824 | Library | 5500.3 | 1052.1 | 12171.1 | 2151.2 | Point mutant | I86G |
| | | | | | | (active site | |
| | | | | | | saturation | |
| | | | | | | mutation) | |
| t523857 | Library | 5976.2 | 1170.2 | 12278 | 494.6 | Point mutant | L311R |
| | | | | | | (bioinformatics) | |
| t523882 | Library | 3786.1 | 1373.2 | 7576.8 | 2504.1 | Point mutant | M67L |
| | | | | | | (bioinformatics) | |
| t523914 | Library | 2960.6 | 538.7 | 8548.2 | 1009.3 | Point mutant | M67I |
| | | | | | | (bioinformatics) | |
| t524122 | Library | 4656.8 | 1215.4 | 4208.6 | 1064.7 | Point mutant | K41V |
| | | | | | | (bioinformatics) | |
| t524161 | Library | 5759.6 | 569.5 | 5392.8 | 734.7 | Point mutant | V231M |
| | | | | | | (active site | |
| | | | | | | saturation | |
| | | | | | | mutation) | |
| t524208 | Library | 7376.5 | 1263.4 | 8158.5 | 487.9 | Point mutant | F145S |
| | | | | | | (active site | |
| | | | | | | saturation | |
| | | | | | | mutation) | |
| t524217 | Library | 5611.4 | 874.5 | 4839.6 | 762.7 | Point mutant | F151G |
| | | | | | | (active site | |
| | | | | | | saturation | |
| | | | | | | mutation) | |
| t524232 | Library | 5349.7 | 407.5 | 7765.5 | 2138.6 | Point mutant | A149C |
| | | | | | | (active site | |
| | | | | | | saturation | |
| | | | | | | mutation) | |
| t524248 | Library | 5676.7 | 653.9 | 3910.5 | 296.8 | Point mutant | K41I |
| | | | | | | (bioinformatics) | |
| t524280 | Library | 6950.2 | 1147.2 | 8409.8 | 1593.8 | Point mutant | I46A |
| | | | | | | (bioinformatics) | |
| t524288 | Library | 6649.5 | 406.4 | 10055.1 | 733.8 | Point mutant | F145I |
| | | | | | | (active site | |
| | | | | | | saturation | |
| | | | | | | mutation) | |
| t524297 | Library | 5003.2 | 318.8 | 4194.4 | 825.4 | Point mutant | A215Y |
| | | | | | | (active site | |
| | | | | | | saturation | |
| | | | | | | mutation) | |
| t524322 | Library | 5974.3 | 752.8 | 6794.2 | 1626.4 | Point mutant | S174T |
| | | | | | | (active site | |
| | | | | | | saturation | |
| | | | | | | mutation) | |
| t524344 | Library | 6157.4 | 342.3 | 8670.2 | 1921.5 | Point mutant | F145C |
| | | | | | | (active site | |
| | | | | | | saturation | |
| | | | | | | mutation) | |
| t524352 | Library | 5560.2 | 1403.5 | 3449.3 | 852 | Point mutant | A47V |
| | | | | | | (bioinformatics) | |
| t524384 | Library | 4401.5 | 1110.6 | 9762.1 | 1163 | Point mutant | F56L |
| | | | | | | (bioinformatics) | |
| t524385 | Library | 6113 | 378.3 | 10237.2 | 1371.3 | Point mutant | M43V |
| | | | | | | (active site | |
| | | | | | | saturation | |
| | | | | | | mutation) | |
| t524466 | Library | 5566.6 | 371.2 | 7709.8 | 1472.6 | Point mutant | F145V |
| | | | | | | (active site | |
| | | | | | | saturation | |
| | | | | | | mutation) | |
| t524505 | Library | 7714.5 | 1410 | 10760.5 | 285.8 | Point mutant | F145L |
| | | | | | | (active site | |
| | | | | | | saturation | |
| | | | | | | mutation) | |
| t524512 | Library | 7118.9 | 537.2 | 8080.4 | 742.4 | Point mutant | I46G |
| | | | | | | (bioinformatics) | |
| t524536 | Library | 5833.3 | 545.4 | 7849.2 | 968.5 | Point mutant | I46C |
| | | | | | | (bioinformatics) | |
| t524570 | Library | 6721.6 | 759.7 | 8312.4 | 300.2 | Point mutant | S260L |
| | | | | | | (bioinformatics) | |
| t524592 | Library | 6785.2 | 1486.3 | 7637.1 | 743.1 | Point mutant | F318L |
| | | | | | | (bioinformatics) | |
| t524602 | Library | 7249.6 | 731 | 3926.3 | 193.4 | Point mutant | M210Y |
| | | | | | | (bioinformatics) | |
| t524625 | Library | 7070.6 | 946.2 | 8199.6 | 703.6 | Point mutant | V140L |
| | | | | | | (bioinformatics) | |
| t524672 | Library | 6864.9 | 2041.6 | 5207.9 | 427.9 | Point mutant | T244A |
| | | | | | | (bioinformatics) | |
| t524674 | Library | 8432.6 | 650.6 | 7172 | 839 | Point mutant | M87T |
| | | | | | | (active site | |
| | | | | | | saturation | |
| | | | | | | mutation) | |
| t524704 | Library | 6438.5 | 643.6 | 8372.3 | 1913.7 | Point mutant | F145M |
| | | | | | | (active site | |
| | | | | | | saturation | |
| | | | | | | mutation) | |
| t524753 | Library | 5693.3 | 784.2 | 6806.7 | 461.6 | Point mutant | I261A |
| | | | | | | (bioinformatics) | |
| t524761 | Library | 4545.9 | 337.3 | 5722.9 | 289.7 | Point mutant | A136P |
| | | | | | | (bioinformatics) | |
| t524833 | Library | 4375.3 | 530.2 | 3735.2 | 242.8 | Point mutant | F216I |
| | | | | | | (bioinformatics) | |
| t524850 | Library | 8769.6 | 598.6 | 9914 | 1449.5 | Point mutant | T187R |
| | | | | | | (bioinformatics) | |
| t524858 | Library | 3329.1 | 3025.4 | 4902.7 | 373.6 | Point mutant | R197I |
| | | | | | | (bioinformatics) | |
| t524865 | Library | 6270.3 | 533.6 | 8091 | 496.5 | Point mutant | S232R |
| | | | | | | (bioinformatics) | |
| t524874 | Library | 10900.2 | 1462.7 | 11423.9 | 438.6 | Point mutant | L311N |
| | | | | | | (bioinformatics) | |
| t524882 | Library | 5548.2 | 223.6 | 6976.9 | 263 | Point mutant | I142A |
| | | | | | | (bioinformatics) | |
| t525585 | Library | 2194.3 | 587.3 | 2162.1 | 253.8 | Point mutant | F151H |
| | | | | | | (active site | |
| | | | | | | saturation | |
| | | | | | | mutation) | |
| t525616 | Library | 3725.2 | 596.9 | 10220.5 | 1444.9 | Point mutant | S260V |
| | | | | | | (bioinformatics) | |
| t525676 | Library | 3517.7 | 1279.8 | 7602.7 | 2311.7 | Point mutant | S277G |
| | | | | | | (bioinformatics) | |
| t525690 | Library | 5645.1 | 1180.8 | 8380 | 2217.9 | Point mutant | C284L |
| | | | | | | (bioinformatics) | |
| t525713 | Library | 2656.1 | 864.1 | 3887.3 | 544.4 | Point mutant | F72E |
| | | | | | | (bioinformatics) | |
| t525728 | Library | 3323.2 | 1322.5 | 7698.3 | 2661.5 | Point mutant | A136S |
| | | | | | | (bioinformatics) | |
| t525736 | Library | 5208.9 | 2127.4 | 6829.9 | 1066.7 | Point mutant | A199S |
| | | | | | | (bioinformatics) | |
| t525740 | Library | 4431.1 | 2017.7 | 9006.1 | 1042.2 | Point mutant | F141L |
| | | | | | | (bioinformatics) | |
| t525756 | Library | 3452.8 | 1541.9 | 10239.1 | 1007.3 | Point mutant | N272S |
| | | | | | | (bioinformatics) | |
| t525760 | Library | 4323.4 | 1352.3 | 8042.5 | 687.1 | Point mutant | I142L |
| | | | | | | (bioinformatics) | |
| t525762 | Library | 4946.6 | 1411.6 | 7340.7 | 1739.2 | Point mutant | S184Y |
| | | | | | | (bioinformatics) | |
| t525772 | Library | 4346.7 | 1650.5 | 6881.7 | 2472.2 | Point mutant | R197A |
| | | | | | | (bioinformatics) | |
| t525780 | Library | 5828.8 | 904.2 | 9645.6 | 2009 | Point mutant | I273F |
| | | | | | | (bioinformatics) | |
| t525796 | Library | 4835.2 | 1544.9 | 9575.7 | 1467.7 | Point mutant | S184L |
| | | | | | | (bioinformatics) | |
| t525816 | Library | 5418.3 | 655.4 | 7561.8 | 835.9 | Point mutant | H29D |
| | | | | | | (bioinformatics) | |
| t525817 | Library | 21.1 | 17.8 | 3.3 | 6.6 | Point mutant | V39M |
| | | | | | | (active site | |
| | | | | | | saturation | |
| | | | | | | mutation) | |
| t525828 | Library | 5123.7 | 1636.2 | 7572.8 | 2484.3 | Point mutant | P301D |
| | | | | | | (bioinformatics) | |
| t525850 | Library | 3870.3 | 1445.4 | 7035.8 | 905.8 | Point mutant | T289A |
| | | | | | | (bioinformatics) | |
| t525856 | Library | 4873 | 1306.6 | 7188.6 | 1054.9 | Point mutant | S260A |
| | | | | | | (bioinformatics) | |
| t525858 | Library | 5477 | 824.5 | 8331.8 | 429.7 | Point mutant | S260I |
| | | | | | | (bioinformatics) | |
| t525860 | Library | 6912.5 | 1058.8 | 8313.3 | 367.4 | Point mutant | Q267F |
| | | | | | | (bioinformatics) | |
| t525884 | Library | 4732.5 | 941.4 | 5663.6 | 460.6 | Point mutant | H60E |
| | | | | | | (bioinformatics) | |
| t525906 | Library | 5123.2 | 311.3 | 1959.2 | 200.6 | Point mutant | I86F |
| | | | | | | (active site | |
| | | | | | | saturation | |
| | | | | | | mutation) | |
| t525907 | Library | 6191.8 | 424.9 | 8313.5 | 439.8 | Point mutant | F148A |
| | | | | | | (active site | |
| | | | | | | saturation | |
| | | | | | | mutation) | |
| t525908 | Library | 6029 | 624 | 6313.8 | 390.1 | Point mutant | H60D |
| | | | | | | (bioinformatics) | |
| t525916 | Library | 2587.7 | 2992 | 3579.4 | 4145.6 | Point mutant | W68G |
| | | | | | | (bioinformatics) | |
| t525944 | Library | 5577.7 | 691.6 | 6473.7 | 1256 | Point mutant | P301G |
| | | | | | | (bioinformatics) | |
| t525970 | Library | 4480.7 | 170.7 | 6418.1 | 380.5 | Point mutant | A298D |
| | | | | | | (bioinformatics) | |
| t526011 | Library | 5297 | 875.5 | 8787.5 | 808.7 | Point mutant | M110I |
| | | | | | | (bioinformatics) | |
| t526144 | Library | 4225.9 | 1579.2 | 7396.6 | 425.3 | Point mutant | I170T |
| | | | | | | (active site | |
| | | | | | | saturation | |
| | | | | | | mutation) | |
| t526242 | Library | 7393.2 | 1897.7 | 4855.7 | 136.2 | Point mutant | A149E |
| | | | | | | (active site | |
| | | | | | | saturation | |
| | | | | | | mutation) | |
| t526248 | Library | 5767.3 | 1310.6 | 6229.4 | 315.3 | Point mutant | F56T |
| | | | | | | (bioinformatics) | |
| t526250 | Library | 6951.8 | 953 | 4091.3 | 442.2 | Point mutant | V231L |
| | | | | | | (active site | |
| | | | | | | saturation | |
| | | | | | | mutation) | |
| t526252 | Library | 6551.9 | 798.7 | 5670.5 | 316.9 | Point mutant | I223V |
| | | | | | | (active site | |
| | | | | | | saturation | |
| | | | | | | mutation) | |
| t526258 | Library | 6785.2 | 630.9 | 9360.3 | 475.5 | Point mutant | D94E |
| | | | | | | (bioinformatics) | |
| t526260 | Library | 6623.6 | 739 | 9160.7 | 762.1 | Point mutant | F82G |
| | | | | | | (bioinformatics) | |
| t526336 | Library | 6929.2 | 677.4 | 9616.6 | 742.4 | Point mutant | F145T |
| | | | | | | (active site | |
| | | | | | | saturation | |
| | | | | | | mutation) | |
| t526340 | Library | 5645.7 | 466.5 | 9840.5 | 409.9 | Point mutant | C48T |
| | | | | | | (bioinformatics) | |
| t526347 | Library | 5451.1 | 1007.1 | 6698.2 | 465.5 | Point mutant | A149W |
| | | | | | | (active site | |
| | | | | | | saturation | |
| | | | | | | mutation) | |
| t526392 | Library | 5461 | 265.2 | 7818.9 | 2425.7 | Point mutant | F80W |
| | | | | | | (active site | |
| | | | | | | saturation | |
| | | | | | | mutation) | |
| t526393 | Library | 5085.9 | 445.2 | 3980.5 | 517.8 | Point mutant | I170C |
| | | | | | | (active site | |
| | | | | | | saturation | |
| | | | | | | mutation) | |
| t526411 | Library | 5539.3 | 1070.7 | 3555.4 | 1000.9 | Point mutant | S173W |
| | | | | | | (active site | |
| | | | | | | saturation | |
| | | | | | | mutation) | |
| t526424 | Library | 4290.5 | 429.6 | 5715.1 | 204.6 | Point mutant | A149I |
| | | | | | | (active site | |
| | | | | | | saturation | |
| | | | | | | mutation) | |
| t526428 | Library | 5254.1 | 393.3 | 2456.2 | 202.5 | Point mutant | A149Q |
| | | | | | | (active site | |
| | | | | | | saturation | |
| | | | | | | mutation) | |
| t526432 | Library | 5261.9 | 1093.5 | 6209.1 | 965.5 | Point mutant | A149S |
| | | | | | | (active site | |
| | | | | | | saturation | |
| | | | | | | mutation) | |
| t526436 | Library | 5011.6 | 709.5 | 5627.4 | 381.6 | Point mutant | F151T |
| | | | | | | (active site | |
| | | | | | | saturation | |
| | | | | | | mutation) | |
| t526449 | Library | 3334.1 | 366.1 | 4227.2 | 847.4 | Point mutant | R59P |
| | | | | | | (bioinformatics) | |
| t526450 | Library | 4724.1 | 677.4 | 3752.6 | 755.5 | Point mutant | M43A |
| | | | | | | (active site | |
| | | | | | | saturation | |
| | | | | | | mutation) | |
| t526546 | Library | 5046.3 | 751 | 7661.5 | 786.7 | Point mutant | F151I |
| | | | | | | (active site | |
| | | | | | | saturation | |
| | | | | | | mutation) | |
| t526556 | Library | 6262.2 | 272.7 | 5905.4 | 1006 | Point mutant | A149T |
| | | | | | | (active site | |
| | | | | | | saturation | |
| | | | | | | mutation) | |
| t526569 | Library | 5246 | 774.7 | 8236.7 | 1049.6 | Point mutant | F56I |
| | | | | | | (bioinformatics) | |
| t526570 | Library | 6322.3 | 639.4 | 6961.2 | 1204.4 | Point mutant | F151C |
| | | | | | | (active site | |
| | | | | | | saturation | |
| | | | | | | mutation) | |
| t526577 | Library | 5820.3 | 599.3 | 7267.5 | 1042.4 | Point mutant | G52L |
| | | | | | | (bioinformatics) | |
| t526600 | Library | 6083.7 | 934.8 | 6850.2 | 622.7 | Point mutant | S173G |
| | | | | | | (active site | |
| | | | | | | saturation | |
| | | | | | | mutation) | |
| t526633 | Library | 8746.7 | 721.4 | 10086.8 | 688.3 | Point mutant | I147L |
| | | | | | | (active site | |
| | | | | | | saturation | |
| | | | | | | mutation) | |
| t526675 | Library | 4852.1 | 1022.5 | 8613.9 | 484 | Point mutant | S232K |
| | | | | | | (bioinformatics) | |
| t526691 | Library | 6718 | 1009.4 | 9191 | 1.845.5 | Point mutant | F83Y |
| | | | | | | (active site | |
| | | | | | | saturation | |
| | | | | | | mutation) | |
| t526755 | Library | 5185.5 | 257.5 | 4298.8 | 396.3 | Point mutant | P30IT |
| | | | | | | (bioinformatics) | |
| t526763 | Library | 5805.3 | 822.1 | 7985 | 1141.8 | Point mutant | M87I |
| | | | | | | (active site | |
| | | | | | | saturation | |
| | | | | | | mutation) | |
| t526771 | Library | 9529.4 | 1147.9 | 11951.3 | 991.4 | Point mutant | I86V |
| | | | | | | (active site | |
| | | | | | | saturation | |
| | | | | | | mutation) | |
| t526779 | Library | 4687.5 | 938.5 | 7077.4 | 1654.1 | Point mutant | I86C |
| | | | | | | (active site | |
| | | | | | | saturation | |
| | | | | | | mutation) | |
| t526785 | Library | 7110.2 | 692.7 | 10615.9 | 771.7 | Point mutant | Q288R |
| | | | | | | (bioinformatics) | |
| t526804 | Library | 5173.9 | 730.5 | 7101 | 962.9 | Point mutant | I142M |
| | | | | | | (bioinformatics) | |
| t526809 | Library | 8567.5 | 1106.3 | 12813.2 | 474 | Point mutant | M43L |
| | | | | | | (active site | |
| | | | | | | saturation | |
| | | | | | | mutation) | |
| t526825 | Library | 6220.8 | 687.5 | 11910.8 | 1397.8 | Point mutant | L311K |
| | | | | | | (bioinformatics) | |
| t526828 | Library | 6068.2 | 1021.8 | 2292 | 155.8 | Point mutant | S302T |
| | | | | | | (bioinformatics) | |
| t526834 | Library | 5993.4 | 391 | 6244.1 | 438.8 | Point mutant | N167A |
| | | | | | | (bioinformatics) | |
| t526836 | Library | 5301.1 | 381.2 | 5712.1 | 227.7 | Point mutant | M87C |
| | | | | | | (active site | |
| | | | | | | saturation | |
| | | | | | | mutation) | |
| t526842 | Library | 4355.2 | 492.8 | 6387.5 | 434.5 | Point mutant | R197V |
| | | | | | | (bioinformatics) | |
| t526856 | Library | 4088.3 | 1232.7 | 4345.6 | 156.3 | Point mutant | Y163F |
| | | | | | | (bioinformatics) | |
| t526858 | Library | 4854.8 | 667.9 | 4369.3 | 197.2 | Point mutant | M243I |
| | | | | | | (bioinformatics) | |
| t526868 | Library | 4941.4 | 391.9 | 5284.5 | 371.2 | Point mutant | M87Q |
| | | | | | | (active site | |
| | | | | | | saturation | |
| | | | | | | mutation) | |
| t526875 | Library | 5517.1 | 489.7 | 11146.5 | 982.7 | Point mutant | Q162R |
| | | | | | | (bioinformatics) | |
| t526922 | Library | 6205 | 877.3 | 5037.9 | 466 | Point mutant | S258A |
| | | | | | | (bioinformatics) | |
| t526930 | Library | 7072.5 | 605.6 | 8508.1 | 443.3 | Point mutant | F245W |
| | | | | | | (bioinformatics) | |
| t526947 | Library | 5298.9 | 836.5 | 7921.9 | 470.9 | Point mutant | S182L |
| | | | | | | (bioinformatics) | |
| t526953 | Library | 9582.5 | 633.4 | 6273.6 | 1543.5 | Point mutant | C31F |
| | | | | | | (bioinformatics) | |
| t526954 | Library | 8065.6 | 558 | 8036 | 607 | Point mutant | F245R |
| | | | | | | (bioinformatics) | |
| t526956 | Library | 8231.3 | 535 | 9192.8 | 1172.5 | Point mutant | M87V |
| | | | | | | (active site | |
| | | | | | | saturation | |
| | | | | | | mutation) | |
| t526961 | Library | 6025.2 | 552.7 | 7618.5 | 1228.1 | Point mutant | H60N |
| | | | | | | (bioinformatics) | |
| t526964 | Library | 4432.5 | 720 | 6492.5 | 477.5 | Point mutant | Y91F |
| | | | | | | (active site | |
| | | | | | | saturation | |
| | | | | | | mutation) | |
| t526971 | Library | 7446.3 | 559.6 | 6271.5 | 188.3 | Point mutant | I142T |
| | | | | | | (bioinformatics) | |
| t523553 | Library | 7185.8 | 2310.6 | 15527.2 | 1357.6 | Point mutant | I86S |
| | | | | | | (active site | |
| | | | | | | saturation | |
| | | | | | | mutation) | |

The set of point mutations carried over from the primary screen to the secondary screen included 75 of the 281 point mutations generated using the bioinformatics analysis discussed in Example 1, and 52 of the 646 point mutations generated using the active site saturation-mutagenesis discussed in Example 1. Therefore, the bioinformatics analysis substantially improved hit rate (˜3.4×) for identifying potentially relevant point mutations compared to the exhaustive mutational scan procedure of saturation mutagenesis. Also, by mapping the point mutations onto a homology model for CsPT4, it was found that the mutations identified through bioinformatics analysis were dispersed throughout the protein structure, in contrast to those identified by saturation mutagenesis, which were localized around the active site. This suggested that the bioinformatics analysis could identify mutations at positions that may improve protein stability and expression in addition to catalytic activity.

Active-site saturation mutagenesis identified multiple point mutations at position 186 in SEQ ID NO: 5 (Table 5). This residue is located in an apposing face of a helix that forms part of the active site of CsPT4. Without wishing to be bound by any theory, substitution mutations at a residue corresponding to position 86 in SEQ ID NO: 5 (e.g., I86S, I86G, I86A) may increase activity of the PT enzyme due to the decreased residue size relative to the corresponding residue in the wildtype protein. Reduction in side-chain volume at this position may lead to a slight shift in the helix, which could increase the volume of the olivetolic/divarinic acid binding pocket. Active-site saturation mutagenesis also identified multiple point mutations at positions F82 (e.g., F82G), F83 (e.g., F83Y), and M87 (e.g., M87T, M87I, M87C. M87Q, and M87V) in SEQ ID NO: 5 (Table 5). Similar to residue 186, residues F83 and M87 are also located in the same apposing face of the helix that forms part of the active site of CsPT4. Additionally, residues F82. F83, and M87 are predicted to interact with residue 186. Without wishing to be bound by any theory, substitutions at residues F82, F83 and M87 may impact activity of the PT enzyme in a similar manner to that discussed above for residue 186. These results suggest that substitution mutations in residues that are not interacting directly with the substrate or cofactor can still lead to modulation of activity of the PT enzyme.

Variant PTs comprising combinations of these beneficial point mutations may further enhance cannabinoid production. The discovery of many point mutations that substantially improve production of CBGA and CBGVA represents a significant improvement in the development and use of membrane-bound PTs.

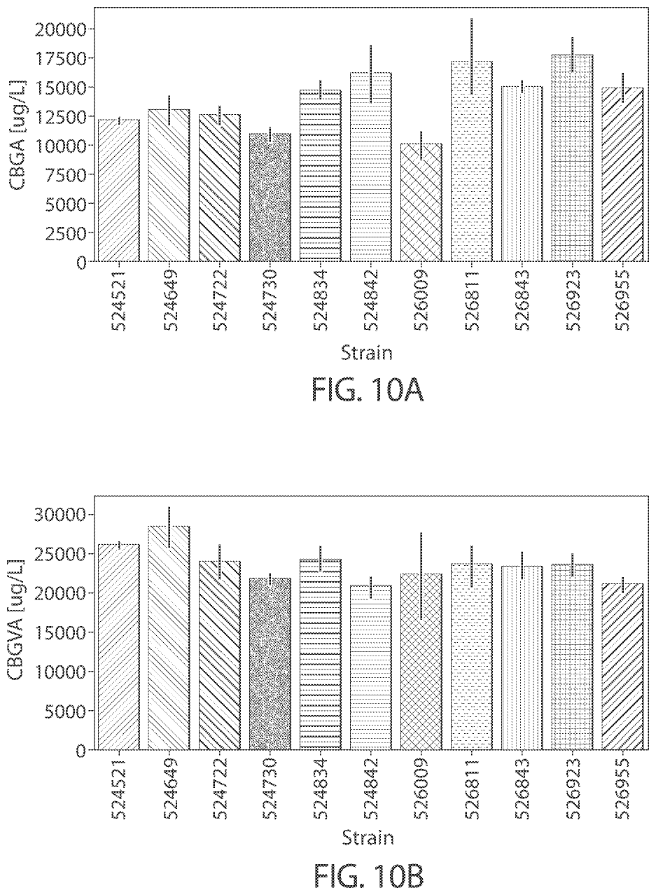

The ERG20-CsPT fusion proteins and the CsPT chimeras assayed in the secondary screen were generally found to produce both CBGA and CBGVA when fed OA and DA, respectively, in the prenyltransferase assay. The fusion proteins were found to generate at least 10000 μg/L CBGA and 20000 μg/L CBGVA in all eleven strains tested (FIGS. 10A and 10B).

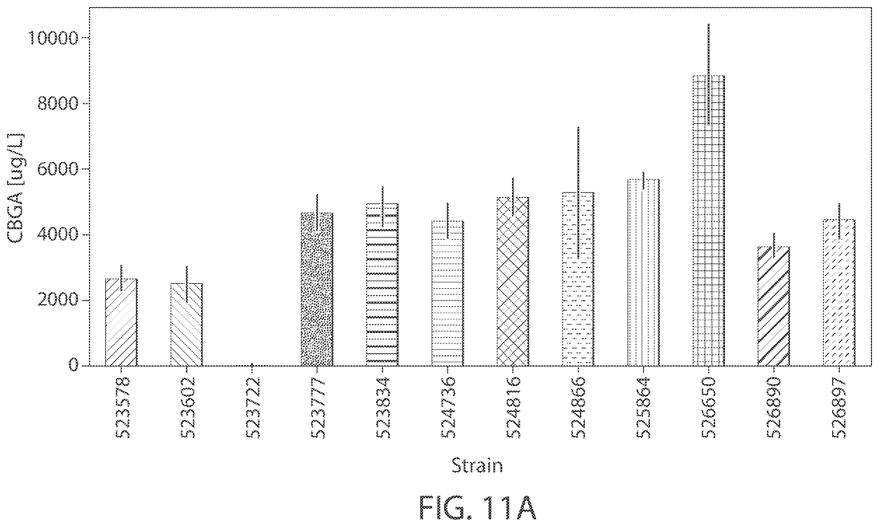

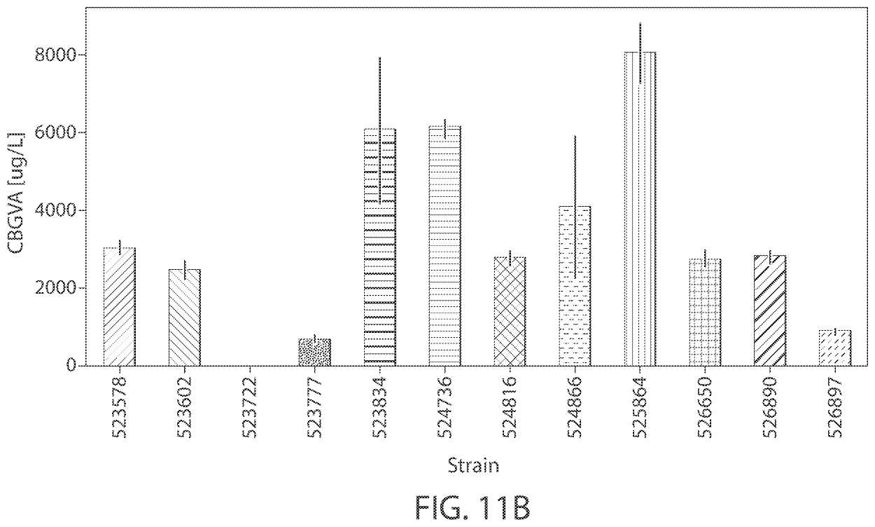

Robust CBGA production was also observed in several of the CsPT chimeras (FIGS. 11A and 11B). Eleven of the twelve chimeras assayed in the secondary screen were found to produce CBGA, with some producing more than 8000 μg/L and 5000 μg/L CBGA (strains t526650; and t525864, 524866 and 524816, respectively; SEQ ID NOs: 119; and 118, 117 and 116). SEQ ID NO: 118 (strain t525864) comprises 85% CsPT4 and 15% CsPT7. SEQ ID NO: 116 (Strain t524816) comprises 64% CsPT4 and 36% CsPT7. SEQ ID NO: 119 (Strain t526650) comprises 83% CsPT4 and 17% CsPT7. SEQ ID NO: 117 (strain t524866) comprises 83% CsPT4 and 17% CsPT6.

Analysis of CsPT chimera hits using a motif identification software identified multiple sequence motifs that were more likely to be found in chimeras that produce CBGA than in chimeras that did not produce CBGA, with a measure of statistical significance based on E-value (Table 6). Thus, sequence motifs were identified that correlate with enhanced CBGA production in chimeric membrane-bound PTs.

TABLE 6
Non-limiting examples of motifs identified in chimeric PTs

| | | | Start site | End site | |
|---|---|---|---|---|---|
| | | | (relative to | (relative | |
| Sequence Motif | | Sequence | SEQ ID | to SEQ | |
| (SEQ ID NO) | E-value | Length | NO: 5) | ID NO: 5) | Motif Location |
| MTVMGMT | 8.90E−06 | 7 | 207 | 213 | chimeric junction, |
| (SEQ ID NO: 11) | | | | | CsPT4 |
| [EV][LMW][RS]P[SAP]F | 1.10E−02 | 12 | 195 | 206 | chimeric junction, |
| [ST]F[L][IL]AF | | | | | CsPT1, CsPT4, CsPT7 |
| (SEQ ID NO: 12) | | | | | |
| QFFEFIW | 8.90E−06 | 7 | 304 | 310 | chimeric junction, |
| (SEQ ID NO: 13) | | | | | CsPT4 |
| HNTNL | 1.90E−03 | 5 | 57 | 61 | CsPT1, CsPT7 |
| (SEQ ID NO: 14) | | | | | |
| TCWKL | 8.90E−06 | 5 | 30 | 34 | CsPT4, CsPT7 |
| (SEQ ID NO: 15) | | | | | |
| M[IL]LSHAILAFC | 6.30E−03 | 11 | 274 | 284 | chimeric junction, |
| (SEQ ID NO: 16) | | | | | CsPT4 |
| HVG[LV][AN]FT[SCF]Y | 1.30E−04 | 16 | 175 | 190 | chimeric junction, |
| [YS]A[ST][RT][AS]A[LF] | | | | | csPT4 |
| (SEQ ID NO: 17) | | | | | |
| GLIVT | 5.50E−04 | 5 | 126 | 130 | chimeric junction, |
| (SEQ ID NO: 18) | | | | | CsPT4 |
| L[YH]YAEY[LE]V | 4.30E−02 | 8 | 312 | 319 | chimeric junction, |
| (SEQ ID NO: 19) | | | | | CsPT1, CsPT4, CsPT7 |
| KAFFAL | 1.70E−02 | 6 | 69 | 74 | chimeric junction, |
| (SEQ ID NO: 20) | | | | | CsPT4 |
| KLGARNMT | 8.90E−06 | 00 | 237 | 244 | CsPT4 |
| (SEQ ID NO: 21) | | | | | |
| QAF[NK]SN | 2.70E−02 | 6 | 267 | 272 | chimeric junction, |
| (SEQ ID NO: 22) | | | | | CsPT1, CsPT7 |
| LIFQT | 8.90E−06 | 5 | 285 | 289 | chimeric junction, |
| (SEQ ID NO: 23) | | | | | CsPT1, CsPT4, CsPT7 |
| SIIVALT | 8.90E−06 | 7 | 119 | 125 | chimeric junction, |
| (SEQ ID NO: 24) | | | | | CsPT4, CsPT7 |
| MSIETAW | 8.90E−06 | 7 | 110 | 116 | chimeric junction, |
| (SEQ ID NO: 25) | | | | | CsPT4, CsPT7 |
| VVSGV | 8.90E−06 | 5 | 246 | 250 | chimeric junction, |
| (SEQ ID NO: 26) | | | | | CsPT4 |
| RPYVV | 8.90E−06 | 5 | 36 | 40 | chimeric junction, |
| (SEQ ID NO: 27) | | | | | CsPT4 |
| KPDLP | 8.90E−06 | 5 | 100 | 104 | CsPT1, CsPT4, CsPT7 |
| (SEQ ID NO: 28) | | | | | |
| RWKQY | 8.90E−06 | 5 | 159 | 163 | CsPT4 |
| (SEQ ID NO: 29) | | | | | |
| FLITI | 8.90E−06 | 5 | 168 | 172 | chimeric junction, |
| (SEQ ID NO: 30) | | | | | CsPT4 |
| DIEGD | 8.90E−06 | 5 | 222 | 226 | CsPT4, CsPT7 |
| (SEQ ID NO: 31) | | | | | |
| KYGVST | 8.90E−06 | 6 | 228 | 233 | CsPT4 |
| (SEQ ID NO: 32) | | | | | |

### Example 3: Functional Expression of Additional Chimeric PTs

Multiple chimeras from Examples 1 and 2 (corresponding to strains t526897, t523777, t524736, t523834, t526650, t524816, and t523722) were modified to carry point mutations that were found to be associated with increasing CBGAS activity in Example 1. As shown in Table 7, the following point mutations were tested in the context of chimeras either alone or in combination: C31F, F245R, and S232R, as described in Examples 1 and 2, and F246R and S233R. For the point mutations F246R and S233R, the amino acid numbering corresponds to residue position in the sequence of the parent chimera strain. Strain t612567 comprises the chimera from parent strain t523722 with a F246R substitution. Strain t612571 comprises the chimera from parent strain t523777 with a S233R substitution. The corresponding residues to F246 and S233 in CsPT4 are F245 and S232.

The standard deviation (SD) values reported in Table 7 were generally higher than the average CBGA values reported for a given strain. Without wishing to be bound by any theory, several factors related to the assay conditions may contribute to causing the high SD values. For example, when calculating the SD of control samples dispersed across multiple plates, qualitatively high SD values may be caused by aggregating error associated with plate-to-plate variability in performance, sample processing during screening, sample processing during analytics, and other factors. These errors compound to generate high dispersion in titer data for these controls and consequently high SD. Another source of high dispersion may be in the occasional sample dropout. For example, if a given strain fails to grow from a glycerol stock when inoculated (e.g., due to an error during liquid transfer of culture into media), but its replicates do, this can create artificially high dispersion in the data.

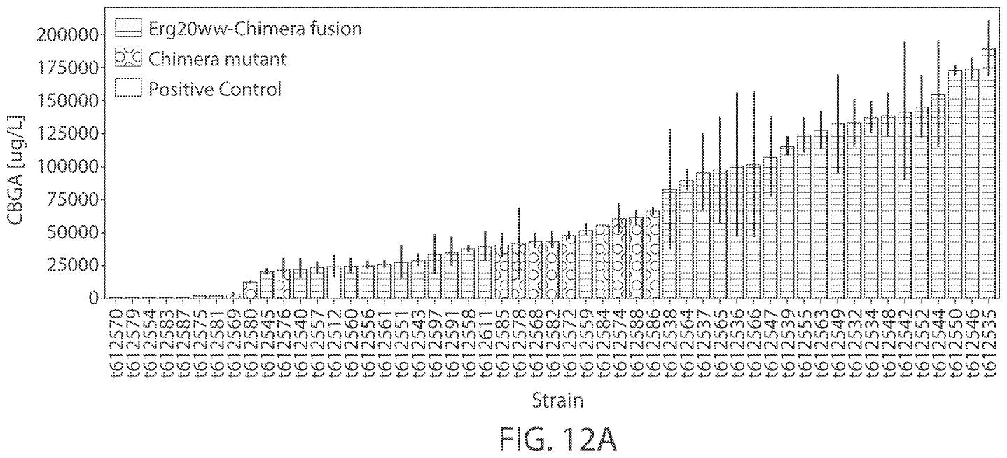

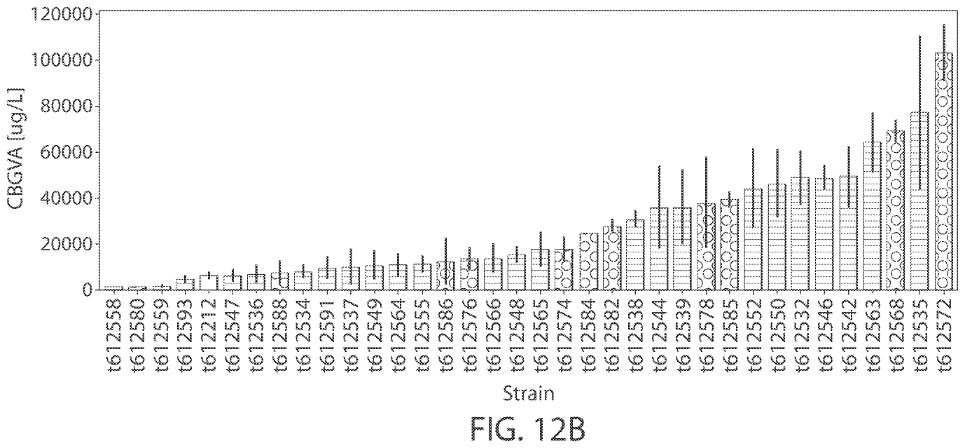

The chimeric PTs with point mutations described above were screened for activity in a library (Gen 2 library). Strain t612212, expressing a truncated CsPT4 protein (SEQ 1D NO: 5), was included as a positive control. The assay used to assess CBGAS activity was the same as the assay described in Example 1 except that 1 mM olivetolic acid and 1 mM divaric acid were separately used as substrates in parallel assays, and both CBGA and CBGVA production were measured using LC-MS on three biological replicates. Table 7 and FIGS. 12A-B show the results of the Gen. 2 PT library screen. Sequences of the chimeric fusions are provided in Table 14. Sequences of individual portions of representative chimeric PTs are provided in Table 15.

TABLE 7
Activity data of Gen2 library members in S. cerevisiae

| | Strain type | | | Standard | | Standard |
|---|---|---|---|---|---|---|
| | PT type | Parent | Average | Deviation | Average | Deviation |
| | Mutation (if | chimera | CBGA | CBGA | CBGVA | CBGVA |
| Strain ID | applicable) | strain | [μg/L] | [μg/L] | [μg/L] | [μg/L] |
| t612212 | Positive control | N/A | 12130.94 | 13905.38 | 2882.431 | 3226.651 |
| t612567 | Library | t523777 | 0 | 0 | 0 | 0 |
| | Chimera; F246R | | | | | |
| t612571 | Library | t523777 | 0 | 0 | 0 | 0 |
| | Chimera; S233R | | | | | |
| t612573 | Library | t526897 | 0 | 0 | 0 | 0 |
| | Chimera; C31F | | | | | |
| t612577 | Library | t523777 | 0 | 0 | 0 | 0 |
| | Chimera; C31F | | | | | |
| t612589 | Library | t526897 | 0 | 0 | 0 | 0 |
| | Chimera; F245R | | | | | |
| t612570 | Library | t526897 | 194.8158 | 315.0592 | 0 | 0 |
| | Chimera; S232R | | | | | |
| t612583 | Library | t524736 | 254.0501 | 407.9202 | 0 | 0 |
| | Chimera; C31F | | | | | |
| t612587 | Library | t523834 | 265.0743 | 413.0831 | 0 | 0 |
| | Chimera; F245R | | | | | |
| t612579 | Library | t523834 | 320.3343 | 360.6222 | 0 | 0 |
| | Chimera; C31F | | | | | |
| t612575 | Library | t523834 | 1127.725 | 1239.832 | 0 | 0 |
| | Chimera; S232R | | | | | |
| t612581 | Library | t524736 | 1323.91 | 1450.387 | 0 | 0 |
| | Chimera; F245R | | | | | |
| t612569 | Library | t524736 | 1387.059 | 1790.107 | 0 | 0 |
| | Chimera; S232R | | | | | |
| t612580 | Library | t523722 | 6481.111 | 7153.546 | 246.4287 | 603.6247 |
| | Chimera; C31F | | | | | |
| t612584 | Library | t524816 | 9324.939 | 22841.34 | 4098.149 | 10038.37 |
| | Chimera; C31F | | | | | |
| t612576 | Library | t523722 | 11279.32 | 13450.5 | 6622.208 | 7872.582 |
| | Chimera; S232R | | | | | |
| t612585 | Library | t524816 | 20251.26 | 22878.32 | 19647.84 | 21633.9 |
| | Chimera; F245R | | | | | |
| t612578 | Library | t524816 | 21078.15 | 28541.1 | 18798.61 | 24164.75 |
| | Chimera; C31F; | | | | | |
| | S232R | | | | | |
| t612568 | Library | t526650 | 21935.64 | 24298.67 | 34451.92 | 37874.88 |
| | Chimera; S232R | | | | | |
| t612582 | Library | t526650 | 21964.82 | 24353.01 | 13889.79 | 15321.91 |
| | Chimera; F245R | | | | | |
| t612572 | Library | t524816 | 23763.55 | 26113.94 | 51399.02 | 56871.95 |
| | Chimera; S232R | | | | | |
| t612574 | Library | t526650 | 30387.66 | 34066.63 | 8672.353 | 10046.56 |
| | Chimera; C31F | | | | | |
| t612588 | Library | t524816 | 30551.87 | 33695.77 | 3560.022 | 5094.13 |
| | Chimera; | | | | | |
| | C31F; F245R | | | | | |
| t612586 | Library | t524816 | 32959.86 | 36147.22 | 5985.625 | 9205.13 |
| | Chimera; | | | | | |
| | C31F; F245R; | | | | | |
| | S232R | | | | | |
| t612533 | Library | t523722 | 0 | 0 | 0 | 0 |
| | (Chimeric | | | | | |
| | fusion) | | | | | |
| t612541 | Library | t523722 | 0 | 0 | 0 | 0 |
| | (Chimeric | | | | | |
| | fusion) | | | | | |
| t612553 | Library | t523722 | 0 | 0 | 0 | 0 |
| | (Chimeric | | | | | |
| | fusion) | | | | | |
| t612562 | Library | t523722 | 0 | 0 | 0 | 0 |
| | (Chimeric | | | | | |
| | fusion) | | | | | |
| t612554 | Library | t523722 | 116.9348 | 286.4306 | 0 | 0 |
| | (Chimeric | | | | | |
| | fusion) | | | | | |
| t612545 | Library | t526897 | 10311.92 | 11383.54 | 0 | 0 |
| | (Chimeric | | | | | |
| | fusion) | | | | | |
| t612540 | Library | t526897 | 11429.6 | 13401.96 | 0 | 0 |
| | (Chimeric | | | | | |
| | fusion) | | | | | |
| t612557 | Library | t526897 | 11538.03 | 12971.28 | 0 | 0 |
| | (Chimeric | | | | | |
| | fusion) | | | | | |
| t612560 | Library | t526897 | 12463.34 | 14132.09 | 0 | 0 |
| | (Chimeric | | | | | |
| | fusion) | | | | | |
| t612556 | Library | t523777 | 12620.98 | 13927.45 | 0 | 0 |
| | (Chimeric | | | | | |
| | fusion) | | | | | |
| t612561 | Library | t523777 | 13079.52 | 14441.16 | 0 | 0 |
| | (Chimeric | | | | | |
| | fusion) | | | | | |
| t612551 | Library | t526897 | 13722.02 | 17028.25 | 0 | 0 |
| | (Chimeric | | | | | |
| | fusion) | | | | | |
| t612543 | Library | t523777 | 14500.09 | 16215.38 | 0 | 0 |
| | (Chimeric | | | | | |
| | fusion) | | | | | |
| t612558 | Library | t523777 | 18786.8 | 20659.6 | 375.0181 | 584.81 |
| | (Chimeric | | | | | |
| | fusion) | | | | | |
| t612559 | Library | t523777 | 25827.83 | 28473.21 | 508.7528 | 794.432 |
| | (Chimeric | | | | | |
| | fusion) | | | | | |
| t612538 | Library | t526650 | 41083.81 | 53439.9 | 15215.75 | 16847.43 |
| | (Chimeric | | | | | |
| | fusion) | | | | | |
| t612564 | Library | t523834 | 44532.26 | 49042.75 | 5336.139 | 6681.855 |
| | (Chimeric | | | | | |
| | fusion) | | | | | |
| t612537 | Library | t526650 | 47840.43 | 55578.1 | 4927.458 | 7431.708 |
| | (Chimeric | | | | | |
| | fusion) | | | | | |
| t612565 | Library | t523834 | 48333.47 | 58786.05 | 8663.469 | 10621.93 |
| | (Chimeric | | | | | |
| | fusion) | | | | | |
| t612536 | Library | t524816 | 50281.31 | 65111.86 | 3244.597 | 4443.507 |
| | (Chimeric | | | | | |
| | fusion) | | | | | |
| t612566 | Library | t526650 | 50357.21 | 65226.16 | 6777.715 | 8496.571 |
| | (Chimeric | | | | | |
| | fusion) | | | | | |
| t612547 | Library | t524816 | 53558.28 | 61762.84 | 3049.673 | 3782.629 |
| | (Chimeric | | | | | |
| | fusion) | | | | | |
| t612539 | Library | t524736 | 57572.61 | 63207.54 | 17919.95 | 22103.85 |
| | (Chimeric | | | | | |
| | fusion) | | | | | |
| t612555 | Library | t524816 | 61742.04 | 68165.03 | 5539.507 | 6516.889 |
| | (Chimeric | | | | | |
| | fusion) | | | | | |
| t612563 | Library | t524736 | 63519.61 | 70172.29 | 31992.32 | 35998.68 |
| | (Chimeric | | | | | |
| | fusion) | | | | | |
| t612549 | Library | t524816 | 66007.6 | 76076.68 | 5204.919 | 7052.566 |
| | (Chimeric | | | | | |
| | fusion) | | | | | |
| t612532 | Library | t524736 | 66244.47 | 73441.78 | 24263.57 | 27624.32 |
| | (Chimeric | | | | | |
| | fusion) | | | | | |
| t612534 | Library | t524816 | 68480.87 | 75414.48 | 3807.088 | 4581.189 |
| | (Chimeric | | | | | |
| | fusion) | | | | | |
| t612548 | Library | t526650 | 69087.54 | 76426.63 | 7602.177 | 8644.452 |
| | (Chimeric | | | | | |
| | fusion) | | | | | |
| t612542 | Library | t524736 | 70521.38 | 83974.33 | 24560.97 | 28229.23 |
| | (Chimeric | | | | | |
| | fusion) | | | | | |
| t612552 | Library | t523834 | 72365.77 | 80657.95 | 21770.95 | 26275.44 |
| | (Chimeric | | | | | |
| | fusion) | | | | | |
| t612544 | Library | t526650 | 77279.31 | 88492.62 | 17820.36 | 22635.91 |
| | (Chimeric | | | | | |
| | fusion) | | | | | |
| t612550 | Library | t524736 | 86331.69 | 94606.46 | 23037.33 | 26968.89 |
| | (Chimeric | | | | | |
| | fusion) | | | | | |
| t612546 | Library | t523834 | 86637.57 | 95059.89 | 24272.24 | 26793.25 |
| | (Chimeric | | | | | |
| | fusion) | | | | | |
| t612535 | Library | t523834 | 94164.23 | 104013.7 | 38477.85 | 47236.42 |
| | (Chimeric | | | | | |
| | fusion) | | | | | |

Out of the chimeric PTs with point mutations that were screened, the following strains produced at least 20.000 μg/L CBGA and/or at least 3000 μg/L CBGVA, as shown in Table 7: strain t612585, which was based on the chimeric PT sequence within strain t524816 described in Examples 1 and 2, and also contained a F245R substitution; strain t612578, which was based on the chimeric PT sequence within strain t524816 described in Examples 1 and 2, and also contained C31F and S232R substitutions; strain t612568, which was based on the chimeric PT sequence within strain t526650 described in Examples 1 and 2, and also contained a S232R substitution; strain t612582, which was based on the chimeric PT sequence within strain t526650 described in Examples 1 and 2, and also contained a F245R substitution; strain t612572, which was based on the chimeric PT sequence within strain t524816 described in Examples 1 and 2, and also contained a S232R substitution; strain t612574, which was based on the chimeric PT sequence within strain t526650 described in Examples 1 and 2, and also contained a C31F substitution; strain t612588, which was based on the chimeric PT sequence within strain t524816 described in Examples 1 and 2, and also contained C31F and F245R substitutions; and strain t612586, which was based on the chimeric PT sequence within strain t524816 described in Examples 1 and 2, and also contained C31F, F245R, and S232R substitutions.

### Example 4: Functional Expression of Chimeric Fusions

CsPT chimeras from strains from Examples 1 and 2 (corresponding to strains t523578, t523602, t523722, t523777, t523834, t524736, t524816, t524866, t525864, t526650, t526890, and t526897) were fused with ERG20ww and screened for activity.

The chimeric fusions were screened for activity as part of the Gen 2 library. Strain t612212, expressing a truncated CsPT4 protein (SEQ ID NO: 5), was included as a positive control. The assay used to assess CBGAS activity was the same as the assay described in Example 1 except that 1 mM olivetolic acid and 1 mM divaric acid were separately used as substrates in parallel assays, and both CBGA and CBGVA production were measured using LC-MS on three biological replicates. Table 7 and FIGS. 12A-B show the results of the Gen. 2 PT library screen. Sequences of the chimeric fusions are provided in Table 14. Sequences of individual portions of representative chimeric PTs are provided in Table 15.

Out of the chimeric fusions that were screened, the following strains produced at least 13,000 μg/L CBGA and/or at least 3000 μg/L CBGVA, as shown in Table 7: strain t612561, which was based on the chimeric PT sequence within strain t524816 described in Examples 1 and 2; strain t612551, which was based on the chimeric PT sequence within strain t526897 described in Examples 1 and 2; strain t612543, which was based on the chimeric PT sequence within strain t523777 described in Examples 1 and 2; strain t612558, which was based on the chimeric PT sequence within strain t523777 described in Examples 1 and 2; strain t612559, which was based on the chimeric PT sequence within strain t523777 described in Examples 1 and 2; strain t612538, which was based on the chimeric PT sequence within strain t526650 described in Examples 1 and 2; strain t612564, which was based on the chimeric PT sequence within strain t523834 described in Examples 1 and 2; strain t612537, which was based on the chimeric PT sequence within strain t526650 described in Examples 1 and 2; strain t612565, which was based on the chimeric PT sequence within strain t523834 described in Examples 1 and 2; strain t612536, which was based on the chimeric PT sequence within strain t524816 described in Examples 1 and 2; strain t612566, which was based on the chimeric PT sequence within strain t526650 described in Examples 1 and 2; strain t612547, which was based on the chimeric PT sequence within strain t524816 described in Examples 1 and 2; strain t612539, which was based on the chimeric PT sequence within strain t524736 described in Examples 1 and 2; strain t612555, which was based on the chimeric PT sequence within strain t524816 described in Examples 1 and 2; strain t612563, which was based on the chimeric PT sequence within strain t524736 described in Examples 1 and 2; strain t612549, which was based on the chimeric PT sequence within strain t524816 described in Examples 1 and 2; strain t612532, which was based on the chimeric PT sequence within strain t524736 described in Examples 1 and 2; strain t612534, which was based on the chimeric PT sequence within strain t524816 described in Examples 1 and 2; strain t612548, which was based on the chimeric PT sequence within strain t526650 described in Examples 1 and 2; strain t612542, which was based on the chimeric PT sequence within strain t524736 described in Examples 1 and 2; strain t612552, which was based on the chimeric PT sequence within strain t523834 described in Examples 1 and 2; strain t612544, which was based on the chimeric PT sequence within strain t526650 described in Examples 1 and 2; strain t612550, which was based on the chimeric PT sequence within strain t524736 described in Examples 1 and 2; strain t612546, which was based on the chimeric PT sequence within strain t523834 described in Examples 1 and 2; and strain t612535, which was based on the chimeric PT sequence within strain t523834 described in Examples 1 and 2.

### Example 5: Further Engineering of Chimeric Fusions

Chimeric fusions expressed by strains t612534 and t612535 from the Gen 2 PT library described in Example 3, and a chimeric PT expressed by strain t524866 from the library described in Example 1, were used as templates for additional engineering to generate a Gen 3 library. All chimeric PTs in the Gen 3 library included portions of two different CsPT proteins and all members of the library were expressed as ERG20ww-PT chimeric fusions. Strain t612534 from the Gen 2 library was created based on strain t524816, which was one of the high-performing chimeras shown in Table 5. Strain t612535 from the Gen 2 library was created based on strain t523834, which was one of the high-performing chimeras shown in Table 5. Strain t524866 was one of the high-performing chimeras shown in Table 5.

The performance of the chimeric PTs with point mutations that were screened in the Gen 2 library was used to inform the incorporation of additional mutations that were implicated in improving CBGA titer in Example 1. Specifically, the following point mutations were tested in the context of chimeric fusions, either alone or in combination, as shown in Table 8: M43L, I86S, Q288R, S232R, I147L, C31F, F245R, M87V, D94E, I86V, L311R, L311N, I86A, and Q162R.

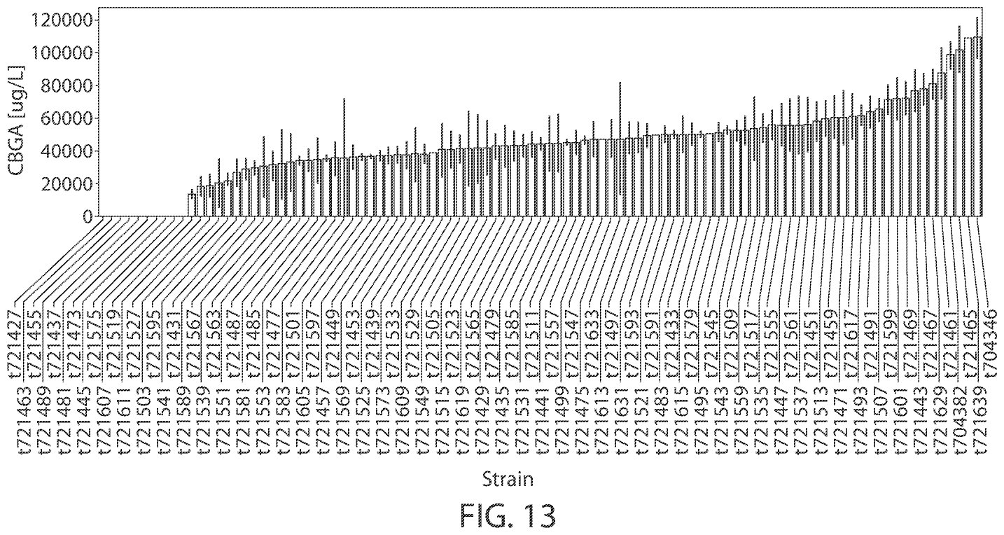

The assay used to assess CBGAS activity of the Gen 3 library was the same as the assay described in Example 3 except that four biological replicates of each strain were screened. Table 8 and FIG. 13 show the results of the Gen 3 library screen. Sequences are provided in Table 16.

TABLE 8
Activity data of Gen 3 library members in S. cerevisiae

| | | | Average | Standard |
|---|---|---|---|---|
| Strain | Strain type | | CBGA | Deviation CBGA |
| ID | PT type | Point mutations | [μg/L] | [μg/L] |
| t704346 | Library | N/A | 109743.3 | 15182.41 |
| | (Chimeric fusion) | | | |
| t721519 | Library | M43L I86S Q288R | 13889.06 | 3081.353 |
| | (Chimeric fusion) | S232R I147L C31F | | |
| | | F245R M87V D94E | | |
| t721611 | Library | I86V M43L Q288R | 18489.32 | 6955.197 |
| | (Chimeric fusion) | L311R S232R I147L | | |
| | | C31F F245R M87V | | |
| t721527 | Library | M43L I86S Q288R | 18839.52 | 7950.718 |
| | (Chimeric fusion) | L311R S232R I147L | | |
| | | F245R M87V D94E | | |
| t721503 | Library | L311N M43L I86S | 20501.72 | 16284.37 |
| | (Chimeric fusion) | Q288R S232R I147L | | |
| | | C31F F245R M87V | | |
| t721595 | Library | I86V M43L Q288R | 22533.01 | 4246.082 |
| | (Chimeric fusion) | L311R S232R I147L | | |
| | | C31F M87V D94E | | |
| t721541 | Library | M43L I86S Q288R | 26768.28 | 9243.536 |
| | (Chimeric fusion) | L311R S232R I147L | | |
| | | C31F M87V D94E | | |
| t721431 | Library | I86A M43L Q288R | 28851.15 | 6522.44 |
| | (Chimeric fusion) | L311R S232R I147L | | |
| | | C31F M87V D94E | | |
| t721589 | Library | L311N I86A M43L | 29596.66 | 5303.2 |
| | (Chimeric fusion) | Q288R S232R I147L | | |
| | | C31F M87V D94E | | |
| t721567 | Library | I86V Q162R | 30288.03 | 21028.47 |
| | (Chimeric fusion) | | | |
| t721539 | Library | L311N I86V M43L | 31171.05 | 9936.614 |
| | (Chimeric fusion) | Q288R S232R I147L | | |
| | | C31F M87V D94E | | |
| t721563 | Library | I86V Q288R L311R | 31919.31 | 24025.56 |
| | (Chimeric fusion) | S232R I147L C31F | | |
| | | F245R M87V D94E | | |
| t721551 | Library | I86V M43L I147L | 33198.67 | 19866.86 |
| | (Chimeric fusion) | | | |
| t721487 | Library | L311N M43L I86S | 34051.27 | 3019.408 |
| | (Chimeric fusion) | Q288R S232R I147L | | |
| | | C31F M87V D94E | | |
| t721581 | Library | L311N I86A M43L | 34162.05 | 9029.252 |
| | (Chimeric fusion) | Q288R S232R I147L | | |
| | | F245R M87V D94E | | |
| t721485 | Library | M43L I86S I147L | 34590.32 | 15526.48 |
| | (Chimeric fusion) | | | |
| t721553 | Library | M43L I86S Q288R | 35227.77 | 1870.526 |
| | (Chimeric fusion) | L311R S232R I147L | | |
| | | C31F F245R M87V | | |
| t721477 | Library | L311N M43L I86S | 35358.3 | 11073.72 |
| | (Chimeric fusion) | Q288R S232R I147L | | |
| | | F245R M87V D94E | | |
| t721583 | Library | I86A S232R I147L | 35715.61 | 50509.5 |
| | (Chimeric fusion) | | | |
| t721501 | Library | M43L I86S Q162R | 36055.5 | 9026.377 |
| | (Chimeric fusion) | | | |
| t721605 | Library | L311N I86A M43L | 36423.18 | 1899.968 |
| | (Chimeric fusion) | Q288R S232R I147L | | |
| | | C31F F245R M87V | | |
| t721597 | Library | L311N I86A M43L | 36593.2 | 1185.784 |
| | (Chimeric fusion) | Q288R S232R I147L | | |
| | | C31F F245R D94E | | |
| t721457 | Library | Q162R I147L | 36926.83 | 3370.906 |
| | (Chimeric fusion) | | | |
| t721449 | Library | I86A M43L Q288R | 37045.45 | 5332.656 |
| | (Chimeric fusion) | L311R S232R I147L | | |
| | | C31F F245R M87V | | |
| t721569 | Library | I86V M43L Q162R | 37409.64 | 5798.489 |
| | (Chimeric fusion) | | | |
| t721453 | Library | M43L Q162R I147L | 37548.49 | 9343.732 |
| | (Chimeric fusion) | | | |
| t721525 | Library | M43L S232R | 37879.24 | 18383.01 |
| | (Chimeric fusion) | | | |
| t721439 | Library | I86A M43L Q288R | 38033.7 | 6513.884 |
| | (Chimeric fusion) | L311R S232R I147L | | |
| | | C31F F245R D94E | | |
| t721573 | Library | L311N I86A M43L | 38813.25 | N/A |
| | (Chimeric fusion) | S232R I147L C31F | | |
| | | F245R M87V D94E | | |
| t721533 | Library | I86V Q288R I147L | 40638.64 | 18125.19 |
| | (Chimeric fusion) | | | |
| t721609 | Library | I86A Q162R | 40819.5 | 12533.58 |
| | (Chimeric fusion) | | | |
| t721529 | Library | M43L Q162R | 41080.92 | 9743.81 |
| | (Chimeric fusion) | | | |
| t721549 | Library | M43L I86S Q288R | 41320.79 | 31673.68 |
| | (Chimeric fusion) | L311R S232R I147L | | |
| | | C31F F245R D94E | | |
| t721505 | Library | M43L Q288R L311R | 41547.59 | 23797.39 |
| | (Chimeric fusion) | S232R I147L C31F | | |
| | | F245R M87V D94E | | |
| t721515 | Library | M43L I147L | 41788.8 | 18840.06 |
| | (Chimeric fusion) | | | |
| t721523 | Library | I86V Q162R I147L | 42614.72 | 8505.742 |
| | (Chimeric fusion) | | | |
| t721619 | Library | I86A Q288R L311R | 42649.28 | 17652.2 |
| | (Chimeric fusion) | S232R I147L C31F | | |
| | | F245R M87V D94E | | |
| t721565 | Library | L311N I86A Q288R | 42939.04 | 9899.911 |
| | (Chimeric fusion) | S232R I147L C31F | | |
| | | F245R M87V D94E | | |
| t721429 | Library | I86A M43L Q162R | 42984.76 | 7672.967 |
| | (Chimeric fusion) | | | |
| t721479 | Library | M43L Q288R Q162R | 43970.59 | 8510.879 |
| | (Chimeric fusion) | | | |
| t721435 | Library | I86A M43L Q288R | 44255.76 | 4267.035 |
| | (Chimeric fusion) | | | |
| t721585 | Library | I86V M43L | 44462.92 | 18971.29 |
| | (Chimeric fusion) | | | |
| t721531 | Library | L311N I86V M43L | 44675.43 | 20221.1 |
| | (Chimeric fusion) | Q288R S232R I147L | | |
| | | F245R M87V D94E | | |
| t721511 | Library | M43L I86S L311R | 45033.76 | 1508.976 |
| | (Chimeric fusion) | S232R I147L C31F | | |
| | | F245R M87V D94E | | |
| t721441 | Library | I86S Q288R Q162R | 45057.23 | 8202.466 |
| | (Chimeric fusion) | | | |
| t721557 | Library | L311N I86V M43L | 46319.13 | 2184.859 |
| | (Chimeric fusion) | Q288R S232R I147L | | |
| | | C31F F245R M87V | | |
| t721499 | Library | I86S Q162R | 46805.05 | 11932.21 |
| | (Chimeric fusion) | | | |
| t721547 | Library | L311N I86V M43L | 46986.1 | N/A |
| | (Chimeric fusion) | Q288R S232R I147L | | |
| | | C31F F245R D94E | | |
| t721475 | Library | Q288R Q162R | 47333.96 | 12957.93 |
| | (Chimeric fusion) | | | |
| t721633 | Library | I86A M43L Q288R | 47493.71 | 41309.64 |
| | (Chimeric fusion) | S232R I147L C31F | | |
| | | F245R M87V D94E | | |
| t721613 | Library | Q288R S232R I147L | 47798.65 | 11325.36 |
| | (Chimeric fusion) | | | |
| t721497 | Library | I86S Q288R L311R | 48022.9 | 10656.39 |
| | (Chimeric fusion) | S232R I147L C31F | | |
| | | F245R M87V D94E | | |
| t721631 | Library | I86A M43L S232R | 49208.88 | 8037.554 |
| | (Chimeric fusion) | | | |
| t721593 | Library | I86A I147L | 49970.05 | N/A |
| | (Chimeric fusion) | | | |
| t721521 | Library | L311N I86V M43L | 50084.12 | 5360.024 |
| | (Chimeric fusion) | S232R I147L C31F | | |
| | | F245R M87V D94E | | |
| t721591 | Library | I86A Q162R I147L | 50121.85 | 2669.096 |
| | (Chimeric fusion) | | | |
| t721483 | Library | I86S I147L | 50166.32 | 12366.79 |
| | (Chimeric fusion) | | | |
| t721433 | Library | I86S Q288R S232R | 50239.79 | 7384.03 |
| | (Chimeric fusion) | | | |
| t721615 | Library | I86A Q288R Q162R | 50407.14 | 1235.417 |
| | (Chimeric fusion) | | | |
| t721579 | Library | I86V M43L Q288R | 50585.97 | N/A |
| | (Chimeric fusion) | S232R I147L C31F | | |
| | | F245R M87V D94E | | |
| t721495 | Library | L311N M43L I86S | 51175.07 | 6278.16 |
| | (Chimeric fusion) | Q288R S232R I147L | | |
| | | C31F F245R D94E | | |
| t721545 | Library | M43L I86S | 52487.65 | 3181.85 |
| | (Chimeric fusion) | | | |
| t721543 | Library | I86V Q288R Q162R | 52510.84 | 6859.685 |
| | (Chimeric fusion) | | | |
| t721509 | Library | M43L I86S Q288R | 52789.12 | 9812.321 |
| | (Chimeric fusion) | | | |
| t721559 | Library | I86V S232R | 53665.3 | 22292.97 |
| | (Chimeric fusion) | | | |
| t721517 | Library | I86V S232R I147L | 53934.61 | 9895.052 |
| | (Chimeric fusion) | | | |
| t721535 | Library | M43L Q288R | 55672.1 | 10047.52 |
| | (Chimeric fusion) | | | |
| t721555 | Library | I86V I147L | 55733 | 14721.98 |
| | (Chimeric fusion) | | | |
| t721447 | Library | M43L S232R I147L | 55837.27 | 17797.46 |
| | (Chimeric fusion) | | | |
| t721561 | Library | I86V M43L S232R | 55904.73 | 20109.23 |
| | (Chimeric fusion) | | | |
| t721537 | Library | I86V Q288R S232R | 56153.05 | 18905.88 |
| | (Chimeric fusion) | | | |
| t721451 | Library | L311N I86S Q288R | 58149.64 | 13257.07 |
| | (Chimeric fusion) | S232R I147L C31F | | |
| | | F245R M87V D94E | | |
| t721513 | Library | L311N I86V Q288R | 59769.79 | 12244.96 |
| | (Chimeric fusion) | S232R I147L C31F | | |
| | | F245R M87V D94E | | |
| t721459 | Library | L311N M43L Q288R | 60443.06 | 14663.24 |
| | (Chimeric fusion) | S232R I147L C31F | | |
| | | F245R M87V D94E | | |
| t721471 | Library | M43L Q288R S232R | 60636.01 | 19027.41 |
| | (Chimeric fusion) | | | |
| t721617 | Library | I86A Q288R | 61026.93 | 15840.07 |
| | (Chimeric fusion) | | | |
| t721493 | Library | M43L I86S S232R | 61690.62 | 6827.457 |
| | (Chimeric fusion) | | | |
| t721491 | Library | I86S S232R | 63843.77 | 11066.42 |
| | (Chimeric fusion) | | | |
| t721507 | Library | I86S Q288R | 65105.45 | 7777.364 |
| | (Chimeric fusion) | | | |
| t721599 | Library | I86A Q288R I147L | 71340.32 | 11852.97 |
| | (Chimeric fusion) | | | |
| t721601 | Library | I86A S232R | 71918.54 | 18251.01 |
| | (Chimeric fusion) | | | |
| t721469 | Library | L311N M43L I86S | 72470.49 | 11594.61 |
| | (Chimeric fusion) | S232R I147L C31F | | |
| | | F245R M87V D94E | | |
| t721443 | Library | S232R I147L | 77020.02 | 14516.1 |
| | (Chimeric fusion) | | | |
| t721467 | Library | Q288R S232R | 77867.5 | 11337.85 |
| | (Chimeric fusion) | | | |
| t721629 | Library | I86S S232R I147L | 80761.42 | 11126.84 |
| | (Chimeric fusion) | | | |
| t721461 | Library | M43L Q288R I147L | 87461.94 | 17781.56 |
| | (Chimeric fusion) | | | |
| t704382 | Library | F245R | 98603.55 | 11623.97 |
| | (Chimeric fusion) | | | |
| t721465 | Library | Q288R I147L | 102133.6 | 16464.64 |
| | (Chimeric fusion) | | | |
| t721639 | Library | I86S Q288R I147L | 109375.4 | N/A |
| | (Chimeric fusion) | | | |
| t721427 | Library | N/A | 0 | 0 |
| | (Chimeric fusion) | | | |
| t721437 | Library | N/A | 0 | 0 |
| | (Chimeric fusion) | | | |
| t721445 | Library | N/A | 0 | 0 |
| | (Chimeric fusion) | | | |
| t721455 | Library | | 0 | 0 |
| | (Chimeric fusion) | | | |
| t721463 | Library | N/A | 0 | 0 |
| | (Chimeric fusion) | | | |
| t721473 | Library | N/A | 0 | 0 |
| | (Chimeric fusion) | | | |
| t721481 | Library | N/A | 0 | 0 |
| | (Chimeric fusion) | | | |
| t721489 | Library | N/A | 0 | 0 |
| | (Chimeric fusion) | | | |
| t721575 | Library | I86V M43L Q288R | 0 | 0 |
| | (Chimeric fusion) | | | |
| t721607 | Library | I86A Q288R S232R | 0 | 0 |
| | (Chimeric fusion) | | | |

Strain t704346 was used as the benchmark for determining hits in the Gen 3 library. Specifically, strains with CBGA production above 75-95% of the average CBGA titer of t704346 were considered hits.

### Example 6: Identification of ERG20 Homologs for Use Infusion Proteins with Chimeric PTs

A library of candidate ERG20 homologs was generated to identify additional fusion partners for chimeric PTs. The ERG20 homologs were engineered to contain tryptophan at residues corresponding to amino acid positions F96 and/or N127 in S. cerevisiae ERG20. Engineered ERG20 homologs were fused C-terminally to the chimeric PT expressed by strain t524816, described in Examples 1 and 2, comprising portions of CsPT4 and CsPT7, to create a library of 2,487 strains. Protein sequences were recoded in silico for expression in S. cerevisiae and synthesized in the replicative yeast expression vector shown in FIG. 8. Strain t756349, comprising a fusion of ERG20ww and the chimeric PT expressed by strain t524816 was used as a positive control and to establish hit ranking. Strain t756346, expressing a fusion of wildtype ERG20 and the chimeric PT expressed by strain t524816 was used to assess the improvement in CBGA production due to the presence of the two tryptophan substitutions in ERG20ww relative to wildtype ERG20. Strain t756347, expressing a fusion of the fluorescent protein RFP and the PT chimera harbored by strain t524816, was used as a negative control.

This chimeric fusion library was assayed for activity in a primary screen using a prenyltransferase assay which was conducted as follows: each thawed glycerol stock was stamped into a well of YEP medium+4% dextrose media. Samples were incubated at 30° C. in a shaking incubator for 2 days. A portion of each of the resulting cultures was stamped into a well of YEP medium+2% raffinose+2% galactose+1 mM olivetolic acid (C6). Samples were incubated at 30° C. and shaken in a shaking incubator for 4 days. A portion of each of the resulting production cultures was stamped into a well of phosphate buffered saline (PBS). Optical measurements were taken on a plate reader, with absorbance measured at 600 nm and fluorescence at 558 nm with 605 nm excitation. A portion of each of the production cultures was stamped into a well of 100% methanol in half-height deepwell plates. Plates were heat sealed and frozen. Samples were then thawed for 30 minutes and spun down at 4° C. A portion of the supernatant was stamped into half-area 96 well plates. CBGA production in the samples was measured via liquid chromatography-mass spectrometry (LC-MS) by measuring relative peak areas. CBGA production was quantified in μg/L by comparing LC/MS peak areas to a standard curve for CBGA.

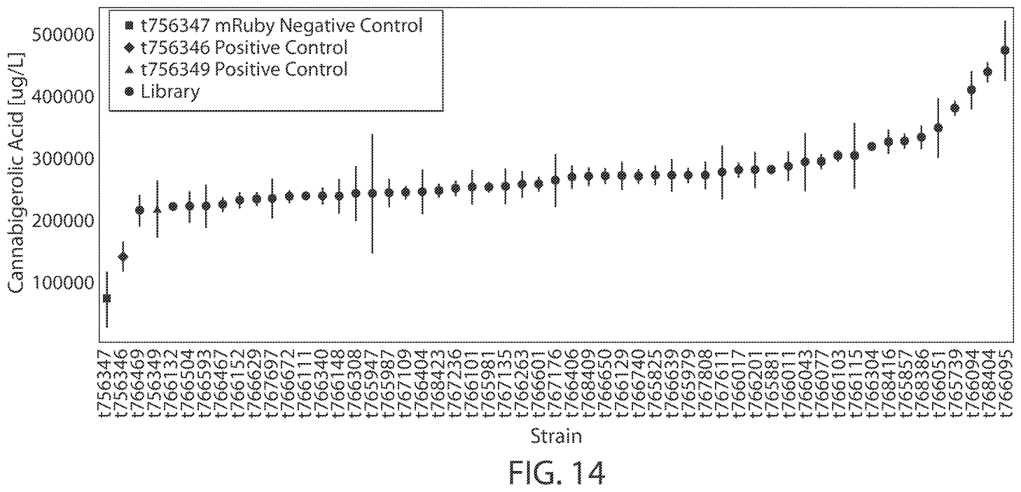

LC-MS analysis revealed that 232 strains out of 2,487 strains generated higher CBGA titers than either of the two positive control strains. Of these, 156 strains were elevated to a secondary assay to confirm their activity. The secondary assay was performed in the same manner as the primary assay with the exception that three biological replicates were included for each strain. Table 9 provides data for the 51 strains identified in the secondary screen that demonstrated higher mean CBGA titers than either positive control (FIG. 14). These 51 strains were found to generate at least 200000 μg/L CBGA, with strains t768404 and t766095 generating more than 400000 μg/L CBGA. The ERG20 homologs expressed by these strains represent promising candidates for N-terminal fusion partners for the PTs described in Examples 1-4. In particular the ERG20 homologs sourced from Kwoniella bestiolae (UniProt accession A0A1B9FXJ1; strain t766469), Pseudogymnoascus sp (UniProt accession A0A094HBN6; strain t766201), and Debaryomyces hansenii (UniProt accession Q6BM51; strain t766095) were selected for further analysis.

TABLE 9
Screening activity data of ERG20 homolog
library described in Example 6

| | | Average | Standard |
|---|---|---|---|
| Strain | Strain Type/UniProt | CBGA | Deviation CBGA |
| ID | Accession No. | [μg/L] | [μg/L] |
| t756347 | RFP Negative Control | 71454.58 | 45276.26 |
| t756346 | ERG20 Positive Control | 139335.35 | 22784.51 |
| | UniProt Accession No. | | |
| | P08524 | | |
| t756349 | ERG20ww Positive | 217038.20 | 45255.36 |
| | Control | | |
| | UniProt Accession No. | | |
| | P08524 | | |
| t766469 | Library | 214618.91 | 29131.89 |
| | UniProt Accession No. | | |
| | A0A1B9FXJ1 | | |
| t766132 | Library | 220512.78 | 4294.99 |
| | UniProt Accession No. | | |
| | A0A0L139D1 | | |
| t766504 | Library | 220615.17 | 29851.95 |
| | UniProt Accession No. | | |
| | A8PB79 | | |
| t766593 | Library | 221529.52 | 38864.81 |
| | UniProt Accession No. | | |
| | G1X2B3 | | |
| t766467 | Library | 223414.45 | 11951.96 |
| | UniProt Accession No. | | |
| | A0A1C7NP81 | | |
| t766152 | Library | 230281.35 | 14362.33 |
| | UniProt Accession No. | | |
| | A0A0U5GM00 | | |
| t766629 | Library | 232560.14 | 10690.82 |
| | UniProt Accession No. | | |
| | A0A093ZCS9 | | |
| t767697 | Library | 233588.88 | 44030.98 |
| | UniProt Accession No. | | |
| | M5GG98 | | |
| t766672 | Library | 236749.70 | 11755.96 |
| | UniProt Accession No. | | |
| | H6BT51 | | |
| t766111 | Library | 237007.65 | 888.75 |
| | UniProt Accession No. | | |
| | A0A225B7V9 | | |
| t766340 | Library | 237353.70 | 15551.58 |
| | UniProt Accession No. | | |
| | K2RVP5 | | |
| t766148 | Library | 237647.35 | 30159.01 |
| | UniProt Accession No. | | |
| | A0A1B7P2J8 | | |
| t766308 | Library | 241289.81 | 49458.69 |
| | UniProt Accession No. | | |
| | A0A1B8DWN7 | | |
| t765947 | Library | 241617.80 | 109375.71 |
| | UniProt Accession No. | | |
| | G8ZRX5 | | |
| t765987 | Library | 242172.50 | 24234.55 |
| | UniProt Accession No. | | |
| | A0A2H3H5S3 | | |
| t767109 | Library | 242937.43 | 11536.39 |
| | UniProt Accession No. | | |
| | A0A1G4KG41 | | |
| t766404 | Library | 243961.52 | 39580.10 |
| | UniProt Accession No. | | |
| | A0A1A0HBK2 | | |
| t768423 | Library | 246190.88 | 10681.19 |
| | UniProt Accession No. | | |
| | R7S2J9 | | |
| t767236 | Library | 249887.24 | 11503.32 |
| | UniProt Accession No. | | |
| | W1QIU7 | | |
| t766101 | Library | 251351.45 | 31159.81 |
| | UniProt Accession No. | | |
| | B8MAT2 | | |
| t765981 | Library | 251465.24 | 8111.58 |
| | UniProt Accession No. | | |
| | A0A151N659 | | |
| t767135 | Library | 252456.18 | 32252.26 |
| | UniProt Accession No. | | |
| | A0A1Q5UIR3 | | |
| t766263 | Library | 256417.46 | 23459.79 |
| | UniProt Accession No. | | |
| | A0A165DW64 | | |
| t766601 | Library | 256635.65 | 11627.73 |
| | UniProt Accession No. | | |
| | B6K405 | | |
| t767176 | Library | 262466.12 | 47023.13 |
| | UniProt Accession No. | | |
| | A0A1E3QBG8 | | |
| t766406 | Library | 267311.70 | 21804.03 |
| | UniProt Accession No. | | |
| | A0A1R0GPX8 | | |
| t768409 | Library | 269071.54 | 14545.34 |
| | UniProt Accession No. | | |
| | P08524 | | |
| t766650 | Library | 269596.06 | 12599.25 |
| | UniProt Accession No. | | |
| | A0A093XCN7 | | |
| t766129 | Library | 269769.02 | 23305.89 |
| | UniProt Accession No. | | |
| | G1X2B3 | | |
| t766740 | Library | 269964.77 | 13013.87 |
| | UniProt Accession No. | | |
| | A0A0C7NDP3 | | |
| t765825 | Library | 270522.15 | 17858.87 |
| | UniProt Accession No. | | |
| | W6YHT5 | | |
| t766639 | Library | 270534.32 | 28222.77 |
| | UniProt Accession No. | | |
| | A0A0F7TT74 | | |
| t765979 | Library | 270560.02 | 13113.97 |
| | UniProt Accession No. | | |
| | A0A1L9RD60 | | |
| t767808 | Library | 271451.55 | 25968.15 |
| | UniProt Accession No. | | |
| | A0A0C3S0Z8 | | |
| t767611 | Library | 276121.67 | 47485.23 |
| | UniProt Accession No. | | |
| | W0T5C0 | | |
| t766017 | Library | 278992.34 | 14797.49 |
| | UniProt Accession No. | | |
| | A0A1Y2HMF7 | | |
| t766201 | Library | 279773.20 | 31427.85 |
| | UniProt Accession No. | | |
| | A0A094HBN6 | | |
| t765881 | Library | 280115.24 | 6376.44 |
| | UniProt Accession No. | | |
| | A0A1B8D7F8 | | |
| t766011 | Library | 285446.12 | 25280.37 |
| | UniProt Accession No. | | |
| | W9YHW7 | | |
| t766043 | Library | 292288.94 | 50804.41 |
| | UniProt Accession No. | | |
| | C4QY32 | | |
| t766077 | Library | 292936.87 | 12638.64 |
| | UniProt Accession No. | | |
| | A0A1G4MGY0 | | |
| t766103 | Library | 302283.63 | 7255.66 |
| | UniProt Accession No. | | |
| | T0Q3I5 | | |
| t766115 | Library | 302770.65 | 58626.99 |
| | UniProt Accession No. | | |
| | A0A0F9Z7F4 | | |
| t766304 | Library | 317453.64 | 3294.56 |
| | UniProt Accession No. | | |
| | A0A2H0ZLN6 | | |
| t768416 | Library | 324513.69 | 25839.79 |
| | UniProt Accession No. | | |
| | W7I4W9 | | |
| t765857 | Library | 325501.06 | 13171.54 |
| | UniProt Accession No. | | |
| | G0W361 | | |
| t768386 | Library | 331626.97 | 19113.89 |
| | UniProt Accession No. | | |
| | A0A1D2VIM1 | | |
| t766051 | Library | 347717.12 | 52075.70 |
| | UniProt Accession No. | | |
| | G8JX22 | | |
| t765739 | Library | 378842.07 | 11804.91 |
| | UniProt Accession No. | | |
| | A0A1E4RE25 | | |
| t766094 | Library | 407946.27 | 32968.01 |
| | UniProt Accession No. | | |
| | B6K405 | | |
| t768404 | Library | 437126.53 | 15533.53 |
| | UniProt Accession No. | | |
| | A0A1L0BIM2 | | |
| t766095 | Library | 471588.30 | 53964.99 |
| | UniProt Accession No. | | |
| | Q6BM51 | | |

Analysis of ERG20 homologs using a motif identification software identified multiple sequence motifs that were enriched in chimeric fusions that produce CBGA (Table 10). Table 17 provides sequence information for the ERG20 homologs contained within the chimeric fusions described in this Example. Table 18 provides sequence information for the chimeric fusions described in this Example.

TABLE 10
Non-limiting examples of ERG20 homolog motifs

| | Reference sequence | | | | |
|---|---|---|---|---|---|
| | for amino acid | | | | |
| | numbering | | | | |
| | (UniProt P08524; | | | | Erg20 |
| | SEQ ID NO: 424) | | Motif sequence in | | SEQ |
| Motif | start | end | strain | Strain | ID NO |
| NVPGGKLNR (SEQ | 47 | 55 | NVPGGKLNR (SEQ | t766132 | 426 |
| ID NO: 647) | | | ID NO: 647) | t766504 | 427 |
| | | | | t766467 | 429 |
| | | | | t766152 | 430 |
| | | | | t768423 | 442 |
| | | | | t765979 | 457 |
| | | | | 1767808 | 458 |
| FYLPVALA[LM]H | 203 | 212 | FYLPVALALH (SEQ | 1766629 | 431 |
| (SEQ ID NO: 648) | | | ID NO: 649) | t766672 | 433 |
| | | | | t766148 | 436 |
| | | | | t766308 | 437 |
| | | | | t765987 | 439 |
| | | | | t766650 | 452 |
| | | | | t765979 | 457 |
| | | | | t766201 | 461 |
| | | | | t765881 | 462 |
| | | | | t766011 | 463 |
| | | | FYLPVALAMH (SEQ | t766467 | 429 |
| | | | ID NO: 650) | t768423 | 442 |
| | | | | t766263 | 447 |
| | | | | t766051 | 472 |
| A[EH]D[IV]LIPLG | 225 | 233 | AEDILIPLG (SEQ ID | t765987 | 439 |
| (SEQ ID NO: 651) | | | NO: 652) | t766650 | 452 |
| | | | AHDILIPLG (SEQ ID | t766132 | 426 |
| | | | NO: 653) | t766467 | 429 |
| | | | | t766152 | 430 |
| | | | | t766672 | 433 |
| | | | | t766148 | 436 |
| | | | | t767135 | 446 |
| | | | | t766639 | 456 |
| | | | | t766011 | 463 |
| | | | | t768386 | 471 |
| | | | AHDVLIPLG (SEQ ID | t765947 | 438 |
| | | | NO: 654) | t766129 | 453 |
| | | | | t766593 | 428 |
| | | | | t767176 | 449 |
| LGW[CL][ITV]ELLQ | 85 | 97 | LGWLTELLQAYFL | t766201 | 461 |
| A[FY]FL (SEQ ID NO: | | | (SEQ ID NO: 656) | t766308 | 437 |
| 655) | | | LGWLTELLQAFFL | t765979 | 457 |
| | | | (SEQ ID NO: 657) | t766011 | 463 |
| | | | | t766132 | 426 |
| | | | LGWCIELLQAYFL | t765739 | 473 |
| | | | (SEQ ID NO: 658) | t765857 | 470 |
| | | | | t765947 | 438 |
| | | | | t766077 | 465 |
| | | | | t766095 | 476 |
| | | | | t767611 | 459 |
| | | | | t768386 | 471 |
| | | | | t768404 | 475 |
| | | | | t768409 | 451 |
| | | | LGWCVELLQAYFL | t766043 | 464 |
| | | | (SEQ ID NO: 659) | t766051 | 472 |
| | | | LGWCVELLQAFFL | t766263 | 447 |
| | | | (SEQ ID NO: 660) | t767697 | 432 |
| | | | | t767808 | 458 |
| | | | LGWCIELLQAFFL | t766094 | 474 |
| | | | (SEQ ID NO: 661) | t768423 | 442 |
| | | | | t766129 | 453 |
| | | | LGWCTELLQAFFL | t768416 | 469 |
| | | | (SEQ ID NO: 662) | | |
| KKEV[FL][ET][SA]FL | 336 | 349 | KKEVFESFLAKIYK | t766639 | 456 |
| [AGN]KIYK (SEQ ID | | | (SEQ ID NO: 664) | t767135 | 446 |
| NO: 663) | | | KKEVFEAFLGKIYK | t766152 | 430 |
| | | | (SEQ ID NO: 665) | | |
| | | | KKEVLTSFLNKIYK | t768416 | 469 |
| | | | (SEQ ID NO: 666) | | |
| QRK[VI]L[DE]ENYG | 279 | 288 | QRKVLDENYG (SEQ | t766672 | 433 |
| (SEQ ID NO: 667) | | | ID NO: 668) | t766263 | 447 |
| | | | | t766601 | 448 |
| | | | | t766740 | 454 |
| | | | | t767611 | 459 |
| | | | | t766011 | 463 |
| | | | | t765857 | 470 |
| | | | | t766094 | 474 |
| | | | | t768404 | 475 |
| | | | QRKILDENYG (SEQ | t767176 | 449 |
| | | | ID NO: 669) | t765881 | 462 |
| | | | QRKILEENYG (SEQ | t765987 | 439 |
| | | | ID NO: 670) | | |
| | | | QRKVLEENYG (SEQ | t766629 | 431 |
| | | | ID NO: 671) | t766308 | 437 |
| | | | | t766650 | 452 |
| | | | | t766201 | 461 |
| VGMIAIWD (SEQ ID | 121 | 128 | VGMIAIWD (SEQ ID | t767697 | 432 |
| NO: 672) | | | NO: 672) | t766340 | 435 |
| | | | | t766148 | 436 |
| | | | | t766308 | 437 |
| | | | | t765987 | 439 |
| | | | | t766101 | 444 |
| | | | | t767176 | 449 |
| | | | | t766406 | 450 |
| | | | | t765825 | 455 |
| | | | | t766201 | 461 |
| | | | | t766011 | 463 |
| | | | | t766115 | 467 |
| TDI[QK]DNKCSW | 217 | 226 | TDIQDNKCSW (SEQ | t766152 | 430 |
| (SEQ ID NO: 673) | | | ID NO: 674) | t766111 | 434 |
| | | | | t766340 | 435 |
| | | | | t766148 | 436 |
| | | | | t765947 | 438 |
| | | | | t767109 | 440 |
| | | | | t766101 | 444 |
| | | | | t765981 | 445 |
| | | | | t767135 | 446 |
| | | | | t768409 | 451 |
| | | | | t766740 | 454 |
| | | | | t766639 | 456 |
| | | | | t767611 | 459 |
| | | | | t766043 | 464 |
| | | | | t766077 | 465 |
| | | | | t766103 | 466 |
| | | | | t765857 | 470 |
| | | | | t768386 | 471 |
| | | | | t766051 | 472 |
| | | | | t766132 | 426 |
| | | | TDIKDNKCSW (SEQ | t765987 | 439 |
| | | | ID NO: 675) | t766404 | 441 |
| | | | | t766406 | 450 |
| | | | | t765979 | 457 |
| | | | | t766115 | 467 |
| | | | | t766304 | 468 |
| | | | | t765739 | 473 |
| | | | | t768404 | 475 |
| | | | | t766095 | 476 |
| TAYYSFYLP (SEQ ID | 198 | 206 | TAYYSFYLP (SEQ ID | t766132 | 426 |
| NO: 676) | | | NO: 676) | t766504 | 427 |
| | | | | t766593 | 428 |
| | | | | t766467 | 429 |
| | | | | t766152 | 430 |
| | | | | t766629 | 431 |
| | | | | t767697 | 432 |
| | | | | t766672 | 433 |
| | | | | t766111 | 434 |
| | | | | t766340 | 435 |
| | | | | t766148 | 436 |
| | | | | t766308 | 437 |
| | | | | t765947 | 438 |
| | | | | t765987 | 439 |
| | | | | t767109 | 440 |
| | | | | t766404 | 441 |
| | | | | t767236 | 443 |
| | | | | t766101 | 444 |
| | | | | t767135 | 446 |
| | | | | t766263 | 447 |
| | | | | t766601 | 448 |
| | | | | t767176 | 449 |
| | | | | t768409 | 451 |
| | | | | t766650 | 452 |
| | | | | t766129 | 453 |
| | | | | t766740 | 454 |
| | | | | t765825 | 455 |
| | | | | t766639 | 456 |
| | | | | t765979 | 457 |
| | | | | t767611 | 459 |
| | | | | t766201 | 461 |
| | | | | t765881 | 462 |
| | | | | t766011 | 463 |
| | | | | t766043 | 464 |
| | | | | t766077 | 465 |
| | | | | t766304 | 468 |
| | | | | t768416 | 469 |
| | | | | t765857 | 470 |
| | | | | t768386 | 471 |
| | | | | t766051 | 472 |
| | | | | t765739 | 473 |
| | | | | t766094 | 474 |
| | | | | t768404 | 475 |
| | | | | t766095 | 476 |
| GKIGTDI[QK]DNKCS | 253 | 266 | GKIGTDIQDNKCSW | t766152 | 430 |
| W (SEQ ID NO: 677) | | | (SEQ ID NO: 678) | t766111 | 434 |
| | | | | t766340 | 435 |
| | | | | t766148 | 436 |
| | | | | t765947 | 438 |
| | | | | t767109 | 440 |
| | | | | t766101 | 444 |
| | | | | t765981 | 445 |
| | | | | t767135 | 446 |
| | | | | t768409 | 451 |
| | | | | t766740 | 454 |
| | | | | t766639 | 456 |
| | | | | t767611 | 459 |
| | | | | t766043 | 464 |
| | | | | t766077 | 465 |
| | | | | t766103 | 466 |
| | | | | t765857 | 470 |
| | | | | t768386 | 471 |
| | | | | t766051 | 472 |
| | | | GKIGTDIKDNKCSW | t766132 | 426 |
| | | | (SEQ ID NO: 679) | t765987 | 439 |
| | | | | t766404 | 441 |
| | | | | t765979 | 457 |
| | | | | t766115 | 467 |
| | | | | t766304 | 468 |
| | | | | t765739 | 473 |
| | | | | t768404 | 475 |
| | | | | t766095 | 476 |
| ILIP[LM] GEYFQ | 228 | 237 | ILIPLGEYFQ (SEQ ID | t766504 | 427 |
| (SEQ ID NO: 680) | | | NO: 681) | t766467 | 429 |
| | | | | t767697 | 432 |
| | | | | t766672 | 433 |
| | | | | t766111 | 434 |
| | | | | t765987 | 439 |
| | | | | t768423 | 442 |
| | | | | t766101 | 444 |
| | | | | t766263 | 447 |
| | | | | t767808 | 458 |
| | | | | t766011 | 463 |
| | | | | t766043 | 464 |
| | | | | t766115 | 467 |
| | | | | t768386 | 471 |
| | | | | t765739 | 473 |
| | | | | t766095 | 476 |
| | | | ILIPMGEYFQ (SEQ | t766340 | 435 |
| | | | ID NO: 682) | | |
| IL[VM][EP][ML]G | 228 | 237 | ILVPMGEYFQ (SEQ | t765825 | 455 |
| [ETHYF]FQ (SEQ ID | | | ID NO: 684) | | |
| NO: 683) | | | | | |
| AKIYKRSK (SEQ ID | 345 | 352 | AKIYKRSK (SEQ ID | t766672 | 433 |
| NO: 685) | | | NO: 685) | t765987 | 439 |
| | | | | t766011 | 463 |
| DPEVIGKI (SEQ ID | 248 | 255 | DPEVIGKI (SEQ ID | t766152 | 430 |
| NO: 686) | | | NO: 686) | | |
| RGQPCW[YF]RVP | 110 | 120 | RGQPCWYRVPE | t767109 | 440 |
| [EQ] (EQ ID NO: 687) | | | (SEQ ID NO: 688) | t766740 | 454 |
| IVKYKTA[YF]Y[ST]F | 193 | 206 | IVKYKTAFYSFYLP | t765981 | 445 |
| YLP (SEQ ID NO: | | | (SEQ ID NO: 690) | | |
| 689) | | | IVKYKTAYYSFYLP | t766111 | 434 |
| | | | (SEQ ID NO: 691) | t766101 | 444 |
| WC[IV]E[LW]LQA | 87 | 100 | WCIELLQAFFLVAD | t765981 | 445 |
| [YF][WF]LV[ALW]D | | | (SEQ ID NO: 693) | | |
| (SEQ ID NO: 692) | | | WCIELLQAFWLVAD | t766094 | 474 |
| | | | (SEQ ID NO: 694) | | |
| | | | WCIELLQAYFLVAD | t766601 | 448 |
| | | | (SEQ ID NO: 695) | t765947 | 438 |
| | | | | t768409 | 451 |
| | | | | t767611 | 459 |
| | | | | t766077 | 465 |
| | | | | t765857 | 470 |
| | | | | t768386 | 471 |
| | | | | t765739 | 473 |
| | | | | t768404 | 475 |
| | | | | t766095 | 476 |
| | | | WCIELLQAYWLVAD | t767109 | 440 |
| | | | (SEQ ID NO: 696) | t766740 | 454 |
| | | | WCIEWLQAFFLVAD | t766406 | 450 |
| | | | (SEQ ID NO: 697) | t766103 | 466 |
| | | | WCVELLQAYFLVAD | t766043 | 464 |
| | | | (SEQ ID NO: 698) | t766051 | 472 |
| CSWLV[VN]Q[AC]L | 264 | 279 | CSWLVVQALARATP | t766103 | 466 |
| [AQ][RI][AC][ST]P | | | EQ (SEQ ID NO: 700) | | |
| [ED]Q | | | | | |
| (SEQ ID NO: 699) | | | | | |

### Example 7: Functional Expression of Additional Chimeric PTs

To further improve the CBGA and CBGVA titer production of chimeric PTs, chimeric PTs from strains t523834 (SEQ ID NO: 114, corresponding to a CsPT1-CsPT4 chimera) and t524816 (SEQ ID NO: 116, corresponding to a CsPT4-CsPT7 chimera), described in Examples 1 and 2, were modified to include point mutations that were characterized in Example 1. The modified chimeric PTs were screened in a Gen 4 library.

Example 1 above describes the identification of 74 point mutations that improved CBGA production and 23 point mutations that improved CBGVA production. All 23 of the point mutations that improved CBGVA production also improved CBGA production. These mutations were ranked using a productivity score comprised of the sum of their CBGA and CBGVA titers normalized to those from a truncated CsPT4 (strain t612212: SEQ ID NO: 5). Subsets of the top hits of point mutations were selected for screening based on the ranked productivity score. Combinations of the selected point mutations were introduced into SEQ ID NO: 114 and SEQ ID NO: 116 to produce new chimeric PTs.

Point mutations in the chimeric PTs corresponding to SEQ ID NOs: 114 and 116 were generated at positions where the native residue in the chimera is the same as in CsPT4. For SEQ ID NO: 116, mutational loads between 2-4 mutations were generated by stacking all combinations of the top 8 ranked point-mutations, and all combinations of the top 11 ranked point-mutations where all inter-residue distances were greater than 6 Angstroms. For SEQ ID NO: 114, mutational loads of 9-10 mutations were generated by stacking all combinations of the top 15 ranked point-mutations, and all combinations of the top 23 ranked point-mutations where all inter-residue distances were greater that 6 Angstroms.

Protein sequences were recoded in silico for expression in S. cerevisiae and synthesized in the replicative yeast expression vector shown in FIG. 8. Each chimeric PT expression construct was transformed into an S. cerevisiae CEN.PK strain that was engineered to overproduce GPP. Strain t819232, comprising a fluorescent protein (RFP), was included in the library as a negative control. Strain t827885, expressing a chimeric PT corresponding to SEQ ID NO: 324 (the same chimeric PT expressed in strain t721639, except that it is not a fusion), was used as a positive control and for establishing hit ranking.

The Gen4 library was assayed for activity in a primary screen using a prenyltransferase assay which was conducted as follows: each thawed glycerol stock of PT transformants was stamped into a well of YPD (yeast extract peptone dextrose)+4% dextrose media. Samples were incubated at 30° C. in a shaking incubator for 2 days. A portion of each of the resulting cultures was stamped into a well of YEP (yeast extract+dextrose)+2% raffinose+2% galactose+1 mM olivetolic acid (C6). Samples were incubated at 30° C. in a shaking incubator for 4 days. A portion of each of the resulting production cultures was stamped into a well of PBS. Optical measurements were taken on a plate reader, with absorbance measured at 600 nm and fluorescence at 528 nm with 485 nm excitation. A portion of each of the production cultures was stamped into a well of 100% methanol in half-height deepwell plates. Plates were heat sealed and frozen. Samples were then thawed and spun down at 4° C. A portion of the supernatant was stamped into half-area 96 well plates. CBGA production in the samples was quantified via LC-MS by measuring relative peak areas. CBGA production was quantified in μg/L by comparing LC/MS peak areas to a standard curve for CBGA.

112 chimeric PT variants were elevated to a secondary screen to verify their CBGAS activity and to further quantify the production of other cannabinoids. A total of 20 variants of the chimeric PT corresponding to SEQ ID NO: 116 and 23 variants of the chimeric PT corresponding to SEQ ID NO. 114 were carried over from the primary screen to the secondary screen. As shown in Table 11, the following point mutations were tested in the chimeric PTs, either alone or in combination: M43L, M87T, M87I, I86G, I86S, F82G, F151T, S119A, V122S, V122F, I86V, I86T, D94E, M87V, C31F, F151G, I147L, I86A, F245R, and F83Y were tested in the chimeric PT corresponding to SEQ ID NO: 116 and Q288R, M43L, F245W, F P145T, C31F, F245R, I86G, I86S, F82G, F145L, Q267, I147L, L311K, L311R, M43V, L311N, D94E, E113R, I86V, F145S, M87V, I86A, and I46C were tested in the chimeric PT corresponding to SEQ ID NO: 114.

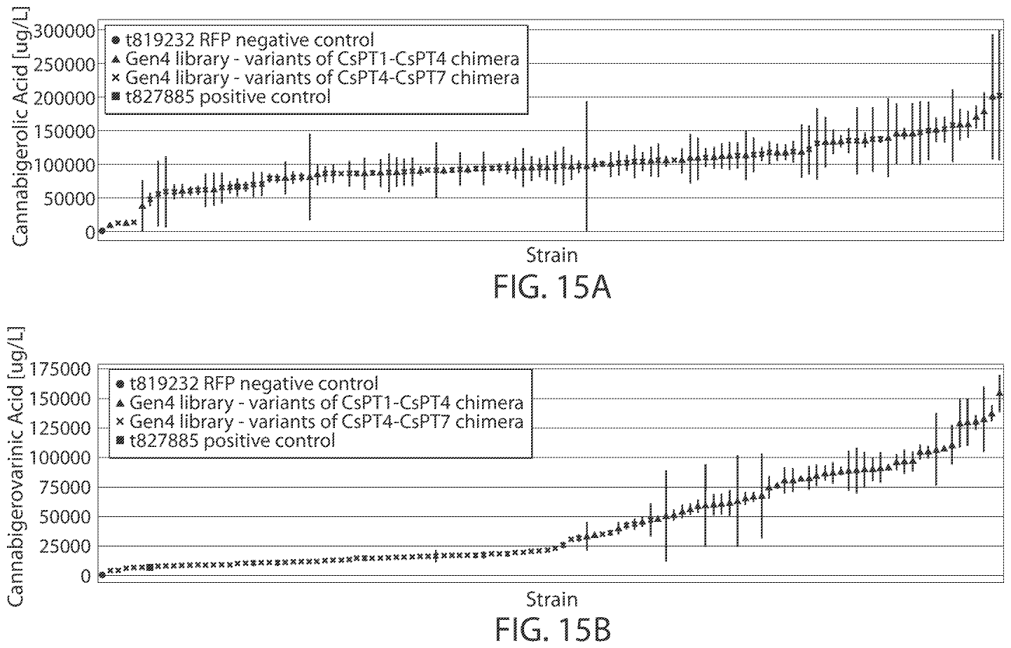

In addition to screening for activity on olivetolic acid (C6), a parallel experiment was performed to screen the set of enzymes tested in the secondary screen on the C4 substrate divaric acid (DA), by substituting 1 mM divaric acid for the 1 mM olivetolic acid (OA) in the prenyltransferase assay described above. The resulting products, CBGA and cannabigerovarinic acid (CBGVA), were quantified in μg/L by comparing LC-MS peak areas to the respective standard curve for CBGA and CBGVA. See, Example 1. The experimental protocols for the secondary screen were the same as the assays used in primary screen except that both CBGA and CBGVA production were measured using LC-MS on four biological replicates incubated with OA or DA, respectively (FIG. 15, Table 11).

TABLE 11
Activity data of Gen 4 library members in S. cerevisiae

| | Strain type | | | Standard | | Standard |
|---|---|---|---|---|---|---|
| | PT type | Parent | Average | Deviation | Average | Deviation |
| | Mutation | chimera | CBGA | CBGA | CBGVA | CBGVA |
| Strain ID | (if applicable) | Strain | [μg/L] | [μg/L] | [μg/L] | [μg/L] |
| t827885 | Positive control | t721639 | 65297.254 | 12696.216 | 6507.709 | 716.085 |
| | chimeric PT | | | | | |
| t819232 | Negative control | N/A | 0 | 0 | 0 | 0 |
| t817911 | Library Chimera; C31F | t524816 | 96205 | 32827.12 | 34495.802 | 2161.756 |
| | F82G D94E F245R | | | | | |
| t817917 | Library Chimera, M43L | t524816 | 92043 | 29941.79 | 13117.368 | 1269.355 |
| | I86S I147L F245R | | | | | |
| t817954 | Library Chimera; C31F | t523834 | 100379.8 | 19850.32 | 136697.660 | 7865.282 |
| | M43V M87V D94E | | | | | |
| | E113R F145L F245W | | | | | |
| | Q267F Q288R | | | | | |
| t817955 | Library Chimera; C31F | t523834 | 109018 | 40816.12 | 131903.743 | 31952.635 |
| | M43L F82G D94E E113R | | | | | |
| | F145T F245R Q267F | | | | | |
| | Q288R | | | | | |
| t817960 | Library Chimera; M43L | t523834 | 113041.4 | 42038.45 | 104161.702 | 6220.732 |
| | F82G D94E E113R | | | | | |
| | F145S F245R Q267F | | | | | |
| | Q288R L311K | | | | | |
| t817962 | Library Chimera; C31F | t523834 | 139537.9 | 68090.21 | 51029.723 | 5570.915 |
| | M43V M87V D94E | | | | | |
| | E113R F245R Q267F | | | | | |
| | Q288R L311N | | | | | |
| t817963 | Library Chimera; I86A | t524816 | 93245.51 | 28187.41 | 19731.337 | 2071.111 |
| | D94E I147L F245R | | | | | |
| t817977 | Library Chimera; C31F | t523834 | 86621.15 | 25445.17 | 66671.475 | 5282.953 |
| | I46C I86A D94E E113R | | | | | |
| | F145T F245R Q267F | | | | | |
| | Q288R | | | | | |
| t817985 | Library Chimera; C31F | t523834 | 106164.5 | 19913.03 | 34201.160 | 876.195 |
| | M43V M87V D94E | | | | | |
| | F145T F245R Q267F | | | | | |
| | Q288R L311N | | | | | |
| t817996 | Library Chimera; C31F | t523834 | 132865.1 | 44482.93 | 106858.047 | 2103.630 |
| | I86G D94E E113R F145T | | | | | |
| | F245W Q267F Q288R | | | | | |
| | L311N | | | | | |
| t818002 | Library Chimera; I86V | t524816 | 105606.3 | 28247.86 | 9766.031 | 1161.223 |
| | F245R | | | | | |
| t818007 | Library Chimera; C31F | t523834 | 79588.36 | 27299.71 | 61287.525 | 11879.118 |
| | I46C I86G D94E E113R | | | | | |
| | F245R Q267F Q288R | | | | | |
| | L311N | | | | | |
| t818009 | Library Chimera; C31F | t523834 | 95077.48 | 21940.2 | 80236.181 | 12882.087 |
| | M43V M87V D94E | | | | | |
| | E113R F145L F245R | | | | | |
| | Q288R L311R | | | | | |
| t818014 | Library Chimera; C31F | t524816 | 157958.3 | 60683.91 | 16825.813 | 1271.625 |
| | M43L I86A I147L | | | | | |
| t818015 | Library Chimera; I86S | t524816 | 64426.3 | 10734.56 | 8455.435 | 1500.672 |
| | D94E F245R | | | | | |
| t818033 | Library Chimera; C31F | t523834 | 94781.41 | 27521.67 | 81666.892 | 2215.417 |
| | M43L I86S D94E E113R | | | | | |
| | F145S F245R Q267F | | | | | |
| | L311K | | | | | |
| t818043 | Library Chimera; C31F | t524816 | 118537.3 | 18987.01 | 11397.707 | 1038.077 |
| | M43L I86V D94E | | | | | |
| t818044 | Library Chimera; C31F | t523834 | 119012.7 | 45314.1 | 58929.539 | 39289.466 |
| | I46C F82G D94E E113R | | | | | |
| | I147L Q267F Q288R | | | | | |
| | L311N | | | | | |
| t818058 | Library Chimera; C31F | t524816 | 69877.66 | 24545.27 | 12600.668 | 48.783 |
| | M43L I86A F245R | | | | | |
| t818067 | Library Chimera; C31F | t524816 | 58799.62 | 12325.36 | 8842.980 | 658.628 |
| | I86A F245R | | | | | |
| t818093 | Library Chimera; C31F | t523834 | 111724.4 | 24758.13 | 86022.676 | 7682.185 |
| | M43L I86S E113R F145L | | | | | |
| | F245R Q267F Q288R | | | | | |
| | L311R | | | | | |
| t818098 | Library Chimera; C31F | t523834 | 94659.97 | 32406.3 | 47879.602 | 2438.941 |
| | I46C M87V D94E E113R | | | | | |
| | F145L Q267F Q288R | | | | | |
| | L311N | | | | | |
| t818130 | Library Chimera; I86S | t524816 | 68599.8 | 21345.91 | 8936.075 | 364.916 |
| | D94E | | | | | |
| t818140 | Library Chimera; 186T | t524816 | 63747.82 | 25619.76 | 6121.095 | 630.620 |
| | M87I F151T | | | | | |
| t818171 | Library Chimera; M43L | t524816 | 95177.7 | 27671.48 | 8571.350 | 1915.307 |
| | I86S D94E | | | | | |
| t818180 | Library Chimera; F83Y | t524816 | 87390.44 | 32825.62 | 10826.062 | 311.695 |
| | I86A M87T | | | | | |
| t818195 | Library Chimera; C31F | t523834 | 87593.61 | 26092.39 | 59658.404 | 9742.367 |
| | M43L F82G I86G D94E | | | | | |
| | L311N | | | | | |
| | F145L 1147L F245R | | | | | |
| | L311N | | | | | |
| t818196 | Library Chimera; I86V | t524816 | 46874.88 | 11423.02 | 7766.267 | 631.558 |
| | M87T | | | | | |
| t818198 | Library Chimera: I86V | t524816 | 61359.01 | 28412.76 | 10611.003 | 3223.715 |
| | I147L F245R | | | | | |
| t818205 | Library Chimera; C31F | t524816 | 65670.66 | 9344.088 | 14609.435 | 1883.339 |
| | I86V D94E I147L | | | | | |
| t818206 | Library Chimera; C31F | t523834 | 59764.32 | 11215.17 | 91106.141 | 4284.420 |
| | I46C I86A E113R 1147L | | | | | |
| | F245W Q267F Q288R | | | | | |
| | L311N | | | | | |
| t818207 | Library Chimera: I86A | t524816 | 87654.22 | 23691.22 | 16726.277 | 897.206 |
| | M87I | | | | | |
| t818208 | Library Chimera; C31F | t523834 | 61943.88 | 27862.96 | 96611.578 | 9790.778 |
| | M43L I86A D94E E113R | | | | | |
| | F145S F245R Q267F | | | | | |
| | L311K | | | | | |
| t818210 | Library Chimera; C31F | t523834 | 105308.1 | 25589.21 | 129878.457 | 7239.716 |
| | M43L I86G D94E E113R | | | | | |
| | F145L F245R Q288R | | | | | |
| | L311R | | | | | |
| t818214 | Library Chimera; M43L | t524816 | 78738.16 | 9345.376 | 16374.522 | 1160.505 |
| | I86A D94E | | | | | |
| t818215 | Library Chimera; C31F | t523834 | 110533.7 | 18237.31 | 90748.824 | 14334.815 |
| | I46C I86G D94E E113R | | | | | |
| | F145L F245R Q288R | | | | | |
| | L311N | | | | | |
| t818223 | Library Chimera; C31F | t523834 | 94254.93 | 17979.98 | 75788.092 | 3482.879 |
| | M43V I86G E113R | | | | | |
| | F145L F245R Q267F | | | | | |
| | Q288R L311K | | | | | |
| t818230 | Library Chimera; C31F | t523834 | 84126.01 | 18011.56 | 89495.046 | 17265.633 |
| | F82G 186V M87V D94E | | | | | |
| | F145L 1147L F245R | | | | | |
| | L311K | | | | | |
| t818247 | Library Chimera; I86A | t524816 | 85367.09 | 16396.53 | 18065.417 | 2836.827 |
| | D94E I147L | | | | | |
| t818248 | Library Chimera; I86A | t524816 | 88500.44 | 24359.6 | 15341.923 | 927.223 |
| | D94E | | | | | |
| t818257 | Library Chimera; F83Y | t524816 | 101846.9 | 12313.55 | 15222.154 | 1308.820 |
| | I86A M87I F151T | | | | | |
| t818260 | Library Chimera; I86A | t524816 | 117133.9 | 18461.13 | 16851.578 | 2742.636 |
| | M87V S119A F151G | | | | | |
| t818375 | Library Chimera; I86A | t524816 | 90544.21 | 48006.38 | 16023.666 | 5181.576 |
| | S119A | | | | | |
| t818379 | Library Chimera; M43L | t524816 | 114207.3 | 9725.586 | 15945.918 | 1449.010 |
| | I86S M87V | | | | | |
| t818383 | Library Chimera; I86T | t524816 | 60717.56 | 7243.179 | 3843.038 | 498.596 |
| | S119A F151T | | | | | |
| t818388 | Library Chimera; C31F | t524816 | 109365.3 | 14620.93 | 31591.201 | 4161.662 |
| | F82G | | | | | |
| t818392 | Library Chimera; C31F | t524816 | 60222.61 | 6829.267 | 14529.480 | 1420.716 |
| | M43L I86A D94E | | | | | |
| t818408 | Library Chimera; C31F | t524816 | 100345.2 | 5771.459 | 11121.141 | 1825.115 |
| | I86V D94E | | | | | |
| t818426 | Library Chimera; I86G | t524816 | 12698.72 | 2569.555 | 30318.849 | 1879.873 |
| | F245R | | | | | |
| t818427 | Library Chimera; M43L | t524816 | 11479.94 | 2355.54 | 7636.819 | 1404.282 |
| | I86S F245R | | | | | |
| t818547 | Library Chimera; I86T | t524816 | 85918.24 | 21144.45 | 8369.546 | 2048.626 |
| | M87T | | | | | |
| t818555 | Library Chimera; C31F | t523834 | 87176.65 | 22239.93 | 154370.365 | 17278.608 |
| | M43L I86A D94E E113R | | | | | |
| | I147L F245R Q267F | | | | | |
| | Q288R | | | | | |
| t818565 | Library Chimera; C31F | t523834 | 145825.9 | 51826.35 | 128554.450 | 24145.052 |
| | M43L I86V M87V D94E | | | | | |
| | F145L 1147L F245R | | | | | |
| | L311N | | | | | |
| t818573 | Library Chimera; C31F | t523834 | 117621.2 | 13190.54 | 89583.345 | 11087.193 |
| | M43L I86S M87V D94E | | | | | |
| | F145L 1147L F245R | | | | | |
| | L311K | | | | | |
| t818606 | Library Chimera; C31F | t523834 | 105800.5 | 11058.4 | 55729.409 | 4889.757 |
| | I46C F82G D94E E113R | | | | | |
| | F145L Q267F Q288R | | | | | |
| | L311N | | | | | |
| t818614 | Library Chimera; I86G | t524816 | 96490.61 | 12045.81 | 22687.205 | 992.142 |
| | D94E | | | | | |
| t818626 | Library Chimera; F82G | t524816 | 91035.18 | 6848.612 | 15793.979 | 867.994 |
| | V122F | | | | | |
| t818726 | Library Chimera; C31F | t523834 | 80961.28 | 74786.05 | 96275.047 | 11643.525 |
| | M43L F82G E113R | | | | | |
| | F145S F245R Q267F | | | | | |
| | Q288R L311R | | | | | |
| t818728 | Library Chimera; C31F | t523834 | 92194.67 | 9002.436 | 67336.939 | 41237.172 |
| | M43L I86S D94E E113R | | | | | |
| | F245R Q267F Q288R | | | | | |
| | L311K | | | | | |
| t818733 | Library Chimera; C31F | t523834 | 134028.9 | 9997.258 | 39318.704 | 5920.956 |
| | M43V M87V D94E | | | | | |
| | E113R F145T F245R | | | | | |
| | Q267F L311N | | | | | |
| t818738 | Library Chimera; C31F | t523834 | 12124.77 | 1942.756 | 104396.291 | 6226.342 |
| | F82G I86V M87V D94E | | | | | |
| | F145L I147L F245R | | | | | |
| | L311N | | | | | |
| t818739 | Library Chimera; C31F | t524816 | 54867.49 | 56826.87 | 10515.517 | 926.653 |
| | I86A D94E F245R | | | | | |
| t818742 | Library Chimera; I46C | t523834 | 134979 | 12945.05 | 65057.841 | 7262.812 |
| | F82G D94E E113R I147L | | | | | |
| | F245R Q267F Q288R | | | | | |
| | L311N | | | | | |
| t818743 | Library Chimera; C31F | t523834 | 100259.9 | 10232.15 | 53725.636 | 6423.225 |
| | M43L I86G M87V D94E | | | | | |
| | F145L I147L F245R | | | | | |
| | L311N | | | | | |
| t818744 | Library Chimera; M43L | t523834 | 178286.7 | 33634.01 | 105939.535 | 35272.096 |
| | I86A D94E E113R 1147L | | | | | |
| | F245R Q267F Q288R | | | | | |
| | L311N | | | | | |
| t818745 | Library Chimera; M43L | t524816 | 113033.8 | 22100.38 | 9102.830 | 1025.283 |
| | I86S | | | | | |
| t818758 | Library Chimera; M43L | t523834 | 151737.1 | 20071.94 | 82124.224 | 11849.533 |
| | F82G I86V M87V D94E | | | | | |
| | F145L I147L F245R | | | | | |
| | L311N | | | | | |
| t818759 | Library Chimera; C31F | t524816 | 85907.68 | 4676.471 | 12100.114 | 1286.633 |
| | I86V M87V | | | | | |
| t818763 | Library Chimera: I86G | t524816 | 85954.37 | 5347.524 | 16587.503 | 1649.829 |
| | V122S | | | | | |
| t818767 | Library Chimera; C31F | t523834 | 133839.5 | 20240.97 | 86377.390 | 9845.302 |
| | M43V F82G D94E | | | | | |
| | E113R F145S F245R | | | | | |
| | Q288R L311R | | | | | |
| t818770 | Library Chimera; C31F | t523834 | 170289.3 | 15901.92 | 95481.050 | 7697.973 |
| | M43L F82G D94E E113R | | | | | |
| | F145T F245R Q267F | | | | | |
| | L311N | | | | | |
| t818772 | Library Chimera; F83Y | t524816 | 146497 | 55243.54 | 9870.588 | 1939.730 |
| | I86T M87V | | | | | |
| t818781 | Library Chimera; I86G | t524816 | 58488.96 | 60757 | 12716.431 | 201.102 |
| | M87I | | | | | |
| t818786 | Library Chimera; C31F | t524816 | 93999.79 | 14635.79 | 43201.330 | 6680.789 |
| | F82G D94E I147L | | | | | |
| t818801 | Library Chimera; F83Y | t524816 | 137288.7 | 7852.222 | 19269.023 | 2157.093 |
| | I86S M87I F151T | | | | | |
| t818804 | Library Chimera; I86A | t524816 | 85313.6 | 12665.81 | 11171.684 | 1479.019 |
| | V122S | | | | | |
| t818805 | Library Chimera; I86T | t524816 | 152249.5 | 21654.77 | 5986.563 | 627.624 |
| | S119A | | | | | |
| t818806 | Library Chimera; C31F | t523834 | 89754.48 | 8341.692 | 50259.419 | 43670.467 |
| | I46C I86A D94E E113R | | | | | |
| | I147L Q267F Q288R | | | | | |
| | L311N | | | | | |
| t818810 | Library Chimera; C31F | t523834 | 145327.7 | 52449.11 | 60175.267 | 9573.993 |
| | I46C I86S D94E E113R | | | | | |
| | I147L F245R Q288R | | | | | |
| | L311R | | | | | |
| t818836 | Library Chimera; C31F | t523834 | 117405.2 | 14389.15 | 45597.093 | 4833.547 |
| | I46C M87V D94E E113R | | | | | |
| | F145T F245R Q288R | | | | | |
| | L311R | | | | | |
| t818843 | Library Chimera; C31F | t523834 | 112285.1 | 23074.45 | 87794.850 | 4324.295 |
| | F82G I86V M87V D94E | | | | | |
| | F145L I147L F245R | | | | | |
| | L311R | | | | | |
| t818844 | Library Chimera; M43L | t524816 | 103534.1 | 29134.34 | 17960.347 | 2264.724 |
| | I147L F245R | | | | | |
| t818877 | Library Chimera; F83Y | t524816 | 134547.1 | 57137.76 | 6444.313 | 2470.421 |
| | I86A M87T F151T | | | | | |
| t818880 | Library Chimera; I86A | t524816 | 136959.1 | 54340.33 | 20912.700 | 2369.053 |
| | M87V I147L | | | | | |
| t818893 | Library Chimera: I86V | t524816 | 113641.7 | 28978.84 | 11482.957 | 1630.415 |
| | M87T S119A | | | | | |
| t818902 | Library Chimera; F83Y | t524816 | 86845 | 5214.612 | 15234.672 | 2019.525 |
| | I86G F151T | | | | | |
| t81891 | Library Chimera; C31F | t524816 | 134217.9 | 20833.8 | 8572.731 | 1660.680 |
| | I86V D94E F245R | | | | | |
| t818922 | Library Chimera; F82G | t524816 | 130625.1 | 60046.68 | 17853.565 | 1816.667 |
| | F245R | | | | | |
| t818975 | Library Chimera; C31F | t523834 | 145649 | 10805.58 | 80375.540 | 11429.056 |
| | M43L F82G I86V D94E | | | | | |
| | F145L 1147L F245R | | | | | |
| | L311N | | | | | |
| t818980 | Library Chimera: F82G | t524816 | 103590.1 | 15074.63 | 46502.938 | 16492.434 |
| | D94E I147L | | | | | |
| t818982 | Library Chimera; C31F | t523834 | 109349.8 | 35122.57 | 74404.043 | 10322.270 |
| | M43L I86S M87V D94E | | | | | |
| | F145L 1147L F245R | | | | | |
| | L311R | | | | | |
| t818989 | Library Chimera; C31F | t523834 | 8701.955 | 1934.449 | 88733.262 | 21711.018 |
| | M43L I86G D94E E113R | | | | | |
| | F145S Q267F Q288R | | | | | |
| | L311N | | | | | |
| t819008 | Library Chimera; I86G | t524816 | 105827.3 | 3458.263 | 19807.781 | 640.441 |
| | D94E I147L F245R | | | | | |
| t819030 | Library Chimera; C31F | t523834 | 90598.75 | 6558.066 | 88458.076 | 19104.811 |
| | M43L I86S M87V D94E | | | | | |
| | F145L I147L F245R | | | | | |
| | L311N | | | | | |
| t819037 | Library Chimera; F83Y | t524816 | 121600 | 42042.81 | 14336.611 | 2896.208 |
| | I86S M87V F151T | | | | | |
| t819066 | Library Chimera; I86V | t524816 | 90480.64 | 3791.432 | 10364.688 | 695.025 |
| | M87I S119A F151T | | | | | |
| t819073 | Library Chimera; M43L | t524816 | 93001.93 | 6946.516 | 20567.543 | 2748.245 |
| | I86G D94E F245R | | | | | |
| t819074 | Library Chimera; F82G | t524816 | 80104.1 | 10936.4 | 14410.497 | 1834.672 |
| | I86T V122F | | | | | |
| t819122 | Library Chimera; C31F | t523834 | 96685.34 | 111703.7 | 62813.484 | 43965.335 |
| | M87V D94E E113R | | | | | |
| | F145T F245W Q267F | | | | | |
| | Q288R L311K | | | | | |
| t819126 | Library Chimera; C31F | t523834 | 37748.66 | 43742.31 | 109992.287 | 19110.958 |
| | M43V D94E E113R | | | | | |
| | I147L F245W Q267F | | | | | |
| | Q288R L311R | | | | | |
| t819132 | Library Chimera; M43L | t523834 | 200736.2 | 107694.8 | 129066.626 | 23068.776 |
| | F82G D94E E113R I147L | | | | | |
| | F245R Q267F Q288R | | | | | |
| | L311K | | | | | |
| t819161 | Library Chimera; C31F | t524816 | 202538 | 112038.9 | 10264.084 | 3433.508 |
| | M43L I86S D94E | | | | | |
| t819169 | Library Chimera; C31F | t524816 | 93841.47 | 7649.135 | 42149.871 | 4422.194 |
| | F82G I147L | | | | | |
| t819172 | Library Chimera; C31F | t524816 | 94967.7 | 15345.72 | 35702.271 | 3062.617 |
| | F82G D94E | | | | | |
| t819173 | Library Chimera; C31F | t524816 | 149775.2 | 50333.67 | 7368.217 | 1854.777 |
| | M43L D94E F245R | | | | | |
| t819179 | Library Chimera; I86A | t524816 | 93595.48 | 6178.192 | 25443.790 | 3084.082 |
| | M87V | | | | | |
| t819193 | Library Chimera; I86T | t524816 | 78764.49 | 6879.207 | 4262.752 | 476.775 |
| | M87V F151G | | | | | |
| t819225 | Library Chimera; C31F | t524816 | 80286.85 | 7944.894 | 17149.563 | 4138.798 |
| | I86V M87V I147L | | | | | |
| t819336 | Library Chimera; C31F | t523834 | 96293.33 | 22287.83 | 32895.116 | 13535.689 |
| | I46C I86S D94E F145S | | | | | |
| | F245W Q267F Q288R | | | | | |
| | L311R | | | | | |
| t819343 | Library Chimera; C31F | t523834 | 159495 | 26555.01 | 58263.719 | 6555.215 |
| | I46C I86A D94E I147L | | | | | |
| | F245R Q267F Q288R | | | | | |
| | L311K | | | | | |
| t819372 | Library Chimera; C31F | t523834 | 159729.9 | 23659.25 | 84059.141 | 10331.573 |
| | M43V M87V D94E | | | | | |
| | E113R F145S Q267F | | | | | |
| | Q288R L311R | | | | | |
| t819375 | Library Chimera; I86V | t524816 | 100660.6 | 20554.36 | 13991.775 | 2219.405 |
| | D94E I147L F245R | | | | | |

All strains tested in the Gen 4 library produced more CBGA in the presence of olivetolic acid or more CBGVA in the presence of divaric acid than strain t827885, except for the following strains, as shown in Table 11: t818015, t818067, t818140, t818198, t818206, t818208, t818383, t818392, t818426, t818427, t818739, t818781, and t819126 for CBGA, and t818140, t818383, t818805, t818877, and t819193 for CBGVA.

Some strains, e.g., t818140 (including amino acid substitutions 186T, M87I and F151T) and t818383 (including amino acid substitutions 186T, S119A and F151T) produced lower titers of both CBGA and CBGVA.

Some strains, e.g., t819126 (including amino acid substitutions C31F, M43V. D94E, E113R, I147L, F245W, Q267F, Q288R and L311R), t818738 (including amino acid substitutions C31F, F82G, I86V, M87V, D94E, F145L, I147L, F245R and L311N), t818208 (including amino acid substitutions C31F, M43L, I86A, D94E, E113R, F145S, F245R, Q267F and L311K), t818206 (including amino acid substitutions C31F, I46C, 186A, E113R, I147L, F245W, Q267F, Q288R and L311N), t818989 (including amino acid substitutions C31F, M43L, I86G, D94E, E113R, F145S, Q267F, Q288R and L311N), t818426 (including amino acid substitutions I86G and F245R) and t818392 (including amino acid substitutions C31F, M43L, I86A and D94E) produced a decreased amount of CBGA and an increased amount of CBGVA, while other strains, e.g., t818805 (including amino acid substitutions 186T and S119A) and t818877 (including amino acid substitutions F83Y, I86A, M87T and F151T) produced a decreased amount of CBGVA and an increased amount of CBGA, suggesting that some substitutions may alter substrate/product specificity.

24 strains (21%) demonstrated CBGA titers greater than two-fold higher than that produced by strain t827885 when cultured in the presence of olivetolic acid, whereas 83 strains (74%) demonstrated CBGVA titers greater than two-fold higher than that produced by strain t827885 in the presence of divaric acid.

The following strains produced both CBGA titers and CBGVA titers greater than two-fold higher than strain t827885: (1) strain t817962, which was based on the chimeric PT sequence within strain t523834 (SEQ ID NO: 114) described in Examples 1 and 2, and further contained C31F, M43V, M87V, D94E, E113R, F245R, Q267F, Q288R, and L311N substitutions; (2) strain t817996, which was based on the chimeric PT sequence within strain t523834 (SEQ ID NO: 114) described in Examples 1 and 2, and further contained C31F, I86G, D94E, E113R, F145T, F245W, Q267F, Q288R, and L311N substitutions; (3) strain t818014, which was based on the chimeric PT sequence within strain t524816 (SEQ ID NO: 116) described in Examples 1 and 2, and further contained C31F, I86G, D94E, E113R, F145T, F245W, Q267F, Q288R, and L311N substitutions; (4) strain t818565, which was based on the chimeric PT sequence within strain t523834 (SEQ ID NO: 114) described in Examples 1 and 2, and further contained C31F, M43L, I86V, M87V, D94E, F145L, I147L, F245R, and L311N substitutions; (5) strain t818733, which was based on the chimeric PT sequence within strain t523834 (SEQ ID NO: 114) described in Examples 1 and 2, and further contained C31F, M43V, M87V, D94E, E113R, F145T, F245R, Q267F, and L311N substitutions; (6) strain t818744, which was based on the chimeric PT sequence within strain t523834 (SEQ ID NO: 114) described in Examples 1 and 2, and further contained M43L, I86A, D94E, E113R, I147L, F245R, Q267F, Q288R, and L311N substitutions; (7) strain t818758, which was based on the chimeric PT sequences within strain t523834 (SEQ ID NO: 114) described in Examples 1 and 2, and further contained M43L, F82G, I86V, M87V, D94E, F145L, I147L, F245R, and L311N substitutions; (8) strain t818767, which was based on the chimeric PT sequence within strain t523834 (SEQ ID NO: 114) described in Examples 1 and 2, and further contained C311F, M43V. F82G, D94E, E113R, F145S, F245R, Q288R, and L311R substitutions; (9) strain t818770, which was based on the chimeric PT sequence within strain t523834 (SEQ ID NO: 114) described in Examples 1 and 2, and further contained C31F, M43L, F82G, D94E, E113R, F145T, F245R, Q267F, and L311N substitutions; (10) strain t818801, which was based on the chimeric PT sequence within strain t524816 (SEQ ID NO: 116) described in Examples 1 and 2, and further contained F83Y, I86S, M87I, and F151T substitutions; (11) strain t818810, which was based on the chimeric PT sequence within strain t523834 (SEQ ID NO: 114) described in Examples 1 and 2, and further contained C31 F, I46C, I86S, D94E, E113R, I147L, F245R, Q288R, and L311R substitutions; (12) strain t818880, which was based on the chimeric PT sequence within strain t524816 (SEQ ID NO: 116) described in Examples 1 and 2, and further contained 186A, M87V, and I147L substitutions; (13) strain t818742, which was based on the chimeric PT sequence within strain t523834 (SEQ ID NO: 114) described in Examples 1 and 2, and further contained I46C, F82G, D94E, E113R, I147L, F245R, Q267F, Q288R and L311N substitutions; (14) strain t818922, which was based on the chimeric PT sequence within strain t524816 (SEQ ID NO: 116) described in Examples 1 and 2, and further contained F82G and F245R substitutions; (15) strain t818975, which was based on the chimeric PT sequence within strain t523834 (SEQ ID NO: 114) described in Examples 1 and 2, and further contained C31F, M43L, F82G, I86V, D94E, F145L, I147L, F245R and L311N substitutions; (16) strain t819132, which was based on the chimeric PT sequence within strain t523834 (SEQ ID NO: 114) described in Examples 1 and 2, and further contained M43L, F82G, D94E, E113R, I147L, F245R, Q267F, Q288R and L311K substitutions; (17) strain t819343, which was based on the chimeric PT sequence within strain t523834 (SEQ ID NO: 114) described in Examples 1 and 2, and further contained C31F, I46C, I86A, D94E, I147L, F245R, Q267F, Q288R and L311K substitutions; and (18) strain t819372, which was based on the chimeric PT sequence within strain t523834 (SEQ ID NO: 114) described in Examples 1 and 2, and further contained C31F, M43V, M87V, D94E, E113R, F145S, Q267F, Q288R, and L311R substitutions. Overall, variants of SEQ ID NO: 114, which is a chimera of CsPT1 and CsPT4 produced higher CBGVA titers than variants of SEQ ID NO: 116, which is a chimera of CsPT4 and CsPT7 (FIG. 15B). Sequence information for strains described in this Example is provided in Table 19.

### Example 8: Functional Expression of Additional Chimeric PTs

To further improve the CBGA titer of chimeric PTs, several of the top CBGA and/or CBGVA producing strains from the Gen 4 library described in Example 7 were selected. Additional point mutations were introduced into the chimeric PTs expressed in these strains to generate a Gen 5 library. The strains selected from the Gen 4 library were: strain t818980 (corresponding to a CsPT4-CsPT7 chimera based on parent chimera strain t524816) and strains t819132, t818744, t818565, t818555, and t817954 (corresponding to CsPT1-CsPT4 chimeras based on parent chimera strain t523834). The number of additional mutations applied to the Gen 4 templates to produce the Gen 5 PT variants ranged from 1 to 16 point mutations. The modified chimeric PTs were screened in a Gen 5 library.

Protein sequences were recoded in silico for expression in S. cerevisiae and synthesized in the replicative yeast expression vector shown in FIG. 8. Each chimeric PT expression construct was transformed into an S. cerevisiae CEN.PK strain that was engineered to overproduce GPP. Strain t819140, comprising a fluorescent protein (RFP), was included in the library as a negative control. Strain t818980, expressing one of the best-performing CsPT4-CsPT7 chimeras in the Gen 4 library, and strain t819132, expressing one of the best-performing CsPT1-CsPT4 chimeras in the Gen 4 library, were used as positive controls and for establishing hit ranking.

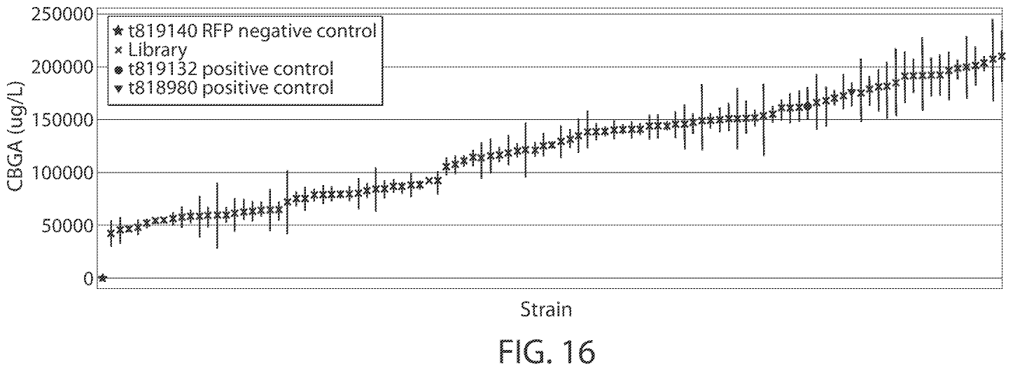

The Gen 5 library was assayed for activity in a primary screen using the same assay described in Example 7. 100 chimeric PT variants were elevated to a secondary screen to verify their CBGAS activity and to further quantify the production of CBGA. Table 12 and FIG. 16 show the results of the Gen 5 library screen. Sequences of the chimeric PTs are provided in Table 20.

As shown in Table 12, the following point mutations were tested in PT chimeras based on parent chimera strain t524816 (corresponding to a CsPT4-CsPT7 chimera): V39T, L62I, L68F, M75I, M75V, F82G, I86G, D94E, I117L, I140L, I140T, I147L, F151I, A152I, I172F, I172L, G177L, F190V, M196I, M196L, P199A, L204I, V209L, M212I, T213V, A227K, A227R, V231I, V234F, V234L, R241K, V2461, V246L, V247S, V250A, V250I, V250T, T254A, T254C, T254L, T254N, S257G, L260I, A262G, I264F, L275I, and C284W.

As also shown in Table 12, the following point mutations were tested in PT chimeras based on parent chimera strain t523834: T30A, C31F, L34F, Q35A, Q35S, Q35T, V39T, V401, M43L, M43V, S45F, S451, S45L, I46A, I46C, I46G, A47S, G49A, G49C, G49I, G49S, G52A, S63N, F72A, F72Q, F72V, A73G, V75I, P76A, S79C, S79L, F82A, F82G, A85N, I86A, I86G, I86V, M87I, M87L, M87V, D94E, D102Y, L105I, V106A, M110I, M110L, E113R, L118I, I121S, L124V, I128L, V129I, V129L, F139A, F139I, F139L, V140F, V140I, V140L, V140T, F141A, F141C, F141G, F141I, F141S, F141V, I142L, F145L, I147L, F148L, A149I, A149L, F151A, F151T, A152F, A152I, A152L, A152V, N167A, L169A, L169I, T171I, I172F, I172L, I172V, S173I, S173L, S173T, S174V, G177I, G177L, G177T, G177V, A179N, A179P, T181V, S182F, S182V, R197S, F200L, I204T, M207V, V209L, M210F, G211A, G211S, G211T, M212I, M212L, T213A, T213G, T213V, F216I, A217T, I220L, I223V, A227K, A227R, K228A, Y229F, Y229H, V231I, V234F, V234L, V234M, T236A, T236V, A240V, R241K, N242T, M243A, M2431, M243S, M243T, F245R, F245W, V246A, V246F, V2461, V246L, V247C, V247G, V250F, V250I, V250L, L252I, L256V, V257G, V257L, S258A, I264F, I264N, Q267F, S271G, S271K, S271L, L276F, L276G, L276P, A2791, L281A, F283S, C284F, C284I, C284S, C284V, C284W, I286F, Q288R, T289A, L311K, and L311N.

21 strains (21%) demonstrated CBGA titers greater than that produced by positive control strains t818980 and/or 1819132 when cultured in the presence of olivetolic acid. 21 strains (21%; 3 CsPT1-CsPT4 PT chimeras and 18 CsPT4-CsPT7 PT chimeras) in the Gen 5 library produced higher CBGA titers than strain t819132, one of the best performing CsPT1-CsPT4 PT chimeras in the Gen 4 library. 16 strains (16%; 2 CsPT1-CsPT4 PT chimeras and 14 CsPT4-CsPT7 chimeras) in the Gen 5 library produced higher CBGA titers than strain t818980, one of the best performing CsPT4-CsPT7 PT chimeras in the Gen 4 library.

The following strains produced CBGA titers 10% higher than the best Gen 4 strain t818980: (1) strain t879474, which is based on the CsPT4-CsPT7 chimeric PT sequence within strain t524816, and further contained the amino acid substitutions M75V, F82G, D94E, I147L and T254N; (2) strain t879304, which is also based on the CsPT4-CsPT7 chimeric PT sequence within strain t524816, and further contained the amino acid substitutions F82G, D94E, I140L and I147L; (3) strain t879340, which is also based on the CsPT4-CsPT7 chimeric PT sequence within strain t524816, and further contained the amino acid substitutions F82G, D94E, I147L, A227K and T254N; (4) strain t879750, which is based on the CsPT4-CsPT7 chimeric PT sequence within strain t524816, and further contained the amino acid substitutions L62I F82G D94E I147L; (5) strain t879685, which is based on the CsPT4-CsPT7 chimeric PT sequence within strain t524816, and further contained the amino acid substitutions L68F F82G D94E I147L; (6) strain t879725, which is based on the CsPT4-CsPT7 chimeric PT sequence within strain t524816, and further contained the amino acid substitutions F82G D94E I147L M196L; and (7) strain t879774, which is based on the CsPT4-CsPT7 chimeric PT sequence within strain t524816, and further contained the amino acid substitutions F82G D94E I147L M196I.

The following CsPT1-CsPT4 chimera variant strains produced higher CBGA titers than the best Gen 4 strain t818980; (1) strain t879592, which is based on the CsPT1-CsPT4 chimeric PT sequence within strain t523834, and further contained amino acid substitutions L34F, Q35T, M43L, G49S, I86A, D94E, D102Y, E113R, F139L, I147L, A149L, S182V, T213V, A227R, V234L, T236V, F245R, V247G, V250L, L256V, V257G, Q267F, F283S, Q288R, L311N; and (2) strain t879357, which is based on the CsPT1-CsPT4 chimera template sequence within strain t523834, and further contained the amino acid substitutions M43L, F82G, A85N, I86G, M87I, D94E, V106A, E113R, F141S, I142L. I147L, A149L, T171I, A179N, A227K, Y229H, V234L, R241K, F245R, V250F, V257L, S258A, Q267F, Q288R and L311K.

The following CsPT4-CsPT7 chimera variant strains produced higher CBGA titers than the Gen 4 strain t819132: (1) strain t879001, which is based on the CsPT4-CsPT7 chimera PT sequence within strain t523834, and further contained amino acid substitutions F82G D94E I140T I147L; (2) strain t879340, which is based on the CsPT4-CsPT7 chimera PT sequence within strain t523834, and further contained amino acid substitutions F82G D94E I147L A227K T254N; (3) strain t879474, which is based on the CsPT4-CsPT7 chimera PT sequence within strain t523834, and further contained amino acid substitutions M75V F82G D94E I147L T254N; (4) strain t879750, which is based on the CsPT4-CsPT7 chimera PT sequence within strain t523834, and further contained amino acid substitutions L62I F82G D94E I147L; (5) strain t879685, which is based on the CsPT4-CsPT7 chimera PT sequence within strain t523834, and further contained amino acid substitutions L68F F82G D94E I147L; (6) strain t879670, which is based on the CsPT4-CsPT7 chimera PT sequence within strain t523834, and further contained amino acid substitutions F82G D94E I147L I172L; (7) strain t879624, which is based on the CsPT4-CsPT7 chimera PT sequence within strain t523834, and further contained amino acid substitutions F82G D94E I147L V250I T254N; (8) strain t879758, which is based on the CsPT4-CsPT7 chimera PT sequence within strain t523834, and further contained amino acid substitutions F82G D94E I147L R241K; (9) strain t879725, which is based on the CsPT4-CsPT7 chimera PT sequence within strain t523834, and further contained amino acid substitutions F82G D94E I147L M196L; (10) strain t879768, which is based on the CsPT4-CsPT7 chimera PT sequence within strain t523834, and further contained amino acid substitutions F82G D94E I147L C284W; (11) strain t879304, which is based on the CsPT4-CsPT7 chimera PT sequence within strain t523834, and further contained amino acid substitutions F82G D94E I140L I147L; (12) strain t879151, which is based on the CsPT4-CsPT7 chimera PT sequence within strain t523834, and further contained amino acid substitutions F82G D94E I147L V250A T254N; (13) strain t879774, which is based on the CsPT4-CsPT7 chimera PT sequence within strain t523834, and further contained amino acid substitutions F82G D94E I147L M196I; (14) strain t879949, which is based on the CsPT4-CsPT7 chimera PT sequence within strain t523834, and further contained amino acid substitutions F82G D94E I147L V246I; (15) strain t879660, which is based on the CsPT4-CsPT7 chimera PT sequence within strain t523834, and further contained amino acid substitutions F82G D94E I147L T254A; (16) strain t879522, which is based on the CsPT4-CsPT7 chimera PT sequence within strain t523834, and further contained amino acid substitutions M75V F82G D94E I147L; (17) strain t879240, which is based on the CsPT4-CsPT7 chimera PT sequence within strain t523834, and further contained amino acid substitutions F82G D94E I147L T254C; and (18) strain t879205, which is based on the CsPT4-CsPT7 chimera PT sequence within strain t523834, and further contained amino acid substitutions F82G D94E I147L F190V.

Based on the data from the Gen 5 library, at least the following amino acid substitutions appeared to contribute to improving CBGA titers within the PT chimeras: F82G, D94E, I147L, T254N, I140L, and A227K. Homology modeling analysis was performed to investigate the potential effects of amino acid substitutions at these positions.

The three-dimensional conformational structure of transmembrane PT proteins corresponds to a helical bundle that includes nine transmembrane helices. Without wishing to be bound by any theory, amino acid position 82 is located on the second transmembrane helix of the nine transmembrane helical bundle of the PT structure. Specifically, position 82 is situated on the face of transmembrane helix 2 that apposes the putative enzymatic active site. The amino acid in position 82 may affect the overall helical bundle structure of the PT protein through contacts with neighboring transmembrane helix 1 and transmembrane helix 3. Transmembrane helix 1 faces the active site, so contact with transmembrane helix 1 may affect active site shape. Transmembrane helix 3 does not directly participate in formation of the active site. Interaction with transmembrane helix 3 may impact overall stabilization of the protein structure and may contribute to supporting a structure that is conducive for catalysis. The substantial reduction of side chain volume achieved when a Gly (G) residue is substituted for a Phe (F) residue at position 82 may modulate the helical bundle structure of the PT protein to produce subtle changes in active site shape, and therefore improve substrate binding capabilities and catalysis.

Without wishing to be bound by any theory, amino acid position 94 is located on a short loop between transmembrane helix 2 and transmembrane helix 3 that is peripheral to the metal ions within the active site of the PT structure. The substitution from Asp (D) to Glu (E) at position 94 increases the side-chain length, which may better position the carboxylate group for favorable hydrogen bonding with neighboring polar/basic amino acids such as R97, Q162, and K228. Favorable hydrogen bonding may stabilize this loop and, in turn, act to stabilize the metal-binding site and proximal active site within the chimeric PT structure.

Without wishing to be bound by any theory, amino acid position 147 is located at the approximate midpoint of transmembrane helix 4 within the membrane. Transmembrane helix 4 is one of the transmembrane helices that form the enzyme active site. Amino acid position 147 faces outward, away from the active site, and is positioned to make contacts with neighboring transmembrane helix 2 and transmembrane helix 3 as well as to interact with lipid chains within the membrane. The substitution of the β-branched Ile (I) with Leu (L), which has a different geometric shape, may improve side chain packing of neighboring hydrophobic residues. This, in turn, may help to stabilize the interactions between transmembrane helices 2-4 and thereby improve active site shape and stability.

Without wishing to be bound by any theory, amino acid position 254 is located at the approximate midpoint of transmembrane helix 7 and faces transmembrane helix 6 and transmembrane helix 8 in a region that is distal from the active site. This area of the PT structure is not lipid-facing. Amino acids located at the interface between transmembrane helices 6-8 are overwhelmingly occupied by hydrophobic residues. However, polar amino acids T213 on transmembrane helix 6 and S277 on transmembrane helix 8 are well-positioned for forming hydrogen bonds with a polar amino acid at position 254. The substitution from Thr (T) to Asp (N) may facilitate better hydrogen bonding between the transmembrane helices 6-8 and thereby improve protein helical packing and stability.

Without wishing to be bound by any theory, amino acid position 140 is located on transmembrane helix 4 in a location that is distal to the active site. Position 140 faces outward, away from the active site, and is positioned to make contacts with neighboring transmembrane helix 3 and with hydrophobic lipid chains within the membrane. The substitution of the β-branched Ile (I) with Leu (L), which has a different geometric shape, may improve the side chain packing between position 140 and the side chains of neighboring hydrophobic residues. This may help stabilize the interactions between transmembrane helices 2 and 4 and thereby improve active site shape and stability.

Without wishing to be bound by any theory, amino acid position 227 is located on a short helix that connects transmembrane helix 6 with transmembrane helix 7. This short helix may be important for positioning metal ions within the active site. The helix contains D222 and D226, either of which may chelate one of the divalent metals of the di-metal binding site. Position 227 lies on the apposing side of the helix and faces away from the active site. The substitution of alanine for the flexible, positively charged side chain of lysine may provide additional hydrogen bonding interactions with neighboring charged and polar side chains such as E224 and T236. Such interactions could help to stabilize the local structure and thereby improve metal ion coordination by the short helix and active site shape and stability within the chimeric PT structure.

TABLE 12
Activity data of Gen 5 library members in S. cerevisiae

| | | | Average | Standard |
|---|---|---|---|---|
| Strain | Strain type, PT type | Parent chimera | CBGA | Deviation CBGA |
| ID | Mutation (if applicable) | strain | [μg/L] | [μg/L] |
| t818980 | Positive control Chimeric PT | t524816 | 175694.494 | 22582.501 |
| t819132 | Positive control Chimeric PT | t523834 | 163210.436 | 28489.014 |
| t819140 | Negative control | N/A | 0.000 | 0.000 |
| t880043 | Library Chimera; T30A L34F | t523834 | 170493.541 | 8410.356 |
| | M43L S45L I86A M87I D94E | | | |
| | E113R V140I I142L I147L | | | |
| | A149L S182V G211S M212I | | | |
| | V234L R241K F245R V250L | | | |
| | L256V S258A I264N Q267F | | | |
| | Q288R L311N | | | |
| t879667 | Library Chimera; F82G D94E | t524816 | 152277.473 | 7184.982 |
| | I147L A227R | | | |
| t879993 | Library Chimera; V39T F82G | t524816 | 126186.758 | 3921.598 |
| | D94E I147L | | | |
| t879001 | Library Chimera; F82G D94E | t524816 | 173439.736 | 20599.208 |
| | I140T I147L | | | |
| t879539 | Library Chimera; F82G D94E | t524816 | 144431.229 | 13241.256 |
| | I147L V246L | | | |
| t879989 | Library Chimera; F82G D94E | t524816 | 144533.233 | 3931.854 |
| | I147L A262G | | | |
| t879340 | Library Chimera; F82G D94E | t524816 | 201914.203 | 20657.612 |
| | I147L A227K T254N | | | |
| t880030 | Library Chimera; F82G D94E | t524816 | 141342.713 | 8896.624 |
| | I147L L204I | | | |
| t879474 | Library Chimera; M75V F82G | t524816 | 211057.531 | 28129.096 |
| | D94E I147L T254N | | | |
| t879791 | Library Chimera; F82G D94E | t524816 | 147559.694 | 11212.613 |
| | I147L T254L | | | |
| t879562 | Library Chimera; F82G D94E | t524816 | 150333.559 | 13526.336 |
| | I147L T254N | | | |
| t880029 | Library Chimera; T30A M43L | t523834 | 141702.729 | 6421.816 |
| | I86A D94E E113R I121S | | | |
| | V129I F141S I147L F148L | | | |
| | A149L T171I S173L S182V | | | |
| | G211S M212L T213V R241K | | | |
| | F245R V247C V250L Q267F | | | |
| | I286F Q288R L311N | | | |
| t879750 | Library Chimera; L62I F82G | t524816 | 196738.135 | 27490.758 |
| | D94E I147L | | | |
| t879685 | Library Chimera; L68F F82G | t524816 | 204562.128 | 7265.318 |
| | D94E I147L | | | |
| t879512 | Library Chimera; F82G D94E | t524816 | 140821.971 | 5063.581 |
| | I147L T254N S257G | | | |
| t879297 | Library Chimera; F82G D94E | t524816 | 151858.309 | 17158.912 |
| | I147L I172F | | | |
| t879827 | Library Chimera; F82G D94E | t524816 | 138789.773 | 8532.164 |
| | I147L T213V T254N | | | |
| t879670 | Library Chimera; F82G D94E | t524816 | 192735.876 | 22103.663 |
| | I147L I172L | | | |
| t879624 | Library Chimera; F82G D94E | t524816 | 166374.236 | 28897.902 |
| | I147L V250I T254N | | | |
| t879758 | Library Chimera; F82G D94E | t524816 | 181965.274 | 23613.108 |
| | I147L R241K | | | |
| t879503 | Library Chimera; F82G D94E | t524816 | 121978.530 | 6827.481 |
| | I147L L275I | | | |
| t879068 | Library Chimera; F82G D94E | t524816 | 146099.097 | 22420.368 |
| | I147L V250T T254N | | | |
| t879840 | Library Chimera; F82G D94E | t524816 | 149209.493 | 33824.100 |
| | I147L L260I | | | |
| t879356 | Library Chimera; F82G D94E | t524816 | 145873.036 | 14741.449 |
| | I147L P199A | | | |
| t879725 | Library Chimera; F82G D94E | t524816 | 199132.512 | 11106.953 |
| | I147L M196L | | | |
| t879071 | Library Chimera; F82G D94E | t524816 | 161595.561 | 15585.876 |
| | I117L I147 | | | |
| t879768 | Library Chimera; F82G D94E | t524816 | 167873.094 | 24066.282 |
| | I147L C284W | | | |
| t879836 | Library Chimera; F82G D94E | t524816 | 129222.358 | 16372.137 |
| | I147L V250I | | | |
| t880054 | Library Chimera, F82G D94E | t524816 | 153869.073 | 36388.819 |
| | I147L A227K | | | |
| t879626 | Library Chimera; F82G D94E | t524816 | 161554.177 | 11284.066 |
| | I147L F151I | | | |
| t879983 | Library Chimera; F82G D94E | t524816 | 114331.302 | 8822.396 |
| | I147L V209L | | | |
| t879726 | Library Chimera; F82G D94E | t524816 | 121721.151 | 29724.035 |
| | I147L A152I | | | |
| t879529 | Library Chimera; F82G D94E | t524816 | 151369.222 | 32685.240 |
| | I147L S257G | | | |
| t879304 | Library Chimera; F82G D94E | t524816 | 200335.440 | 35457.308 |
| | I140L I147L | | | |
| t879708 | Library Chimera; F82G D94B | t524816 | 150966.550 | 14695.071 |
| | I147L I264F | | | |
| t879602 | Library Chimera; F82G D94E | t524816 | 144242.121 | 13314.593 |
| | I147L V247S | | | |
| t879151 | Library Chimera; F82G D94E | t524816 | 185466.132 | 37760.532 |
| | I147L V250A T254N | | | |
| t879382 | Library Chimera; F82G D94E | t524816 | 125149.387 | 11121.786 |
| | I147L V234L | | | |
| t879774 | Library Chimera; F82G D94E | t524816 | 208158.959 | 43196.788 |
| | I147L M196I | | | |
| t879650 | Library Chimera; F82G D94E | t524816 | 139292.023 | 4545.558 |
| | I147L V231I | | | |
| t879418 | Library Chimera; F82G D94E | t524816 | 162123.189 | 17784.632 |
| | I147L V234F | | | |
| t879399 | Library Chimera; F82G I86G | t524816 | 111168.478 | 6471.574 |
| | D94E I147L | | | |
| t879949 | Library Chimera; F82G D94E | t524816 | 192476.165 | 43008.487 |
| | I147L V246I | | | |
| t879660 | Library Chimera; F82G D94E | t524816 | 179503.103 | 15093.285 |
| | I147L T254A | | | |
| t879522 | Library Chimera; M75V F82G | t524816 | 192820.446 | 20860.992 |
| | D94E I147L | | | |
| t879193 | Library Chimera; F82G D94E | t524816 | 135008.127 | 18683.450 |
| | I147L T213V | | | |
| t879977 | Library Chimera; L34F M43L | t523834 | 87712.533 | 13657.490 |
| | I46A G49S I86A M87I D94E | | | |
| | M110L E113R V140I F141S | | | |
| | I147L A149L I172F G177T | | | |
| | A179P A227K V234L F245R | | | |
| | L256V S258A Q267F L276F | | | |
| | Q288R L311N | | | |
| t879357 | Library Chimera; M43L F82G | t523834 | 192417.151 | 19315.025 |
| | A85N I86G M87I D94E | | | |
| | V106A E113R F141S I142L | | | |
| | I147L A149L T171I A179N | | | |
| | A227K Y229H V234L R241K | | | |
| | F245R V250F V257L S258A | | | |
| | Q267F Q288R L311K | | | |
| t879819 | Library Chimera; M751 F82G | t524816 | 149529.245 | 8652.322 |
| | D94E I147L | | | |
| t879233 | Library Chimera; F82G D94E | t524816 | 131379.102 | 10439.037 |
| | I147L V234L T254N | | | |
| t879240 | Library Chimera; F82G D94E | t524816 | 191051.721 | 30083.686 |
| | I147L T254C | | | |
| t879205 | Library Chimera; F82G D94E | t524816 | 182552.424 | 32995.758 |
| | I147L F190V | | | |
| t879397 | Library Chimera; F82G D94E | t524816 | 107599.193 | 10415.264 |
| | I147L G177L T254N | | | |
| t879014 | Library Chimera; F82G D94E | t524816 | 138417.704 | 20454.970 |
| | I147L M212I | | | |
| t879150 | Library Chimera; T30A V39T | t523834 | 155619.152 | 7756.253 |
| | M43L F82G A85N M87I | | | |
| | D94E E113R V129I V140F | | | |
| | I142L I147L A149L T171I | | | |
| | G211T A227K V234L M243T | | | |
| | F245R V246I V247C V250L | | | |
| | Q267F Q288R L311K | | | |
| t879592 | Library Chimera; L34F Q35T | t523834 | 175801.493 | 36415.331 |
| | M43L G49S I86A D94E | | | |
| | D102Y E113R F139L I147L | | | |
| | A149L S182V T213V A227R | | | |
| | V234L T236V F245R V247G | | | |
| | V250L L256V V257G Q267F | | | |
| | F283S Q288R L311N | | | |
| t879184 | Library Chimera; T30A C31F | t523834 | 116598.214 | 8498.857 |
| | M43V I46G G49A I86G | | | |
| | M87V D94E L105I E113R | | | |
| | F139L F141I F145L L169A | | | |
| | T171I S173L S182V M210F | | | |
| | T213A V234L F245W V250L | | | |
| | S258A Q267F Q288R | | | |
| t879918 | Library Chimera; T30A C31F | t523834 | 78895.027 | 8685.767 |
| | M43L S45I A73G V75I I86A | | | |
| | M87L D94E M110L E113R | | | |
| | I142L I147L G177V M212L | | | |
| | K228A V234L N242T F245R | | | |
| | V247C V250L V257L S258A | | | |
| | Q267F Q288R | | | |
| t879813 | Library Chimera; T30A M43L | t523834 | 118559.394 | 16644.042 |
| | G52A I86A D94E E113R | | | |
| | I121S V129L F141S I147L | | | |
| | T171I S182V G211A A227R | | | |
| | V234F F245R V247C V250L | | | |
| | V257G S258A Q267F S271K | | | |
| | C284W Q288R L311N | | | |
| t879338 | Library Chimera; M43L F82G | t523834 | 92127.782 | 1620.022 |
| | A85N I86G M87I D94E | | | |
| | E113R F141I I147L T171I | | | |
| | G177T S182V R197S M212L | | | |
| | T213A A227K V234L M243S | | | |
| | F245R V250L Q267F L276P | | | |
| | F283S Q288R L311K | | | |
| t879042 | Library Chimera; M43L I46G | t523834 | 140688.126 | 11763.228 |
| | F82G M87I D94E V106A | | | |
| | M110L E113R V129L I147L | | | |
| | A149I T171I A179N M212L | | | |
| | Y229F V234L R241K F245R | | | |
| | V246I V247C V250L S258A | | | |
| | Q267F Q288R L311K | | | |
| t879155 | Library Chimera; T30A M43L | t523834 | 88132.230 | 4692.516 |
| | G49S I86A M87I D94E | | | |
| | D102Y V106A E113R I121S | | | |
| | V129L V140L I147L A179N | | | |
| | R197S T213V A227K M243T | | | |
| | F245R V246L V247C V250L | | | |
| | Q267F Q288R L311N | | | |
| t879940 | Library Chimera; C31F M43L | t523834 | 63125.890 | 11123.768 |
| | I86A M87I D94E V106A | | | |
| | E113R I121S V129I F139L | | | |
| | F141C I147L T171I S173I | | | |
| | A179P M212I A227K V234L | | | |
| | R241K F245R V246I V247C | | | |
| | Q267F C284F Q288R | | | |
| t879345 | Library Chimera; M43L S45L | t523834 | 120438.544 | 6780.953 |
| | I86A D94E V106A E113R | | | |
| | F139A I147L F148L A149L | | | |
| | T171I T213V I223V V234L | | | |
| | T236V A240V R241K M243T | | | |
| | F245R V246L V247C V257G | | | |
| | Q267F Q288R L311N | | | |
| t879857 | Library Chimera; T30A C31F | t523834 | 114389.228 | 21090.908 |
| | M43L I86V M87V D94E | | | |
| | F139L F141S F145L I147L | | | |
| | F148L T171I G177I T181V | | | |
| | R197S M207V G211S V234L | | | |
| | F245R V246L V247C V250L | | | |
| | L276G C284S L311N | | | |
| t879788 | Library Chimera; T30A C31F | t523834 | 79227.020 | 3851.553 |
| | Q3ST M43L S45L I46C V75I | | | |
| | I86A D94E V106A E113R | | | |
| | V129I F139I I147L A149L | | | |
| | F151A I172F T213A V234L | | | |
| | F245R V246I V250L S258A | | | |
| | Q267F Q288R | | | |
| t879606 | Library Chimera; L34F M43L | t523834 | 79951.765 | 8436.423 |
| | S45L F82G I86G D94E E113R | | | |
| | F139L V140T I147L G177T | | | |
| | A179N S182F M212I A227K | | | |
| | M243T F245R V246L V247C | | | |
| | V250L S258A Q267F L276F | | | |
| | Q288R L311K | | | |
| t879579 | Library Chimera; C31F V39T | t523834 | 57679.345 | 13043.316 |
| | M43V I46G F82A A85N | | | |
| | M87V D94E E113R V129L | | | |
| | F139L F145L A149L I172L | | | |
| | G177I M210F A227K F245W | | | |
| | V246L V247C V257G S258A | | | |
| | Q267F C284W Q288R | | | |
| t879488 | Library Chimera; T30A C31F | t523834 | 78734.692 | 9536.254 |
| | V39T M43V S45L I46G G49C | | | |
| | F72A S79L M87V D94E | | | |
| | E113R I121S I128L F145L | | | |
| | S182V A227K Y229F V234L | | | |
| | F245W V247C V250L Q267F | | | |
| | F283S Q288R | | | |
| t879191 | Library Chimera; Q35A M43L | t523834 | 86979.624 | 5693.817 |
| | V75I I86A M87I D94E L105I | | | |
| | E113R L118I F139L V140L | | | |
| | I142L I147L F151A T213V | | | |
| | V234M M243I F245R L256V | | | |
| | V257G S258A I264F Q267F | | | |
| | Q288R L311N | | | |
| t879379 | Library Chimera; C31F L34F | t523834 | 82667.992 | 7613.952 |
| | Q35S V39T M43L A47S | | | |
| | F72V A85N I86V M87V | | | |
| | D94E V129L F141S F145L | | | |
| | I147L A152V T171I S182V | | | |
| | M207V G211S T213V A227K | | | |
| | F245R V250L L311N | | | |
| t879066 | Library Chimera; T30A C31F | t523834 | 75354.780 | 12510.620 |
| | M43L I86A D94E M110I | | | |
| | E113R L118I I121S V129I | | | |
| | I147L F151A M212I T213G | | | |
| | I223V V234L A240V R241K | | | |
| | N242T F245R V246L S258A | | | |
| | Q267F L281A Q288R | | | |
| t879874 | Library Chimera; T30A C31F | t523834 | 61341.164 | 16431.817 |
| | V39T M43L S79C I86V | | | |
| | M87V D94E V106A F141S | | | |
| | F145L I147L A149L T171I | | | |
| | S174V M210F M212L T213V | | | |
| | F245R V246L V247C V250L | | | |
| | V257G S258A L311N | | | |
| t879638 | Library Chimera; C31F M43V | t523834 | 84304.795 | 24914.102 |
| | S45L G49I F72V F82A M87V | | | |
| | D94E V106A E113R V129L | | | |
| | F141V I142L F145L G211S | | | |
| | A227K V234L R241K F245W | | | |
| | L256V S258A Q267F A279I | | | |
| | C284W Q288R | | | |
| t879848 | Library Chimera; C31F M43L | t523834 | 64360.787 | 22050.354 |
| | S45F V75I I86V M87V D94E | | | |
| | L105I V106A F139L V140T | | | |
| | F141V F145L I147L A152I | | | |
| | T171I G177L A179P M207V | | | |
| | A227R V234L F245R V247G | | | |
| | S258A L311N | | | |
| t879358 | Library Chimera; C31F M43V | t523834 | 78679.866 | 5682.462 |
| | M87V D94E M110L E113R | | | |
| | F139L V140T F141S F145L | | | |
| | L169I I172L G177L A227K | | | |
| | V234F F245W V246I V247C | | | |
| | V250L L252I L256V V257G | | | |
| | S258A Q267F Q288R | | | |
| t879809 | Library Chimera; T30A C31F | t523834 | 92187.080 | 11927.825 |
| | M43L I86A M87I D94E | | | |
| | E113R V129L I142L I147L | | | |
| | A152F T171I G177L M210F | | | |
| | G211S T213V A227K V234L | | | |
| | R241K F245R V246I S258A | | | |
| | Q267F C284W Q288R | | | |
| t879226 | Library Chimera; T30A C31F | t523834 | 54910.466 | 3519.672 |
| | V40I M43L S45L I46G G49S | | | |
| | I86A D94E V106A M110L | | | |
| | E113R V129I I147L A149L | | | |
| | T171I G211S M212L T213V | | | |
| | A227R V234L F245R V250L | | | |
| | Q267F Q288R | | | |
| t879141 | Library Chimera; T30A M43L | t523834 | 84415.083 | 10229.372 |
| | S79L A85N I86A D94E | | | |
| | E113R V129I F139L I147L | | | |
| | A149L T171I S182V G211S | | | |
| | Y229F A240V R241K M243T | | | |
| | F245R V247C V250L Q267F | | | |
| | C284I Q288R L311N | | | |
| t879439 | Library Chimera; C31F L34F | t523834 | 75220.691 | 6905.057 |
| | M43L I86A D94E V106A | | | |
| | E113R L118I F141C I147L | | | |
| | A149L F151A S174V S182V | | | |
| | M207V G211S A227K R241K | | | |
| | F245R V246A L256V S258A | | | |
| | Q267F C284V Q288R | | | |
| t879243 | Library Chimera; V40I M43L | t523834 | 64286.707 | 7175.765 |
| | S45L I86A M87I D94E | | | |
| | V106A E113R I147L A149L | | | |
| | F151T N167A T171I S173T | | | |
| | T213V A227K R241K M243T | | | |
| | F245R V246L V247C L256V | | | |
| | Q267F Q288R L311N | | | |
| t879134 | Library Chimera; M43L F82G | t523834 | 115617.059 | 22697.688 |
| | D94E E113R F139I F141S | | | |
| | I142L I147L A149L A179N | | | |
| | S182V M212I I223V V231I | | | |
| | V234L A240V R241K F245R | | | |
| | V247C S258A Q267F S271G | | | |
| | F283S Q288R L311K | | | |
| t879557 | Library Chimera; C31F V39T | t523834 | 58629.854 | 25074.814 |
| | M43V S45I S63N M87V | | | |
| | D94E E113R F139L V140T | | | |
| | F141V F145L A152I S182V | | | |
| | R197S I204T A227K R241K | | | |
| | F245W V246A L256V V257G | | | |
| | S258A Q267F Q288R | | | |
| t879202 | Library Chimera; M43L S45L | t523834 | 58177.285 | 7276.930 |
| | I86A M87I D94E E113R | | | |
| | V129L I142L I147L A149L | | | |
| | T171I I172V G177I A179N | | | |
| | S182V I204T M210F T213V | | | |
| | V234L R241K F245R V250L | | | |
| | Q267F Q288R L311N | | | |
| t879067 | Library Chimera; T30A C31F | t523834 | 45523.355 | 16077.836 |
| | M43L I46G I86V M87V D94E | | | |
| | M110I V129L F139L F145L | | | |
| | I147L A149L T171I G177L | | | |
| | A179N A227K Y229F R241K | | | |
| | F245R V246L V247C V250L | | | |
| | I264F L311N | | | |
| t879099 | Library Chimera; M43L S45L | t523834 | 48045.631 | 8886.190 |
| | I46A P76A I86A M87I D94E | | | |
| | V106A E113R F141C I147L | | | |
| | F151T A152I M207V M212L | | | |
| | A227R V234L T236A M243I | | | |
| | F245R V247C S258A Q267F | | | |
| | Q288R L311N | | | |
| t879286 | Library Chimera; L34F V39T | t523834 | 59673.669 | 8772.249 |
| | M43L S45L F82G I86G M87I | | | |
| | D94E V106A E113R V140F | | | |
| | I147L T171I G211S T213A | | | |
| | F216I V234L M243A F245R | | | |
| | V246L V247C Q267F C284W | | | |
| | Q288R L311K | | | |
| t878988 | Library Chimera; T30A M43L | t523834 | 80322.940 | 18108.485 |
| | S63N I86A M87I D94E | | | |
| | V106A E113R I147L F151T | | | |
| | N167A A179N S182V M212I | | | |
| | A217T V234L R241K M243T | | | |
| | F245R V246F V247C Q267F | | | |
| | C284W Q288R L311N | | | |
| t879148 | Library Chimera; M43L I46G | t523834 | 105412.794 | 9519.791 |
| | I86A M87I D94E V106A | | | |
| | M110I E113R L124V F139L | | | |
| | I147L F151A T171I G211S | | | |
| | A227K F245R V247C V250L | | | |
| | L256V S258A Q267F C284V | | | |
| | Q288R T289A L311N | | | |
| t879235 | Library Chimera; T30A M43L | t523834 | 59433.595 | 11443.790 |
| | I46G G49S I86A M87I D94E | | | |
| | M110L E113R F139L F141G | | | |
| | I147L T171I I172V S173T | | | |
| | T213A A227K V231I M243A | | | |
| | F245R V247C V250L Q267F | | | |
| | Q288R L311N | | | |
| t879422 | Library Chimera; C31F M43V | t523834 | 59558.511 | 35589.672 |
| | A47S A73G S79C M87V | | | |
| | D94E E113R V129I F141S | | | |
| | F145L A149L A152L T171I | | | |
| | A179N S182V M210F M212L | | | |
| | V234L R241K F245W V247C | | | |
| | S258A Q267F Q288R | | | |
| t879687 | Library Chimera; T30A C31F | t523834 | 52124.680 | 4466.185 |
| | M43L G49S I86A D94E | | | |
| | E113R L118I I121S V129L | | | |
| | F139I I142L I147L G177I | | | |
| | G211S A227K Y229F V234L | | | |
| | R241K F245R V246I V247C | | | |
| | V250L Q267F Q288R | | | |
| t879050 | Library Chimera; T30A M43L | t523834 | 72183.240 | 35538.907 |
| | S45L S79L F82G D94E | | | |
| | E113R V129I F141S I142L | | | |
| | I147L G177I A179N S182V | | | |
| | M212I V234F R241K F245R | | | |
| | V247C V250L S258A Q267F | | | |
| | C284W Q288R L311K | | | |
| t879913 | Library Chimera; T30A M43L | t523834 | 41890.787 | 13854.261 |
| | F82G D94E E113R V129I | | | |
| | V140T F141C I142L I147L | | | |
| | A179N S182V I204T A227R | | | |
| | V234M M243T F245R V246I | | | |
| | V247C V250L S258A Q267F | | | |
| | C284W Q288R L311K | | | |
| t879059 | Library Chimera; M43L F72Q | t523834 | 46615.362 | 2367.213 |
| | F82G M87I D94E E113R | | | |
| | I147L A149L N167A T171I | | | |
| | S173L S182V F200L M212I | | | |
| | V231I V234F M243T F245R | | | |
| | V246I V247C V250L S258A | | | |
| | Q267F Q288R L311K | | | |
| t879037 | Library Chimera; T30A V40I | t523834 | 64424.527 | 10449.887 |
| | M43L F82G M87I D94E | | | |
| | E113R I147L T171I S173L | | | |
| | G177T V209L M212L Y229F | | | |
| | V234L R241K F245R V246I | | | |
| | V247C V250I S258A Q267F | | | |
| | C284W Q288R L311K | | | |
| t879885 | Library Chimera; T30A M43L | t523834 | 62635.148 | 11681.814 |
| | S45L I86A D94E E113R | | | |
| | V129L F141A I142L I147L | | | |
| | A149L F151A N167A T17LI | | | |
| | A179N M210F T213V V231I | | | |
| | R241K F245R V247C V250L | | | |
| | Q267F Q288R L311N | | | |
| t879332 | Library Chimera; T30A C31F | t523834 | 86682.150 | 6367.491 |
| | V39T V40I M43L A85N I86V | | | |
| | M87V D94E V129L V140T | | | |
| | F145L I147L S182V M207V | | | |
| | G211S T213V A227R V234L | | | |
| | F245R V246L V247C V257G | | | |
| | S258A L311N | | | |
| t879116 | Library Chimera; T30A M43L | t523834 | 55977.236 | 8274.306 |
| | S45L G49S S79L I86A D94E | | | |
| | E113R I142L I147L A149L | | | |
| | NI67A T171I G211S T213V | | | |
| | I220L V234L F245R V246I | | | |
| | V247C V250L L256V Q267F | | | |
| | Q288R L311N | | | |
| t879830 | Library Chimera; C31F M43V | t523834 | 54191.912 | 3133.435 |
| | I46G M87V D94E V106A | | | |
| | E113R F139L F141V I142L | | | |
| | F145L I172L S173L S182V | | | |
| | M212L T213V I220L V234L | | | |
| | N242T F245W V247C V257G | | | |
| | Q267F S271L Q288R | | | |

### Example 9: Biosynthesis of Cannabinoids in Engineered S. cerevisiae Host Cells

The activation of an organic acid to its CoA-thioester and the subsequent condensation of this thioester with a number of malonyl-CoA molecules, or other similar polyketide extender units, represent the first two steps in the biosynthesis of all known cannabinoids. To demonstrate the biosynthesis of CBGA (FIG. 1, Formula 8a), CBDA (FIG. 1, Formula 9a), THCA (FIG. 1, Formula 10a), and/or CBCA (FIG. 1, Formula 11a) the cannabinoid biosynthetic pathway show in FIG. 1 is assembled in the genome of a prototrophic S. cerevisiae CEN.PK host cell wherein each enzyme (R1a-R5a) may be present in one or more copies. For example, the S. cerevisiae host cell may express one or more copies of one or more of: an AAE, an OLS, an OAC, a PT, and a TS.

The AAE enzyme used may be a naturally occurring or synthetic AAE that is functionally expressed in S. cerevisiae, or a variant thereof, with activity on hexanaoic acid. The OLS enzyme may be a naturally occurring or synthetic OLS that is functionally expressed in S. cerevisiae. The OAC enzyme may be a naturally occurring or synthetic OAC that is functionally expressed in S. cerevisiae. In instances where a bifunctional OLS is used, a separate OAC enzyme may or may not be omitted.

A PT enzyme, such as a CBGAS enzyme, may be a naturally occurring or synthetic PT that is functionally expressed in S. cerevisiae, or a variant thereof, including a PT from C. sativa or a variant of a PT from C. sativa. The PT enzyme may comprise one or more of the PT enzymes provided in this disclosure.

A TS enzyme may be a naturally occurring or synthetic TS that is functionally expressed in S. cerevisiae, or a variant thereof, including a TS from C. sativa or a variant of a TS from C. sativa. The TS enzyme may be a TS that produces one or more of CBDA, THCA, and CBCA as a majority product.

The cannabinoid fermentation procedure may be similar to the PT assay described in the Examples above, except that the incubation of production cultures may last from, for example, 48-144 hours and production cultures may be supplemented with, for example, 4% galactose and 1 mM sodium hexanoate every 24 hours. Titers of CBGA, CBDA, THCA, and/or CBCA are quantified via LC-MS.

It should be appreciated that sequences provided in this disclosure may or may not contain signal sequences. The sequences provided in this disclosure encompass versions with or without signal sequences. It should also be understood that protein sequences provided in this disclosure may be depicted with or without a start codon (M). Accordingly, in some instances amino acid numbering may correspond to protein sequences containing a start codon, while in other instances, amino acid numbering may correspond to protein sequences that do not contain a start codon. It should also be understood that sequences provided in this disclosure may be depicted with or without a stop codon. Aspects of the disclosure encompass host cells comprising any of the sequences provided in this disclosure, including the sequences within Tables 13-20 and fragments thereof.

### Additional Tables Associated with the Disclosure

TABLE 13
Prenyltransferase sequences associated with Examples 1-2

| | Strain type | PT Protein | PT Nucleic Acid |
|---|---|---|---|
| Strain | PT type | SEQ ID NO: | SEQ ID NO: |
| t523578 | Library | 110 | 133 |
| | (CsPT4-CsPT7 chimera) | | |
| t523602 | Library | 111 | 134 |
| | (CsPT4-CsPT7 chimera) | | |
| t523722 | Library | 112 | 135 |
| | (CsPT4-CsPT6 chimera) | | |
| t523777 | Library | 113 | 136 |
| | (CsPT4-CsPT1 chimera) | | |
| t523834 | Library | 114 | 137 |
| | (CsPT4-CsPT1 chimera) | | |
| t524736 | Library | 115 | 138 |
| | (CsPT4-CsPT1 chimera) | | |
| t524816 | Library | 116 | 139 |
| | (CsPT4-CsPT7 chimera) | | |
| t524866 | Library | 117 | 140 |
| | (CsPT4-CsPT6 chimera) | | |
| t525864 | Library | 118 | 141 |
| | (CsPT4-CsPT1 chimera) | | |
| t526650 | Library | 119 | 142 |
| | (CsPT4-CsPT7 chimera) | | |
| t526890 | Library | 120 | 143 |
| | (CsPT4-CsPT7 chimera) | | |
| t526897 | Library | 121 | 144 |
| | (CsPT4-CsPT1 chimera) | | |
| t524521 | Library | 122 | 145 |
| | (fusion) | | |
| t524649 | Library | 123 | 146 |
| | (fusion) | | |
| t524722 | Library | 124 | 147 |
| | (fusion) | | |
| t524730 | Library | 125 | 148 |
| | (fusion) | | |
| t524834 | Library | 126 | 149 |
| | (fusion) | | |
| t524842 | Library | 127 | 150 |
| | (fusion) | | |
| t526009 | Library | 128 | 151 |
| | (fusion) | | |
| t526811 | Library | 129 | 152 |
| | (fusion) | | |
| t526843 | Library | 130 | 153 |
| | (fusion) | | |
| t526923 | Library | 131 | 154 |
| | (fusion) | | |
| t526955 | Library | 132 | 155 |
| | (fusion) | | |

TABLE 14
Chimeric fusion sequences associated with
the Gen 2 PT library described in Example 3

| | | PT | Nucleic |
|---|---|---|---|
| | | Protein | Acid |
| | Strain type/ | SEQ | SEQ |
| Strain | PT type | ID NO: | ID NO: |
| t612532 | Library | 156 | 191 |
| | (ERG20ww-Chimera fusion; CsPT1-CsPT4) | | |
| t612533 | Library | 157 | 192 |
| | (ERG20ww-Chimera fusion; CsPT4-CsPT6) | | |
| t612534 | Library | 158 | 193 |
| | (ERG20ww-Chimera fusion; CsPT4-CsPT7) | | |
| t612535 | Library | 159 | 194 |
| | (ERG20ww-Chimera fusion; CsPT1-CsPT4) | | |
| t612536 | Library | 160 | 195 |
| | (ERG20ww-Chimera fusion; CsPT4-CsPT7) | | |
| t612537 | Library | 161 | 196 |
| | (ERG20ww-Chimera fusion; CsPT4-CsPT7) | | |
| t612538 | Library | 162 | 197 |
| | (ERG20ww-Chimera fusion; CsPT4-CsPT7) | | |
| t612539 | Library | 163 | 198 |
| | (ERG20ww-Chimera fusion; CsPT1-CsPT4) | | |
| t612540 | Library | 164 | 199 |
| | (ERG20ww-Chimera fusion; CsPT1-CsPT4) | | |
| t612541 | Library | 165 | 200 |
| | (ERG20ww-Chimera fusion; CsPT4-CsPT6) | | |
| t612542 | Library | 166 | 201 |
| | (ERG20ww-Chimera fusion; CsPT1-CsPT4) | | |
| t612543 | Library | 167 | 202 |
| | (ERG20ww-Chimera fusion; CsPT1-CsPT4) | | |
| t612544 | Library | 168 | 203 |
| | (ERG20ww-Chimera fusion; CsPT4-CsPT7) | | |
| t612545 | Library | 169 | 204 |
| | (ERG20ww-Chimera fusion; CsPT1-CsPT4) | | |
| t612546 | Library | 170 | 205 |
| | (ERG20ww-Chimera fusion; CsPT1-CsPT4) | | |
| t612547 | Library | 171 | 206 |
| | (ERG20ww-Chimera fusion; CsPT4-CsPT7) | | |
| t612548 | Library | 172 | 207 |
| | (ERG20ww-Chimera fusion; CsPT4-CsPT7) | | |
| t612549 | Library | 173 | 208 |
| | (ERG20ww-Chimera fusion; CsPT4-CsPT7) | | |
| t612550 | Library | 174 | 209 |
| | (ERG20ww-Chimera fusion; CsPT1-CsPT4) | | |
| t612551 | Library | 175 | 210 |
| | (ERG20ww-Chimera fusion; CsPT1-CsPT4) | | |
| t612552 | Library | 176 | 211 |
| | (ERG20ww-Chimera fusion; CsPT1-CsPT4) | | |
| t612553 | Library | 177 | 212 |
| | (ERG20ww-Chimera fusion; CsPT4-CsPT6) | | |
| t612554 | Library | 178 | 213 |
| | (ERG20ww-Chimera fusion; CsPT4-CsPT6) | | |
| t612555 | Library | 179 | 214 |
| | (ERG20ww-Chimera fusion; CsPT4-CsPT7) | | |
| t612556 | Library | 180 | 215 |
| | (ERG20ww-Chimera fusion; CsPT1-CsPT4) | | |
| t612557 | Library | 181 | 216 |
| | (ERG20ww-Chimera fusion; CsPT1-CsPT4) | | |
| t612558 | Library | 182 | 217 |
| | (ERG20ww-Chimera fusion; CsPT1-CsPT4) | | |
| t612559 | Library | 183 | 218 |
| | (ERG20ww-Chimera fusion; CsPT1-CsPT4) | | |
| t612560 | Library | 184 | 219 |
| | (ERG20ww-Chimera fusion; CsPT1-CsPT4) | | |
| t612561 | Library | 185 | 220 |
| | (ERG20ww-Chimera fusion; CsPT1-CsPT4) | | |
| t612562 | Library | 186 | 221 |
| | (ERG20ww-Chimera fusion; CsPT4-CsPT6) | | |
| t612563 | Library | 187 | 222 |
| | (ERG20ww-Chimera fusion; CsPT1-CsPT4) | | |
| t612564 | Library | 188 | 223 |
| | (ERG20ww-Chimera fusion; CsPT1-CsPT4) | | |
| t612565 | Library | 189 | 224 |
| | (ERG20ww-Chimera fusion; CsPT1-CsPT4) | | |
| t612566 | Library | 190 | 225 |
| | (ERG20ww-Chimera fusion; CsPT4-CsPT7) | | |
| t612591 | Library | 704 | 729 |
| | ERG20ww-Chimera fusion | | |
| t612597 | Library | 710 | 735 |
| | ERG20ww-Chimera fusion | | |
| t612611 | Library | 724 | 749 |
| | ERG20ww-Chimera fusion | | |

TABLE 15
Non-limiting examples of sequences of CsPT chimera portions*

| Representative | Chimera Descriptions | | | | | | | | | |
|---|---|---|---|---|---|---|---|---|---|---|
| Strain ID | X1 | X2 | X3 | X4 | X5 | X6 | X7 | X8 | X9 | X10 |
| t524736 | CsPT4 | CsPT1 | CsPT4 | CsPT1 | CsPT4 | CsPT1 | CsPT4 | CsPT1 | CsPT4 | CsPT1 |
| t612532 | 1-53 | 52-66 | 69-130 | 129-135 | 138-183 | 182-197 | 200-260 | 259-270 | 273-317 | 316-321 |
| t612542 | (SEQ | (SEQ | (SEQ | (SEQ | (SEQ | (SEQ | (SEQ | (SEQ | (SEQ | (SEQ |
| t612550 | ID NO: | ID NO: | ID NO: | ID NO: | ID NO: | ID NO: | ID NO: | ID NO: | ID NO: | ID NO: |
| t612563 | 33) | 40) | 47) | 54) | 61) | 68) | 75) | 82) | 89) | NO: |
| t612539 | | | | | | | | | | |
| t524816 | CsPT4 | CsPT7 | CsPT4 | CsPT7 | CsPT4 | CsPT7 | CsPT4 | CsPT7 | CsPT4 | CsPT7 |
| t612534 | 1-50 | 51-77 | 78-123 | 124-146 | 147-177 | 178-206 | 207-253 | 254-278 | 279-310 | 311-323 |
| t612536 | (SEQ | (SEQ | (SEQ | (SEQ | (SEQ | (SEQ | (SEQ | (SEQ | (SEQ | (SEQ |
| t612547 | ID NO: | ID NO: | ID NO: | ID NO: | ID NO: | ID NO: | ID NO: | ID NO: | ID NO: | ID NO: |
| t612549 | 34) | 41) | 48) | 55) | 62) | 69) | 76) | 83) | 90) | 97) |
| t612555 | | | | | | | | | | |
| t612572^{a} | | | | | | | | | | |
| t612578^{b} | | | | | | | | | | |
| t612585^{c} | | | | | | | | | | |
| t612586^{d} | | | | | | | | | | |
| t612588^{e} | | | | | | | | | | |
| t523834 | CsPT4 | CsPT1 | CsPT4 | CsPT1 | CsPT4 | CsPT1 | CsPT4 | CsPT1 | CsPT4 | CsPT1 |
| t612535 | 1-52 | 51-67 | 70-129 | 128-136 | 139-182 | 181-198 | 201-259 | 258-271 | 274-316 | 315-321 |
| t612546 | (SEQ | (SEQ | (SEQ | (SEQ | (SEQ | (SEQ | (SEQ | (SEQ | (SEQ | (SEQ |
| t612552 | ID NO: | ID NO: | ID NO: | ID NO: | ID NO: | ID NO: | ID NO: | ID NO: | ID NO: | ID NO: |
| t612564 | 35) | 42) | 49) | 56) | 63) | 70) | 77) | 84) | 91) | 98) |
| t612565 | | | | | | | | | | |
| t526650 | CsPT4 | CsPT7 | CsPT4 | CsPT7 | CsPT4 | CsPT7 | CsPT4 | CsPT7 | CsPT4 | CsPT7 |
| t612537 | 1-53 | 54-68 | 69-130 | 131-137 | 138-183 | 184-199 | 200-260 | 261-272 | 273-317 | 318-323 |
| t612538 | (SEQ | (SEQ | (SEQ | (SEQ | (SEQ | (SEQ | (SEQ | (SEQ | (SEQ | (SEQ |
| t612544 | ID NO: | ID NO: | ID NO: | ID NO: | ID NO: | ID NO: | ID NO: | ID NO: | ID NO: | ID NO: |
| t612548 | 36) | 43) | 50) | 57) | 64) | 71) | 78) | 85) | 92) | 99) |
| t612566 | | | | | | | | | | |
| t612568^{f} | | | | | | | | | | |
| t612574^{g} | | | | | | | | | | |
| t612582^{h} | | | | | | | | | | |
| t523777 | CsPT4 | CsPT1 | CsPT4 | CsPT1 | CsPT4 | CsPT1 | CsPT4 | CsPT1 | CsPT4 | CsPT1 |
| t612543 | 1-50 | 49-75 | 78-123 | 122-144 | 147-177 | 176-204 | 207-253 | 252-276 | 279-310 | 309-321 |
| t612556 | (SEQ | (SEQ | (SEQ | (SEQ | (SEQ | (SEQ | (SEQ | (SEQ | (SEQ | (SEQ |
| t612558 | ID NO: | ID NO: | ID NO: | ID NO: | ID NO: | ID NO: | ID NO: | ID NO: | ID NO: | ID NO: |
| t612559 | 37) | 44) | 51) | 58) | 65) | 72) | 79) | 86) | 93) | 100) |
| t526897 | CsPT4 | CsPT1 | CsPT4 | CsPT1 | CsPT4 | CsPT1 | CsPT4 | CsPT1 | CsPT4 | CsPT1 |
| t612551 | 1-49 | 48-76 | 79-122 | 121-145 | 148-176 | 175-205 | 208-252 | 251-277 | 280-309 | 308-321 |
| t612560 | (SEQ | (SEQ | (SEQ | (SEQ | (SEQ | (SEQ | (SEQ | (SEQ | (SEQ | (SEQ |
| | ID NO: | ID NO: | ID NO: | ID NO: | ID NO: | ID NO: | ID NO: | ID NO: | ID NO: | ID NO: |
| | 38) | 45) | 52) | 59) | 66) | 73) | 80) | 87) | 94) | 101) |
| t523722 | CsPT4 | CsPT6 | CsPT4 | CsPT6 | CsPT4 | CsPT6 | CsPT4 | CsPT6 | CsPT4 | CsPT6 |
| t612576ⁱ | 1-54 | 104-116 | 68-131 | 178-182 | 137-184 | 233-247 | 199-261 | 311-320 | 272-318 | 366-371 |
| | (SEQ | (SEQ | (SEQ | (SEQ | (SEQ | (SEQ | (SEQ | (SEQ | (SEQ | (SEQ |
| | ID NO: | ID NO: | ID NO: | ID NO: | ID NO: | ID NO: | ID NO: | ID NO: | ID NO: | ID NO: |
| | 39) | 46) | 53) | 60) | 67) | 74) | 81) | 88) | 95) | 102) |

*The amino acid numbering for CsPT1 is based on SEQ ID NO: 1185; the amino acid numbering for CsPT4 is based on SEQ ID NO: 5; the amino acid numbering for CsPT6 is based on SEQ ID NO: 701; and the amino acid numbering for CsPT7 is based on SEQ ID NO: 702.
^{a}The chimeric PT expressed by this strain additionally contains a S232R substitution relative to SEQ ID NO: 5
^{b}The chimeric PT expressed by this strain additionally contains C31F and S232R substitutions relative to SEQ ID NO: 5
^{c}The chimeric PT expressed by this strain additionally contains F245R substitution relative to SEQ ID NO: 5
^{d}The chimeric PT expressed by this strain additionally contains C31F, F245R, and S232R substitutions relative to SEQ ID NO: 5
^{e}The chimeric PT expressed by this strain additionally contains C31F and F245R substitutions relative to SEQ ID NO: 5
^{f}The chimeric PT expressed by this strain additionally contains a S232R substitution relative to SEQ ID NO: 5
^{g}The chimeric PT expressed by this strain additionally contains a C31F substitution relative to SEQ ID NO: 5
^{h}The chimeric PT expressed by this strain additionally contains a F245R substitution relative to SEQ ID NO: 5
ⁱThe chimeric PT expressed by this strain additionally contains a S232R substitution relative to SEQ ID NO: 5

TABLE 16
Prenyltransferase sequences associated with
Gen 3 CsPT4 library described in Example 5

| | | | PT |
|---|---|---|---|
| | | PT | Nucleic |
| | | Protein | Acid |
| | Strain type | SEQ | SEQ |
| Strain | PT type | ID NO: | ID NO: |
| t704346 | Library | 226 | 325 |
| | (ERG20ww-Chimera fusion; CsPT1-CsPT4) | | |
| t704382 | Library | 227 | 326 |
| | (ERG20ww-Chimera fusion; CsPT1-CsPT4) | | |
| t721427 | Library | 228 | 327 |
| | (ERG20ww-Chimera fusion; CsPT4-CsPT6- | | |
| | CsPT7) | | |
| t721429 | Library | 229 | 328 |
| | (ERG20ww-Chimera fusion; CsPT4-CsPT7) | | |
| t721431 | Library | 230 | 329 |
| | (ERG20ww-Chimera fusion; CsPT1-CsPT4) | | |
| t721433 | Library | 231 | 330 |
| | (ERG20ww-Chimera fusion; CsPT4-CsPT7) | | |
| t721435 | Library | 232 | 331 |
| | (ERG20ww-Chimera fusion; CsPT4-CsPT7) | | |
| t721437 | Library | 233 | 332 |
| | (ERG20ww-Chimera fusion; CsPT4- CsPT6- | | |
| | CsPT7) | | |
| t721439 | Library | 234 | 333 |
| | (ERG20ww-Chimera fusion; CsPT1-CsPT4) | | |
| t721441 | Library | 235 | 334 |
| | (ERG20ww-Chimera fusion; CsPT4-CsPT7) | | |
| t721443 | Library | 236 | 335 |
| | (ERG20ww-Chimera fusion; CsPT4-CsPT7) | | |
| t721445 | Library | 237 | 336 |
| | (ERG20ww-Chimera fusion: CsPT4-CsPT6- | | |
| | CsPT7) | | |
| t721447 | Library | 238 | 337 |
| | (ERG20ww-Chimera fusion: CsPT4-CsPT7) | | |
| t721449 | Library | 239 | 338 |
| | (ERG20ww-Chimera fusion; CsPT1-CsPT4) | | |
| t721451 | Library | 240 | 339 |
| | (ERG20ww-Chimera fusion: CsPT1-CsPT4) | | |
| t721453 | Library | 241 | 340 |
| | (ERG20ww-Chimera fusion; CsPT4-CsPT7) | | |
| t721455 | Library | 242 | 341 |
| | (ERG20ww-Chimera fusion; CsPT1-CsPT4) | | |
| t721457 | Library | 243 | 342 |
| | (ERG20ww-Chimera fusion; CsPT4-CsPT7) | | |
| t721459 | Library | 244 | 343 |
| | (ERG20ww-Chimera fusion; CsPT1-CsPT4) | | |
| t721461 | Library | 245 | 344 |
| | (ERG20ww-Chimera fusion; CsPT4-CsPT7) | | |
| t721463 | Library | 246 | 345 |
| | (ERG20ww-Chimera fusion; CsPT1-CsPT4- | | |
| | CsPT6) | | |
| t721465 | Library | 247 | 346 |
| | (ERG20ww-Chimera fusion; CsPT4-CsPT7) | | |
| t721467 | Library | 248 | 347 |
| | (ERG20ww-Chimera fusion; CsPT4-CsPT7) | | |
| t721469 | Library | 249 | 348 |
| | (ERG20ww-Chimera fusion; CsPT1-CsPT4) | | |
| t721471 | Library | 250 | 349 |
| | (ERG20ww-Chimera fusion; CsPT4-CsPT7) | | |
| t721473 | Library | 251 | 350 |
| | (ERG20ww-Chimera fusion; CsPT1-CsPT4- | | |
| | CsPT6) | | |
| t721475 | Library | 252 | 351 |
| | (ERG20ww-Chimera fusion; CsPT4-CsPT7) | | |
| t721477 | Library | 253 | 352 |
| | (ERG20ww-Chimera fusion; CsPT1-CsPT4) | | |
| t721479 | Library | 254 | 353 |
| | (ERG20ww-Chimera fusion; CsPT4-CsPT7) | | |
| t721481 | Library | 255 | 354 |
| | (ERG20ww-Chimera fusion; CsPT1-CsPT4- | | |
| | CsPT6) | | |
| t721483 | Library | 256 | 355 |
| | (ERG20ww-Chimera fusion; CsPT4-CsPT7) | | |
| t721485 | Library | 257 | 356 |
| | (ERG20ww-Chimera fusion; CsPT4-CsPT7) | | |
| t721487 | Library | 258 | 357 |
| | (ERG20ww-Chimera fusion; CsPT1-CsPT4) | | |
| t721489 | Library | 259 | 358 |
| | (ERG20ww-Chimera fusion; CsPT1-CsPT4- | | |
| | CsPT6) | | |
| t721491 | Library | 260 | 359 |
| | (ERG20ww-Chimera fusion; CsPT4-CsPT7) | | |
| t721493 | Library | 261 | 360 |
| | (ERG20ww-Chimera fusion; CsPT4-CsPT7) | | |
| t721495 | Library | 262 | 361 |
| | (ERG20ww-Chimera fusion; CsPT1-CsPT4) | | |
| t721497 | Library | 263 | 362 |
| | (ERG20ww-Chimera fusion; CsPT1-CsPT4) | | |
| t721499 | Library | 264 | 363 |
| | (ERG20ww-Chimera fusion; CsPT4-CsPT7) | | |
| t721501 | Library | 265 | 364 |
| | (ERG20ww-Chimera fusion; CsPT4-CsPT7) | | |
| t721503 | Library | 266 | 365 |
| | (ERG20ww-Chimera fusion; CsPT1-CsPT4) | | |
| t721505 | Library | 267 | 366 |
| | (ERG20ww-Chimera fusion: CsPT1-CsPT4) | | |
| t721507 | Library | 268 | 367 |
| | (ERG20ww-Chimera fusion; CsPT4-CsPT7) | | |
| t721509 | Library | 269 | 368 |
| | (ERG20ww-Chimera fusion; CsPT4-CsPT7) | | |
| t721511 | Library | 270 | 369 |
| | (ERG20ww-Chimera fusion; CsPT1-CsPT4) | | |
| t721513 | Library | 271 | 370 |
| | (ERG20ww-Chimera fusion; CsPT1-CsPT4) | | |
| t721515 | Library | 272 | 371 |
| | (ERG20ww-Chimera fusion; CsPT4-CsPT7) | | |
| t721517 | Library | 273 | 372 |
| | (ERG20ww-Chimera fusion; CsPT4-CsPT7) | | |
| t721519 | Library | 274 | 373 |
| | (ERG20ww-Chimera fusion; CsPT1-CsPT4) | | |
| t721521 | Library | 275 | 374 |
| | (ERG20ww-Chimera fusion; CsPT1-CsPT4) | | |
| t721523 | Library | 276 | 375 |
| | (ERG20ww-Chimera fusion; CsPT4-CsPT7) | | |
| t721525 | Library | 277 | 376 |
| | (ERG20ww-Chimera fusion; CsPT4-CsPT7) | | |
| t721527 | Library | 278 | 377 |
| | (ERG20ww-Chimera fusion; CsPT1-CsPT4) | | |
| t721529 | Library | 279 | 378 |
| | (ERG20ww-Chimera fusion; CsPT4-CsPT7) | | |
| t721531 | Library | 280 | 379 |
| | (ERG20ww-Chimera fusion; CsPT1-CsPT4) | | |
| t721533 | Library | 281 | 380 |
| | (ERG20ww-Chimera fusion; CsPT4-CsPT7) | | |
| t721535 | Library | 282 | 381 |
| | (ERG20ww-Chimera fusion; CsPT4-CsPT7) | | |
| t721537 | Library | 283 | 382 |
| | (ERG20ww-Chimera fusion; CsPT4-CsPT7) | | |
| t721539 | Library | 284 | 383 |
| | (ERG20ww-Chimera fusion; CsPT1-CsPT4) | | |
| t721541 | Library | 285 | 384 |
| | (ERG20ww-Chimera fusion; CsPT1-CsPT4) | | |
| t721543 | Library | 286 | 385 |
| | (ERG20ww-Chimera fusion; CsPT4-CsPT7) | | |
| t721545 | Library | 287 | 386 |
| | (ERG20ww-Chimera fusion; CsPT4-CsPT7) | | |
| t721547 | Library | 288 | 387 |
| | (ERG20ww-Chimera fusion; CsPT1-CsPT4) | | |
| t721549 | Library | 289 | 388 |
| | (ERG20ww-Chimera fusion; CsPT1-CsPT4) | | |
| t721551 | Library | 290 | 389 |
| | (ERG20ww-Chimera fusion; CsPT4-CsPT7) | | |
| t721553 | Library | 291 | 390 |
| | (ERG20ww-Chimera fusion; CsPT1-CsPT4) | | |
| t721555 | Library | 292 | 391 |
| | (ERG20ww-Chimera fusion; CsPT4-CsPT7) | | |
| t721557 | Library | 293 | 392 |
| | (ERG20ww-Chimera fusion; CsPT1-CsPT4) | | |
| t721559 | Library | 294 | 393 |
| | (ERG20ww-Chimera fusion; CsPT4-CsPT7) | | |
| t721561 | Library | 295 | 394 |
| | (ERG20ww-Chimera fusion; CsPT4-CsPT7) | | |
| t721563 | Library | 296 | 395 |
| | (ERG20ww-Chimera fusion; CsPT1-CsPT4) | | |
| t721565 | Library | 297 | 396 |
| | (ERG20ww-Chimera fusion; CsPT1-CsPT4) | | |
| t721567 | Library | 298 | 397 |
| | (ERG20ww-Chimera fusion; CsPT4-CsPT7) | | |
| t721569 | Library | 299 | 398 |
| | (ERG20ww-Chimera fusion; CsPT4-CsPT7) | | |
| t721573 | Library | 300 | 399 |
| | (ERG20ww-Chimera fusion; CsPT1-CsPT4) | | |
| t721575 | Library | 301 | 400 |
| | (ERG20ww-Chimera fusion; CsPT4-CsPT7) | | |
| t721579 | Library | 302 | 401 |
| | (ERG20ww-Chimera fusion; CsPT1-CsPT4) | | |
| t721581 | Library | 303 | 402 |
| | (ERG20ww-Chimera fusion; CsPT1-CsPT4) | | |
| t721583 | Library | 304 | 403 |
| | (ERG20ww-Chimera fusion; CsPT4-CsPT7) | | |
| t721585 | Library | 305 | 404 |
| | (ERG20ww-Chimera fusion; CsPT4-CsPT7) | | |
| t721589 | Library | 306 | 405 |
| | (ERG20ww-Chimera fusion; CsPT1-CsPT4) | | |
| t721591 | Library | 307 | 406 |
| | (ERG20ww-Chimera fusion: CsPT4-CsPT7) | | |
| t721593 | Library | 308 | 407 |
| | (ERG20ww-Chimera fusion; CsPT4-CsPT7) | | |
| t721595 | Library | 309 | 408 |
| | (ERG20ww-Chimera fusion; CsPT1-CsPT4) | | |
| t721597 | Library | 310 | 409 |
| | (ERG20ww-Chimera fusion; CsPT1-CsPT4) | | |
| t721599 | Library | 311 | 410 |
| | (ERG20ww-Chimera fusion: CsPT4-CsPT7) | | |
| t721601 | Library | 312 | 411 |
| | (ERG20ww-Chimera fusion; CsPT4-CsPT7) | | |
| t721605 | Library | 313 | 412 |
| | (ERG20ww-Chimera fusion; CsPT1-CsPT4) | | |
| t721607 | Library | 314 | 413 |
| | (ERG20ww-Chimera fusion; CsPT4-CsPT7) | | |
| t721609 | Library | 315 | 414 |
| | (ERG20ww-Chimera fusion; CsPT4-CsPT7) | | |
| t721611 | Library | 316 | 415 |
| | (ERG20ww-Chimera fusion; CsPT1-CsPT4) | | |
| t721613 | Library | 317 | 416 |
| | (ERG20ww-Chimera fusion; CsPT4-CsPT7) | | |
| t721615 | Library | 318 | 417 |
| | (ERG20ww-Chimera fusion; CsPT4-CsPT7) | | |
| t721617 | Library | 319 | 418 |
| | (ERG20ww-Chimera fusion; CsPT4-CsPT7) | | |
| t721619 | Library | 320 | 419 |
| | (ERG20ww-Chimera fusion; CsPT1-CsPT4) | | |
| t721629 | Library | 321 | 420 |
| | (ERG20ww-Chimera fusion; CsPT4-CsPT7) | | |
| t721631 | Library | 322 | 421 |
| | (ERG20ww-Chimera fusion; CsPT4-CsPT7) | | |
| t721633 | Library | 323 | 422 |
| | (ERG20ww-Chimera fusion; CsPT1-CsPT4) | | |
| t721639 | Library | 324 | 423 |
| | (ERG20ww-Chimera fusion; CsPT4-CsPT7) | | |

TABLE 17
ERG20 homolog sequences associated with chimeric
fusion library described in Example 6

| | | Protein | Nucleic Acid |
|---|---|---|---|
| Strain | Strain type | SEQ ID NO: | SEQ ID NO: |
| t756346 | ERG20 Positive Control | 424 | 477 |
| t756349 | ERG20ww Positive Control | 425 | 478 |
| t766132 | Library | 426 | 479 |
| t766504 | Library | 427 | 480 |
| t766593 | Library | 428 | 481 |
| t766467 | Library | 429 | 482 |
| t766152 | Library | 430 | 483 |
| t766629 | Library | 431 | 484 |
| t767697 | Library | 432 | 485 |
| t766672 | Library | 433 | 486 |
| t766111 | Library | 434 | 487 |
| t766340 | Library | 435 | 488 |
| t766148 | Library | 436 | 489 |
| t766308 | Library | 437 | 490 |
| t765947 | Library | 438 | 491 |
| t765987 | Library | 439 | 492 |
| t767109 | Library | 440 | 493 |
| t766404 | Library | 441 | 494 |
| t768423 | Library | 442 | 495 |
| t767236 | Library | 443 | 496 |
| t766101 | Library | 444 | 497 |
| t765981 | Library | 445 | 498 |
| t767135 | Library | 446 | 499 |
| t766263 | Library | 447 | 500 |
| t766601 | Library | 448 | 501 |
| t767176 | Library | 449 | 502 |
| t766406 | Library | 450 | 503 |
| t768409 | Library | 451 | 504 |
| t766650 | Library | 452 | 505 |
| t766129 | Library | 453 | 506 |
| t766740 | Library | 454 | 507 |
| t765825 | Library | 455 | 508 |
| t766639 | Library | 456 | 509 |
| t765979 | Library | 457 | 510 |
| t767808 | Library | 458 | 513 |
| t767611 | Library | 459 | 512 |
| t766017 | Library | 460 | 513 |
| t766201 | Library | 461 | 514 |
| t765881 | Library | 462 | 515 |
| t766011 | Library | 463 | 516 |
| t766043 | Library | 464 | 517 |
| t766077 | Library | 465 | 518 |
| t766103 | Library | 466 | 519 |
| t766115 | Library | 467 | 520 |
| t766304 | Library | 468 | 521 |
| t768416 | Library | 469 | 522 |
| t765857 | Library | 470 | 523 |
| t768386 | Library | 471 | 524 |
| t766051 | Library | 472 | 525 |
| t765739 | Library | 473 | 526 |
| t766094 | Library | 474 | 527 |
| t768404 | Library | 475 | 528 |
| t766095 | Library | 476 | 529 |
| t766469 | Library | 753 | 754 |

TABLE 18
ERG20w homolog - Chimeric PT fusion library
sequences described in Example 6

| | | Protein | Nucleic Acid |
|---|---|---|---|
| Strain | Strain type | SEQ ID NO: | SEQ ID NO: |
| t756346 | ERG20 Positive Control | 530 | 583 |
| t756349 | ERG20ww Positive Control | 531 | 584 |
| t766132 | Library | 532 | 585 |
| t766504 | Library | 533 | 586 |
| t766593 | Library | 534 | 587 |
| t766467 | Library | 535 | 588 |
| t766152 | Library | 536 | 589 |
| t766629 | Library | 537 | 590 |
| t767697 | Library | 538 | 591 |
| t766672 | Library | 539 | 592 |
| t766111 | Library | 540 | 593 |
| t766340 | Library | 541 | 594 |
| t766148 | Library | 542 | 595 |
| t766308 | Library | 543 | 596 |
| t765947 | Library | 544 | 597 |
| t765987 | Library | 545 | 598 |
| t767109 | Library | 546 | 599 |
| t766404 | Library | 547 | 600 |
| t768423 | Library | 548 | 601 |
| t767236 | Library | 549 | 602 |
| t766101 | Library | 550 | 603 |
| t765981 | Library | 551 | 604 |
| t767135 | Library | 552 | 605 |
| t766263 | Library | 553 | 606 |
| t766601 | Library | 554 | 607 |
| t767176 | Library | 555 | 608 |
| t766406 | Library | 556 | 609 |
| t768409 | Library | 557 | 610 |
| t766650 | Library | 558 | 611 |
| t766129 | Library | 559 | 612 |
| t766740 | Library | 560 | 613 |
| t765825 | Library | 561 | 614 |
| t766639 | Library | 562 | 615 |
| t765979 | Library | 563 | 616 |
| t767808 | Library | 564 | 617 |
| t767611 | Library | 565 | 618 |
| t766017 | Library | 566 | 619 |
| t766201 | Library | 567 | 620 |
| t765881 | Library | 568 | 621 |
| t766011 | Library | 569 | 622 |
| t766043 | Library | 570 | 623 |
| t766077 | Library | 571 | 624 |
| t766103 | Library | 572 | 625 |
| t766115 | Library | 573 | 626 |
| t766304 | Library | 574 | 627 |
| t768416 | Library | 575 | 628 |
| t765857 | Library | 576 | 629 |
| t768386 | Library | 577 | 630 |
| t766051 | Library | 578 | 631 |
| t765739 | Library | 579 | 632 |
| t766094 | Library | 580 | 633 |
| t768404 | Library | 581 | 634 |
| t766095 | Library | 582 | 635 |
| t766469 | Library | 755 | 756 |

TABLE 19
Prenyltransferase sequences associated with Example 7

| | Strain type | PT Protein | PT Nucleic Acid |
|---|---|---|---|
| Strain | PT type | SEQ ID NO: | SEQ ID NO: |
| t817911 | Library | 757 | 869 |
| | (CsPT4-CsPT7 chimera; C31F F82G D94E F245R) | | |
| t817917 | Library | 758 | 870 |
| | (CsPT4-CsPT7 chimera; M43L I86S I147L F245R) | | |
| t817954 | Library | 759 | 871 |
| | (CsPT4-CsPT1 chimera; C31F M43V M87V D94E | | |
| | E113R F145L F245W Q267F Q288R) | | |
| t817955 | Library | 760 | 872 |
| | (CsPT4-CsPT1 chimera; C31F M43L F82G D94E | | |
| | E113R F14ST F245R Q267F Q288R) | | |
| t817960 | Library | 761 | 873 |
| | (CsPT4-CsPT1 chimera; M43L F82G D94E E113R | | |
| | F145S F245R Q267F Q288R L311K) | | |
| t817962 | Library | 762 | 874 |
| | (CsPT4-CsPT1 chimera: C31F M43V M87V D94E | | |
| | E113R F245R Q267F Q288R L311N) | | |
| t817963 | Library | 763 | 875 |
| | (CsPT4-CsPT7 chimera; I86A D94E I147L F245R) | | |
| t817977 | Library | 764 | 876 |
| | (CsPT4-CsPT1 chimera; C31F I46C I86A D94E | | |
| | E113R F14ST F245R Q267F Q288R) | | |
| t817985 | Library | 765 | 877 |
| | (CsPT4-CsPT1 chimera; C31F M43V M87V D94E | | |
| | F145T F245R Q267F Q288R L311N) | | |
| t817996 | Library | 766 | 878 |
| | (CsPT4-CsPT1 chimera: C31F I86G D94E E113R | | |
| | F145T F245W Q267F Q288R L311N) | | |
| t818002 | Library | 767 | 879 |
| | (CsPT4-CsPT7 chimera; I86V F245R) | | |
| t818007 | Library | 768 | 880 |
| | (CsPT4-CsPT1 chimera; C31F I46C I86G D94E | | |
| | E113R F245R Q267F Q288R L311N) | | |
| t818009 | Library | 769 | 881 |
| | (CsPT4-CsPT1 chimera; C31F M43V M87V D94B | | |
| | E113R F145L F245R Q288R L311R) | | |
| t818014 | Library | 770 | 882 |
| | (CsPT4-CsPT7 chimera; C31F M43L I86A I147L) | | |
| t818015 | Library | 771 | 883 |
| | (CsPT4-CsPT7 chimera; I86S D94E F245R) | | |
| t818033 | Library | 772 | 884 |
| | (CsPT4-CsPT1 chimera; C31F M43L I86S D94E | | |
| | E113R F145S F245R Q267F L311K) | | |
| t818043 | Library | 773 | 885 |
| | (CsPT4-CsPT7 Chimera; C31F M43L I86V D94E) | | |
| t818044 | Library | 774 | 886 |
| | (CsPT4-CsPT1 Chimera; C31F I46C F82G D94E | | |
| | E113R I147L Q267F Q288R L311N) | | |
| t818058 | Library | 775 | 887 |
| | (CsPT4-CsPT7 Chimera; C31F M43L I86A F245R) | | |
| t818067 | Library | 776 | 888 |
| | (CsPT4-CsPT7 Chimera; C31F I86A F245R) | | |
| t818093 | Library | 777 | 889 |
| | (CsPT4-CsPT1 Chimera; C31F M43L I86S E113R | | |
| | F145L F245R Q267F Q288R L311R) | | |
| t818098 | Library | 778 | 890 |
| | (CsPT4-CsPT1 Chimera; C31F I46C M87V D94E | | |
| | E113R F145L Q267F Q288R L311N) | | |
| t818130 | Library | 779 | 891 |
| | (CsPT4-CsPT7 Chimera; I86S D94E) | | |
| t818140 | Library | 780 | 892 |
| | (CsPT4-CsPT7 Chimera; I86T M87I F151T) | | |
| t818171 | Library | 781 | 893 |
| | (CsPT4-CsPT7 Chimera; M43L I86S D94E) | | |
| t818180 | Library | 782 | 894 |
| | (CsPT4-CsPT7 Chimera; F83Y I86A M87T) | | |
| t818195 | Library | 783 | 895 |
| | (CsPT4-CsPT1 Chimera; C31F M43L F82G I86G | | |
| | D94E F145L I147L F245R L311N) | | |
| t818196 | Library | 784 | 896 |
| | (CsPT4-CsPT7 Chimera; I86V M87T) | | |
| t818198 | Library | 785 | 897 |
| | (CsPT4-CsPT7 Chimera; I86V I147L F245R) | | |
| t818205 | Library | 786 | 898 |
| | (CsPT4-CsPT7 Chimera; C31F I86V D94E I147L) | | |
| t818206 | Library | 787 | 899 |
| | (CsPT4-CsPT1 Chimera; C31F I46C I86A E113R | | |
| | I147L F245W Q267F Q288R L311N) | | |
| t818207 | Library | 788 | 900 |
| | (CsPT4-CsPT7 Chimera; I86A M87I) | | |
| t818208 | Library | 789 | 901 |
| | (CsPT4-CsPT1 Chimera; C31F M43L I86A D94E | | |
| | E113R F145S F245R Q267F L311K) | | |
| t818210 | Library | 790 | 902 |
| | (CsPT4-CsPT1 Chimera; C31F M43L I86G D94E | | |
| | E113R F145L F245R Q288R L311R) | | |
| t818214 | Library | 791 | 903 |
| | (CsPT4-CsPT7 Chimera; M43L I86A D94E) | | |
| t818215 | Library | 792 | 904 |
| | (CsPT4-CsPT1 Chimera; C31F I46C I86G D94E | | |
| | E113R F145L F245R Q288R L311N) | | |
| t818223 | Library | 793 | 905 |
| | (CsPT4-CsPT1 Chimera; C31F M43V I86G E113R | | |
| | F145L F245R Q267F Q288R L311K) | | |
| t818230 | Library | 794 | 906 |
| | (CsPT4-CsPT1 Chimera; C31F F82G I86V M87V | | |
| | D94E F145L I147L F245R L311K) | | |
| t818247 | Library | 795 | 907 |
| | (CsPT4-CsPT7 Chimera; I86A D94E I147L) | | |
| t818248 | Library | 796 | 908 |
| | (CsPT4-CsPT7 Chimera; I86A D94E) | | |
| t818257 | Library | 797 | 909 |
| | (CsPT4-CsPT7 Chimera; F83Y I86A M87I F151T) | | |
| t818260 | Library | 798 | 910 |
| | (CsPT4-CsPT7 Chimera; I86A M87V S119A F151G) | | |
| t818375 | Library | 799 | 911 |
| | (CsPT4-CsPT7 Chimera; I86A S119A) | | |
| t818379 | Library | 800 | 912 |
| | (CsPT4-CsPT7 Chimera; M43L I86S M87V) | | |
| t818383 | Library | 801 | 913 |
| | (CsPT4-CsPT7 Chimera; I86T S119A F151T) | | |
| t818388 | Library | 802 | 914 |
| | (CsPT4-CsPT7 Chimera; C31F F82G) | | |
| t818392 | Library | 803 | 915 |
| | (CsPT4-CsPT7 Chimera; C31F M43L I86A D94E) | | |
| t818408 | Library | 804 | 916 |
| | (CsPT4-CsPT7 Chimera; C31F I86V D94E) | | |
| t818426 | Library | 805 | 917 |
| | (CsPT4-CsPT7 Chimera; I86G F245R) | | |
| t818427 | Library | 806 | 918 |
| | (CsPT4-CsPT7 Chimera; M43L I86S F245R) | | |
| t818547 | Library | 807 | 919 |
| | (CsPT4-CsPT7 Chimera; I86T M87T) | | |
| t818555 | Library | 808 | 920 |
| | (CsPT4-CsPT1 Chimera; C31F M43L I86A D94E | | |
| | E113R I147L F245R Q267F Q288R) | | |
| t818565 | Library | 809 | 921 |
| | (CsPT4-CsPT1 Chimera; C31F M43L I86V M87V | | |
| | D94E F145L I147L F245R L311N) | | |
| t818573 | Library | 810 | 922 |
| | (CsPT4-CsPT1 Chimera; C31F M43L I86S M87V | | |
| | D94E F145L I147L F245R L311K) | | |
| t818606 | Library | 811 | 923 |
| | (CsPT4-CsPT1 Chimera; C31F I46C F82G D94E | | |
| | E113R F145L Q267F Q288R L311N) | | |
| t818614 | Library | 812 | 924 |
| | (CsPT4-CsPT7 Chimera; I86G D94E) | | |
| t818626 | Library | 813 | 925 |
| | (CsPT4-CsPT7 Chimera; F82G V122F) | | |
| t818726 | Library | 814 | 926 |
| | (CsPT4-CsPT1 Chimera; C31F M43L F82G E113R | | |
| | F145S F245R Q267F Q288R L311R) | | |
| t818728 | Library | 815 | 927 |
| | (CsPT4-CsPT1 Chimera; C31F M43L I86S D94E | | |
| | E113R F245R Q267F Q288R L311K) | | |
| t818733 | Library | 816 | 928 |
| | (CsPT4-CsPT1 Chimera; C31F M43V M87V D94E | | |
| | E113R F145T F245R Q267F L311N) | | |
| t818738 | Library | 817 | 929 |
| | (CsPT4-CsPT1 Chimera; C31F F82G I86V M87V | | |
| | D94E F145L I147L F245R L311N) | | |
| t818739 | Library | 818 | 930 |
| | (CsPT4-CsPT7 Chimera; C31F I86A D94E F245R) | | |
| t818742 | Library | 819 | 931 |
| | (CsPT4-CsPT1 Chimera; I46C F82G D94E E113R | | |
| | I147L F245R Q267F Q288R L311N) | | |
| t818743 | Library | 820 | 932 |
| | (CsPT4-CsPT1 Chimera; C31F M43L I86G M87V | | |
| | D94E F145L I147L F245R L311N) | | |
| t818744 | Library | 821 | 933 |
| | (CsPT4-CsPT1 Chimera; M43L I86A D94E E113R | | |
| | I147L F245R Q267F Q288R L311N) | | |
| t818745 | Library | 822 | 934 |
| | (CsPT4-CsPT7 Chimera; M43L I86S) | | |
| t818758 | Library | 823 | 935 |
| | (CsPT4-CsPT1 Chimera; M43L F82G I86V M87V | | |
| | D94E F145L I147L F245R L311N) | | |
| t818759 | Library | 824 | 936 |
| | (CsPT4-CsPT7 Chimera; C31F I86V M87V) | | |
| t818763 | Library | 825 | 937 |
| | (CsPT4-CsPT7 Chimera; I86G V122S) | | |
| t818767 | Library | 826 | 938 |
| | (CsPT4-CsPT1 Chimera; C31F M43V F82G D94E | | |
| | E113R F145S F245R Q288R L311R) | | |
| t818770 | Library | 827 | 939 |
| | (CsPT4-CsPT1 Chimera; C31F M43L F82G D94E | | |
| | E113R F14ST F245R Q267F L311N) | | |
| t818772 | Library | 828 | 940 |
| | (CsPT4-CsPT7 Chimera; F83Y I86T M87V) | | |
| t818781 | Library | 829 | 941 |
| | (CsPT4-CsPT7 Chimera; I86G M87I) | | |
| t818786 | Library | 830 | 942 |
| | (CsPT4-CsPT7 Chimera; C31F F82G D94E I147L) | | |
| t818801 | Library | 831 | 943 |
| | (CsPT4-CsPT7 Chimera; F83Y I86S M871 F151T) | | |
| t818804 | Library | 832 | 944 |
| | (CsPT4-CsPT7 Chimera; I86A V122S) | | |
| t818805 | Library | 833 | 945 |
| | (CsPT4-CsPT7 Chimera; I86T S119A) | | |
| t818806 | Library | 834 | 946 |
| | (CsPT4-CsPT1 Chimera; C31F I46C I86A D94E | | |
| | E113R I147L Q267F Q288R L311N) | | |
| t818810 | Library | 835 | 947 |
| | (CsPT4-CsPT1 Chimera; C31F I46C I86S D94E | | |
| | E113R I147L F245R Q288R L311R) | | |
| t818836 | Library | 836 | 948 |
| | (CsPT4-CsPT1 Chimera; C31F I46C M87V D94E | | |
| | E113R F145T F245R Q288R L311R) | | |
| t818843 | Library | 837 | 949 |
| | (CsPT4-CsPT1 Chimera; C31F F82G I86V M87V | | |
| | D94E F145L I147L F245R L311R) | | |
| t818844 | Library | 838 | 950 |
| | (CsPT4-CsPT7 Chimera; M43L I147L F245R) | | |
| t818877 | Library | 839 | 951 |
| | (CsPT4-CsPT7 Chimera; F83Y I86A M87T F151T) | | |
| t818880 | Library | 840 | 952 |
| | (CsPT4-CsPT7 Chimera; I86A M87V I147L) | | |
| t818893 | Library | 841 | 953 |
| | (CsPT4-CsPT7 Chimera; I86V M87T S119A) | | |
| t818902 | Library | 842 | 954 |
| | (CsPT4-CsPT7 Chimera; F83Y I86G F151T) | | |
| t818911 | Library | 843 | 955 |
| | (CsPT4-CsPT7 Chimera; C31F I86V D94E F245R) | | |
| t818922 | Library | 844 | 956 |
| | (CsPT4-CsPT7 Chimera: F82G F245R) | | |
| t818975 | Library | 845 | 957 |
| | (CsPT4-CsPT1 Chimera; C31F M43L F82G I86V | | |
| | D94E F145L I147L F245R L311N) | | |
| t818980 | Library | 846 | 958 |
| | (CsPT4-CsPT7 Chimera; F82G D94E I147L) | | |
| t818982 | Library | 847 | 959 |
| | (CsPT4-CsPT1 Chimera; C31F M43L I86S M87V | | |
| | D94E F145L I147L F245R L311R) | | |
| t818989 | Library | 848 | 960 |
| | (CsPT4-CsPT1 Chimera; C31F M43L I86G D94E | | |
| | E113R F145S Q267F Q288R L311N) | | |
| t819008 | Library | 849 | 961 |
| | (CsPT4-CsPT7 Chimera; I86G D94E I147L F245R) | | |
| t819030 | Library | 850 | 962 |
| | (CsPT4-CsPT1 Chimera; C31F M43L I86S M87V | | |
| | D94E F145L I147L F245R L311N) | | |
| t819037 | Library | 851 | 963 |
| | (CsPT4-CsPT7 Chimera; F83Y I86S M87V F151T) | | |
| t819066 | Library | 852 | 964 |
| | (CsPT4-CsPT7 Chimera; I86V M87I S119A F151T) | | |
| t819073 | Library | 853 | 965 |
| | (CsPT4-CsPT7 Chimera; M43L I86G D94E F245R) | | |
| t819074 | Library | 854 | 966 |
| | (CsPT4-CsPT7 Chimera; F82G I86T V122F) | | |
| t819122 | Library | 855 | 967 |
| | (CsPT4-CsPT1 Chimera; C31F M87V D94E E113R | | |
| | F145T F245W Q267F Q288R L311K) | | |
| t819126 | Library | 856 | 968 |
| | (CsPT4-CsPT1 Chimera; C31F M43V D94E E113R | | |
| | I147L F245W Q267F Q288R L311R) | | |
| t819132 | Library | 857 | 969 |
| | (CsPT4-CsPT1 Chimera; M43L F82G D94E E113R | | |
| | I147L F245R Q267F Q288R L311K) | | |
| t819161 | Library | 858 | 970 |
| | (CsPT4-CsPT7 Chimera; C31F M43L I86S D94E) | | |
| t819169 | Library | 859 | 971 |
| | (CsPT4-CsPT7 Chimera; C31F F82G I147L) | | |
| t819172 | Library | 860 | 972 |
| | (CsPT4-CsPT7 Chimera; C31F F82G D94E) | | |
| t819173 | Library | 861 | 973 |
| | (CsPT4-CsPT7 Chimera; C31F M43L D94E F245R) | | |
| t819179 | Library | 862 | 974 |
| | (CsPT4-CsPT7 Chimera; I86A M87V) | | |
| t819193 | Library | 863 | 975 |
| | (CsPT4-CsPT7 Chimera; I86T M87V F151G) | | |
| t819225 | Library | 864 | 976 |
| | (CsPT4-CsPT7 Chimera; C31F I86V M87V I147L) | | |
| t819336 | Library | 865 | 977 |
| | (CsPT4-CsPT1 ChimeraC31F I46C I86S D94E F145S | | |
| | F245W Q267F Q288R L311R) | | |
| t819343 | Library | 866 | 978 |
| | (CsPT4-CsPT1 Chimera; C31F I46C I86A D94E | | |
| | I147L F245R Q267F Q288R L311K) | | |
| t819372 | Library | 867 | 979 |
| | (CsPT4-CsPT1 Chimera; C31F M43V M87V D94E | | |
| | E113R F145S Q267F Q288R L311R) | | |
| t819375 | Library | 868 | 980 |
| | (CsPT4-CsPT7 Chimera; I86V D94E I147L F245R) | | |

TABLE 20
Prenyltransferase sequences associated with Example 8

| | Strain type | PT Protein | PT Nucleic Acid |
|---|---|---|---|
| Strain | PT type | SEQ ID NO: | SEQ ID NO: |
| t880043 | Library | 982 | 1083 |
| | (CsPT1-CsPT4 chimera; T30A L34F M43L S45L I86A | | |
| | M87I D94E E113R V140I I142L I147L A149L S182V | | |
| | G211S M212I V234L R241K F245R V250L L256V | | |
| | S258A I264N Q267F Q288R L311N) | | |
| t879667 | Library | 983 | 1084 |
| | (CsPT4-CsPT7 chimera; F82G D94E I147L A227R) | | |
| t879993 | Library | 984 | 1085 |
| | (CsPT4-CsPT7 chimera; V39T F82G D94E I147L) | | |
| t879001 | Library | 985 | 1086 |
| | (CsPT4-CsPT7 chimera; F82G D94E I140T I147L) | | |
| t879539 | Library | 986 | 1087 |
| | (CsPT4-CsPT7 chimera; F82G D94E I147L V246L) | | |
| t879989 | Library | 987 | 1088 |
| | (CsPT4-CsPT7 chimera; F82G D94E I147L A262G) | | |
| t879340 | Library | 988 | 1089 |
| | (CsPT4-CsPT7 chimera; F82G D94E I147L A227K | | |
| | T254N) | | |
| t880030 | Library | 989 | 1090 |
| | (CsPT4-CsPT7 chimera; F82G D94E I147L L204I) | | |
| t879474 | Library | 990 | 1091 |
| | (CsPT4-CsPT7 chimera; M75V F82G D94E I147L | | |
| | T254N) | | |
| t879791 | Library | 991 | 1092 |
| | (CsPT4-CsPT7 chimera; F82G D94E I147L T254L) | | |
| t879562 | Library | 992 | 1093 |
| | (CsPT4-CsPT7 chimera; F82G D94E I147L T254N) | | |
| t880029 | Library | 993 | 1094 |
| | (CsPT1-CsPT4 chimera; T30A M43L I86A D94E | | |
| | E113R I121S V129I F141S I147L F148L A149L T171I | | |
| | S173L S182V G211S M212L T213V R241K F245R | | |
| | V247C V250L Q267F I286F Q288R L311N) | | |
| t879750 | Library | 994 | 1095 |
| | (CsPT4-CsPT7 chimera; L62I F82G D94E I147L) | | |
| t879685 | Library | 995 | 1096 |
| | (CsPT4-CsPT7 chimera; L68F F82G D94E I147L) | | |
| t879512 | Library | 996 | 1097 |
| | (CsPT4-CsPT7 chimera; F82G D94E I147L T254N | | |
| | S257G) | | |
| t879297 | Library | 997 | 1098 |
| | (CsPT4-CsPT7 chimera; F82G D94E I147L 1172F) | | |
| t879827 | Library | 998 | 1099 |
| | (CsPT4-CsPT7 chimera; F82G D94E I147L T213V | | |
| | T254N) | | |
| t879670 | Library | 999 | 1100 |
| | (CsPT4-CsPT7 chimera; F82G D94E I147L I172L) | | |
| t879624 | Library | 1000 | 1101 |
| | (CsPT4-CsPT7 chimera; F82G D94E I147L V250I | | |
| | T254N) | | |
| t879758 | Library | 1001 | 1102 |
| | (CsPT4-CsPT7 chimera; F82G D94E I147L R241K) | | |
| t879503 | Library | 1002 | 1103 |
| | (CsPT4-CsPT7 chimera; F82G D94E I147L L275I) | | |
| t879068 | Library | 1003 | 1104 |
| | (CsPT4-CsPT7 chimera; F82G D94E I147L V250T | | |
| | T254N) | | |
| t879840 | Library | 1004 | 1105 |
| | (CsPT4-CsPT7 chimera; F82G D94E I147L L260I) | | |
| t879356 | Library | 1005 | 1106 |
| | (CsPT4-CsPT7 chimera; F82G D94E I147L P199A) | | |
| t879725 | Library | 1006 | 1107 |
| | (CsPT4-CsPT7 chimera; F82G D94E I147L M196L) | | |
| t879071 | Library | 1007 | 1108 |
| | (CsPT4-CsPT7 chimera; F82G D94E I117L I147) | | |
| t879768 | Library | 1008 | 1109 |
| | (CsPT4-CsPT7 chimera; F82G D94E I147L C284W) | | |
| t879836 | Library | 1009 | 1110 |
| | (CsPT4-CsPT7 chimera; F82G D94E I147L V250I) | | |
| t880054 | Library | 1010 | 1111 |
| | (CsPT4-CsPT7 chimera; F82G D94E I147L A227K) | | |
| t879626 | Library | 1011 | 1112 |
| | (CsPT4-CsPT7 chimera; F82G D94E I147L F151I) | | |
| t879983 | Library | 1012 | 3133 |
| | (CsPT4-CsPT7 chimera; F82G D94E I147L V209L) | | |
| t879726 | Library | 1013 | 1114 |
| | (CsPT4-CsPT7 chimera; F82G D94E I147L A152D) | | |
| t879529 | Library | 1014 | 1115 |
| | (CsPT4-CsPT7 chimera; F82G D94E I147L S257G) | | |
| t879304 | Library | 1015 | 1116 |
| | (CsPT4-CsPT7 chimera; F82G D94E I140L I147L) | | |
| t879708 | Library | 1016 | 1117 |
| | (CsPT4-CsPT7 chimera; F82G D94E I147L 1264F) | | |
| t879602 | Library | 1017 | 1118 |
| | (CsPT4-CsPT7 chimera; F82G D94E I147L V247S) | | |
| t879151 | Library | 1018 | 1119 |
| | (CsPT4-CsPT7 chimera; F82G D94E I147L V250A | | |
| | T254N) | | |
| t879382 | Library | 1019 | 1120 |
| | (CsPT4-CsPT7 chimera; F82G D94E II47L V234L) | | |
| t879774 | Library | 1020 | 1121 |
| | (CsPT4-CsPT7 chimera; F82G D94E I147L M196I) | | |
| t879650 | Library | 1021 | 1122 |
| | (CsPT4-CsPT7 chimera; F82G D94E I147L V231I) | | |
| t879418 | Library | 1022 | 1123 |
| | (CsPT4-CsPT7 chimera; F82G D94E I147L V234F) | | |
| t879399 | Library | 1023 | 1124 |
| | (CsPT4-CsPT7 chimera; F82G I86G D94E I147L) | | |
| t879949 | Library | 1024 | 1125 |
| | (CsPT4-CsPT7 chimera; F82G D94E I147L V246D) | | |
| t879660 | Library | 1025 | 1126 |
| | (CsPT4-CsPT7 chimera; F82G D94E I147L T254A) | | |
| t879522 | Library | 1026 | 1127 |
| | (CsPT4-CsPT7 chimera; M75V F82G D94E I147L) | | |
| t879193 | Library | 1027 | 1128 |
| | (CsPT4-CsPT7 chimera; F82G D94E I147L T213V) | | |
| t879977 | Library | 1028 | 1129 |
| | (CsPT1-CsPT4 chimera; L34F M43L I46A G49S I86A | | |
| | M87I D94E M110L E113R V140I F141S I147L A149L | | |
| | I172F G177T A179P A227K V234L F245R L256V | | |
| | S258A Q267F L276F Q288R L311N) | | |
| t879357 | Library | 1029 | 1130 |
| | (CsPT1-CsPT4 chimera; M43L F82G A85N I86G M87I | | |
| | D94E V106A E113R F141S I142L I147L A149L T171I | | |
| | A179N A227K Y229H V234L R241K F245R V250F | | |
| | V257L S258A Q267F Q288R L311K) | | |
| t879819 | Library | 1030 | 1131 |
| | (CsPT4-CsPT7 chimera; M75I F82G D94E I147L) | | |
| t879233 | Library | 1031 | 1132 |
| | (CsPT4-CsPT7 chimera; F82G D94E I147L V234L | | |
| | T254N) | | |
| t879240 | Library | 1032 | 1133 |
| | (CsPT4-CsPT7 chimera; F82G D94E I147L T254C) | | |
| t879205 | Library | 1033 | 1134 |
| | (CsPT4-CsPT7 chimera; F82G D94E I147L F190V) | | |
| t879397 | Library | 1034 | 1135 |
| | (CsPT4-CsPT7 chimera; F82G D94E I147L G177L | | |
| | T254N) | | |
| t879014 | Library | 1035 | 1136 |
| | (CsPT4-CsPT7 chimera; F82G D94E I147L M212I) | | |
| t879150 | Library | 1036 | 1137 |
| | (CsPT1-CsPT4 chimera; T30A V39T M43L F82G | | |
| | A85N M87I D94E E113R V129I V140F I142L I147L | | |
| | A149L TI71I G211T A227K V234L M243T F245R | | |
| | V246I V247C V250L Q267F Q288R L311K) | | |
| t879592 | Library | 1037 | 1138 |
| | (CsPT1-CsPT4 chimera; L34F Q35T M43L G49S I86A | | |
| | D94E D102Y E113R F139L I147L A149L S182V | | |
| | T213V A227R V234L T236V F245R V247G V250L | | |
| | L256V V257G Q267F F283S Q288R L311N) | | |
| t879184 | Library | 1038 | 1139 |
| | (CsPT1-CsPT4 chimera; T30A C31F M43V I46G | | |
| | G49A I86G M87V D94E L105I E113R F139L F141I | | |
| | F145L L169A T171I S173L S182V M210F T213A | | |
| | V234L F245W V250L S258A Q267F Q288R) | | |
| t879918 | Library | 1039 | 1140 |
| | (CsPT1-CsPT4 chimera; T30A C31F M43L S451 A73G | | |
| | V75I I86A M87L D94E M110L E113R I142L I147L | | |
| | G177V M212L K228A V234L N242T F245R V247C | | |
| | V250L V257L S258A Q267F Q288R) | | |
| t879813 | Library | 1040 | 1141 |
| | (CsPT1-CsPT4 chimera; T30A M43L G52A I86A | | |
| | D94E E113R I121S V129L F141S I147L T171I S182V | | |
| | G211A A227R V234F F245R V247C V250L V257G | | |
| | S258A Q267F S271K C284W Q288R L311N) | | |
| t879338 | Library | 1041 | 1142 |
| | (CsPT1-CsPT4 chimera; M43L F82G A85N I86G M87I | | |
| | D94E E113R F141I I147L T171I G177T S182V R197S | | |
| | M212L T213A A227K V234L M243S F245R V250L | | |
| | Q267F L276P F283S Q288R L311K) | | |
| t879042 | Library | 1042 | 1143 |
| | (CsPT1-CsPT4 chimera; M43L I46G F82G M87I D94E | | |
| | V106A M110L E113R V129L I147L A149I T171I | | |
| | A179N M212L Y229F V234L R241K F245R V246I | | |
| | V247C V250L S258A Q267F Q288R L311K) | | |
| t879155 | Library | 1043 | 1144 |
| | (CsPT1-CsPT4 chimera; T30A M43L G49S I86A M87I | | |
| | D94E DI02Y V106A E113R I121S V129L V140L | | |
| | I147L A179N R197S T213V A227K M243T F245R | | |
| | V246L V247C V250L Q267F Q288R L311N) | | |
| t879940 | Library | 1044 | 1145 |
| | (CsPT1-CsPT4 chimera; C31F M43L I86A M87I D94E | | |
| | V106A E113R I121S V129I F139L F141C I147L T171I | | |
| | S1731 A179P M212I A227K V234L R241K F245R | | |
| | V246I V247C Q267F C284F Q288R) | | |
| t879345 | Library | 1045 | 1146 |
| | (CsPT1-CsPT4 chimera; M43L S45L I86A D94E | | |
| | V106A E113R F139A I147L F148L A149L T171I | | |
| | T213V I223V V234L T236V A240V R241K M243T | | |
| | F245R V246L V247C V257G Q267F Q288R L311N) | | |
| t879857 | Library | 1046 | 1147 |
| | (CsPT1-CsPT4 chimera; T30A C31F M43L I86V | | |
| | M87V D94E F139L F141S F145L I147L F148L T171I | | |
| | G177I T181V R197S M207V G211S V234L F245R | | |
| | V246L V247C V250L L276G C284S L311N) | | |
| t879788 | Library | 1047 | 1148 |
| | (CsPT1-CsPT4 chimera; T30A C31F Q35T M43L S45L | | |
| | I46C V75I I86A D94E V106A E113R V129I F139I | | |
| | I147L A149L F151A I172F T213A V234L F245R | | |
| | V246I V250L S258A Q267F Q288R) | | |
| t879606 | Library | 1048 | 1149 |
| | (CsPT4-CsPT7 chimera; L34F M43L S45L F82G I86G | | |
| | D94E E113R F139L V140T I147L G177T A179N | | |
| | S182F M212I A227K M243T F245R V246L V247C | | |
| | V250L S258A Q267F L276F Q288R L311K) | | |
| t879579 | Library | 1049 | 1150 |
| | (CsPT1-CsPT4 chimera; C31F V39T M43V I46G F82A | | |
| | A85N M87V D94E E113R V129L F139L F145L | | |
| | A149L I172L G1771 M210F A227K F245W V246L | | |
| | V247C V257G S258A Q267F C284W Q288R) | | |
| t879488 | Library | 1050 | 11.51 |
| | (CsPT1-CsPT4 chimera; T30A C31F V39T M43V | | |
| | S45L I46G G49C F72A S79L M87V D94E E113R | | |
| | I121S I128L F145L S182V A227K Y229F V234L | | |
| | F245W V247C V250L Q267F F283S Q288R) | | |
| t879191 | Library | 1051 | 1152 |
| | (CsPT1-CsPT4 chimera; Q35A M43L V75I I86A M87I | | |
| | D94E L105I E113R L118I F139L V140L I142L I147L | | |
| | F151A T213V V234M M243I F245R L256V V257G | | |
| | S258A I264F Q267F Q288R L311N) | | |
| t879379 | Library | 1052 | 1153 |
| | (CsPT1-CsPT4 chimera; C31F L34F Q35S V39T M43L | | |
| | A47S F72V A85N I86V M87V D94E V129L F141S | | |
| | F145L I147L A152V T171I S182V M207V G211S | | |
| | T213V A227K F245R V250L L311N) | | |
| t879066 | Library | 1053 | 1154 |
| | (CsPT1-CsPT4 chimera; T30A C31F M43L I86A D94E | | |
| | M110I E113R L118I I121S V129I I147L F151A M212I | | |
| | T213G I223V V234L A240V R241K N242T F245R | | |
| | V246L S258A Q267F L281A Q288R) | | |
| t879874 | Library | 1054 | 1155 |
| | (CsPT1-CsPT4 chimera; T30A C31F V39T M43L | | |
| | S79C I86V M87V D94E V106A F141S F145L I147L | | |
| | A149L T171I S174V M210F M212L T213V F245R | | |
| | V246L V247C V250L V257G S258A L311N) | | |
| t879638 | Library | 1055 | 1156 |
| | (CsPT1-CsPT4 chimera; C31F M43V S45L G49I F72V | | |
| | F82A M87V D94E V106A E113R V129L F141V | | |
| | I142L F145L G211S A227K V234L R241K F245W | | |
| | L256V S258A Q267F A279I C284W Q288R) | | |
| t879848 | Library | 1056 | 1157 |
| | (CsPT1-CsPT4 chimera; C31F M43L S45F V75I I86V | | |
| | M87V D94E L105I V106A F139L V140T F141V | | |
| | F145L I147L A152I T171I G177L A179P M207V | | |
| | A227R V234L F245R V247G S258A L311N) | | |
| t879358 | Library | 1057 | 1158 |
| | (CsPT1-CsPT4 chimera; C31F M43V M87V D94E | | |
| | M110L E113R F139L V140T F141S F145L L169I | | |
| | I172L G177L A227K V234F F245W V246I V247C | | |
| | V250L L252I L256V V257G S258A Q267F Q288R) | | |
| t879809 | Library | 1058 | 1159 |
| | (CsPT4-CsPT7 chimera; T30A C31F M43L I86A M87I | | |
| | D94E E113R V129L I142L I147L A152F T171I G177L | | |
| | M210F G211S T213V A227K V234L R241K F245R | | |
| | V246I S258A Q267F C284W Q288R) | | |
| t879226 | Library | 1059 | 1160 |
| | (CsPT4-CsPT7 chimera; T30A C31F V40I M43L S45L | | |
| | I46G G49S I86A D94E V106A M110L E113R V129I | | |
| | I147L A149L T171I G211S M212L T213V A227R | | |
| | V234L F245R V250L Q267F Q288R) | | |
| t879141 | Library | 1060 | 1161 |
| | (CsPT4-CsPT7 chimera; T30A M43L S79L A85N I86A | | |
| | D94E E113R V129I F139L I147L A149L T171I S182V | | |
| | G211S Y229F A240V R241K M243T F245R V247C | | |
| | V250L Q267F C284I Q288R L311N) | | |
| t879439 | Library | 1061 | 1162 |
| | (CsPT4-CsPT7 chimera; C31F L34F M43L I86A D94E | | |
| | V106A E113R L118I F141C I147L A149L F151A | | |
| | S174V S182V M207V G211S A227K R241K F245R | | |
| | V246A L256V S258A Q267F C284V Q288R) | | |
| t879243 | Library | 1062 | 1163 |
| | (CsPT4-CsPT7 chimera; V40I M43L S45L I86A M87I | | |
| | D94E V106A E113R I147L A149L F151T NI67A | | |
| | T171I S173T T213V A227K R241K M243T F245R | | |
| | V246L V247C L256V Q267F Q288R L311N) | | |
| t879134 | Library | 1063 | 1164 |
| | (CsPT4-CsPT7 chimera; M43L F82G D94E E113R | | |
| | F139I F141S I142L I147L A149L A179N S182V | | |
| | M212I I223V V231I V234L A240V R241K F245R | | |
| | V247C S258A Q267F S271G F283S Q288R L311K) | | |
| t879557 | Library | 1064 | 1165 |
| | (CsPT4-CsPT7 chimera; C31F V39T M43V S45I S63N | | |
| | M87V D94E E113R F139L V140T F141V F145L | | |
| | A152I S182V R197S I204T A227K R241K F245W | | |
| | V246A L256V V257G S258A Q267F Q288R) | | |
| t879202 | Library | 1065 | 1166 |
| | (CsPT4-CsPT7 chimera; M43L S45L I86A M87I D94E | | |
| | E113R V129L I142L I147L A149L T171I I172V G177I | | |
| | A179N S182V 1204T M210F T213V V234L R241K | | |
| | F245R V250L Q267F Q288R L311N) | | |
| t879067 | Library | 1066 | 1167 |
| | (CsPT4-CsPT7 chimera; T30A C31F M43L I46G I86V | | |
| | M87V D94E M110I V129L F139L F145L I147L | | |
| | A149L T171I G177L A179N A227K Y229F R241K | | |
| | F245R V246L V247C V250L I264F L311N) | | |
| t879099 | Library | 1067 | 1168 |
| | (CsPT4-CsPT7 chimera; M43L S45L I46A P76A I86A | | |
| | M87I D94E V106A E113R F141C I147L F151T A152I | | |
| | M207V M212L A227R V234L T236A M243I F245R | | |
| | V247C S258A Q267F Q288R L311N) | | |
| t879286 | Library | 1068 | 1169 |
| | (CsPT4-CsPT7 chimera; L34F V39T M43L S45L F82G | | |
| | I86G M87I D94E V106A E113R V140F I147L T171I | | |
| | G211S T213A F216I V234L M243A F245R V246L | | |
| | V247C Q267F C284W Q288R L311K) | | |
| t878988 | Library | 1069 | 1170 |
| | (CsPT4-CsPT7 chimera; T30A M43L S63N I86A M87I | | |
| | D94E V106A E113R I147L F151T N167A A179N | | |
| | S182V M212I A217T V234L R241K M243T F245R | | |
| | V246F V247C Q267F C284W Q288R L311N) | | |
| t879148 | Library | 1070 | 1171 |
| | (CsPT4-CsPT7 chimera; M43L I46G I86A M87I D94E | | |
| | V106A M110I E113R L124V F139L I147L F151A | | |
| | T171I G211S A227K F245R V247C V250L L256V | | |
| | S258A Q267F C284V Q288R T289A L311N) | | |
| t879235 | Library | 1071 | 1172 |
| | (CsPT4-CsPT7 chimera; T30A M43L I46G G49S I86A | | |
| | M87I D94E M110L E113R F139L F141G I147L T171I | | |
| | I172V S173T T213A A227K V231I M243A F245R | | |
| | V247C V250L Q267F Q288R L311N) | | |
| t879422 | Library | 1072 | 1173 |
| | (CsPT4-CsPT7 chimera; C31F M43V A47S A73G | | |
| | S79C M87V D94E E113R V129I F141S F145L A149L | | |
| | A152L T171I A179N S182V M210F M212L V234L | | |
| | R241K F245W V247C S258A Q267F Q288R) | | |
| t879687 | Library | 1073 | 1174 |
| | (CsPT4-CsPT7 chimera; T30A C31F M43L G49S I86A | | |
| | D94E E113R L118I I121S V129L F139I I142L I147L | | |
| | G177I G211S A227K Y229F V234L R241K F245R | | |
| | V246I V247C V250L Q267F Q288R) | | |
| t879050 | Library | 1074 | 1175 |
| | (CsPT4-CsPT7 chimera; T30A M43L S45L S79L F82G | | |
| | D94E E113R V129I F141S I142L I147L G177I A179N | | |
| | S182V M212I V234F R241K F245R V247C V250L | | |
| | S258A Q267F C284W Q288R L311K) | | |
| t879913 | Library | 1075 | 1176 |
| | (CsPT4-CsPT7 chimera; T30A M43L F82G D94E | | |
| | E113R V129I V140T F141C I142L I147L A179N | | |
| | S182V I204T A227R V234M M243T F245R V246I | | |
| | V247C V250L S258A Q267F C284W Q288R L311K) | | |
| t879059 | Library | 1076 | 1177 |
| | (CsPT4-CsPT7 chimera; M43L F72Q F82G M87I | | |
| | D94E E113R I147L A149L N167A T171I S173L | | |
| | S182V F200L M212I V231I V234F M243T F245R | | |
| | V246I V247C V250L S258A Q267F Q288R L311K) | | |
| t879037 | Library | 1077 | 1178 |
| | (CsPT4-CsPT7 chimera; T30A V40I M43L F82G M87I | | |
| | D94E E113R I147L T171I S173L G177T V209L | | |
| | M212L Y229F V234L R241K F245R V246I V247C | | |
| | V250I S258A Q267F C284W Q288R L311K) | | |
| t879885 | Library | 1078 | 1179 |
| | (CsPT4-CsPT7 chimera; T30A M43L S45L I86A D94E | | |
| | E113R V129L F141A I142L I147L A149L F151A | | |
| | N167A T171I A179N M210F T213V V231I R241K | | |
| | F245R V247C V250L Q267F Q288R L311N) | | |
| t879332 | Library | 1079 | 1180 |
| | (CsPT4-CsPT7 chimera; T30A C31F V39T V40I M43L | | |
| | A85N I86V M87V D94E V129L V140T F145L I147L | | |
| | S182V M207V G211S T213V A227R V234L F245R | | |
| | V246L V247C V257G S258A L311N) | | |
| t879116 | Library | 1080 | 1181 |
| | (CsPT4-CsPT7 chimera; T30A M43L S45L G49S S79L | | |
| | I86A D94E E113R I142L I147L A149L N167A T171I | | |
| | G211S T213V I220L V234L F245R V246I V247C | | |
| | V250L L256V Q267F Q288R L311N) | | |
| t879830 | Library | 1081 | 1182 |
| | (CsPT4-CsPT7 chimera; C31F M43V I46G M87V | | |
| | D94E V106A E113R F139L F141V I142L F145L | | |
| | I172L S173L S182V M212L T213V I220L V234L | | |
| | N242T F245W V247C V257G Q267F S271L Q288R) | | |

### EQUIVALENTS

Those skilled in the art will recognize, or be able to ascertain using no more than routine experimentation, many equivalents to the specific embodiments of the invention described here. Such equivalents are intended to be encompassed by the following claims.

All references, including patent documents, are incorporated by reference in their entirety.

1. A chimeric prenyltransferase (PT), wherein the chimeric PT comprises one or more portions of CsPT1, CsPT4, CsPT6, and/or CsPT7 and wherein the chimeric PT is capable of producing a CBG-type cannabinoid from a resorcylic acid.

2. The chimeric PT of claim 1, wherein the CBG-type cannabinoid and the resorcylic acid are: cannabigerolic acid (CBGA) and olivetolic acid; or cannabigerovarinic acid (CBGVA) and divaric acid (DA).

3.-6. (canceled)

7. The chimeric PT of claim 1, wherein the chimeric PT comprises multiple transmembrane helices, and wherein at least one transmembrane helix of the multiple transmembrane helices comprises both a portion of CsPT4 and a portion of CsPT1, CsPT6 or CsPT7.

8.-9. (canceled)

10. The chimeric PT of claim 1, wherein the chimeric PT comprises one or more of the following motifs:
(i)
(SEQ ID NO: 11)
MTVMGMT;
(ii)
(SEQ ID NO: 12)
[EV][LMW][RS]P[SAP]F[ST]F[IL][IL]AF;
(iii)
(SEQ ID NO: 13)
QFFEFIW;
(iv)
(SEQ ID NO: 14)
HNTNL;
(v)
(SEQ ID NO: 15)
TCWKL;
(vi)
(SEQ ID NO: 16)
M[IL]LSHAILAFC;
(vii)
(SEQ ID NO: 17)
HVG[LV][AN]FT[SCF]Y[YS]A[ST][RT][AS]A[LF];
(viii)
(SEQ ID NO: 18)
GLIVT;
(ix)
(SEQ ID NO: 19)
L[YH]Y AEY[LF]V;
(x)
(SEQ ID NO: 20)
KAFFAL;
(xi)
(SEQ ID NO: 21)
KLGARNMT;
(xii)
(SEQ ID NO: 22)
QAF[NK]SN;
(xiii)
(SEQ ID NO: 23)
LIFQT;
(xiv)
(SEQ ID NO: 24)
SIIVALT;
(xv)
(SEQ ID NO: 25)
MSIETAW;
(xvi)
(SEQ ID NO: 26)
VVSGV;
(xvii)
(SEQ ID NO: 27)
RPYVV;
(xviii)
(SEQ ID NO: 28)
KPDLP;
(xix)
(SEQ ID NO: 29)
RWKQY;
(xx)
(SEQ ID NO: 30)
FLITI;
(xxi)
(SEQ ID NO: 31)
DIEGD;
and
(xxii)
(SEQ ID NO: 32)
KYGVST.

11.-15. (canceled)

16. The chimeric PT of claim 1, wherein the chimeric PT comprises: X1-X2-X3-X4-X5-X6-X7-X8-X9-X10, wherein:
(i) The sequence of X1 comprises any of SEQ ID NOs: 33-39 or a sequence that comprises no more than 2 amino acid substitutions, insertions, additions or deletions relative to any one of SEQ ID NOs: 33-39;
(ii) The sequence of X2 comprises any of SEQ ID NOs: 40-46 or a sequence that comprises no more than 2 amino acid substitutions, insertions, additions or deletions relative to any one of SEQ ID NOs: 40-46;
(iii) The sequence of X3 comprises any of SEQ ID NOs: 47-53 or a sequence that comprises no more than 2 amino acid substitutions, insertions, additions or deletions relative to any one of SEQ ID NOs: 47-53;
(iv) The sequence of X4 comprises any of SEQ ID NOs: 54-60 or a sequence that comprises no more than 2 amino acid substitutions, insertions, additions or deletions relative to any one of SEQ ID NOs: 54-60;
(v) The sequence of X5 comprises any of SEQ ID NOs: 61-67 or a sequence that comprises no more than 2 amino acid substitutions, insertions, additions or deletions relative to any one of SEQ ID NOs: 61-67;
(vi) The sequence of X6 comprises any of SEQ ID NOs: 68-74 or a sequence that comprises no more than 2 amino acid substitutions, insertions, additions or deletions relative to any one of SEQ ID NOs: 68-74;
(vii) The sequence of X7 comprises any of SEQ ID NOs: 75-81 or a sequence that comprises no more than 2 amino acid substitutions, insertions, additions or deletions relative to any one of SEQ ID NOs: 75-81;
(viii) The sequence of X8 comprises any of SEQ ID NOs: 82-88 or a sequence that comprises no more than 2 amino acid substitutions, insertions, additions or deletions relative to any one of SEQ ID NOs: 82-88;
(ix) The sequence of X9 comprises any of SEQ ID NOs: 89-95 or a sequence that comprises no more than 2 amino acid substitutions, insertions, additions or deletions relative to any one of SEQ ID NOs: 89-95; and/or
(x) The sequence of X10 comprises any of SEQ ID NOs: 96-102 or a sequence that comprises no more than 2 amino acid substitutions, insertions, additions or deletions relative to any one of SEQ ID NOs: 96-102.

17. The chimeric PT of claim 1, wherein the chimeric PT comprises a sequence that is at least 85% identical to any one of SEQ ID NOs: 114, 116, 759, 760, 808, 844, 857, 982 and 998.

18. The chimeric PT of claim 17, wherein the chimeric PT comprises any one of SEQ ID NOs: 114, 116, 759, 760, 808, 844, 857, 982 and 998.

19. The chimeric PT of claim 17, wherein the chimeric PT comprises;
(i) an amino acid substitution relative to SEQ ID NO: 114 at one or more of the following positions within SEQ ID NO: 114: T30, C31, L34, M43, S45, F82, I86, M87, D94, E113, V140, I142, F145, I147, A149, I172, S182, G211, M212, V234, R241, F245, V247, V250, L256, S258, I264, Q267, Q288, and L311; or
(ii) an amino acid substitution relative to SEQ ID NO: 116 at one or more of the following positions within SEQ ID NO: 116: F82, D94, I147, T213, F245, and T254.

20. The chimeric PT of claim 19, wherein the chimeric PT comprises;
(i) one or more of the following amino acid substitutions relative to SEQ ID NO: 114: T30A, C31F, L34F, M43L, S45L, F82G, I86A, M87I, D94E, E113R, V140I, I142L, F145T, I147L, A149L, I172V, S182V, G211S, M212I, V234L, R241K, F245R, V247C, V250L, L256V, S258A, I264N, Q267F, Q288R, L311N, and L311K; or
(ii) one or more of the following amino acid substitutions relative to SEQ ID NO: 116: F82G, D94E, I147L, T213V, F245R, and T254N.

21.-24. (canceled)

25. A fusion protein comprising the chimeric prenyltransferase of claim 1, wherein the fusion protein further comprises a farnesyl pyrophosphate synthase.

26.-35. (canceled)

36. A host cell comprising the chimeric PT of claim 1.

37.-40. (canceled)

41. The host cell of claim 36, wherein the host cell is a yeast cell.

42. The host cell of claim 41, wherein the yeast cell is a Saccharomyces cell, a Yarrowia cell, a Komagataella cell, or a Pichia cell.

43.-47. (canceled)

48. A method comprising culturing the host cell of claim 36.

49.-71. (canceled)

72. The chimeric PT of claim 10, wherein the chimeric PT comprises all of the following motifs:
(i)
(SEQ ID NO: 11)
MTVMGMT;
(ii)
(SEQ ID NO: 13)
QFFEFIW;
(iii)
(SEQ ID NO: 15)
TCWKL;
(iv)
(SEQ ID NO: 17)
HVG[LV][AN]FT[SCF]Y[YS]A[ST][RT][AS]A[LF];
(v)
(SEQ ID NO: 18)
GLIVT;
(vi)
(SEQ ID NO: 19)
L[YH]YAEY[LF]V;
(vii)
(SEQ ID NO: 20)
KAFFAL;
(viii)
(SEQ ID NO: 24)
SIIVALT;
(ix)
(SEQ ID NO: 25)
MSIETAW;
(x)
(SEQ ID NO: 27)
RPYVV;
(xi)
(SEQ ID NO: 28)
KPDLP;
and
(xii)
(SEQ ID NO: 30)
FLITI.

73. The chimeric PT of claim 10, wherein the chimeric PT comprises all of the following motifs:
(i)
(SEQ ID NO: 11)
MTVMGMT;
(ii)
(SEQ ID NO: 12)
[EV][LMW][RS]P[SAP]F[ST]F[IL][IL]AF;
(iii)
(SEQ ID NO: 13)
QFFEFIW;
(iv)
(SEQ ID NO: 14)
HNTNL;
(v)
(SEQ ID NO: 16)
M[IL]LSHAILAFC;
(vi)
(SEQ ID NO: 17)
HVG[LV][AN]FT[SCF]Y[YS]A[ST][RT][AS]A[LF];
(vii)
(SEQ ID NO: 19)
L[YH]YAEY[LF]V;
(viii)
(SEQ ID NO: 21)
KLGARNMT;
(ix)
(SEQ ID NO: 24)
SIIVALT;
(x)
(SEQ ID NO: 26)
VVSGV;
(xi)
(SEQ ID NO: 27)
RPYVV;
(xii)
(SEQ ID NO: 28)
KPDLP;
(xiii)
(SEQ ID NO: 29)
RWKQY;
(xiv)
(SEQ ID NO: 30)
FLITI;
(xv)
(SEQ ID NO: 31)
DIEGD;
and
(xvi)
(SEQ ID NO: 32)
KYGVST.

74. A host cell that comprises a heterologous polynucleotide encoding a chimeric prenyltransferase (PT), wherein the chimeric PT comprises one or more portions of at least two different PTs, wherein the chimeric PT is capable of producing a CBG-type cannabinoid from a resorcylic acid, and wherein the chimeric PT comprises a sequence that is at least 85% identical to the sequence of any one of SEQ ID NOs: 114, 116, 759, 760, 808, 844, 857, 982 and 998.

75. The host cell of claim 74, wherein the chimeric PT comprises:
(i) an amino acid substitution relative to SEQ ID NO: 114 at one or more of the following positions within SEQ ID NO: 114: T30, C31, L34, M43, S45, F82, I86, M87, D94, E113, V140, I142, F145, I147, A149, I172, S182, G211, M212, V234, R241, F245, V247, V250, L256, S258, I264, Q267, Q288, and L311; or
(ii) an amino acid substitution relative to SEQ ID NO: 116 at one or more of the following positions within SEQ ID NO: 116: F82, D94, I147, T213, F245, and T254.

76. A host cell that comprises a heterologous polynucleotide encoding a chimeric prenyltransferase (PT), wherein the chimeric PT comprises one or more portions of at least two different PTs, wherein the chimeric PT is capable of producing a CBG-type cannabinoid from a resorcylic acid, and wherein the chimeric PT comprises a sequence that is at least 85% identical to the sequence of any one of SEQ ID NOs: 111, 114, 115, 118, 119 and 120.
